(19) United States
(12) Patent Application Publication
(10) Pub. No.: US 2024/0374714 A1
(43) Pub. Date: Nov. 14, 2024

(54) COMPOSITIONS AND METHODS FOR MULTIVALENT IMMUNE RESPONSES

(71) Applicant: HDT Bio Corp., Seattle, WA (US)

(72) Inventors: Malcolm S. Duthie, Sammamish, WA (US); Jesse Erasmus, Port Orchard, WA (US); Lars Peter Aksel BERGLUND, Seattle, WA (US)

(21) Appl. No.: 18/763,524

(22) Filed: Jul. 3, 2024

Related U.S. Application Data
(63) Continuation of application No. PCT/US2023/060225, filed on Jan. 6, 2023.
(60) Provisional application No. 63/297,304, filed on Jan. 7, 2022.

(51) Int. Cl. A61K 39/215; A61K 39/00; A61K 39/12; A61K 47/26; A61K 47/64; A61K 47/69; A61P 37/04

(52) U.S. Cl. CPC A61K 39/215; A61K 39/0011; A61K 39/12; A61K 47/26; A61K 47/64; A61K 47/6925; A61P 37/04; A61K 2039/53; A61K 2039/55505; A61K 2039/70

(57) ABSTRACT

The disclosure provides nanoparticle and nucleic acid compositions and methods of making and using the same to deliver multiple bioactive agents such as a nucleic acid encoding a viral antigen or a tumor antigen to a subject. Various nanoparticle carriers are described. Various nucleic acids encoding viral and tumor antigens are described. In some instances, the nanoparticle component may include a hydrophobic core having an inorganic particle, and optionally a membrane having a cationic lipid.

### CROSS REFERENCE

This application is a continuation of International Application No. PCT/US2023/060225, filed Jan. 6, 2023, which claims the benefit of priority to U.S. Provisional Patent Application No. 63/297,304, filed Jan. 7, 2022, the contents of each of which are incorporated herein by reference in their entirety.

### SEQUENCE LISTING

The instant application contains a Sequence Listing which has been submitted electronically in ST.26 format and is hereby incorporated by reference in its entirety. Said copy, created on Feb. 9, 2023, is named 199827-720601_SL.xml and is 364 kilobytes in size.

### BACKGROUND

Current vaccine therapeutics are formulated with single antigens to protect against infections by one species of a pathogenic microorganism. Such vaccine formulations typically do not permit for the large-scale manufacture of vaccines that protect against variant infectious strains or different pathogenic species in a single dose. Therefore, there is a need for enhanced nucleic acid-encoded protein therapeutics and vaccines that yield a therapeutically effective level of protein expression for multiple microbial antigens to stimulate an immune response in subjects to fend more than one infectious strain and/or species.

### SUMMARY

Provided herein are compositions, wherein the compositions comprise: nanoparticles, wherein the nanoparticles comprise a surface; and nucleic acids, wherein the nucleic acids comprise sequences encoding an RNA polymerase and a viral protein antigen, wherein at least two of the nucleic acids encode for non-identical viral protein antigens, and wherein the nucleic acids complex to the surface to form nucleic acid-nanoparticle complexes. In some embodiments, each of the nucleic acids comprises a sequence encoding for a single viral protein antigen. In some embodiments, the nucleic acid comprises an RNA or a DNA. In some embodiments, the viral protein antigen is derived from a coronavirus, a picornavirus, an enterovirus, an influenza A virus, an influenza B virus, a respiratory syncytial virus (RSV), a paramyxovirus, a bunyavirus, or a Zika virus. In some embodiments, the nucleic acids comprise at least two sequences each encoding viral protein antigens of the same viral species. In some embodiments, the nucleic acids comprise at least two sequences each encoding viral protein antigens from different viral species. In some embodiments, the nucleic acids comprise sequences encoding two or more coronavirus spike (S) proteins or functional variants thereof. In some embodiments, the nucleic acids comprise sequences encoding antigens derived from two or more picornavirus. In some embodiments, the nucleic acids comprise sequences encoding antigens derived from two or more enterovirus species. In some embodiments, the nucleic acids comprise sequences encoding one or more of a coronavirus spike protein or functional variant thereof, an influenza virus hemagglutinin protein or functional variant thereof, an RSV glycoprotein (G) or functional variant thereof, or an RSV fusion (F) protein or functional variant thereof. In some embodiments, wherein the nucleic acids comprise sequences encoding two or more of a coronavirus spike protein or functional variant thereof, an influenza virus hemagglutinin protein or functional variant thereof, an RSV glycoprotein (G) or functional variant thereof, or an RSV fusion (F) protein or functional variant thereof in some embodiments, the nucleic acids comprise sequences encoding three or more of a coronavirus spike protein or functional variant thereof, an influenza virus hemagglutinin protein or functional variant thereof, an RSV glycoprotein (G) or functional variant thereof, or an RSV fusion (F) protein or functional variant thereof. In some embodiments, the nucleic acids comprise sequences encoding a coronavirus spike protein or functional variant thereof, an influenza virus hemagglutinin protein or functional variant thereof, an RSV glycoprotein (G) or functional variant thereof, and an RSV-F protein or functional variant thereof. In some embodiments, the coronavirus spike protein is a SARS-CoV-2 spike protein. In some embodiments, the SARS-CoV-2 spike protein is derived from the alpha variant of SARS-CoV-2. In some embodiments, the SARS-CoV-2 spike protein is derived from the beta variant of SARS-CoV-2. In some embodiments, the SARS-CoV-2 spike protein is derived from the delta variant of SARS-CoV-2. In some embodiments, the SARS-CoV-2 spike protein is derived from the mu variant of SARS-CoV-2. In some embodiments, the SARS-CoV-2 spike protein is derived from a variant of SARS-CoV-2 comprising an amino acid substitution of D614G or D627G. In some embodiments, the one or more of the nucleic acids comprise a sequence set forth in any one of SEQ ID NOS: 1-7, 67-73. In some embodiments, wherein the influenza virus hemagglutinin protein is hemagglutinin H2, hemagglutinin H3, hemagglutinin H5, hemagglutinin H6, hemagglutinin H7, hemagglutinin H8, or hemagglutinin H9. In some embodiments, the influenza virus hemagglutinin protein has a nucleic acid sequence set forth in SEQ ID NO: 13 or SEQ ID NO: 76. In some embodiments, the RSV-G comprises a nucleic acid sequence set forth in SEQ ID NO: 9 or SEQ ID NO: 75. In some embodiments, the RSV-F comprises a nucleic acid sequence set forth in SEQ ID NO: 8 or SEQ ID NO: 74. In some embodiments, the RNA polymerase is a self-replicating RNA polymerase. In some embodiments, the self-replicating RNA polymerase is under that control of an internal ribosome entry side (IRES). In some embodiments, the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase. In some embodiments, the RNA polymerase is Venezuelan equine encephalitis virus (VEEV) RNA polymerase. In some embodiments, the nucleic acid encoding for the RNA polymerase comprises the nucleic acid sequence of SEQ ID NO: 62. In some embodiments, the composition comprises at least one of the nucleic acids at a different concentration that the other nucleic acids. In some embodiments, the composition comprises a first nucleic acid and a second nucleic acid, wherein the first nucleic acid is present at a concentration that is different from the second nucleic acid concentration (e.g., the first nucleic acid is present at a concentration of 0.5 μg and the second nucleic acid is present at a concentration of 2 μg). In some embodiments, the nanoparticles comprise a cationic lipid. In some embodiments, the cationic lipid comprises 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP), dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, N1,N3,N5-tris(3-(didodecylamino)propyl)benzene-1,3,5-tricarboxamide, or a combination thereof. In some embodiments, the nanoparticles comprise a hydrophobic core. In some embodiments, the hydrophobic core comprises a lipid. In some embodiments, the hydrophobic core comprises an oil. In some embodiments, the hydrophobic core comprises a lipid in liquid phase at 25 degrees Celsius. In some embodiments, the lipid comprises α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, soy lecithin, soybean oil, sunflower oil, a triglyceride, vitamin E, or a combination thereof. In some embodiments, the triglyceride comprises capric triglyceride, caprylic triglyceride, a caprylic and capric triglyceride, a triglyceride ester, myristic acid triglycerin, or a combination thereof. In some embodiments, the hydrophobic core comprises a lipid in solid phase at 25 degrees Celsius. In some embodiments, the hydrophobic core comprises glyceryl trimyristate-dynasan. In some embodiments, the nanoparticles comprise an inorganic particle. In some embodiments, the inorganic particle is within the hydrophobic core of the nanoparticle. In some embodiments, the inorganic particle comprises a metal. In some embodiments, the metal comprises a metal salt, a metal oxide, a metal hydroxide, a metal phosphate, or a combination thereof. In some embodiments, the metal oxide comprises aluminum oxide, aluminum oxyhydroxide, iron oxide, titanium dioxide, silicon dioxide, or a combination thereof. In some embodiments, the nanoparticles comprise a cationic lipid, an oil, and an inorganic particle. In some embodiments, the nanoparticles further comprise a surfactant. In some embodiments, the surfactant is a hydrophobic surfactant. In some embodiments, the hydrophobic surfactant comprises sorbitan monolaurate, sorbitan monopalmitate, sorbitan monostearate, sorbitan monooleate, sorbitan trioleate, or a combination thereof. In some embodiments, the surfactant is a hydrophilic surfactant. In some embodiments, the hydrophilic surfactant comprises a polysorbate. In some embodiments, the nanoparticles comprise a cationic lipid, an oil, an inorganic particle, and a surfactant. In some embodiments, the nanoparticles do not comprise trimyristin. In some embodiments, the composition is lyophilized. In some embodiments, the composition is a suspension. In some embodiments, the suspension is a homogeneous suspension.

Provided herein are compositions, wherein the compositions comprise: nanoparticles, wherein the nanoparticles comprise a surface; and nucleic acids, wherein the nucleic acids comprise sequences encoding an RNA polymerase and a tumor protein antigen, wherein at least two of the nucleic acids encode for non-identical tumor protein antigens, and wherein the nucleic acids complex to the surface to form nucleic acid-nanoparticle complexes. In some embodiments, each of the nucleic acids comprises a sequence encoding for a single tumor protein antigen. In some embodiments, the nucleic acid comprises an RNA or a DNA. In some embodiments, the nucleic acids encode for a tumor protein antigen comprising epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Esol), Malignant melanoma (MAGE), MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MART-1/Melan-A, Tyrosinase, tyrosinase related protein 1 (TRYP-1), glycoprotein 100 (GP100), breast cancer WT1, Herceptin, lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), or programmed cell death protein (PD-1). In some embodiments, the RNA polymerase is a self-replicating RNA polymerase. In some embodiments, the self-replicating RNA polymerase is under that control of an internal ribosome entry side (IRES). In some embodiments, the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase. In some embodiments, the RNA polymerase is Venezuelan equine encephalitis virus (VEEV) RNA polymerase. In some embodiments, the nucleic acid encoding for the RNA polymerase comprises the nucleic acid sequence of SEQ ID NO: 62. In some embodiments, the composition comprises at least one of the nucleic acids at a different concentration that the other nucleic acids. In some embodiments, the composition comprises a first nucleic acid and a second nucleic acid, wherein the first nucleic acid is present at a concentration that is different from the second nucleic acid concentration. In some embodiments, the nanoparticles comprise a cationic lipid. In some embodiments, the cationic lipid comprises 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9″′,9″″,9′″″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, N1,N3,N5-tris(3-(didodecylamino)propyl)benzene-1,3,5-tricarboxamide, or a combination thereof. In some embodiments, the nanoparticles comprise a hydrophobic core. In some embodiments, the hydrophobic core comprises a lipid. In some embodiments, the hydrophobic core comprises an oil. In some embodiments, the hydrophobic core comprises a lipid in liquid phase at 25 degrees Celsius. In some embodiments, the lipid comprises α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, vitamin E, or a combination thereof. In some embodiments, the triglyceride comprises capric triglyceride, caprylic triglyceride, a caprylic and capric triglyceride, a triglyceride ester, myristic acid triglycerin, or a combination thereof. In some embodiments, the hydrophobic core comprises a lipid in solid phase at 25 degrees Celsius. In some embodiments, the hydrophobic core comprises glyceryl trimyristate-dynasan. In some embodiments, the nanoparticles comprise an inorganic particle. In some embodiments, the inorganic particle is within the hydrophobic core of the nanoparticle. In some embodiments, the inorganic particle comprises a metal. In some embodiments, the metal comprises a metal salt, a metal oxide, a metal hydroxide, a metal phosphate, or a combination thereof. In some embodiments, the metal oxide comprises aluminum oxide, aluminum oxyhydroxide, iron oxide, titanium dioxide, silicon dioxide, or a combination thereof. In some embodiments, the nanoparticles comprise a cationic lipid, an oil, and an inorganic particle. In some embodiments, the nanoparticles further comprise a surfactant. In some embodiments, the surfactant is a hydrophobic surfactant. In some embodiments, the hydrophobic surfactant comprises sorbitan monolaurate, sorbitan monopalmitate, sorbitan monostearate, sorbitan monooleate, sorbitan trioleate, or a combination thereof. In some embodiments, the surfactant is a hydrophilic surfactant. In some embodiments, the hydrophilic surfactant comprises a polysorbate. In some embodiments, the nanoparticles comprise a cationic lipid, an oil, an inorganic particle, and a surfactant. In some embodiments, the nanoparticles do not comprise trimyristin. In some embodiments, the composition is lyophilized. In some embodiments, the composition is a suspension. In some embodiments, the suspension is a homogeneous suspension.

Provided herein are pharmaceutical compositions, wherein the pharmaceutical compositions comprise any one of the compositions provided herein and a pharmaceutically acceptable excipient.

Provided herein are methods, wherein the methods comprise: administering to a subject an effective amount of a composition provided herein or a pharmaceutical composition provided herein, wherein the administering produces an immune response in the subject to one or more viral protein antigens. In some embodiments, the viral protein antigen is a coronavirus spike protein, an influenza virus hemagglutinin protein, an RSV glycoprotein (G), an RSV fusion (F) protein, HPV protein, or a variant thereof. In some embodiments, the administering is local administration or systemic administration. In some embodiments, the administering is via intramuscular injection, intranasal administration, oral administration, subcutaneous administration, intratumoral administration, or intravenous injection. In some embodiments, the subject is at risk of developing one or more infectious diseases. In some embodiments, the infectious disease is enterovirus infection, Severe acute respiratory syndrome (SARS), COVID 19, the flu, or Zika fever. In some embodiments, the infection disease is enterovirus infection. In some embodiments, the disease comprises one or more of acute flaccid myelitis and hand, food, and mouth disease. In some embodiments, when administered in an effective amount to the subject, the administering elicits antibody titers to at least one viral protein antigen equal to or greater than the composition administered to a subject without the plurality of nucleic acids comprising a sequence encoding for an RNA polymerase.

Provided herein are methods, wherein the methods comprise: administering to a subject an effective amount of a composition provided herein or a pharmaceutical composition provided herein, wherein the administering produces an immune response in the subject to one or more tumor protein antigens. In some embodiments, the tumor protein antigen comprises epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Esol), Malignant melanoma (MAGE), MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MART-1/Melan-A, Tyrosinase, tyrosinase related protein 1 (TRYP-1), glycoprotein 100 (GP100), breast cancer WT1, Herceptin, lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), or programmed cell death protein (PD-1). In some embodiments, the administering is local administration or systemic administration. In some embodiments, the administering is via intramuscular injection, intranasal administration, oral administration, subcutaneous administration, intratumoral administration, or intravenous injection. In some embodiments, the subject has a solid tumor or a blood cancer. In some embodiments, the solid tumor is a carcinoma, a melanoma, or a sarcoma. In some embodiments, wherein the blood cancer is lymphoma or leukemia. In some embodiments, the subject has lung cancer or melanoma. In some embodiments, the lung cancer is adenocarcinoma, squamous cell carcinoma, small cell cancer or non-small cell cancer.

Provided herein are methods of preventing or treating infection, wherein the methods comprise: administering to a subject having an infection, a composition provided herein or a pharmaceutical composition provided herein.

Provided herein are methods of treating cancer, wherein the methods comprise: administering to a subject having cancer, a composition provided herein or a pharmaceutical composition provided herein.

Provided herein are compositions, wherein the composition comprises nanoparticles, wherein the nanoparticles comprise a surface; and nucleic acids, wherein the nucleic acids comprise sequences encoding for an RNA polymerase and antigens, and wherein at least two of the nucleic acids encode for non-identical antigens, and wherein the nucleic acids complex to the surface to form nucleic acid-nanoparticle complexes. In some embodiments, the antigens are viral protein antigens or tumor protein antigens.

Provided herein are compositions, wherein the compositions comprise: nanoparticles, wherein the nanoparticles comprise a surface; and nucleic acids, wherein the nucleic acids comprise sequences encoding an RNA polymerase and a viral protein antigen derived from a non-enveloped virus, wherein at least two of the nucleic acids encode for non-identical viral protein antigens, wherein the nucleic acids complex to the surface to form nucleic acid-nanoparticle complexes, and wherein at least one of the nucleic acids is derived from a non-enveloped virus. In some embodiments, each of the nucleic acids comprises a sequence encoding for a single viral protein antigen. In some embodiments, the nucleic acid comprises an RNA or a DNA. In some embodiments, the non-enveloped virus is from a family comprising adenoviridae, iridoviridae, papiiomaviridae, poivonaviridae, anellovirus, circoviridae, parvoviridae, birnaviridae, picobirnaviridae, reoviridae, picornaviridae, astroviridae, caliciviridae, hepevirus, and nodaviridae. In some embodiments, the non-enveloped virus is an enterovirus. In some embodiments, the nucleic acids comprise at least two sequences each encoding viral protein antigens of the same viral species. In some embodiments, the nucleic acids comprise at least two sequences each encoding viral protein antigens from different viral species. In some embodiments, the RNA polymerase is a self-replicating RNA polymerase. In some embodiments, the self-replicating RNA polymerase is under that control of an internal ribosome entry side (IRES). In some embodiments, the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase. In some embodiments, the RNA polymerase is Venezuelan equine encephalitis virus (VEEV) RNA polymerase. In some embodiments, the nucleic acid encoding for the RNA polymerase comprises the nucleic acid sequence of SEQ ID NO: 62. In some embodiments, the composition comprises a first nucleic acid and a second nucleic acid, wherein the first nucleic acid is present at a concentration that is different from the second nucleic acid concentration. In some embodiments, the nanoparticles comprise a cationic lipid. In some embodiments, the cationic lipid comprises 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9″′″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, N1,N3,N5-tris(3-(didodecylamino)propyl)benzene-1,3,5-tricarboxamide, or a combination thereof. In some embodiments, the nanoparticles comprise a hydrophobic core. In some embodiments, the hydrophobic core comprises a lipid. In some embodiments, the hydrophobic core comprises an oil. In some embodiments, the hydrophobic core comprises a lipid in liquid phase at 25 degrees Celsius. In some embodiments, the lipid comprises α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, vitamin E, or a combination thereof. In some embodiments, the triglyceride comprises capric triglyceride, caprylic triglyceride, a caprylic and capric triglyceride, a triglyceride ester, myristic acid triglycerin, or a combination thereof. In some embodiments, the hydrophobic core comprises a lipid in solid phase at 25 degrees Celsius. In some embodiments, the hydrophobic core comprises glyceryl trimyristate-dynasan. In some embodiments, the nanoparticles comprise an inorganic particle. In some embodiments, the inorganic particle is within the hydrophobic core of the nanoparticle. In some embodiments, the inorganic particle comprises a metal. In some embodiments, the metal comprises a metal salt, a metal oxide, a metal hydroxide, a metal phosphate, or a combination thereof. In some embodiments, the metal oxide comprises aluminum oxide, aluminum oxyhydroxide, iron oxide, titanium dioxide, silicon dioxide, or a combination thereof. In some embodiments, the nanoparticles comprise a cationic lipid, an oil, and an inorganic particle. In some embodiments, the nanoparticles further comprise a surfactant. In some embodiments, the surfactant is a hydrophobic surfactant. In some embodiments, the hydrophobic surfactant comprises sorbitan monolaurate, sorbitan monopalmitate, sorbitan monostearate, sorbitan monooleate, sorbitan trioleate, or a combination thereof. In some embodiments, the surfactant is a hydrophilic surfactant. In some embodiments, the hydrophilic surfactant comprises a polysorbate. In some embodiments, the nanoparticles comprise a cationic lipid, an oil, an inorganic particle, and a surfactant. In some embodiments, the nanoparticles do not comprise trimyristin. In some embodiments, the composition is lyophilized. In some embodiments, the composition is a suspension. In some embodiments, the suspension is a homogeneous suspension. Provided herein are pharmaceutical compositions, wherein the pharmaceutical compositions comprise any one of the compositions provided herein and a pharmaceutically acceptable excipient. Also, provided herein are methods, wherein the methods comprise administering to a subject an effective amount of any one of the compositions described herein or the pharmaceutical composition described herein, wherein the administering produces an immune response in the subject to one or more viral protein antigens. In some embodiments, the administering is local administration or systemic administration. In some embodiments, the administering is via intramuscular injection, intranasal administration, oral administration, subcutaneous administration, intratumoral administration, or intravenous injection. In some embodiments, the subject is at risk of developing one or more infectious diseases. In some embodiments, the infection disease is enterovirus infection. In some embodiments, the disease comprises one or more of acute flaccid myelitis and hand, food, and mouth disease. In some embodiments, when administered in an effective amount to the subject, the administering elicits antibody titers to at least one viral protein antigen equal to or greater than the composition administered to a subject without the plurality of nucleic acids comprising a sequence encoding for an RNA polymerase.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1I show schematic representations of nanoparticle (NP) carriers. FIG. 1A shows an oil-in-water emulsion and a plurality of nucleic acids. FIG. 1B shows a nanostructured lipid carrier and a plurality of nucleic acids. FIG. 1C shows a lipid inorganic nanoparticle and a plurality of nucleic acids. FIG. 1D shows a nanoparticle comprising a cationic lipid membrane, a hydrophobic core, and a plurality of nucleic acids. FIG. 1E shows a nanoparticle comprising a cationic lipid membrane, a hydrophobic core, inorganic nanoparticles within the membrane of the nanoparticle, and a plurality of nucleic acids. FIG. 1F shows a nanoparticle having a cationic lipid membrane, a liquid oil core (e.g., squalene), and two or more RNA or DNA molecules. FIG. 1G shows a nanoparticle having a cationic lipid membrane, inorganic particles, a liquid oil core, and two or more RNA or DNA molecules. FIG. 1H shows a nanoparticle having a cationic lipid membrane, a solid core (e.g., glyceryl trimyristate-dynasan), and two or more RNA or DNA molecules. FIG. 1I shows a nanoparticle having a cationic lipid membrane (e.g., phospholipids, PEG-lipid), a solid core (e.g., cholesterol, ionizable cationic lipid), and two or more RNA or DNA molecules. Schematics are not to scale.

FIG. 2 shows the time measurements of nanoparticle size as measured by dynamic light scattering (DLS). X axis is weeks and Y axis is nm diameter. Three-time courses correspond to storage at 4, 25, and 42 degrees Celsius.

FIGS. 3A-3C show graphs demonstrating anti-spike or receptor binding domain (RBD) IgG levels in C57BL/6 mice 14 days after being injected intramuscularly (IM) with NP-1+self-replicating RNA (repRNA) encoding RNA-1; (2) NP-1+repRNA encoding RNA-2; (3) or combination of NP-1+RNA-1+RNA-2 bivalent vaccine composition. FIG. 3A shows IgG titers in response to D614G spike. FIG. 3B shows IgG titers fin response to wild type spike RBD. FIG. 3C shows IgG titers in response to the SA triple mutant spike.

FIGS. 4A-4C show graphs demonstrating anti-spike or RBD IgG levels in C57BL/6 mice 28 days after being injected IM with repRNA. FIG. 4A shows IgG titers in response to D614G spike at 28 days post immunization. FIG. 4B shows IgG titers in response to wild type spike RBD and 28 days post-immunization. FIG. 4C shows IgG titers in response to the SA triple mutant spike at 28 days post-immunization.

FIGS. 5A-5D show graphs demonstrating anti-RBD IgG levels in C57BL/6 mice 14 days after completion of a prime-boost immunization regimen with repRNA. FIG. 5A shows IgG titers in response to wild type (WT) spike at 42 days post immunization. FIG. 5B shows IgG titers in response to UK spike 42 days post-immunization. FIG. 5C shows IgG titers in response to the SA triple mutant spike at 42 days post-immunization. FIG. 5D shows IgG titers in response to India spike 42 days post-immunization.

FIGS. 6A-6B show graphs demonstrating anti-RBD IgG levels in C57BL/6 mice 105 days after completion of a prime-boost immunization regimen with repRNA. FIG. 6A shows IgG titers in response to WT spike. FIG. 6B shows IgG titers in response to SA spike.

FIGS. 7A-7E show graphs demonstrating serum IgG response in mice on day 14 after immunization with either 1 microgram (mcg, μg) single replicon, a COVID combination of 2 replicons (each at 1 mcg in a total of 2 mcg injection) or a 5 mcg combination of 5 replicons (each at 1 mcg in a total 5 mcg injection). FIG. 7A shows IgG titers in response to WT SARS-CoV-2 RBD. FIG. 7B shows IgG titers in response to response to SA SARS-CoV-2 RBD. FIG. 7C shows IgG titers in response to RSV-G. FIG. 7D shows IgG titers in response to RSV-F. FIG. 7E shows IgG titers in response to H3N2. Data are presented as mean and SEM.

FIGS. 8A-8E show graphs demonstrating serum IgG response in mice on day 28 after immunization with either 1 mcg single replicon, a COVID combination of 2 replicons (each at 1 mcg in a total of 2 mcg injection) or a 5 mcg combination of 5 replicons (each at 1 mcg in a total 5 mcg injection). IgG concentration was determined in ELISA, with the coating antigen indicated in the plot title. FIG. 8A shows IgG titers in response to WT SARS-CoV-2 RBD. FIG. 8B shows IgG titers in response to response to SA SARS-CoV-2 RBD. FIG. 8C shows IgG titers in response to RSV-G. FIG. 8D shows IgG titers in response to RSV-F. FIG. 8E shows IgG titers in response to H3N2. Data are presented as mean and SEM.

FIGS. 9A-9E show graphs demonstrating serum IgG response in mice on experiment day 42 (i.e., 14 days after a second immunization with either 1 mcg single replicon, a COVID combination of 2 replicons (each at 1 mcg in a total of 2 mcg injection) or a 5 mcg combination of 5 replicons (each at 1 mcg in a total 5 mcg injection)). FIG. 9A shows IgG titers in response to WT SARS-CoV-2 RBD. FIG. 9B shows IgG titers in response to response to SA SARS-CoV-2 RBD. FIG. 9C shows IgG titers in response to RSV-G. FIG. 9D shows IgG titers in response to RSV-F. FIG. 9E shows IgG titers in response to H3N2. Data are presented as mean and SEM.

FIG. 10 show a graph demonstrating EV-D68 neutralizing antibody response in mice on experiment day 42. Data are presented as mean and SEM.

### DETAILED DESCRIPTION OF THE INVENTION

Provided herein are compositions and methods, and uses thereof for treatment of various conditions. Briefly, further described herein are (1) nucleic acids coding for antigens, and RNA polymerases; (2) nanoparticle carrier systems; (3) combination compositions; (4) pharmaceutical compositions; (5) dosing; (6) administration; and (7) therapeutic applications.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, “optional” or “optionally” means that the subsequently described circumstance may or may not occur, so that the description includes instances where the circumstance occurs and instances where it does not.

Unless specifically stated or apparent from context, as used herein, the term “about” in reference to a number or range of numbers is understood to mean the stated number and numbers +/−20% thereof, or 20% below the lower listed limit and 20% above the higher listed limit for the values listed for a range.

The term “effective amount” or “therapeutically effective amount” refers to an amount that is sufficient to achieve or at least partially achieve the desired effect.

### Nucleic Acids Encoding Antigens

Provided herein are compositions comprising nucleic acids. In some embodiments, the compositions comprise at least one, at least two, at least three, at least four, at least five, at least six, or at least seven nucleic acids encoding protein antigens. In some embodiments, each of the nucleic acids comprises a sequence encoding for a single protein antigen. In some embodiments, the compositions comprise at least two, at least three, at least four, at least five, at least six, or at least seven nucleic acids that are identical. In some embodiments, the compositions comprise at least two, at least three, at least four, at least five, at least six, or at least seven nucleic acids encoding for the identical antigens. In some embodiments, the compositions comprise at least one, at least two, at least three, at least four, at least five, at least six, or at least seven nucleic acids encoding for non-identical antigens. In some embodiments, the protein antigen comprises a bacterial protein antigen, a viral protein antigen, a tumor protein antigen, an RNA polymerase, or a combination thereof. In some embodiments, the nucleic acids complex to the surface to form nucleic acid-nanoparticle complexes.

Also, provided herein is a composition comprising a plurality of nucleic acids encoding for antigen sequences. In some embodiments, the plurality of nucleic acid comprises at least one, at least two, at least three, at least four, at least five, at least six, or at least seven nucleic acids. In some embodiments, each nucleic acid comprises a sequence coding for an RNA polymerase and an antigen. In some embodiments, each nucleic acid comprises a sequence coding for an RNA polymerase and a viral antigen. In some embodiments, nucleic acids provided herein code for a plurality of viral antigens. In some embodiments, each nucleic acid comprises a sequence coding for an RNA polymerase and a tumor antigen. In some embodiments, nucleic acids provided herein code for a plurality of tumor antigens. In some embodiments, nucleic acids provided herein are complexed to the surface of each nanoparticle. In some embodiments, nucleic acids provided herein are in complex with the membrane of the nanoparticle. In some embodiments, nucleic acids provided herein are in complex with an exterior hydrophilic surface of the nanoparticle.

In some embodiments, nucleic acids provided herein are deoxyribonucleic acids (DNA) or ribonucleic acids (RNA). Nucleic acids provided herein may be linear or include a secondary structure (e.g., a hairpin). In some embodiments, nucleic acids provided herein are polynucleotides comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. In some embodiments, compositions provided herein comprise one or more nucleic acids. In some embodiments, compositions provided herein comprise two or more nucleic acids. In some embodiments, compositions provided herein comprise at least one DNA. In some embodiments, compositions provided herein comprise at least one RNA. In some embodiments, compositions provided herein comprise at least one DNA and at least one RNA. In some embodiments, nucleic acids provided herein are present in an amount of above 5 ng to about 1 mg. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg, 5 mg, 10 mg, 12.5 mg, 15 mg, 25 mg, 40 mg, 50 mg or more. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg, 5 mg, 10 mg, 12.5 mg, 15 mg, 25 mg, 40 mg, 50 mg, 100 mg, or 200 mg. In some embodiments, nucleic acids provided herein are present in an amount of up to about 25, 50, 75, 100, 150, 175 ng. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

Provided here are compositions comprising nanoparticles. In some embodiments, the compositions are capable of inducing multivalent immune responses. In some embodiments, the nanoparticles comprise one or more nucleic acids. In some embodiments, the nanoparticles comprise at least two, at least three, at least four, at least five, at least six, or at least seven identical nanoparticles. In some embodiments, the nanoparticles comprise at least two, at least three, at least four, at least five, at least six, or at least seven non-identical nanoparticles. In some embodiments, each of the non-identical nanoparticles comprise one or more nucleic acids. In some embodiments, one or more nucleic acids of the non-identical nanoparticles encode proteins or functional fragments thereof that are non-identical.

Provided here is a composition comprising a plurality of nucleic acids each coding for a protein or a functional fragment thereof. In some embodiments, the protein is an antigen. In some embodiments, the antigen is derived from a microbial organism. In some embodiments, the antigen is a microbial antigen. In some embodiments, the microbial antigen is a viral protein antigen (e.g., a viral antigen) or a bacterial protein antigen (e.g., a bacterial antigen). Accordingly, in some embodiments, the antigen is a viral antigen or a bacterial antigen. In some embodiments, the viral antigen is a surface protein or a transmembrane protein. In some embodiments, the viral antigen is a cytosolic protein. In some embodiments, the viral antigen is a nuclear protein. In some embodiments, the viral antigen is a spike protein, a glycoprotein, or an envelope protein. In some embodiments, the viral antigen is derived from: an alphavirus, a retrovirus, a lentivirus, a coronavirus, an enterovirus, a flavivirus, a picornavirus, a rhabdovirus, a rotavirus, a norovirus, a paramyxovirus, a orthomyxovirus, a bunyavirus, an arenavirus, a reovirus, a retrovirus, a rabies virus, a papillomavirus, a parvovirus, a herpesvirus, a poxvirus, a hepadnavirus, a spongiform virus, an iridovirus, an influenza virus, a morbillivirus, a togavirus, a variola virus, a varicella virus, a zika virus, a SARS-CoV-2 virus, a respiratory syncytial virus (RSV), a Middle East Respiratory Syndrome (MERS) coronavirus, human immunodeficiency virus (HIV), a human T-Cell leukemia virus, an Epstein-Barr virus, a cytomegalovirus, a papovavirus, an adenovirus, and a bunyvirus. Non-limiting examples of viral antigens include: Zika virus envelope protein (ZIKV E), Zika virus precursor membrane and envelope proteins (prM-ENV), SARS-CoV-2 spike (S) protein and envelope (E) proteins, HIV p24 antigen and Nef protein, influenza virus hemagglutinin (HA) antigen (H2, H3, H5, H6, H7, H8 and H9), influenza virus neuraminidase, rubella E1 and E2 antigens, rotavirus VP7sc antigen, RSV M2 protein, cytomegalovirus envelope glycoprotein B, the S, M, and L proteins of hepatitis B virus, rabies glycoprotein, rabies nucleoprotein, Crimean-Congo hemorrhagic fever glycoprotein Gc and or Gn, Nipah henipavirus glycoprotein, Hendra virus glycoprotein, human papillomavirus E6 protein, human papillomavirus E7 protein, human papillomavirus L1 protein, or human papillomavirus L2 protein. Severe acute respiratory syndrome coronavirus 2 (SARS-CoV-2) is the virus that causes COVID-19 (coronavirus disease 2019). Variants of the SARS-CoV-2 virus include the alpha, beta, delta, and mu variants. The Alpha (B.1.1.7), Beta (B.1.351, B.1.351.2, B.1.351.3), Delta (B.1.617.2, AY.1, AY.2, AY.3), Gamma (P.1, P.1.1, P.1.2), and Omicron (B.1.1.529) variants circulating worldwide are classified as variants of concern. SARS-CoV-2 has four structural proteins, known as the S (spike), E (envelope), M (membrane), and N (nucleocapsid) proteins; the N protein holds the RNA genome, and the S, E, and M proteins together create the viral envelope. In SARS-CoV-2, for example, the spike protein is the protein responsible for allowing the virus to attach to and fuse with the membrane of a host cell. In some embodiments, compositions provided herein comprise a plurality of nucleic acids encoding one or more coronavirus antigens or variants thereof. In some embodiments, compositions provided herein comprise nucleic acids, wherein at least one encodes one or more coronavirus antigens or functional variants thereof. In some embodiments, a coronavirus antigen or a functional variant thereof comprises one or more coronavirus spike proteins or functional variants thereof. In some embodiments, a coronavirus spike protein comprises a wild-type (WT), a prefusion-stabilized (PreF), a furin cleavage site-deleted (Furmut), or a combination of the PreF and Furmut modifications of the full-length coronavirus spike protein. In some embodiments, compositions provided herein comprise a plurality of nucleic acids encoding wild-type (WT), prefusion-stabilized (PreF), furin cleavage site-deleted (Furmut), or a combination of the PreF and Furmut modifications of the full-length spike of a coronavirus spike protein. In some embodiments, coronavirus spike proteins provided herein are SARS-CoV-2 spike proteins. In some embodiments, SARS-CoV-2 spike proteins provided herein are derived from the alpha variant of SARS-CoV-2. In some embodiments, SARS-CoV-2 spike proteins provided herein are derived from the beta variant of SARS-CoV-2. In some embodiments, SARS-CoV-2 spike proteins provided herein are derived from the delta variant of SARS-CoV-2. In some embodiments, SARS-CoV-2 spike proteins provided herein are derived from the mu (p) variant of SARS-CoV-2. In some embodiments, SARS-CoV-2 spike proteins provided herein are derived from a variant of SARS-CoV-2 comprising an amino acid substitution of D614G or D627G.

In some embodiments, compositions provided herein comprise antigen derived from non-enveloped virus. Non-enveloped viruses do not use the host secretory system to enter or leave a host cell during infection. Instead, non-enveloped viruses enter the cytosol by directly penetrating the plasma membrane, as well as through a variety of endocytic mechanisms leading to penetration of internal membrane(s), the Golgi, and the endoplasmic reticulum of the host cell.

Enteroviruses enter the host cell by receptor-mediate endocytosis. Following endocytosis, uncoating of the virion occurs in the endosome and the positive-stranded RNA along with the covalently-linked VPg protein is released into the cytoplasm. Viral RNA is translated by host ribosomes making a single polyprotein that is catalytically cleaved by enterovirus proteases 2Apro and 3Cpro. After production and accumulation of non-structural proteins, including the viral polymerase, viral RNA is then replicated using the virally-encoded RNA-dependent RNA polymerase to generate a double-stranded RNA. The negative sense RNA serves as the template to make more positive sense RNA. Newly produced RNA can be the template to produce more positive sense RNAs or serve as the genome for progeny viruses. Capsid proteins assemble and newly synthesized positive-stranded viral RNA is packaged into virion. Finally, new progeny virions are released either by non-lytic release, where virions are released in vesicles, or are released when the cell undergoes lysis (lytic release).

In some embodiments, the non-enveloped virus is a double-stranded DNA virus. In some embodiments, the double-stranded DNA virus is selected from adenoviridae, iridoviridae, papiliomiaviridae, and polyonaviridae. In some embodiments, the non-enveloped virus is a single-stranded DNA virus. In some embodiments, the single-stranded DNA virus is selected from anellovirus, circoviridae, and parvoviridae. In some embodiments, the non-enveloped virus is a double-stranded RNA virus. In some embodiments, the double-stranded RNA virus is selected from birnaviridae, picobirnaviridae, and reoviridae. In some embodiments, the non-enveloped virus is a single-stranded RNA virus. In some embodiments, the single-stranded RNA virus is selected from picornaviridae, astroviridae, caliciviridae, hepevirus, and nodaviridae.

In some embodiments, the viral antigen is derived from a picornavirus. In some embodiments, the viral antigen is derived from an enterovirus, a coxsackievirus, a rhinovirus, a poliovirus, an echovirus, or a parechovirus. In some embodiments, the viral antigen is derived from an enterovirus. Enteroviruses have a single open reading frame divided into the P1 and nonstructural P2-P3 polyproteins. P1 is divided into capsid proteins VP1, VP2, VP3, and VP4. P3 contains a 3CD protease which cleaves P1 into the four capsid monomers. In some embodiments, the enterovirus is an enterovirus D68 (EV-D68), an enterovirus A71 (EV-A71), a coxsackievirus A6 (CV-A6), or a coxsackievirus B3 (CV-B3). In some embodiments, the enterovirus is enterovirus D68 (EV-D68). In some embodiments, the EV-D68 belongs to clade A. In some embodiments, the EV-D68 belongs to clade B. In some embodiments, the EV-D68 belongs to clade C. In some embodiments, the EV-D68 belongs to clade D. In some embodiments, the EV-D68 is US/MO/14-18947-EV-D68. In some embodiments, compositions provided herein comprise a nucleic acid encoding for a protein, an antibody, or an antibody fragment that binds to an antigen from a picornavirus. In some embodiments, compositions provided herein comprise a nucleic acid encoding for a protein, an antibody, or an antibody fragment that binds to an enterovirus or an enterovirus antigen. In some embodiments, compositions provided herein comprise a nucleic acid encoding for a protein, an antibody, or an antibody fragment that binds to a VP1 capsid protein. In some embodiments, compositions provided herein comprise a nucleic acid encoding for a protein, an antibody, or an antibody fragment that binds to a VP2 capsid protein. In some embodiments, compositions provided herein comprise a nucleic acid encoding for a protein, an antibody, or an antibody fragment that binds to a VP3 capsid protein. In some embodiments, compositions provided herein comprise a nucleic acid encoding for a protein, an antibody, or an antibody fragment that binds to a VP4 capsid protein.

Further provided herein are nucleic acids encoding for a structural protein from a non-enveloped virus and a 3CD protease. In some embodiments, the nucleic acids encoding for a structural protein from a non-enveloped virus and a 3CD protease further comprise an IRES sequence. In some embodiments, the nucleic acids encoding for a structural protein from a non-enveloped virus and a 3CD protease further comprise a non-structural protein from an alphavirus.

In some embodiments, compositions provided herein comprise a plurality of nucleic acids encoding a plurality of viral antigens of the same viral species. In some embodiments, compositions provided herein comprise a plurality of nucleic acids encoding a plurality of viral antigens of different viral species.

In some embodiments, compositions provided herein comprise nucleic acids encoding one or more antigens (e.g., a viral antigen or a bacterial antigen). In some embodiments, compositions provided herein comprise a plurality of nucleic acids each encoding a plurality of antigens. In some embodiments, at least two, at least three, at least four, at least five, at least six, or at least seven of the antigens are from the same species. In some embodiments, at least two, at least three, at least four, at least five, at least six, or at least seven of the plurality of antigens are from different species.

In some embodiments, compositions provided herein comprise nucleic acids comprising sequences encoding one or more, two or more, three or more, or four or more of a coronavirus spike protein or functional variant thereof, an influenza virus hemagglutinin protein or functional variant thereof, an RSV glycoprotein (G) or functional variant thereof, and an RSV fusion (F) protein or a functional variant thereof. In some embodiments, compositions provided herein comprise a plurality of nucleic acids comprising sequences encoding one or more of: (a) a coronavirus spike protein, (b) an influenza virus hemagglutinin protein, (c) an RSV glycoprotein (G), (d) an RSV fusion (F) protein, (e) an enterovirus virus-like particle (VLP), or (f) a variant thereof. In some embodiments, compositions provided herein comprise a plurality of nucleic acids comprising sequences encoding two or more of: (a) a coronavirus spike protein, (b) an influenza virus hemagglutinin protein, (c) an RSV glycoprotein (G), (d) an RSV fusion (F) protein, (e) an enterovirus virus-like particle (VLP), or (f) a variant thereof. In some embodiments, compositions provided herein comprise a plurality of nucleic acids comprising sequences encoding three or more of: (a) a coronavirus spike protein, (b) an influenza virus hemagglutinin protein, (c) an RSV glycoprotein (G), (d) an RSV fusion (F) protein, (e) an enterovirus virus-like particle (VLP), or (f) a variant thereof. In some embodiments, compositions provided herein comprise a plurality of nucleic acids comprising sequences encoding four or more of: (a) a coronavirus spike protein, (b) an influenza virus hemagglutinin protein, (c) an RSV glycoprotein (G), (d) an RSV fusion (F) protein, (e) an enterovirus virus-like particle (VLP), or (f) a variant thereof. In some embodiments, compositions provided herein comprise a plurality of nucleic acids comprising sequences encoding (a) a coronavirus spike protein, (b) an influenza virus hemagglutinin protein, (c) an RSV glycoprotein (G), and (d) an RSV fusion (F) protein, (e) an enterovirus virus-like particle (VLP), or (f) a variant thereof. In some embodiments, compositions provided herein comprise a plurality of nucleic acids comprising sequences encoding (a) an enterovirus antigen (e.g., EV-68D VP1); (b) an influenza virus hemagglutinin protein (e.g., H3 antigen); and (c) an RSV fusion (F) protein.

In some embodiments, the influenza virus hemagglutinin protein is hemagglutinin H2, hemagglutinin H3, hemagglutinin H5, hemagglutinin H6, hemagglutinin H7, hemagglutinin H8, or hemagglutinin H9.

In some embodiments, compositions provided herein comprise a nucleic acid sequence encoding a zika virus envelope (E) protein.

In some embodiments, compositions provided herein comprise nucleic acids encoding one or more protein sequences or functional fragments thereof, wherein at least one of the protein sequences or functional fragments thereof comprises at least 80%, 85%, 90%, 95%, 96%, 97%, 98%, or 99% sequence identity to any one of the sequences listed in Table 1. In some embodiments, compositions provided herein comprise two or more nucleic acids each encoding one or more protein sequences or functional fragments thereof, wherein at least one of the protein sequences or functional fragments thereof comprises at least 80%, 85%, 90%, 95%, 96%, 97%, 98%, or 99% sequence identity to any one of the sequences listed in Table 1. Percent (%) sequence identity for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

In some embodiments, nucleic acids provided herein code for a protein sequence or a functional fragment of the protein sequence listed in Table 1. In some embodiments, compositions provided herein comprise two or more nucleic acids encoding different sequences listed in Table 1. In some embodiments, each nucleic acid provided herein comprises at least 80%, 85%, 90%, 95%, 96%, 97%, 98%, or 99% sequence identity to an antigen sequence listed in Table 1. In some embodiments, compositions provided herein comprise two or more, three or more, four or more, five or more, six or more, or up to seven or more nucleic acids encoding different sequences listed in Table 1. Exemplary nucleic acid sequences encoding antigens and antigen amino acid sequences are listed in Table 1.

TABLE 1
Nucleic Acid and Amino Acid Viral Antigen Sequences.

| SEQ ID NO: | Name | Organism | Variant |
|---|---|---|---|
| SARS CoV-2 Antigen RNA Sequences | | | |
| SEQ ID NO: 1 | Delta V5 | severe acute respiratory | A.1 |
| | | syndrome coronavirus | |
| | | 2 (SARS-CoV-2) | |
| SEQ ID NO: 2 | K995P-V996P | severe acute respiratory | A.1-preF |
| | | syndrome coronavirus | |
| | | 2 (SARS-CoV-2) | |
| SEQ ID NO: 3 | D614G | severe acute respiratory | B.1 |
| | | syndrome coronavirus | |
| | | 2 (SARS-CoV-2) | |
| SEQ ID NO: 4 | B.1.351-PP-D614G | severe acute respiratory | Beta-preF |
| | | syndrome coronavirus | |
| | | 2 (SARS-CoV-2) | |
| SEQ ID NO: 5 | B.1.1.7-PP-D614G | severe acute respiratory | Alpha-preF |
| | | syndrome coronavirus | |
| | | 2 (SARS-CoV-2) | |
| SEQ ID NO: 6 | Delta.AY1-S2P-wtFur | severe acute respiratory | Delta-preF |
| | | syndrome coronavirus | |
| | | 2 (SARS-CoV-2) | |
| SEQ ID NO: 7 | Delta.AY1-S2P-wtFur- | severe acute respiratory | Delta-preF- |
| | newKozak | syndrome coronavirus | kozak |
| | | 2 (SARS-CoV-2) | |
| RSV Antigen RNA Sequences | | | |
| SEQ ID NO: 8 | RSV fusion protein | human respiratory | |
| | (RSV- F) | syncytial virus (RSV) | |
| SEQ ID NO: 9 | RSV glycoprotein | human respiratory | |
| | (RSV-G) | syncytial virus (RSV) | |

RSV Antigen Amino Acid Sequences

| | | |
|---|---|---|
| SEQ ID NO: 10 | RSV-F (full length) | human respiratory |
| | | syncytial virus (RSV) |
| SEQ ID NO: 11 | RSV-F (partial) | human respiratory |
| | | syncytial virus (RSV) |
| SEQ ID NO: 12 | RSV-G | human respiratory |
| | | syncytial virus (RSV) |

Influenza Antigen RNA Sequence

| | | |
|---|---|---|
| SEQ ID NO: 13 | Influenza | Influenza A virus |
| | Haemagglutinin (HA) | |

Influenza Antigen Amino Acid Sequences

| | | | |
|---|---|---|---|
| SEQ ID NO: 14 | Influenza A | Influenza A virus | H3N2 (New |
| | Haemagglutinin (HA) | | York) |
| SEQ ID NO: 15 | Influenza B | Influenza B virus | Lee 1940 |
| | Haemagglutinin (HA) | | |

Zika Virus Antigen Amino Acid Sequence

| | | |
|---|---|---|
| SEQ ID NO: 16 | Zika pre-membrane and | Zika virus |
| | Envelope (prM-E) | |

Enterovirus Antigen Amino Acid Sequence

| | | | |
|---|---|---|---|
| SEQ ID NO: 84 | VEE-EVD68-IRES-VLP | Enterovirus | EV-D68 |
| SEQ ID NO: 85 | VEE-IL-18952-p1-IRES- | Enterovirus | IL-18952 |
| | 3cd | | |
| SEQ ID NO: 86 | VEE-JPOC10-396-p1- | Enterovirus | JPOC10-396 |
| | IRES-3cd | | |
| SEQ ID NO: 87 | VEE-KY-14-18953-p1- | Enterovirus | KY-14-18953 |
| | IRES-3cd | | |
| SEQ ID NO: 88 | VEE-NZ-2010-541-p1- | Enterovirus | NZ-2010-541 |
| | IRES-3cd | | |
| SEQ ID NO: 89 | VEE-USA-2018-23087- | Enterovirus | USA-2018- |
| | p1-IRES-3cd | | 23087 |

In some embodiments, the nucleic acids provided herein encode for a tumor antigen or functional variant thereof. In some embodiments, the tumor antigen is a surface protein, a cytosolic protein, or a transmembrane protein. Non-limiting examples of tumor antigens include: epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, chronic myelogenous WT1, myelodysplastic syndrome WT1, acute lymphoblastic leukemia PRAME, Chronic lymphocytic leukemia survivin, Non-Hodgkin's lymphoma survivin, multiple myeloma New York esophagus 1 (NY-Esol), malignant melanoma MAGE, MAGE-A1, MAGE-A3, MART-1/Melan-A, MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, Tyrosinase, glycoprotein 100, breast cancer WT1, Herceptin, lung cancer WT1, prostate-specific antigen (PSA), prostatic acid phosphatase, (PAP) carcinoembryonic antigen (CEA), mucins (e.g., MUC-1), renal cell carcinoma (RCC) fibroblast growth factor (FGF), and programmed cell death protein (PD-1). In some embodiments, nucleic acids provided herein encode for an amino acid sequence listed in Table 2. In some embodiments, compositions provided herein comprise two or more, three or more, four or more, five or more, six or more, or up to seven or more nucleic acids encoding different sequences listed in Table 2. In some embodiments nucleic acids provided herein encodes for protein sequence listed in Table 2 and is used as part of a treatment or prevention of melanoma.

TABLE 2
Exemplary Antigens on Melanoma Cells

| SEQ ID NO: | Reference Protein | Amino Acid Sequence |
|---|---|---|
| SEQ ID NO: 17 | Tyrosinase | KCDICTDEY |
| SEQ ID NO: 18 | Tyrosinase | YMDGTMSQV |
| SEQ ID NO: 19 | Tyrosinase | MLLAYLYQL |
| SEQ ID NO: 20 | Tyrosinase | AFLPWHRLF, |
| SEQ ID NO: 21 | Tyrosinase | SEIWRDIDF |
| SEQ ID NO: 22 | gp100/pMEL17 | YLEPGPVTA |
| SEQ ID NO: 23 | gp100/pMEL17 | KTWGQUWQV |
| SEQ ID NO: 24 | gp100/pMEL17 | ITDQVPFSV |
| SEQ ID NO: 25 | gp100/pMEL17 | VLYRYGSFSV |
| SEQ ID NO: 26 | gp100/pMEL17 | LLDGTATLRL |
| SEQ ID NO: 27 | gp100/pMEL17 | ALLAVGATK |
| SEQ ID NO: 28 | gp100/pMEL17 | MLGTHTMEV |
| SEQ ID NO: 29 | gp100/pMEL17 | LIYRRRLMK |
| SEQ ID NO: 30 | gp100/pMEL17 | ALNFPGSQK |
| SEQ ID NO: 31 | MART-1/MelanA | AAGIGILTV^{†} |
| SEQ ID NO: 32 | MART-1/MelanA | ILTVILGVL |
| SEQ ID NO: 33 | gp75/TRP-1 | MSLQRQFLR |
| SEQ ID NO: 34 | TRP-2 | SVYDFFVWL |
| SEQ ID NO: 35 | TRP-2 | LLGPGRPYR |
| SEQ ID NO: 36 | CEA | YLSGANLNL |
| SEQ ID NO: 37 | HER-2/neu | KIFGSLAFL |
| SEQ ID NO: 38 | HER-2/neu | VMAGVGSPYV |
| SEQ ID NO: 39 | HER-2/neu | IISAVVGIL |
| SEQ ID NO: 40 | PSMA | LLHETDSAV |
| SEQ ID NO: 41 | PSMA | ALFDIESKV |
| SEQ ID NO: 42 | MAGE-1 | EADPTGHSY |
| SEQ ID NO: 43 | MAGE-1 | SLFRAVITK |
| SEQ ID NO: 44 | MAGE-1 | SAYGEPRKL |
| SEQ ID NO: 45 | MAGE-2 | KMVELVHFL |
| SEQ ID NO: 46 | MAGE-2 | YLOLVFGIEV |
| SEQ ID NO: 47 | MAGE-3 | EVDPIGHLY |
| SEQ ID NO: 48 | MAGE-3 | FLWGPRALV |
| SEQ ID NO: 49 | MAGE-3 | MEVDPIGHLY |
| SEQ ID NO: 50 | BAGE | AARAVFLAL |
| SEQ ID NO: 51 | GAGE-1,2 | YRPRPRRY |
| SEQ ID NO: 52 | GnT-V | VLPDVFIRC |
| SEQ ID NO: 53 | NY-ESO-1 | QLSLLMWIT |
| SEQ ID NO: 54 | NY-ESO-1 | SLLMWITQC |
| SEQ ID NO: 55 | NY-ESO-1 | ASGPGGGAPR |
| SEQ ID NO: 56 | 43kD protein | QDLTMKYQIF |
| SEQ ID NO: 57 | p15 | (E)AYGLDFYIL |
| SEQ ID NO: 58 | Mutated beta-catenin | SYLDSGIHF^{‡} |
| SEQ ID NO: 59 | Mutated elongation factor 2 | ETVSEQSNV^{§} |
| SEQ ID NO: 60 | Mutated CASP-8 | FPSDSWCYF |
| | (FLICE/MACH) | |
| SEQ ID NO: 61 | MUM-1 gene product | EEKLIVVLF^{¶} |
| | mutated | |
| | across intron/exon junction | |

^{†}AAGIGILTV (SEQ ID NO: 31) is also recognized by HLA B45-1- restricted cytotoxic T lymphocyte.
^{‡}Phenylalanine (F) at position 9 is the result of mutation. The wild-type sequence is SYLDSGIHS (SEQ ID NO: 90).
^{§}Glutamine (Q) at position 6 is the result of somatic mutation. The wild-type sequence is ETVSEESNV (SEQ ID NO: 91).
^{¶}Isoleucine (I) at position 5 is the result of mutation. The wild-type sequence is EEKLSVVLF (SEQ ID NO: 92).

In some embodiments, compositions provided herein comprise a nucleic acid encoding one or more protein sequences or functional fragments thereof, wherein at least one of the protein sequences or functional fragments thereof comprises at least 80%, 85%, 90%, 95%, 96%, 97%, 98%, or 99% sequence identity to any one of the sequences listed in Table 2. In some embodiments, compositions provided herein comprise two or more nucleic acids each encoding one or more protein sequences or functional fragments thereof, wherein at least one of the protein sequences or functional fragments thereof comprises at least 80%, 85%, 90%, 95%, 96%, 97%, 98%, or 99% sequence identity to any one of the sequences listed in Table 2.

### Nucleic Acids Encoding RNA Polymerase

Provided herein are compositions comprising a self-replicating nucleic acid. In some embodiments, compositions provided herein comprise one or more nucleic acids. In some embodiments, compositions provided herein comprise two or more nucleic acids. In some embodiments, nucleic acids provided herein encode for an RNA polymerase. In some embodiments, nucleic acids provided herein encode for a viral RNA polymerase. In some embodiments, nucleic acids provided herein encode for: (1) a viral RNA polymerase; and (2) a protein, antibody, or functional fragment thereof. In some embodiments, compositions provided herein comprise a first nucleic acid encoding for a viral RNA polymerase; and a second nucleic acid encoding for a protein, antibody, or functional fragment thereof.

Provided herein are compositions comprising a self-replicating RNA. A self-replicating RNA (also called a replicon) includes any genetic element, for example, a plasmid, cosmid, bacmid, phage or virus or a functional genomic fragment thereof that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an RNA virus RNA polymerase, an animal virus RNA polymerase, a togavirus virus RNA polymerase, an alphavirus RNA polymerase, an Eastern equine encephalitis virus (EEEV) RNA polymerase, a Western equine encephalitis virus (WEEV), Venezuelan equine encephalitis virus (VEEV), Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 62. In some embodiments, the nucleic acid encoding for the RNA polymerase comprises at least 90% identity to the nucleic acid sequence of SEQ ID NO: 62. In some embodiments, the nucleic acid encoding for the RNA polymerase comprises at least 95% identity to the nucleic acid sequence of SEQ ID NO: 62. In some embodiments, the nucleic acid encoding for the RNA polymerase comprises at least 99% identity to the nucleic acid sequence of SEQ ID NO: 62. In some embodiments, the nucleic acid encoding for the RNA polymerase is SEQ ID NO: 62.

In some embodiments, the amino acid sequence for VEEV RNA polymerase comprises at least 85% identity to RELPVLDSAAFNVECFKKYACNNEYWETFKENPIRLTEENVVNYITKLKGP (SEQ ID NO: 63) or TQMRELPVLDSAAFNVECFKKYACNNEYWETFKENPIRLTE (SEQ ID NO: 64). In some embodiments, the amino acid sequence for VEEV RNA polymerase comprises at least 90% identity to SEQ ID NO: 63, SEQ ID NO: 64, or SEQ ID NO: 66. In some embodiments, the amino acid sequence for VEEV RNA polymerase comprises at least 95% identity to SEQ ID NO: 63, SEQ ID NO: 64, or SEQ ID NO: 66. In some embodiments, the amino acid sequence for VEEV RNA polymerase comprises at least 99% identity to SEQ ID NO: 63, SEQ ID NO: 64, or SEQ ID NO: 66. In some embodiments, the amino acid sequence for VEEV RNA polymerase is SEQ ID NO: 63, SEQ ID NO: 64, or SEQ ID NO: 66.

### Nanoparticle Carriers Systems

Provided herein are various compositions comprising a carrier, nanoparticle, or a plurality of nanoparticles. Nanoparticles are also referred to herein as carriers or abbreviated as NPs. Nanoparticles provided herein may be an organic, inorganic, or a combination of inorganic and organic materials that are less than about 1 micrometer (μm) in diameter. In some embodiments, nanoparticles provided herein are used as a delivery system for a bioactive agent (e.g., a plurality of nucleic acids encoding an antigen provided herein).

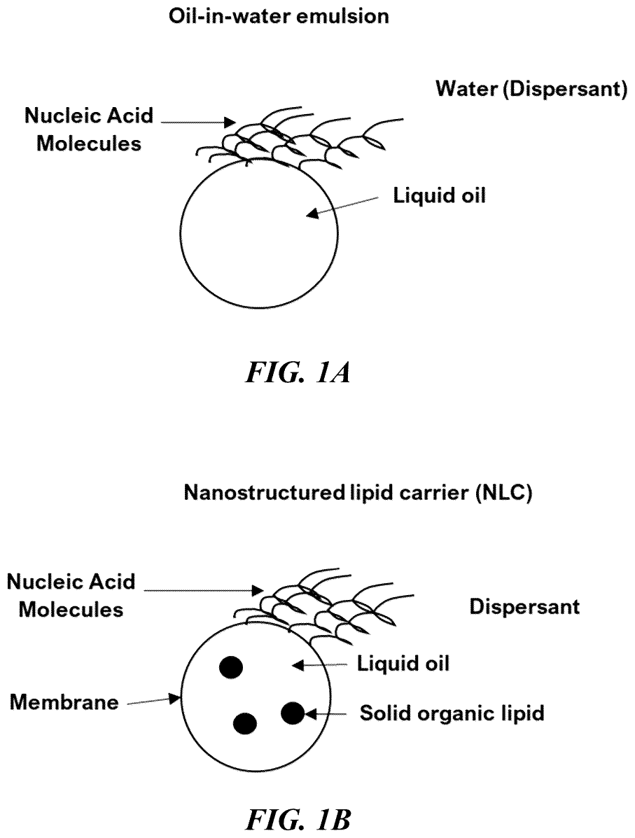

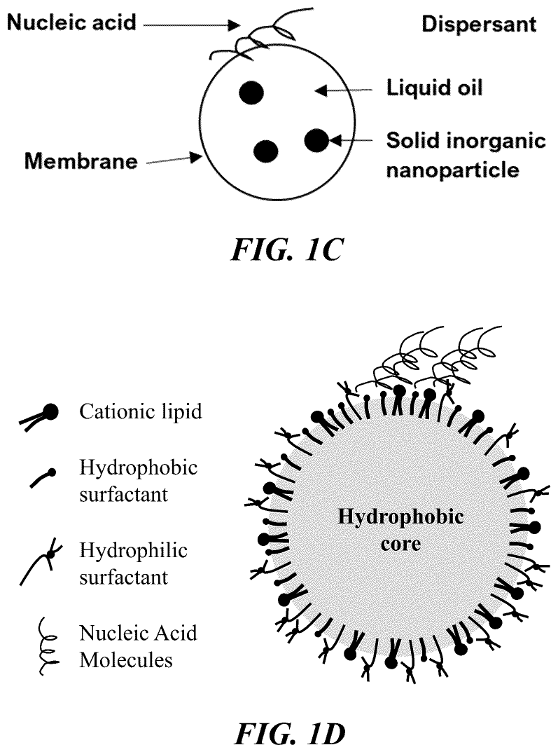

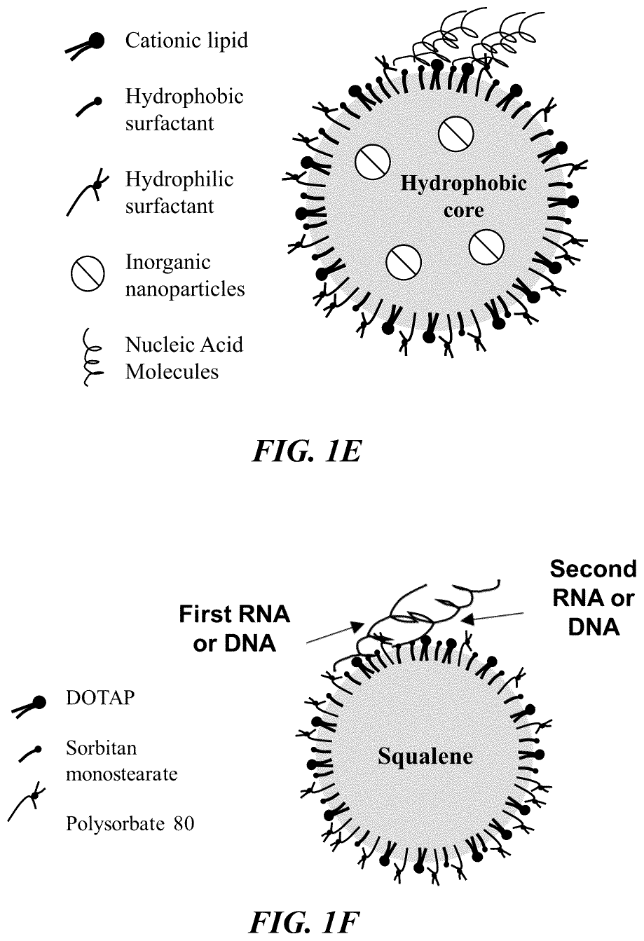

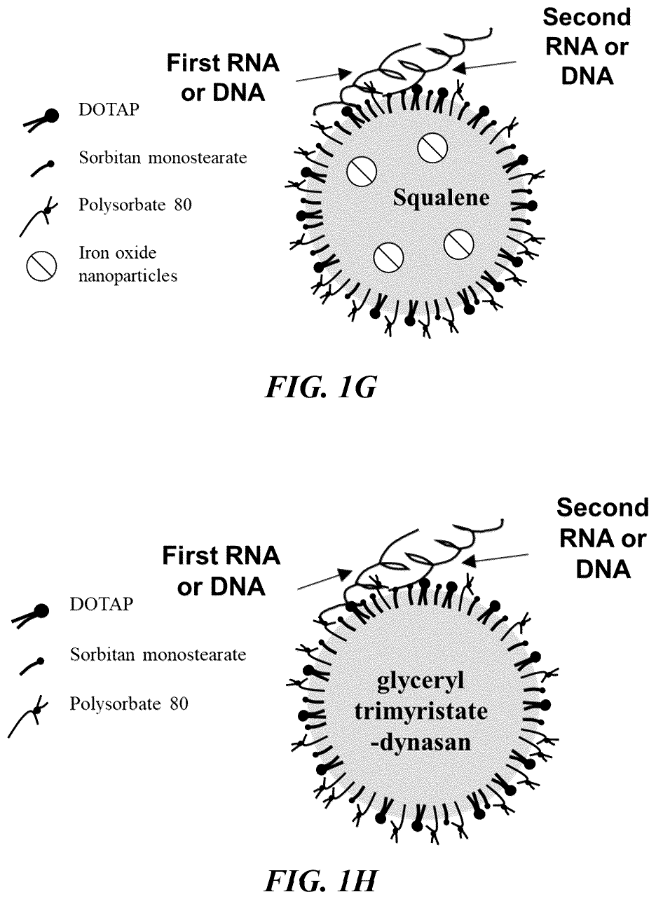

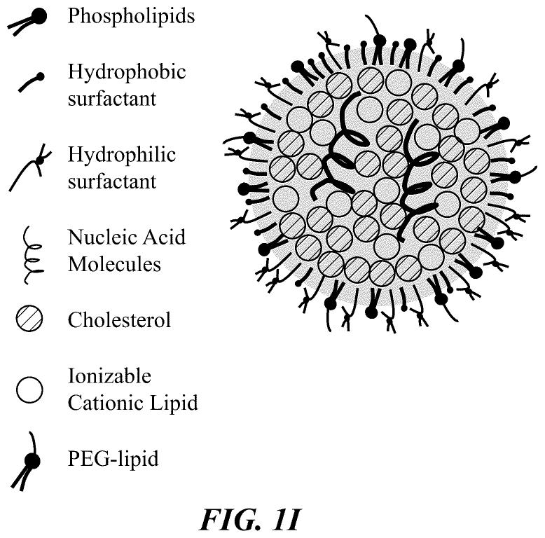

Various nanoparticles and formulations of nanoparticles (i.e., nanoemulsions) are employed. Exemplary nanoparticles are illustrated in FIGS. 1A-1K herein. Nanoparticles or carriers provided herein can include but are not limited to: oil in water emulsions, nanostructured lipid carriers (NLCs), cationic nanoemulsions (CNEs), vesicular phospholipid gels (VPG), polymeric nanoparticles, cationic lipid nanoparticles, liposomes, gold nanoparticles, solid lipid nanoparticles (LNPs or SLNs), mixed phase core NLCs, ionizable lipid carriers, magnetic carriers, polyethylene glycol (PEG)-functionalized carriers, cholesterol-functionalized carriers, polylactic acid (PLA)-functionalized carriers, and polylactic-co-glycolic acid (PLGA)-functionalized lipid carriers.

Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1E (not to scale). FIG. 1C (not to scale) illustrates a lipid nanoparticle with the solid inorganic nanoparticles (e.g., iron oxide nanoparticles) dispersed in liquid oil. The entire nanodroplet is then dispersed as a colloid in the aqueous (water) phase. FIG. 1D (not to scale), illustrates a nanostructured lipid carrier (NLCs) comprising cationic lipids, hydrophobic surfactants, and hydrophilic surfactants forming a hydrophobic core. A surface of the NLC forms a complex with a plurality of nucleic acids (nucleic acid-nanoparticle complexes) The entire nanodroplet is dispersed in the aqueous (water) phase. FIG. 1E (not to scale), illustrates an NLC of FIG. 1B comprising solid inorganic nanoparticles within the hydrophobic core. FIG. 1F illustrates a nanoparticle comprising a cationic lipid membrane (e.g., DOTAP), a liquid oil core (e.g., squalene) without an inorganic particle, and one or more nucleic acids, wherein the one or more nucleic acids are in complex with the membrane (FIG. 1F, not to scale). In some embodiments, nanoparticles of FIG. 1F (not to scale) further comprise iron oxide nanoparticles within the core as shown in FIG. 1G (not to scale). In some embodiments, a nanoparticle provided herein comprises a solid core comprising glyceryl trimyristate-dynasan (FIG. 1H). In some embodiments, a nanoparticle provided herein comprises a solid core comprising an ionizable cationic lipid and cholesterol (FIG. 1I). In some embodiments, the nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, the nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9″′″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); PEGylated lipids including PEGylated version of any of the above lipids (e.g., DSPE-PEGs), and a combination thereof. In some embodiments, the nanoparticle provided herein comprises DOTAP.

In some embodiments, the nanoparticle provided herein comprise a hydrophobic core. In some embodiments, the hydrophobic core comprises an oil. In some embodiments, the hydrophobic core comprises a lipid in liquid phase at 20° C., 25° C., 30° C., 35° C., 40° C., 45° C., or 50° C. In some embodiments, the nanoparticle provided herein comprises an oil. In some embodiments, the oil is in liquid phase. Non-limiting examples of oils that can be used include α-tocopherol, coconut oil, dihydroisosqualene (DHIS), farnesene, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, vitamin E, or combinations thereof. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the nanoparticle comprises a triglyceride ester of saturated coconut or palmkernel oil derived caprylic and capric fatty acids and plant derived glycerol, e.g., Miglyol 812 N.

In some embodiments, the hydrophobic core comprises a lipid in solid phase at 20° C., 25° C., 30° C., 35° C., 40° C., 45° C., or 50° C. In some embodiments, the hydrophobic core comprises glyceryl trimyristate-dynasan or a derivative thereof.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. FIG. 1E illustrates an embodiment where the solid inorganic particle is within the hydrophobic core. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt, a metal oxide, a metal hydroxide, a metal phosphate, or a combination thereof. In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO₂), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, iron sulfate, or a combination thereof. The inorganic particles may be formed from one or more same or different metals (any metals including transition metal).

In some embodiments, the inorganic particle is a transition metal oxide. In some embodiments, the transition metal is magnetite (Fe₃O₄), maghemite (y-Fe₂O₃), wüstite (FeO), hematite (alpha (α)-Fe₂O₃), or a combination thereof. In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, the nanoparticle provided herein comprises a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant. Thus, in some embodiments, the nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85). Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having a HLB value from 1 to 5, or from 4 to 5.

In some embodiments, the nanoparticle provided herein comprises a hydrophilic surfactant, also called an emulsifier. In some embodiments, the nanoparticle provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as Tween, Kolliphor, Scattics, Alkest, or Canarcel; polyoxyethylene sorbitan ester (polysorbate); polysorbate 80 (polyoxyethylene sorbitan monooleate, or Tween 80); polysorbate 60 (polyoxyethylene sorbitan monostearate, or Tween 60); polysorbate 40 (polyoxyethylene sorbitan monopalmitate, or Tween 40); and polysorbate 20 (polyoxyethylene sorbitan monolaurate, or Tween 20). In one embodiment, the hydrophilic surfactant is polysorbate 80.

Nanoparticles provided herein comprise a hydrophobic core surrounded by a lipid membrane (e.g., a cationic lipid such as DOTAP). In some embodiments, the hydrophobic core comprises: one or more inorganic particles; a phosphate-terminated lipid; and a surfactant.

Inorganic solid nanoparticles described herein may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle.

In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA).

In some embodiments, the hydrophobic core comprises a surfactant, wherein the surfactant is a phosphorous-terminated surfactant, a carboxylate-terminated surfactant, a sulfate-terminated surfactant, or an amine-terminated surfactant. Typical carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS).

In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate. In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the nanoparticle provided herein can comprise from about 0.2% to about 40% w/v squalene, from about 0.0010% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80.

In some embodiments the nanoparticle provided herein from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80.

In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80.

In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

Exemplary nanoparticle formulations include any of one or more of the formulations provided in Table 3. In some embodiments, a composition described herein comprises any one or more of a NP-1 to NP-37. In some embodiments, the composition comprises a mixture of different nanoparticle species, e.g., NP-1 and any one of NP-2 to NP-37. In some embodiments, the composition comprises a mixture of the same nanoparticle species, e.g., NP-1. In some embodiments, the nanoparticles provided herein are admixed with a plurality of nucleic acid molecules provided herein. In some embodiments, nanoparticles provided herein are made by homogenization and ultrasonication techniques. In some embodiments, any one or more of NP-1 to NP-37 can be used to make a composition capable of inducing multivalent immune response.

TABLE 3
Nanoparticle Formulations.

| | Cationic Lipid(s) | Oil(s) | Surfactant(s) | Additional Ingredients |
|---|---|---|---|---|
| Name | % (w/v) or mg/ml | % (w/v) or mg/ml | % (w/v) or mg/ml | % (w/v), mg/ml, or mM |
| NP-1 | 30 mg/ml 1,2- | 37.5 mg/ml squalene | 37 mg/ml sorbitan | 0.2 mg Fe/ml |
| | dioleoyl-3- | | monostearate, (2R)- | 12 nm oleic acid- |
| | trimethylammonium- | | 2-[(2R,3R,4S)-3,4- | coated iron oxide |
| | propane (DOTAP) | | Dihydroxyoxolan-2- | nanoparticles |
| | chloride | | yl]-2-hydroxyethyl | 10 mM sodium |
| | | | octadecenoate, C₂₄H₄₆O₆) | citrate dihydrate. |
| | | | (SPAN ® 60) | |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-2 | 30 mg/ml 1,2- | 37.5 mg/ml squalene | 37 mg/ml sorbitan | 1 mg Fe/ml |
| | dioleoyl-3- | | monostearate, (2R)- | 15 nm oleic acid- |
| | trimethylammonium- | | 2-[(2R,3R,4S)-3,4- | coated iron oxide |
| | propane (DOTAP) | | Dihydroxyoxolan-2- | nanoparticles |
| | chloride | | yl]-2-hydroxyethyl | 10 mM sodium |
| | | | octadecenoate, C₂₄H₄₆O₆) | citrate dihydrate. |
| | | | (SPAN ® 60) | |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-3 | 30 mg/ml 1,2- | 37.5 mg/ml Miglyol 812N | 37 mg/ml sorbitan | 0.2 mg Fe/ml |
| | dioleoyl-3- | (triglyceride ester of | monostearate, (2R)- | 15 nm oleic acid- |
| | trimethylammonium- | saturated coconut/ | 2-[(2R,3R,4S)-3,4- | coated iron oxide |
| | propane (DOTAP) | palmkernel oil derived | Dihydroxyoxolan-2- | nanoparticles |
| | chloride | caprylic and capric | yl]-2-hydroxyethyl | 10 mM sodium |
| | | fatty acids and plant | octadecenoate, C₂₄H₄₆O₆) | citrate dihydrate. |
| | | derived glycerol) | (SPAN ® 60) | |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-4 | 30 mg/ml 1,2- | 37.5 mg/ml Miglyol 812N | 37 mg/ml sorbitan | 1 mg Fe/ml |
| | dioleoyl-3- | (triglyceride ester of | monostearate, (2R)- | 15 nm oleic acid- |
| | trimethylammonium- | saturated coconut/ | 2-[(2R,3R,4S)-3,4- | coated iron oxide |
| | propane (DOTAP) | palmkernel oil derived | Dihydroxyoxolan-2- | nanoparticles |
| | chloride | caprylic and capric | yl]-2-hydroxyethyl | 10 mM sodium |
| | | fatty acids and plant | octadecenoate, C₂₄H₄₆O₆) | citrate dihydrate. |
| | | derived glycerol) | (SPAN ® 60) | |
| | | | 37 mg/ml polyoxyethylene | |
| | | | (20) sorbitan monooleate, | |
| | | | C₆₄H₁₂₄O₂₆, Polysorbate 80 | |
| | | | (TWEEN 80 ®) | |
| NP-5 | 30 mg/ml DOTAP | 37.5 mg/ml squalene | 37 mg/ml sorbitan | 1 mg/ml |
| | chloride | | monostearate (SPAN ® 60) | trioctylphosphine |
| | | | 37 mg/ml polysorbate 80 | oxide |
| | | | (TWEEN ® 80) | (TOPO)-coated |
| | | | | aluminum hydroxide |
| | | | | (Alhydrogel ® 2%) |
| | | | | particles |
| | | | | 10 mM sodium |
| | | | | citrate dihydrate. |
| NP-6 | 30 mg/ml DOTAP | 37.5 mg/ml Solanesol | 37 mg/ml sorbitan | 0.2 mg Fe/ml oleic |
| | chloride | (Cayman chemicals), | monostearate (SPAN ® 60) | acid-coated iron |
| | | | 37 mg/ml polysorbate 80 | oxide nanoparticles |
| | | | (TWEEN ® 80) | 10 mM sodium citrate |
| NP-7 | 30 mg/ml DOTAP | 37.5 mg/ml squalene | 37 mg/ml sorbitan | 10 mM sodium citrate |
| | chloride | 2.4 mg/ml Dynasan 114 | monostearate (SPAN ® 60) | |
| | | | 37 mg/ml polysorbate 80 | |
| | | | (TWEEN ® 80 | |
| NP-8 | 4 mg/ml DOTAP | 43 mg/ml squalene | 5 mg/ml Span ® 85 | 10 mM sodium citrate |
| | chloride | | 5 mg/ml Tween ® 80 | |
| NP-9 | 7.5 mg/ml 1,2- | 9.4 mg/ml squalene | 9.3 mg/ml sorbitan | 0.05 mg/ml 15 |
| | dioleoyl-3- | ((6E,10E,14E,18E)- | monostearate, (2R)- | nanometer |
| | trimethylammonium- | 2,6,10,15,19,23- | 2-[(2R,3R,4S)-3,4- | superparamagnetic |
| | propane (DOTAP) | Hexamethyltetracosa- | Dihydroxyoxolan-2- | iron oxide (Fe₃O₄) |
| | chloride | 2,6,10,14,18,22- | yl]-2-hydroxyethyl | 10 mM sodium |
| | | hexaene, C₃₀H₅₀) | octadecenoate, C₂₄H₄₆O₆) | citrate dihydrate |
| | | 0.63 mg/ml glyceryl | (SPAN ® 60) | |
| | | trimyristate-dynasan | 9.3 mg/ml polyoxyethylene | |
| | | (DYNASAN 114 ®) | (20) sorbitan monooleate, | |
| | | | C₆₄H₁₂₄O₂₆, Polysorbate 80 | |
| | | | (TWEEN 80 ®) | |
| NP-10 | 0.4% DOTAP | 0.25% glyceryl | 0.5% sorbitan | |
| | | trimyristate-dynasan | monostearate (SPAN ® 60) | |
| | | (DYNASAN 114 ®) | 0.5% polysorbate 80 | |
| | | 4.75% Squalene | (TWEEN 80 ®) | |
| NP-11 | 3.0% DOTAP | 0.25% glyceryl | 3.7% sorbitan | |
| | | trimyristate-dynasan | monostearate (SPAN ® 60) | |
| | | (DYNASAN 114 ®) | 3.7% polysorbate 80 | |
| | | 3.75% Squalene | (TWEEN 80 ®) | |
| NP-12 | 0.4% DOTAP | 4.3% Squalene | 0.5% sorbitan | |
| | | | trioleate (SPAN ® 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate-dynasan | (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate (SPAN ® 85) | |
| | | (DYNASAN 114 ®) | 2.0% polysorbate 80 | |
| | | 4.08% squalene | (TWEEN ® 80) | |
| NP-15 | 0.4% DOTAP | 0.25% glyceryl | 0.25% sorbitan | |
| | | trimyristate-dynasan | trioleate (SPAN ® 85) | |
| | | (DYNASAN 114 ®) | 2.0% polysorbate 80 | |
| | | 4.08% squalene | (TWEEN ® 80) | |
| NP-16 | 0.4% DOTAP | 5% squalene | 0.5% sorbitan | |
| | | | trioleate (SPAN ® 85) | |
| | | | 2.0% polysorbate 80 | |
| | | | (TWEEN ® 80) | |
| NP-17 | 0.4% DOTAP | 5% squalene | 0.5% sorbitan | |
| | | | monostearate (SPAN ® 60) | |
| | | | 2% polysorbate 80 | |
| | | | (TWEEN ® 80) | |
| NP-18 | 0.4% DOTAP | 0.25% glyceryl | 2% sorbitan trioleate | |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-19 | 0.4% DOTAP | 0.25% glyceryl | 0.5% sorbitan | 1% aluminum |
| | | trimyristate-dynasan | monostearate (SPAN ® 60) | hydroxide |
| | | (DYNASAN 114 ®) | 0.5% polysorbate 80 | |
| | | 4.75% Squalene | (TWEEN 80 ®) | |
| NP-20 | 3.0% DOTAP | 0.25% glyceryl | 3.7% sorbitan | 1% aluminum |
| | | trimyristate-dynasan | monostearate (SPAN ® 60) | hydroxide |
| | | (DYNASAN 114 ®) | 3.7% polysorbate 80 | |
| | | 3.75% Squalene | (TWEEN ® 80) | |
| NP-21 | 0.4% DOTAP | 4.3% Squalene | 0.5% sorbitan | 1% aluminum |
| | | | trioleate (SPAN ® 85) | hydroxide |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum |
| | | trimyristate-dynasan | (TWEEN ® 80) | hydroxide |
| | | (DYNASAN 114 ®) | | |
| | | 4.08% squalene | | |
| NP-23 | 0.4% DOTAP | 0.25% glyceryl | 0.5% sorbitan | 1% aluminum |
| | | trimyristate-dynasan | trioleate (SPAN ® 85) | hydroxide |
| | | (DYNASAN 114 ®) | 2.0% polysorbate 80 | |
| | | 4.08% squalene | (TWEEN ® 80 | |
| NP-24 | 0.4% DOTAP | 0.25% glyceryl | 0.25% sorbitan | 1% aluminum |
| | | trimyristate-dynasan | trioleate (SPAN ® 85) | hydroxide |
| | | (DYNASAN 114 ®) | 2.0% polysorbate 80 | |
| | | 4.08% squalene | (TWEEN ® 80 | |
| NP-25 | 0.4% DOTAP | 5% squalene | 0.5% sorbitan | 1% aluminum |
| | | | trioleate (SPAN ® 85) | hydroxide |
| | | | 2.0% polysorbate 80 | |
| | | | (TWEEN ® 80 | |
| NP-26 | 0.4% DOTAP | 5% squalene | 0.5% sorbitan | 1% aluminum |
| | | | monostearate (SPAN ® 60) | hydroxide |
| | | | 2% polysorbate 80 | |
| | | | (TWEEN ® 80) | |
| NP-27 | 0.4% DOTAP | 0.25% glyceryl | 2% sorbitan | 1% aluminum |
| | | trimyristate-dynasan | trioleate (SPAN ® 85) | hydroxide |
| | | (DYNASAN 114 ®) | 2% polysorbate 80 | |
| | | 4.08% squalene | (TWEEN ® 80) | |
| NP-28 | 0.5-5.0 mg/ml DOTAP | 0.2-10% (v/v) squalene | 0.01-2.5% (v/v) | |
| | | | polysorbate 80 | |
| | | | (TWEEN ® 80) | |
| NP-29 | 0.4% (w/w) DOTAP | 4.3% (w/w) squalene | 0.5% (w/w) sorbitan | |
| | | | trioleate (SPAN ® 85) | |
| | | | 0.5% (w/w) polysorbate 80 | |
| | | | (TWEEN ® 80) | |
| NP-30 | 30 mg/ml DOTAP | 37.5 mg/ml squalene | 37 mg/ml sorbitan | 10 mM sodium citrate |
| | chloride | | monostearate (SPAN ® 60) | |
| | | | 37 mg/ml polysorbate 80 | |
| | | | (TWEEN ® 80) | |
| NP-31 | 30 mg/ml DOTAP | 37.5 mg/ml squalene | 37 mg/ml sorbitan | 0.4 mg Fe/ml |
| | chloride | | monostearate (SPAN ® 60) | 5 nm oleic acid- |
| | | | 37 mg/ml polysorbate 80 | coated iron oxide |
| | | | (TWEEN ® 80) | nanoparticles |
| | | | | 10 mM sodium |
| | | | | citrate dihydrate. |
| NP-32 | 0.8-1.6 mg/ml | 4.5% squalene | 0.5% (w/w) sorbitan | 10 mM sodium citrate |
| | DOTAP chloride | | trioleate (SPAN 85 ®) | |
| | | | 0.5% (w/w) polysorbate 80 | |
| | | | (TWEEN ® 80) | |
| NP-33 | 45-55 mol % ionizable | 35-42 mol % cholesterol | 1.25-1.75 mol % | |
| | cationic lipid | | PEG2000-DMG | |
| | 8-12 mol % | | | |
| | distearoylphosphatidyl | | | |
| | choline (DSPC) | | | |
| NP-34 | 50 mol % D-Lin-MC3- | 38.5% cholesterol | 1.5% PEG-lipid | |
| | DMA (MC3) | | | |
| | 10 mol % | | | |
| | distearoylphosphatidyl | | | |
| | choline (DSPC) | | | |
| NP-35 | 50 mol % Lipid H | 38.5% cholesterol | 1.5 mol % | |
| | (SM-102) | | PEG2000-DMG | |
| | 10 mol % | | | |
| | distearoylphosphatidyl | | | |
| | choline (DSPC) | | | |
| NP-36 | 30 mg/ml 1,2- | 3.75% w/v glyceryl | 37 mg/ml sorbitan | 10 mM sodium |
| | dioleoyl-3- | trimyristate-dynasan | monostearate, (2R)-2- | citrate dihydrate |
| | trimethylammonium- | (DYNASAN 114 ®) | [(2R,3R,4S)-3,4- | |
| | propane (DOTAP) | | Dihydroxyoxolan-2-yl]- | |
| | chloride | | 2-hydroxyethyl | |
| | | | octadecenoate, C₂₄H₄₆O₆) | |
| | | | (SPAN ® 60) | |
| | | | 37 mg/ml polyoxyethylene | |
| | | | (20) sorbitan monooleate, | |
| | | | C₆₄H₁₂₄O₂₆ Polysorbate 80 | |
| | | | (TWEEN ® 80) | |
| NP-37 | 30 mg/ml 1,2- | 3.75% w/v glyceryl | 37 mg/ml sorbitan | 0.2 mgFe/mL or |
| | dioleoyl-3- | trimyristate-dynasan | monostearate, (2R)-2- | 0.02% wFe/v of 5 |
| | trimethylammonium- | (DYNASAN 114 ®) | [(2R,3R,4S)-3,4- | to 15 nm diameter |
| | propane (DOTAP) | | Dihydroxyoxolan-2-yl]- | iron oxide |
| | chloride | | 2-hydroxyethyl | nanoparticles |
| | | | octadecenoate, C₂₄H₄₆O₆) | 10 mM sodium |
| | | | (SPAN ® 60) | citrate dihydrate |
| | | | 37 mg/ml polyoxyethylene | |
| | | | (20) sorbitan monooleate, | |
| | | | C₆₄H₁₂₄O₂₆ Polysorbate 80 | |
| | | | (TWEEN ® 80) | |

In some embodiments, a composition described herein comprises at least two non-identical nanoparticles. In some embodiments, the at least two non-identical nanoparticles can have any one of the formulations of NP-1 to NP-37. In some embodiments, the at least two non-identical nanoparticles can have same nanoparticle formulation but different nucleic acid(s) complex to its surface forming a nucleic acid-nanoparticle complex(es). In some embodiments, the at least two non-identical nanoparticles can have same nucleic acids complex to their surfaces forming nucleic acid-nanoparticle complexes but different nanoparticle formulations.

In some embodiments, nanoparticles provided herein comprise: sorbitan monostearate (e.g., SPAN-60), polysorbate 80 (e.g., TWEEN-80), DOTAP, squalene, and no inorganic solid particles in the core. In some embodiments, nanoparticles provided herein comprise: sorbitan monostearate (e.g., SPAN-60), polysorbate 80 (e.g., TWEEN-80), DOTAP, squalene, and iron oxide particles. In some embodiments, nanoparticles provided herein comprise an immune stimulant. In some embodiments, the immune stimulant is squalene. In some embodiments, the immune stimulant is a medium chain triglyceride. In some embodiments, the immune stimulant is Miglyol 810 or Miglyol 812. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nm to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 200 nm in diameter. In some embodiments, the nanoparticle is 50 to 70 nm in diameter. In some embodiments, the nanoparticle is 40 to 80 nm in diameter. In some embodiments, the inorganic particle within the hydrophobic core of the nanoparticle can be an average diameter (number weighted average diameter) ranging from about 3 nm to about 50 nm. In some embodiments, the inorganic particle comprises an average diameter of about 5 nm, about 10 nm, about 15 nm, about 20 nm, about 25 nm, about 30 nm, about 35 nm, about 40 nm, about 45 nm, or about 50 nm.

Nanoparticles provided herein may be characterized by the polydispersity index (PDI), which is an indication of their quality with respect to size distribution. In some embodiments, the average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, the nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1. In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester. In some embodiments, nanoparticles provided herein comprise a ratio of the esters that yield a hydrophilic-lipophilic balance between 8 and 11. In some embodiments, nucleic acids provided herein are admixed with a lipid carrier provided herein to form a lipid carrier-nucleic acid complex. In some embodiments, the lipid carrier-nucleic acid complex is formed via non-covalent interactions or via reversible covalent interactions.

### Combination Compositions

Provided herein are compositions comprising a plurality of nanoparticles described herein and a plurality of nucleic acids encoding for a plurality of antigens. In some embodiments, the nanoparticles comprise one or more nucleic acids. In some embodiments, the nucleic acids encode one or more antigens. In some embodiments, the compositions induce multivalent immune response. In some embodiments, the nucleic acid further encodes for a self-replicating RNA polymerase. In some embodiments, the nucleic acid encoding the self-replicating RNA polymerase is on the same nucleic acid strand as the nucleic acid sequence encoding the protein (e.g., cis). In some embodiments, the nucleic acid encoding the self-replicating RNA polymerase is on a different nucleic acid strand as the nucleic acid sequence encoding the protein (e.g., trans). In some embodiments, the nucleic acid encoding the self-replicating RNA polymerase is a DNA molecule. In some embodiments, nucleic acid sequences encoding a protein provided herein are DNA or RNA molecules. In some embodiments, proteins provided herein are encoded by DNA. Nanoparticles for inclusion include, without limitation, any one of NP-1 to NP-37. Nucleic acids for inclusion include, without limitation, comprise a region encoding for any one of, or a plurality of, SEQ ID NOS: 1-9, 13 and/or SEQ ID NOS: 67-83. In some embodiments, nanoparticles for inclusion include one or more of NP-1 to NP-37. In some embodiments, nucleic acid for inclusion comprises a region encoding for one or more of SEQ ID NOS: 1-9, 13 and/or SEQ ID NOS: 67-83. In some instances, the nucleic acids further compromise a region encoding for an RNA polymerase, e.g., a region comprising a sequence of SEQ ID NO: 62.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. In some embodiments, the compositions provided herein comprise a N:P ratio of up to about 100:1, 150:1, or 200:1. In some embodiments, the compositions provided herein comprise a N:P ratio of 0.2:1 to 25:1. In some embodiments, the compositions provided herein comprise a N:P ratio of about 200:1, 150:1, 100:1, 80:1, 50:1, 40:1, 25:1, 15:1, 10:1, 8:1, 5:1, 1:1 or 0.2:1. In some embodiments, the compositions provided herein comprise a N:P ratio of up to about 200:1, 150:1, 100:1, 80:1, 50:1, 40:1, 25:1, 15:1, 10:1, 8:1, 5:1. In some embodiments, the compositions provided herein comprise a N:P ratio of at least about 150:1, 100:1, 80:1, 50:1, 40:1, 25:1, 15:1, 10:1, 8:1, 5:1, 1:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene:cationic lipid, hydrophobic surfactant, and hydrophilic surfactant. In some embodiments, the oil-to-surfactant ratio is the molar ratio of squalene:DOTAP, hydrophobic surfactant, and hydrophilic surfactant. In some embodiments, the oil-to-surfactant ratio is the molar ratio of squalene:DOTAP, sorbitan monostearate, and polysorbate 80. In some embodiments, the oil-to-surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to-surfactant molar ratio is at least about 0.4:1 up to 1:1.

Compositions provided herein can be characterized by hydrophilic surfactant-to-cationic lipid ratio. In some embodiments, the hydrophilic surfactant-to-cationic lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1. In some embodiments, the cationic lipid is DOTAP.

Further provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN-60), polysorbate 80 (e.g., TWEEN-80), DOTAP, an immune stimulant, and an RNA.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

### Pharmaceutical Compositions

Provided herein is a lyophilized composition comprising a composition provided herein. Further provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). In some embodiments, the pharmaceutical composition is formulated for inhalation. Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. Dosing may be for veterinary or human therapeutic uses. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human and non-human animal use. Exemplary amounts of total nucleic acid for incorporation in a composition described herein includes about 1, 2, 2.5, 5, 7.5, 10, 12.5, 15, 20, 25, 30, 35, 40, 45, 50 micrograms (μg) or more. In some embodiments, the composition comprises at least two non-identical nucleic acids. In some embodiments, each of the at least two non-identical nucleic acids have different effective dose level. Accordingly, in some embodiments, the composition comprises at least two non-identical nucleic acids at non-identical concentration.

### Administration

Provided herein are compositions and pharmaceutical compositions for administering to a subject in need thereof. In some embodiments, the subject is a human. In some embodiments, the subject is a non-human animal. Further provided herein are compositions and pharmaceutical compositions for veterinary and therapeutic use in non-human animals. Subjects include, without limitation, domesticated animals, farmed animals and insects (including without limitation pigs, cows, horses, donkeys, mules, buffalo, bison, goats, sheep, pigs, ducks, geese, chicken, turkey, fish, camels, alpacas, llamas, rabbits, zebu, deer, guinea pigs, yaks, ferrets, birds, hedgehogs, rodents, turtles, amphibians, bees), wild animals, as well as humans. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via an intratumoral, subcutaneous, intradermal, intramuscular, inhalation, intravenous, intraperitoneal, intracranial, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days or 56 days. In some embodiments, a composition or pharmaceutical composition provided herein is administered to the subject by two doses. In some embodiments, a second dose of a composition or pharmaceutical composition provided herein is administered about 28 days or 56 days after the first dose. In some embodiments, a third dose of a composition or pharmaceutical composition provided herein is administered to a subject.

In some embodiments, nanoparticles described herein can effectively deliver nucleic acids wherein at least one nucleic acid non-identical to the other nucleic acids. In some embodiments, the nucleic acids are RNA or DNA. In some embodiments, the nucleic acids are able to preserve in vivo activity (e.g., inducing immune response). In some embodiments, the nucleic acids are able to induce multivalent immune response in a subject. In some embodiments, the multivalent immune response results in generation of one or more antibodies in the subject. In some embodiments, at least one of the nucleic acids has a large therapeutic window. Accordingly, in some embodiments, at least one of the nucleic acids is administered at a different dose that the other nucleic acids. For example, a first nucleic acid can be present at a concentration of 0.5 μg and a second nucleic acid can be present at a concentration of 2 μg.

### Therapeutic Applications

Provided herein are methods of inducing a multivalent immune response. In some embodiments, the method is used for treating and/or preventing a disease in a subject. In some embodiments, the composition described herein comprises a plurality of nucleic acid, wherein at least two of the plurality of nucleic acid are encoding for different antigens. In some embodiments, the antigen is a viral antigen described herein or a variant thereof in some embodiments, the antigen is a bacterial antigen described herein or a variant thereof. In some embodiments, the antigen is a tumor antigen described herein or a variant thereof.

Provided herein are methods of treating or preventing a disease in a subject. In some embodiments, compositions described herein are used for the treatment of an infection. In some embodiments, the infection is a viral infection. In some embodiments, the viral infection is from a Coronavirus. In some embodiments, the Coronavirus is SARS-CoV-2. In some embodiments, the Coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a Respiratory syncytial virus (RSV). In some embodiments, the virus is an enterovirus, e.g., EVD68.

In some embodiments, compositions described herein are used for enhancing the immune response of a subject to a viral antigen or a tumor antigen provided herein or a variant thereof. In some embodiments, compositions described herein are used for immunizing a subject. In some embodiments, compositions described herein are used for the reduction of severity of an infection in a subject. In some embodiments, compositions described herein provide for reduction of severity or duration of symptoms associated with an infection in a subject. In some embodiments, the infection is a viral infection. In some embodiments, the viral infection is from a Coronavirus. In some embodiments, the Coronavirus is SARS-CoV-2. In some embodiments, administration of a composition describes herein provides for reduction in the severity or duration of COVID-19 symptoms in a subject. In some embodiments, the Coronavirus is MERS or SARS. In some embodiments, the viral infection is from an influenza virus. In some embodiments, the influenza virus is influenza A or influenza B. In some embodiments, the viral infection is from a Zika virus. In some embodiments, the viral infection is from a Respiratory syncytial virus (RSV). In some embodiments, the virus is EVD68.

In some embodiments, compositions described herein are used for the treatment of a cancer. In some embodiments, the cancer is a solid cancer or a hematopoietic cancer. In some embodiments, the solid cancer is a melanoma, lung, liver, head and neck, or pancreatic cancer. In some embodiments, the solid cancer is a melanoma cancer. In some embodiments, a composition described herein is used for reduction of a tumor size. In some embodiments, a composition described herein is used for reduction of a tumor volume. In some embodiments, a composition described herein is used for reduction of a cancer recurrence. In some embodiments, a composition described herein is used for reduction of tumor metastasis.

### Exemplary Embodiments

Provided herein are compositions, wherein the compositions comprise: a plurality of nanoparticles, wherein each nanoparticle comprises a surface; and a plurality of nucleic acids, wherein each nucleic acid is complexed to the surface to form a nucleic acid-nanoparticle complex. In some embodiments, the nucleic acid comprises a sequence encoding a viral antigen. In some embodiments, the nucleic acid comprises an RNA or a DNA. In some embodiments, the plurality of nucleic acids, each comprising a sequence encoding a viral antigen.

Provided herein are compositions, wherein the compositions comprise: a plurality of nanoparticles, wherein each of the nanoparticles comprises a surface; and a plurality of nucleic acids, wherein each nucleic acid comprises a sequence encoding an RNA polymerase and a viral antigen, wherein the plurality of nucleic acids encode for a plurality of viral antigens, and wherein the plurality of nucleic acids are complexed to the surface.

Provided herein are compositions, wherein the compositions comprise: a plurality of nanoparticles, wherein each of the nanoparticles comprises a surface; and a plurality of nucleic acids, wherein each nucleic acid comprises a sequence encoding an RNA polymerase and a tumor antigen, wherein the plurality of nucleic acids encode for a plurality of tumor antigens, and wherein the plurality of nucleic acids are complexed to the surface.

Provided herein are compositions, wherein the compositions comprise: a plurality of nanoparticles, wherein each nanoparticle comprises a surface; and a plurality of nucleic acids, wherein the plurality of nucleic acids comprise (i) nucleic acids encoding for an RNA polymerase and (ii) nucleic acids encoding for a plurality of antigens, and wherein the plurality of nucleic acids are complexed to the surface, optionally wherein the antigens are viral antigens or tumor antigens. Provided herein are compositions, wherein the compositions comprise: a plurality of nanoparticles, wherein each of the nanoparticles comprises a surface; and a plurality of nucleic acids, wherein the plurality of nucleic acids comprises a sequence encoding an RNA polymerase and a viral antigen, wherein the plurality of nucleic acids encode for a plurality of viral antigens, and wherein the plurality of nucleic acids are complexed to the surface. Further provided herein are compositions wherein the plurality of nucleic acids comprise RNA or DNA. Further provided herein are compositions wherein the plurality of nucleic acids comprise sequences encoding viral antigens of the same viral species. Further provided herein are compositions wherein the plurality of nucleic acids comprise sequences encoding two or more coronavirus spike (S) proteins or fragments thereof. Further provided herein are compositions wherein the plurality of nucleic acids comprise sequences encoding viral antigens from different viral species. Further provided herein are compositions wherein the viral antigen is derived from a coronavirus, an enterovirus, an influenza A virus, an influenza B virus, a respiratory syncytial virus (RSV) or a zika virus.

Further provided herein are compositions wherein the plurality of nucleic acids comprise sequences encoding one or more of (a) a coronavirus spike protein, (b) an influenza virus hemagglutinin protein, (c) an RSV glycoprotein (G), (d) an RSV fusion (F) protein, or a variant thereof. Further provided herein are compositions wherein the plurality of nucleic acids comprise sequences encoding two or more of: (a) a coronavirus spike protein; (b) an influenza virus hemagglutinin protein; (c) an RSV glycoprotein (G); or (d) an RSV fusion (F) protein, or a variant thereof. Further provided herein are compositions wherein the plurality of nucleic acids comprise sequences encoding three or more of: (a) a coronavirus spike protein; (b) an influenza virus hemagglutinin protein; (c) an RSV glycoprotein (G); or (d) an RSV fusion (F) protein, or a variant thereof. Further provided herein are compositions wherein the plurality of nucleic acids comprise sequences encoding: (a) a coronavirus spike protein; (b) an influenza virus hemagglutinin protein; (c) an RSV glycoprotein (G); and (d) an RSV-F protein. Further provided herein are compositions wherein the coronavirus spike protein is a SARS-CoV-2 spike protein. Further provided herein are compositions wherein the SARS-CoV-2 spike protein is derived from the alpha variant of SARS-CoV-2. Further provided herein are compositions wherein the SARS-CoV-2 spike protein is derived from the beta variant of SARS-CoV-2. Further provided herein are compositions wherein the SARS-CoV-2 spike protein is derived from the delta variant of SARS-CoV-2. Further provided herein are compositions wherein the SARS-CoV-2 spike protein is derived from the mu variant of SARS-CoV-2. Further provided herein are compositions wherein the SARS-CoV-2 spike protein is derived from a variant of SARS-CoV-2 comprising an amino acid substitution of D614G or D627G. Further provided herein are compositions wherein the one or more of the plurality of nucleic acids comprise a sequence set forth in any one of SEQ ID NOS: 1-7, 67-73. Further provided herein are compositions wherein the influenza virus hemagglutinin protein is hemagglutinin H2, hemagglutinin H3, hemagglutinin H5, hemagglutinin H6, hemagglutinin H7, hemagglutinin H8, or hemagglutinin H9. Further provided herein are compositions wherein the influenza virus hemagglutinin protein has a nucleic acid sequence set forth in any one of SEQ ID NO: 13 or SEQ ID NO: 76 or encodes an amino acid sequence of any one of SEQ ID NOS: 14-15. Further provided herein are compositions wherein the RSV-G has a nucleic acid sequence set forth in SEQ ID NO: 9 or encodes an amino acid sequence of any one of SEQ ID NOS: 11-12. Further provided herein are compositions wherein the RSV-F has a nucleic acid sequence set forth in SEQ ID NO: 8 or SEQ ID NO: 74. Further provided herein are compositions wherein the nucleic acid sequence encodes for an RSV-F antigen comprising an amino acid sequence set forth in SEQ ID NO: 10 or SEQ ID NO: 11. Further provided herein are compositions wherein the RNA polymerase is a self-replicating RNA polymerase. Further provided herein are compositions wherein the self-replicating RNA polymerase is under that control of an internal ribosome entry side (IRES). Further provided herein are compositions wherein the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase. Further provided herein are compositions wherein the RNA polymerase is Venezuelan equine encephalitis virus (VEEV) RNA polymerase. Further provided herein are compositions wherein the nucleic acid encoding the RNA polymerase comprises the nucleic acid sequence of SEQ ID NO: 62. Further provided herein are compositions wherein the nanoparticle is up to 200 nm in diameter. Further provided herein are compositions wherein the nanoparticle is 50 to 70 nm in diameter. Further provided herein are compositions wherein the nanoparticle is 40 to 80 nm in diameter. Further provided herein are compositions wherein the plurality of nanoparticles comprise a cationic lipid. Further provided herein are compositions wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″,9″′″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions wherein the plurality of nanoparticles each comprise a hydrophobic core. Further provided herein are compositions wherein the hydrophobic core comprises a lipid, optionally an oil. Further provided herein are compositions wherein the oil is in liquid phase. Further provided herein are compositions wherein the oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerin. Further provided herein are compositions wherein the plurality of nanoparticles each comprise an inorganic particle. Further provided herein are compositions wherein the inorganic particle is within the hydrophobic core of the nanoparticle. Further provided herein are compositions wherein the inorganic particle comprises a metal. Further provided herein are compositions wherein the metal comprises a metal salt, a metal oxide, a metal hydroxide, or a metal phosphate. Further provided herein are compositions wherein the metal oxide comprises aluminum oxide, aluminum oxyhydroxide, iron oxide, titanium dioxide, or silicon dioxide. Further provided herein are compositions wherein the plurality of nanoparticles each comprise a cationic lipid, an oil, and an inorganic particle. Further provided herein are compositions wherein the plurality of nanoparticles further comprise a surfactant. Further provided herein are compositions wherein the surfactant is a hydrophobic surfactant. Further provided herein are compositions wherein the hydrophobic surfactant is sorbitan monolaurate, sorbitan monopalmitate, sorbitan monostearate, sorbitan monooleate, or sorbitan trioleate. Further provided herein are compositions wherein the surfactant is a hydrophilic surfactant. Further provided herein are compositions wherein the hydrophilic surfactant is a polysorbate. Further provided herein are compositions wherein the plurality of nanoparticles each comprise a cationic lipid, an oil, an inorganic particle, and a surfactant. Further provided herein are compositions wherein the plurality of nanoparticles do not comprise trimyristin. Further provided herein are compositions wherein the composition is lyophilized. Further provided herein are compositions wherein the composition is a suspension. Further provided herein are compositions wherein the suspension is a homogeneous suspension. Provided herein are compositions, wherein the compositions comprise: a plurality of nanoparticles, wherein each of the nanoparticles comprises a surface; and a plurality of nucleic acids, wherein the plurality of nucleic acids comprises a sequence encoding an RNA polymerase and a tumor antigen, wherein the plurality of nucleic acids encode for a plurality of tumor antigens, and wherein the plurality of nucleic acids are complexed to the surface. Further provided herein are compositions wherein the plurality of nucleic acids comprise RNA or DNA. Further provided herein are compositions wherein the plurality of nucleic acids encode for a tumor antigen selected from epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Esol), Malignant melanoma (MAGE), MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A1, MART-1/Melan-A, Tyrosinase, tyrosinase related protein 1 (TRYP-1), glycoprotein 100 (GP100), breast cancer WT1, Herceptin, lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), or programmed cell death protein (PD-1). Further provided herein are compositions wherein the RNA polymerase is a self-replicating RNA polymerase. Further provided herein are compositions wherein the self-replicating RNA polymerase is under that control of an internal ribosome entry side (IRES). Further provided herein are compositions wherein the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase. Further provided herein are compositions wherein the RNA polymerase is Venezuelan equine encephalitis virus (VEEV) RNA polymerase. Further provided herein are compositions wherein the nucleic acid encoding the RNA polymerase comprises the nucleic acid sequence of SEQ ID NO: 62. Further provided herein are compositions wherein the nanoparticle is up to 200 nm in diameter. Further provided herein are compositions wherein the nanoparticle is 50 to 70 nm in diameter. Further provided herein are compositions wherein the nanoparticle is 40 to 80 nm in diameter. Further provided herein are compositions wherein the plurality of nanoparticles each comprise a cationic lipid. Further provided herein are compositions wherein the cationic lipid is 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-TH-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″^{,9″″} ,9′″″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12Z,12″Z,12′″Z)-tetrakis (octadeca-9,12-dienoate); PEG2000-DMG, (R)-2,3-bis(myristoyloxy)propyl-1-(methoxy poly(ethylene glycol)₂₀₀₀) carbamate; TT3, or N1,N3,N5-tris(3-(didodecylamino)propyl)benzene-1,3,5-tricarboxamide. Further provided herein are compositions wherein the plurality of nanoparticles each comprise a hydrophobic core. Further provided herein are compositions wherein the hydrophobic core comprises an oil. Further provided herein are compositions wherein the oil is in liquid phase. Further provided herein are compositions wherein the oil is α-tocopherol, coconut oil, grapeseed oil, lauroyl polyoxylglyceride, mineral oil, monoacylglycerol, palmkernal oil, olive oil, paraffin oil, peanut oil, propolis, squalene, squalane, solanesol, soy lecithin, soybean oil, sunflower oil, a triglyceride, or vitamin E. Further provided herein are compositions wherein the triglyceride is capric triglyceride, caprylic triglyceride, a caprylic and capric triglyceride, a triglyceride ester, or myristic acid triglycerin. Further provided herein are compositions wherein the plurality of nanoparticles each comprise an inorganic particle. Further provided herein are compositions wherein the inorganic particle is within the hydrophobic core of the nanoparticle. Further provided herein are compositions wherein the inorganic particle comprises a metal. Further provided herein are compositions wherein the metal comprises a metal salt, a metal oxide, a metal hydroxide, or a metal phosphate. Further provided herein are compositions wherein the metal oxide comprises aluminum oxide, aluminum oxyhydroxide, iron oxide, titanium dioxide, or silicon dioxide. Further provided herein are compositions wherein the plurality of nanoparticles each comprise a cationic lipid, an oil, and an inorganic particle. Further provided herein are compositions wherein the plurality of nanoparticles further comprise a surfactant. Further provided herein are compositions wherein the surfactant is a hydrophobic surfactant. Further provided herein are compositions wherein the hydrophobic surfactant is sorbitan monolaurate, sorbitan monopalmitate, sorbitan monostearate, sorbitan monooleate, or sorbitan trioleate. Further provided herein are compositions wherein the surfactant is a hydrophilic surfactant. Further provided herein are compositions wherein the hydrophilic surfactant is a polysorbate. Further provided herein are compositions wherein the plurality of nanoparticles each comprise a cationic lipid, an oil, an inorganic particle, and a surfactant. Further provided herein are compositions wherein the plurality of nanoparticles do not comprise trimyristin. Further provided herein are compositions wherein the composition is lyophilized. Further provided herein are compositions wherein the composition is a suspension. Further provided herein are compositions wherein the suspension is a homogeneous suspension. Provided here are dried compositions comprising: a lipid carrier, wherein the lipid carrier is a nanoemulsion comprising a hydrophobic core, and one or more lipids; one or more nucleic acid; and at least one sugar present in amount of (i) at least about 50% by weight of the dried composition, or (ii) present in an amount of least 50 mg. Further provided herein are compositions wherein the composition is lyophilized. Further provided herein are compositions wherein the composition is thermally stable at about 25 degrees Celsius. Further provided herein are compositions wherein the composition is thermally stable at about 45 degrees Celsius. Further provided herein are compositions wherein the composition is thermally stable at about −20 degrees Celsius. Further provided herein are compositions wherein the composition is thermally stable at about 2 degrees Celsius to about 8 degrees Celsius. Further provided herein are compositions wherein the composition is thermally stable for at least 1 week, at least 2 weeks, and/or at least 1 month. Further provided herein are compositions wherein the hydrophobic core comprises an oil. Further provided herein are compositions wherein the oil comprises at least one of α-tocopherol, lauroyl polyoxylglyceride, monoacylglycerol, propolis, squalene, mineral oil, grapeseed oil, olive oil, paraffin oil, peanut oil, soybean oil, sunflower oil, soy lecithin, triglyceride, vitamin E, a caprylic/capric triglyceride, a triglyceride ester of saturated coconut/palmkernel oil derived caprylic and capric fatty acids and plant derived glycerol, dihydroisosqualene (DHIS), famesene and squalane. Further provided herein are compositions wherein the one or more lipids is selected from the group consisting of cationic lipids, anionic lipids, neutral lipids, and any combinations thereof. Further provided herein are compositions wherein the one or more lipids comprises a cationic lipid. Further provided herein are compositions wherein the cationic lipid is selected from the group consisting of 1,2-dioleoyloxy-3-(trimethylammonium)propane (DOTAP); 3β-[N-(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,Ntrimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLin-DMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200), 1,2-dioleoyl-sn-glycero-3-phosphoethanolamine (DOPE); N-decyl-N,N-dimethyldecan-1-aminium bromide (DDAB); 2,3-dioleyloxy-N-[2-(sperminecarboxamido)ethyl]-N,N-dimethyl-1-propanaminium trifluoroacetate (DOSPA); ethylphosphatidylcholine (ePC); and any combinations thereof. Further provided herein are compositions wherein the lipid carrier comprises at least one surfactant. Further provided herein are compositions wherein the at least one surfactant is selected from the group consisting of a hydrophobic surfactant, a hydrophilic surfactant, and any combinations thereof. Further provided herein are compositions wherein the hydrophobic surfactant comprises a sorbitan ester selected from the group consisting of sorbitan monolaurate, sorbitan monopalmitate, sorbitan monostearate, sorbitan tristearate, sorbitan monooleate, and sorbitan trioleate; and the hydrophilic surfactant comprises a polysorbate. Further provided herein are compositions wherein the lipid carrier has a z-average hydrodynamic diameter ranging from about 40 nm to about 150 nm, with an average polydispersity index ranging from about 0.1 to about 0.4. Further provided herein are compositions wherein the one or more nucleic acid is a DNA. Further provided herein are compositions wherein the one or more nucleic acid is a RNA. Further provided herein are compositions wherein the RNA is a self-replicating RNA. Further provided herein, are compositions wherein the hydrophobic core comprises one or more inorganic nanoparticles. Further provided herein are compositions, wherein the one or more inorganic nanoparticles is selected from the group consisting of a metal salt, metal oxide, metal hydroxide, metal phosphate, and any combinations thereof. Further provided herein are compositions wherein the one or more nucleic acid is incorporated or complexed with the lipid carrier to form a lipid carrier-nucleic acid complex. Further provided herein are compositions wherein the lipid carrier-nucleic acid complex is formed via non-covalent interactions or via reversible covalent interactions. Further provided herein are compositions wherein a molar ratio of the lipid carrier to the one or more nucleic acids, characterized by the nitrogen-to-phosphate (N:P) molar ratio, ranges from about 1:1 to about 150:1. Further provided herein are compositions wherein the at least one sugar is selected from the group consisting of sucrose, maltose, trehalose, mannitol, glucose, and any combinations thereof. Further provided herein are compositions wherein the at least one sugar is present in an amount of at least about 50, 60, 70, 80, 90, 100, 110, 120, 130, 140, 150, 160, 170, 180, 190, 200, 210, 220, 230, 240, 250, 260, 270, 280, 290, 300 or more mg. Further provided herein are compositions wherein the at least one sugar is present in an amount of 50 mg to 250 mg. Further provided herein are compositions wherein the at least one sugar is present in an amount of at least about 250 mg. Further provided herein are compositions wherein the sugar is present in amount of the composition by weight of at least 50, 55, 60, 65, 70, 75, 80, 85, 90, 95% or more. Further provided herein are compositions wherein the sugar is present in amount of the composition by weight of 80 to 98%, optionally 94 to 96%. Further provided herein are compositions wherein the sugar is present in amount of the composition by weight of about 95%. Further provided herein are compositions wherein the at least one sugar comprises sucrose. Further provided herein are pharmaceutical compositions, comprising a dried composition disclosed herein reconstituted in a suitable diluent and a pharmaceutically acceptable carrier. Further provided herein are pharmaceutical compositions wherein the diluent is aqueous. Further provided herein are pharmaceutical compositions wherein the diluent is water. Further provided herein are kits comprising a pharmaceutical composition described herein and a delivery system for administration to a subject. Further provided are methods for generating an immune response in a subject, comprising administering a therapeutically effective amount of the pharmaceutical composition described herein. Further provided are methods of treating or preventing a disease in a subject, comprising administering a therapeutically effective amount of the pharmaceutical composition described herein. Further provided herein are methods of imaging and/or tracking delivery of one or more nucleic acids in a subject, comprising administering a therapeutically effective amount of the pharmaceutical composition described herein. Provided herein are pharmaceutical compositions, wherein the pharmaceutical composition comprises: a composition provided herein and a pharmaceutically acceptable excipient.

Provided herein are methods, wherein the methods comprise: administering to a subject an effective amount of the composition provided herein or the pharmaceutical compositions provided herein, wherein the administering produces an immune response in the subject to one or more viral antigens. Further provided herein are methods wherein the viral antigen is a coronavirus spike protein, an influenza virus hemagglutinin protein, an RSV glycoprotein (G), an RSV fusion (F) protein, or a variant thereof. Further provided herein are methods wherein the administering is local administration or systemic administration. Further provided herein are methods wherein the administering is via intramuscular injection, intranasal administration, oral administration, subcutaneous administration, intratumoral administration, or intravenous injection. Further provided herein are methods wherein the subject is at risk of developing one or more infectious diseases. Further provided herein are methods wherein the infectious disease is enterovirus infection, Severe acute respiratory syndrome (SARS), COVID 19, the flu, or zika fever. Further provided herein are methods wherein when administered in an effective amount to the subject, the administering elicits antibody titers to at least on viral antigen equal to or greater than the composition administered to a subject without the plurality of nucleic acids comprising a sequence encoding an RNA polymerase.

Provided herein are methods, wherein the methods comprise: administering to a subject an effective amount of the composition provided herein or the pharmaceutical composition provided herein, wherein the administering produces an immune response in the subject to one or more tumor antigens. Further provided herein are methods wherein the tumor antigen is epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Esol), Malignant melanoma (MAGE), MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A1, MART-1/Melan-A, Tyrosinase, tyrosinase related protein 1 (TRYP-1), glycoprotein 100 (GP100), breast cancer WT1, Herceptin, lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), or programmed cell death protein (PD-1). Further provided herein are methods wherein the administering is local administration or systemic administration. Further provided herein are methods wherein the administering is via intramuscular injection, intranasal administration, oral administration, subcutaneous administration, intratumoral administration, or intravenous injection. Further provided herein are methods wherein the subject has a solid tumor or a blood cancer. Further provided herein are methods wherein the solid tumor is a carcinoma, a melanoma, or a sarcoma. Further provided herein are methods wherein the blood cancer is lymphoma or leukemia. Further provided herein are methods wherein the subject has lung cancer or melanoma. Further provided herein are methods wherein the lung cancer is adenocarcinoma, squamous cell carcinoma, small cell cancer or non-small cell cancer.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### Examples

### Example 1: Manufacture and Stability of Nanoparticle NP-1

Manufacture of NP-1. NP-1 particles comprise 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID), 0.2 mg Fe/ml 12 nm oleic acid-coated iron oxide nanoparticles (ImagionBio) and 10 mM sodium citrate dihydrate (Fisher Chemical). 1 ml of 20 mg Fe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized nanoparticle was terminally filtered with a 200 nm pore-size polyethersulfone (PES) filter and stored at 2-8 degrees C. Iron concentration was determined by ICP-OES. DOTAP and Squalene concentration were measured by RP-HPLC.

Manufacture of NP-3. NP-3 particles comprise 37.5 mg/ml Miglyol 812 N (IOI Oleo GmbH), 37 mg/ml Span® 60 (Millipore Sigma), 37 mg/ml Tween® 80 (Fisher Chemical), 30 mg/ml DOTAP chloride (LIPOID), 0.2 mg Fe/ml 15 nm oleic acid-coated iron oxide nanoparticles (ImagionBio) and 10 mM sodium citrate dihydrate (Fisher Chemical). 1 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65 degree c. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)-reached 40-80 nm with a 0.1-0.3 polydispersity index (PDI). The microfluidized nanoparticle was terminally filtered with a 200 nm pore-size polyethersulfone (PES) filter and stored at 2-8° C. Iron concentration was determined by ICP-OES. DOTAP concentration was measured by RP-HPLC.

Manufacture of NP-30. A lipid carrier without providing inorganic core particles in the core was generated having 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID) and 10 mM sodium citrate. To a 200 ml beaker 3.75 grams squalene, 3.7 grams span 60, and 3.0 grams DOTAP were added to produce the oil phase. The oil phase was sonicated for 45 minutes in a 65 degrees Celsius water bath. Separately, the aqueous phase was prepared by dissolving 19.5 grams Tween 80 in 500 ml of 10 mM sodium citrate buffer prepared in nuclease free water. 96 ml of the aqueous phase was transferred to a separate glass bottle and heated to 65 degrees Celsius for 30 minutes. The oil phase was mixed with the 96 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 μm diamond interaction chamber and an auxiliary H30Z-200 μm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized nanoparticle without inorganic core formulation was terminally filtered with a 200 nm pore-size polyethersulfone (PES) filter and stored at 2-8 degrees Celsius. DOTAP and Squalene concentration were measured by RP-HPLC.

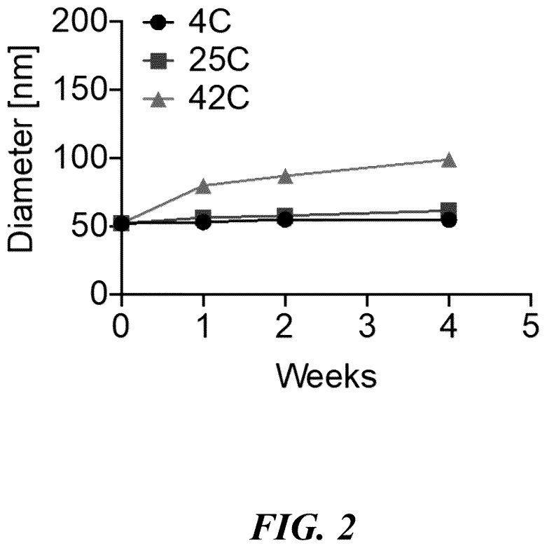

Stability. A nanoparticle according to NP-1 was placed into a stability chamber at the indicated temperatures. The stability was determined by particle size measurement using dynamic light scattering. The results show that the NP-1 formulation formed a stable colloid when stored at 4, 25 and 42 degrees Celsius. Time measurements were taken over 4 weeks. As shown in FIG. 2, the range of nanoparticle size was about 50-100 nm in diameter, and closer to 40-60 nm in diameter for the 4 and 25 degrees Celsius conditions over time.

### Example 2: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 62.

### Example 3: Immunogenicity and Specificity of a SARS-CoV-2 Spike Bivalent Vaccine

A plasmid encoding a VEEV replicon mRNA backbone and single and combination SARS-CoV-2 spike protein antigens were prepared. Each individual replicon was admixed with NP-1 according to Table 4.

TABLE 4
SARS-CoV-2 Spike Protein Antigens.

| | RNA 1 RNA dose | RNA 2 RNA dose | Total RNA dose |
|---|---|---|---|
| Groups | [μg] | [μg] | [μg] |
| 1 | 1 | — | 1 |
| 2 | — | 1 | 1 |
| 3 | 2 | — | 2 |
| 4 | — | 2 | 2 |
| 5 | 0.5 | 0.5 | 1 |
| 6 | 1 | 1 | 2 |
| 7 | 1 | 1 | 2 |
| Unimmunized | — | — | — |
| Control Group | | | |

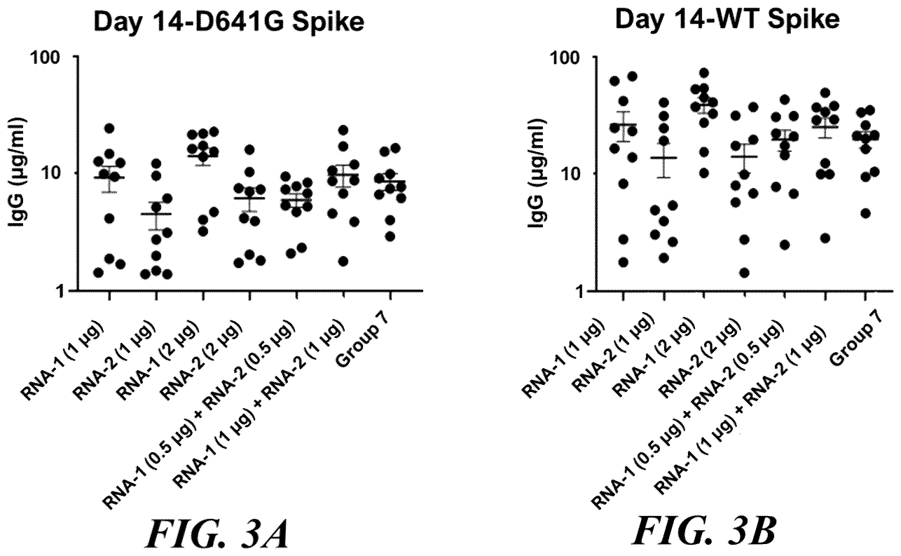

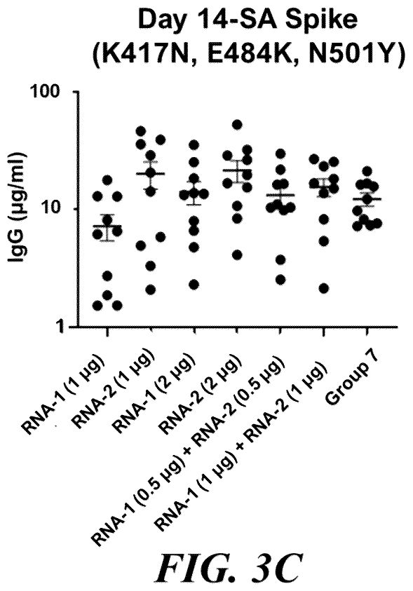

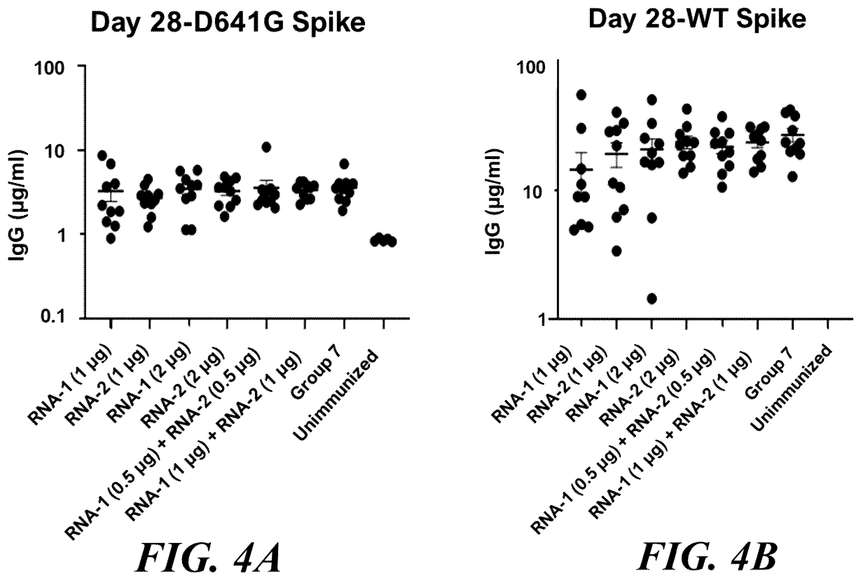

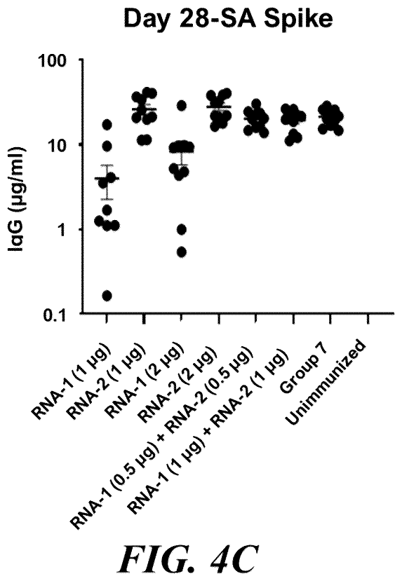

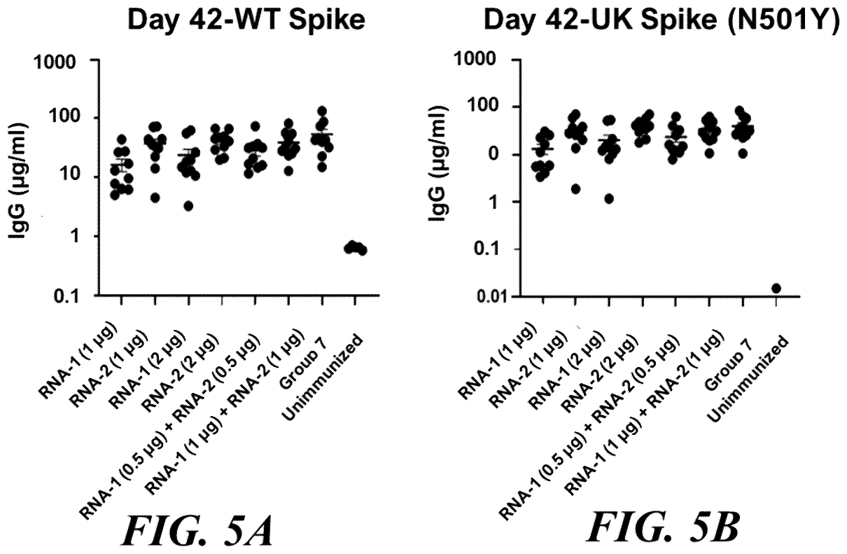

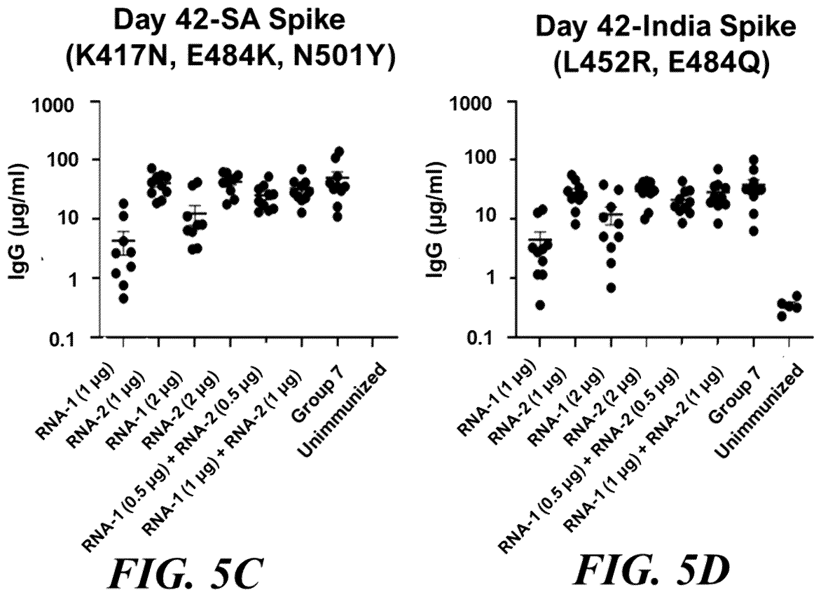

Female C57BL/6 mice were immunized by intramuscular injection with the antigens of Table 4. Blood samples were collected by retro-orbital eye bleed to determine serum antibody responses at 14 days (FIGS. 3A-3C), 28 days (FIGS. 4A-4C), and 42 days (FIGS. 5A-5D) post-injection. Serum IgG serum levels were measured by ELISA in response to wild-type SARS-CoV-2 spike protein (SEQ ID NO: 65) and spike protein variants including B.1.1.7 (also referred to as the United Kingdom or UK variant), B.1.351 (also referred to as the beta variant, South African, or SA variant), and B.1.617.2 (also referred to as the delta variant or India variant). SARS-CoV-2 variants and their sequences are summarized in Table 5.

TABLE 5
SARS-CoV-2 Spike Protein Variants and Amino Acid Sequences.

| | WHO | Spike Protein |
|---|---|---|
| Names | label | Mutations |
| Wild-Type (WT) surface glycoprotein | | — |
| or WT spike | | |
| United Kingdom or UK variant; B.1.1.7 | Alpha | N501Y |
| South Africa Variant or SA spike; | Beta | K417N, E484K, |
| “Triple Mutant”; B.1.351 | | N501Y |
| India Variant; B.1.617.2 | Delta | L452R, E484Q |
| D614G spike | | D614G |

By day 14, replicon RNA-1 rapidly induced responses against wild type RBD as compared to unimmunized animals. Replicon RNA-2 more rapidly induced responses against variant RBD containing E484K/Q mutations as compared to unimmunized animals. By day 28, responses induced by RNA-2 were equal to, or improved, over those elicited by RNA-1. RNA-2 induces more potent responses against spike variant RBD containing E484K/Q mutations (FIG. 5C).

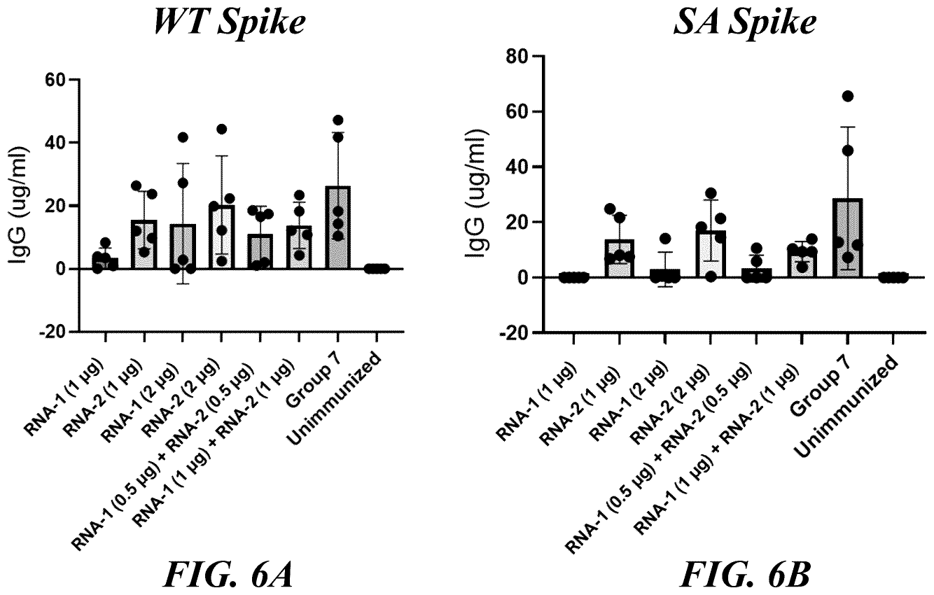

A prime boost immunization was also completed. Mice were immunized on days 0 and 28 by intramuscular injection with the repRNA+SARS-CoV-2 antigens of Table 4. At 105 days after completion of a prime-boost immunization regimen with repRNAs wild-type or SA variant spike proteins were introduced to the sample. Blood was collected and IgG serum levels were measured by ELISA at 133 days (FIGS. 6A-6B).

The combination of independently formulated replicons does not interfere with the immune responses (Group 6: RNA-1+RNA-2 replicons formulated individually with NP-1). Furthermore, the combination of replicons at the time of formulation also does not interfere with immune responses (Group 7: RNA-1+RNA-2 replicons formulated together with NP-1). The RNA-1+RNA-2 bivalent vaccine allowed for dose-sparing of each variant RNA and produced immune responses to wild-type and variant SARS-CoV-2 spike proteins.

### Example 4: Multivalent Respiratory Virus Vaccine Compositions

A plasmid encoding a VEEV replicon mRNA backbone and single and combination viral antigens were prepared. Each individual replicon was admixed with NP-1 in 1 microgram (μg) per dose according to Table 6. Combination compositions were prepared using 1 μg of each replicon per dose, then complexing the replicons with NP-1 as a single complexing event.

TABLE 6
Viral Antigens in Complex with NP-1.

| | | | RNA |
|---|---|---|---|
| | | | dose |
| | Antigen # | | [μg] in |
| Group | in FIGs. | Description | 50 μl |
| 1 | 645 | repRNA encoding RSV-G | 1 |
| 2 | 646 | repRNA encoding RSV (646; RSV-F) | 1 |
| 3 | 673 | repRNA encoding Pre-F B.1.1.7 | 1 |
| | | spike | |
| 4 | 671 | repRNA encoding Pre-F B.1.351 | 1 |
| | | spike | |
| 5 | 686 | Flu - H3 HA | 1 |
| 6 | 673 + | COVID bivalent: repRNA encoding | 1 each of |
| | 671 | Pre-F B.1.1.7 spike AND repRNA | 673 and |
| | | encoding Pre-F B.1.351 spike | 671 |
| 7 | 645 + | COMBO multivalent: repRNA | 1 each of |
| | 646 + | encoding RSV-G; | 645 + |
| | 673 + | repRNA encoding RSV (646; RSV-F); | 646 + |
| | 671 + | AND repRNA encoding Pre-F B.1.1.7 | 673 + |
| | 686 | spike AND | 671 + |
| | | repRNA encoding Pre-F B.1.351 | 686 |
| | | spike Flu - H3 HA | |

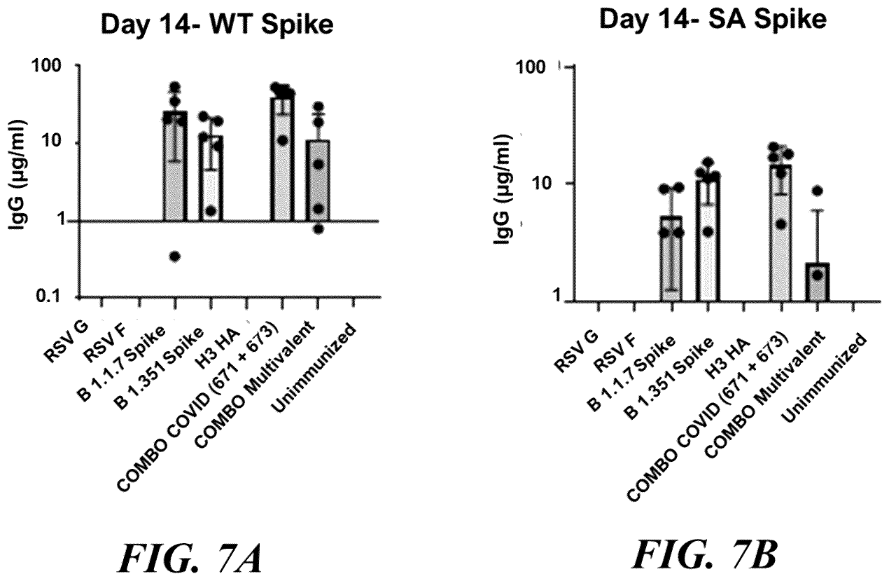

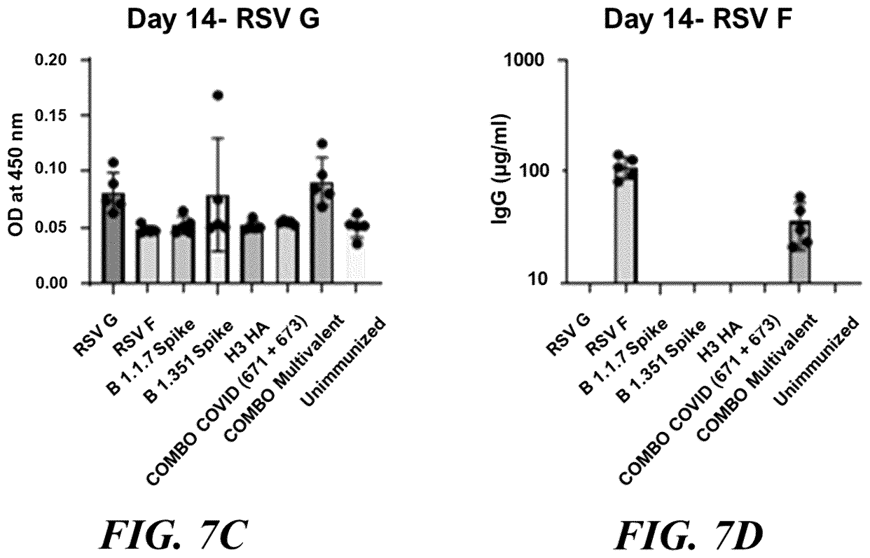

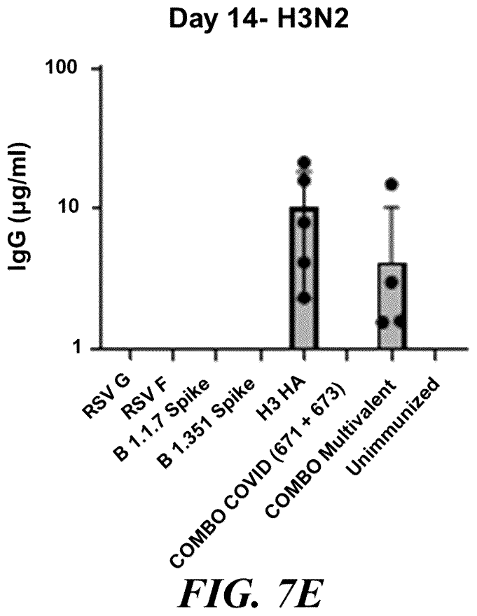

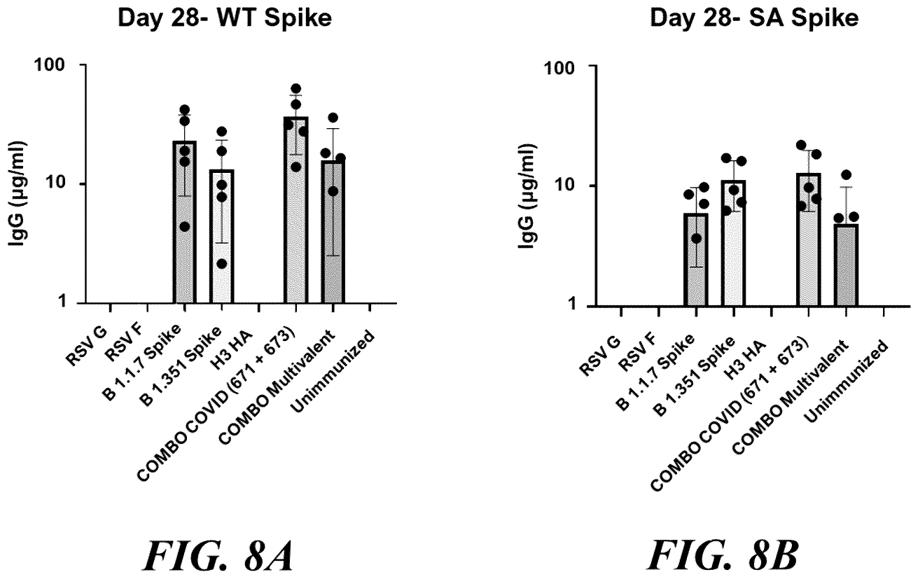

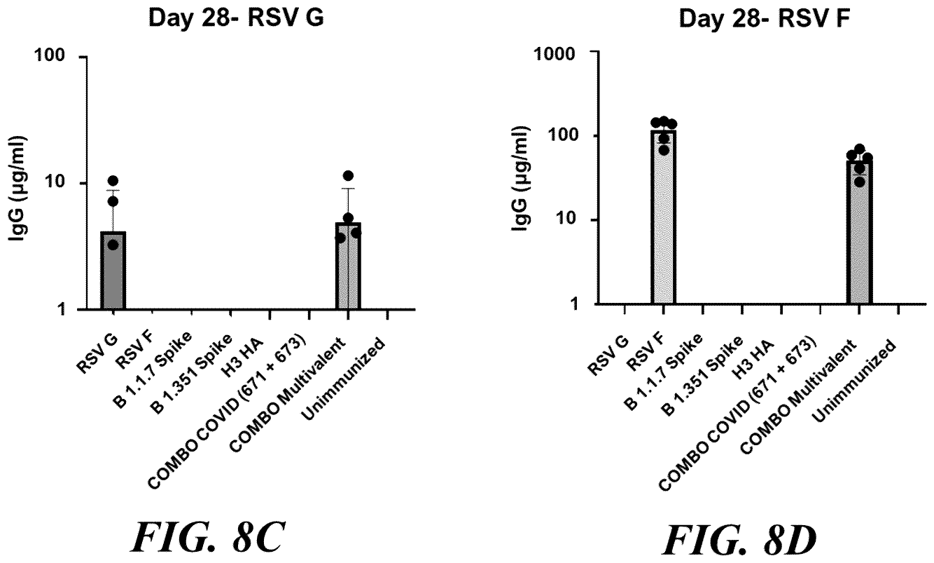

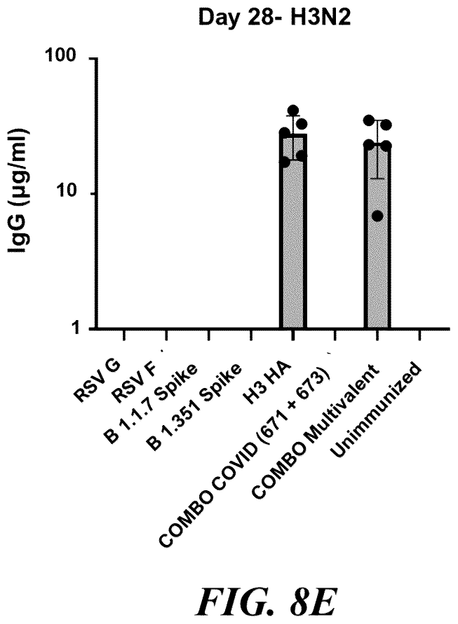

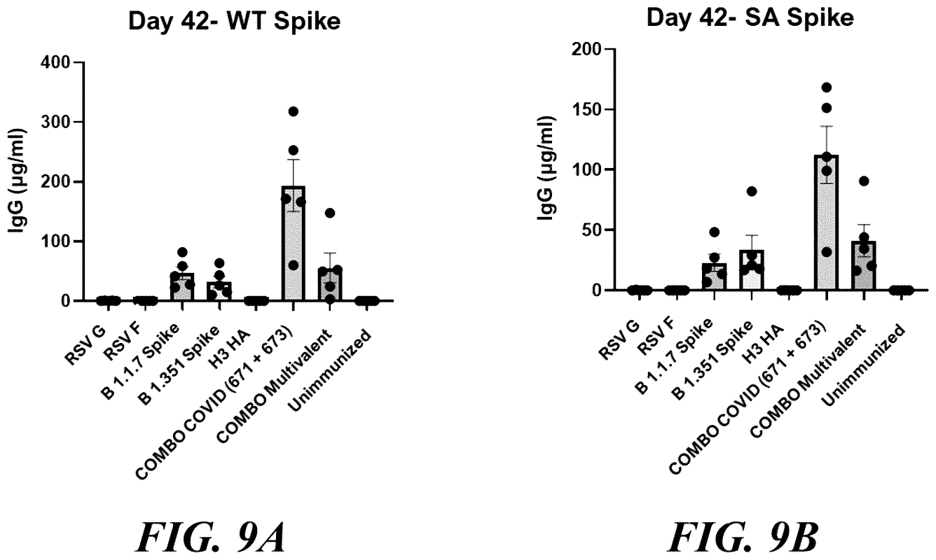

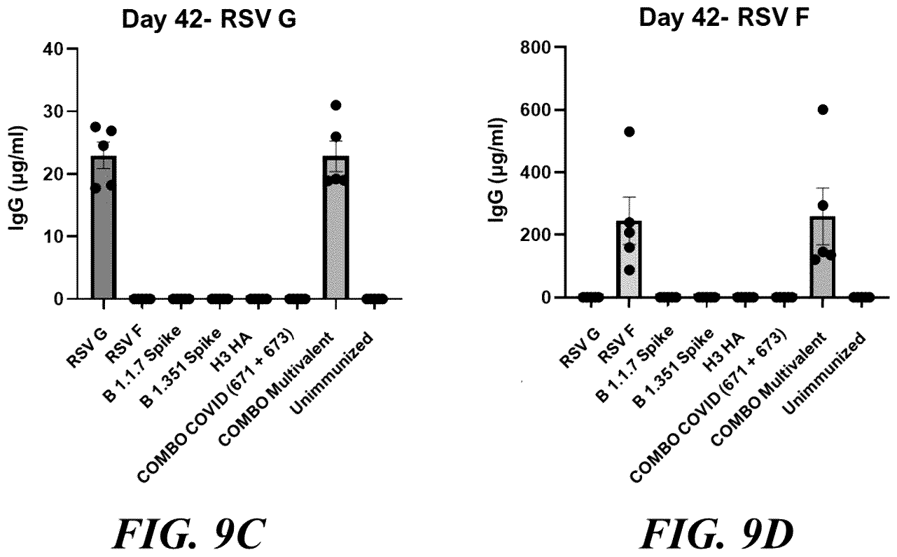

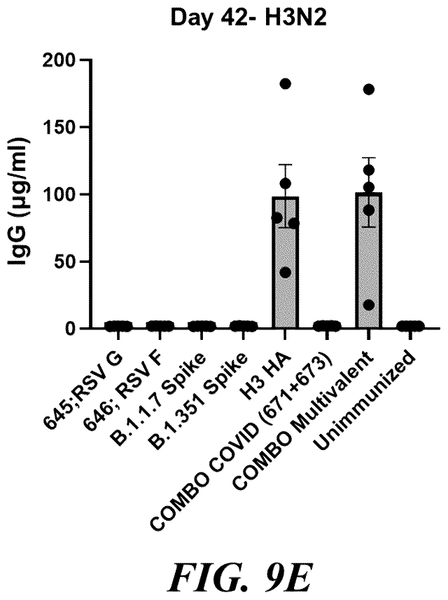

Female BALB/c mice were immunized by intramuscular injection with the antigens of Table 6 on day 0 and day 28. Serum IgG response in mice was determined 14 day after immunization with either 1 mcg single replicon, a COVID combination of 2 replicons (each at 1 mcg in a total of 2 mcg injection) or a 5 mcg combination of 5 replicons (each at 1 mcg in a total 5 meg injection) were determined by ELISA (FIGS. 7A-7E). IgG response was measured against WT SARS-CoV2 spike and SA variant spike proteins (FIGS. 7A and 7, respectively). IgG response was measured against RSV-G and RSV-F (FIGS. 7C and 7D), respectively) and influenza H3N2 (FIG. 7E). Serum IgG response to coated antigens were determined at 28 days post-injection (FIG. 8A-8E), and 42 days post injection (FIGS. 9A-9E) with the antigens of Table 6. Each replicon generated matched antigen-specific antibody responses when complexed with NP-i. Combinations of NP-i and replicons generated responses to each replicon component, comparable to the response generated by a single replicon. An analysis of FIGS. 7A-7E, 8A-8E and 9A-9E indicates that nanoparticles can be used to effectively deliver multiple RNAs with preserved in vivo activity. Additionally, a large therapeutic window also allows for multiple repRNA molecules to be combined at effective dosage levels.

### Example 5: Additional Nanoparticle Formulations

Additional nanoparticle formulations are produced according to the following tables (Table 7 and Table 8).

TABLE 7
mRNA Formulation.
Dosage form:
Liquid solution
Composition:

| | | Concentration |
|---|---|---|
| Each 0.5 ml Vial Contains: | Quantity | (mg/ml) |

| | | | |
|---|---|---|---|
| mRNA | 25 | mcg | 0.05 |
| DOTAP | 0.75 | mg | 1.5 |
| Iron Oxide Nanoparticles | 0.005 | mg | 0.01 |
| Squalene | 0.94 | mg | 1.88 |
| Sorbitan Monostearate | 0.93 | mg | 1.86 |
| Polysorbate 80 | 0.93 | mg | 1.86 |
| Sucrose IP | 50 | mg | 100 |
| Citric Acid Monohydrate | 1.05 | mg | 2.1 |

| | |
|---|---|
| Water for Injection | q.s. to 0.5 ml |

TABLE 8
Lyophilized mRNA Formulation.

Dosage form:
Lyophilized powder

Composition:

| Each 5 dose | | | Concentration | Approximate |
|---|---|---|---|---|
| vial contains: | Quantity | | (mg/ml) | dry weight % |
| mRNA | 50 | mcg | 0.02 | 0.02 |
| DOTAP | 1.5 | mg | 0.6 | 0.57 |
| Squalene | 1.88 | mg | 0.752 | 0.72 |
| Sorbitan | 1.86 | mg | 0.744 | 0.71 |
| Monostearate | | | | |
| Polysorbate 80 | 1.86 | mg | 0.744 | 0.71 |
| Sucrose IP | 250 | mg | 100 | 95.3 |
| Citric Acid | 5.25 | mg | 2.1 | 2 |
| Monohydrate | | | | |

| | |
|---|---|
| Water for Injection | 2.5 ml |
| (for reconstitution) | |

### Example 6: Immunogenicity of Monovalent and Trivalent EV-D68 Vaccines Formulated with NP-30 or Lipid Nanoparticles (LNPs)

A plasmid encoding a VEEV replicon mRNA backbone and single and combination viral antigens were prepared. Four formulations were prepared: (1) an NP-30-formulated monovalent EV-D68 vaccine (SEQ ID NO: 84); (2) a lipid nanoparticle (LNP) formulated monovalent EV-D68 vaccine (SEQ ID NO: 84); (3) an NP-30-formulated trivalent vaccine; and (4) an LNP-formulated trivalent vaccine. Briefly, the LNP was prepared using lipid components dissolved in ethanol at a ratio of 50:10:38:2 (Ionizable lipid (SM-102):Helper Lipid (DSPC):Cholesterol:DMG-PEG 2000) and mixed with RNA buffer at pH 4.5 at an N:P 5.5. The NP-30 and LNP trivalent vaccines were formulated using the antigen RNA sequence for EV-D68 virus-like particle (VLP) (SEQ ID NO: 84), RSV-F (SEQ ID NO: 8), and Influenza virus H3 antigen (SEQ ID NO: 13).

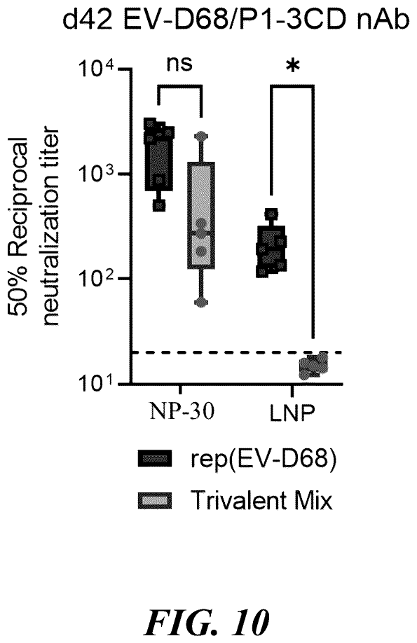

C57BL/6 mice were primed and boosted 28 days apart via intramuscular injection with the vaccine. The EV-D68 vaccine was administered at a dose of 3.3 μg. The trivalent vaccine was administered at a dose of 10 μg. On day 42, animals were bled and EV-D68 neutralizing antibody responses were assayed. The response was calculated as 50% reciprocal neutralization titer which refers to reciprocal dilution of serum required to inhibit viral infection by 50%. FIG. 10 shows that the NP-30-formulated trivalent vaccine produced superior response in neutralization titer relative to the LNP formulations. Furthermore, the NP-30-formulated trivalent vaccine produced a similar response in neutralization as NP-30-formulated EV-D68 vaccine.

SEQUENCES

SEQ ID NO: 1 (Delta V5 Antigen Encoding RNA Sequence)
auguuucugcucacaaccaaacgcacuauguuuuuuuccucgugcugcucccuuugguaagccucucaguguguaa
accugacaacacgaacccaguugccuccagcuuauaccaacucauuuacucgcggaguauauuaucccgauaaggu
cuuuagaaguagcguguugcacucuacacaggaucuguuuugcccuuuuuaguaccccccccacguauuuucauca
auacaugugagcggaacaaauggaacaaaaagauuugacaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauucgacuuucg
aauacgugucccaaccauuccucauggaccuugagggcaaacaggggaauuuuaaaaacuugcccgcgaauuuuuu
uaagaauaucgacggauacuuuaagaucuauaguaaacacacuccuaucaaccucguucgggaucuuccccaaggc
uuuucugcucucgaaccccucguagacuugccaauugggauaaauaucacucgcuuucaaaccuuuguugcccucc
acaggagcuaccugacacccggcgacucuucuucugguuggaccgccggggcugccccccccccuuuuuuuuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccaccaaacucaaugaucccuuuguucaccaagucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaggcaggcaguacuccuuguaacggcuuugagggauuuaacugcuauuuuccuuugcaauccuauggcuuuca
accaacaaacgggguuggcuaucaacccuaucgagugguuguccugagcuuugaacuuuugcacgcuccccccaca
gucugcggaccaaaaaagaguacaaaucuugucaagaauaagugcguaaauuucaauuucaauggccuuacaggaa
caggcgugcugacugagucaaacaagaaguuccugccauuucagcaguuuggggggacuauagcagacacaacuga
cgcuguacgcgauccucagacuuuggagaucuuggacaucacucccuguuuuucccgggggguccaucugucauca
cccggaacuaauacaucaaaucaggucgcuguguuguaccaagaugucaacugcacagaaguccccguugcuauac
acgcagaccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugcccuacacuaugucauugggg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggaucuuuggggcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccauccucuucaguacuaaugau
auuuugagccgauuggacaaaguggaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuacccccaugguacaauuuuuucacca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccaugguacauauggcuuggcuuuaucgcuggcccuuaugccaucguaaugguuacaauca
ugcugugcugcaugaccuccugcuguucuuguuugaaaggguguuguucuugugguaguuguugcaaguuugacga
agaugauuccgaaccuguucuuaaagggguaaagcuucacuauaca
SEQ ID NO: 2 (K995P-V996P Antigen Encoding RNA Sequence)
auguuucugcucacaaccaaacgcacuauguuuuuuuccucgugcugcucccuuugguaagucucagccuguguaa
accugacaacacgaacccaguugccuccagcuuauaccaacucauuuacucgcggaguauauuaucccgauaaggu
cuuuagaaguagcguguugcacucuacacaggaucuguuuugcccuuuuuaguaacguauuucccuccccccauca
auacaugugagcggaacaaauggaacaaaaagauuugacaauccagugcccuuccauuuaaugaugggguuuuuug
ccaguaccgaaaagucaaacauaauccggggguggaucuuuggaaccacuuuggacucuaagacacagucucuccu
cauaguaaacaacgccaccaauguugucauaaaaguaucccccccgcgaauuucaguuuugaagauuuuuggggug
uauuaccauaagaauaauaaauccuggauggagucugaguuccgcccccgguuuauaguagugcuaauauuuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucgggaucuuccccaaggc
uuuucugcucucgaaccccucguagacuugccaauugggauaaauaucacucgccuuucaaacuuuguugcccucc
acaggagcuaccugacacccggcgacucuucuucugguuggaccgccggggcugccccccccccuuuuuuuuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccaccaaacucaaugaucccuuuguucaccaagucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaggcaggcaguacuccuuguaacggcuuugagggauuuaacugcuauuuuccuuugcaauccuauggcuuuca
accaacaaacgggguuggcuaucaacccuaucgagugguuguccugagcuuccugaacuuuugcagcucccccaca
gucugcggaccaaaaaagaguacaaaucuugucaagaauaagugcguaaauuucaauuucaauggccuuacaggaa
caggcgugcugacugagucaaacaagaaguuccugccauuucagcaguuuggggggacuauagcagacacaacuga
cgcuguacgcgauccucagacuuuggagaucuuggacaucacucccuguuuuucggaggggcuaucugucaucacc
cccggaacuaauacaucaaaucaggucgcuguguuguaccaagaugucaacugcacagaaguccccguugcuauac
acgcagaccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugccuacacuaugucauugggcg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggauuuugggcgcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccaucucuucaguacuuaaugau
auuuugagccgauuggacccacccgaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuaaugguacacauugguuuuccacaca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccaugguacauauggcuuggcuuuaucgcuggccuuaugccccaucguaaugguacaauca
ugcugugcugcaugaccuccugcuguucuuguuugaaaggguguuguucuugugguaguuguugcaaguuugacga
agaugauuccgaaccuguucuuaaagggguaaagcuucacuauaca
SEQ ID NO: 3 (D614G Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguuuugcccuuuuccccuaguaacguuaccuguuucauca
auacaugugagcggaacaaauggaacaaaaagauuugacaaucccccaguguuccauuuaaugaugggguuuuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugccccaguuccgggucccccccuuauaguagugaauauau
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucgggaucuuccccaaggc
uuuucugcucucgaaccccucguagacuugccaauugggauaaauaucacuccgcuuucaaacuuuguugcccucc
acaggagcuaccugacacccggcgacucuucuccucugguuggaccccccccgccggggcugccuuuuuuuuaccu
ucagccacgaacauucuugcucaaguauaacgagaauggcaccauuaccgacgccgucgauugugcauuggauccc
uugucugaaacaaaauguaccuugaaguccuuuaccguagagaaaggcauauaccagacuuccaacuuccgaguuc
agccuacagaauccauugugagauuucccaacaucacaaaccucugcccuuucggugaaguauuuaaugcuacacg
cuucgcuucagucuaugccuggaauaggaagcgcauaucaaauugcguggccgauuauucaguccucuauaauagc
gcauccuucaguacuuucaagugcuacggcguuucccccccaccaaacucaaugaucuuuguucaccaagucuaug
cugacaguuuugucauacgaggcgacgaaguacgccagauugcccccgggcagacagguaaaauugcugauuauaa
uuauaaacucccagaugacuuuacuggaugcgucauagccuggaauuccaacaaucuugauuccaagguugguggg
aauuauaauuaccuuuaucgacuguucagaaagaguaacuugaaaccauuugagagagacauauccaccgagauuu
accaacaaacgggguuggcuaucaacccuaucgagugguuguccugagcuuugaacuuuugcacgcucccgccaca
accaacaaacgggguuggcuaucaacccuaucgagugguuguccugagcuuugaacuuuugcacgcucccgccaca
gucugcggaccaaaaaagaguacaaaucuugucaagaauaagugcguaaauuucaauuucaauggccuuacaggaa
caggcgugcugacugagucaaacaagaaguuccugccauuucagcagcuuuggggggauauagcagacacaacuga
cgcuguacgcgauccucagacuuuggagaucuuggacaucacuccccccuguuuuucggagggguaucugucauca
cccggaacuaauacaucaaaucaggucgcuguguuguaccaagaugucaacugcacagaaguccccguugcuauac
acgcaggccagcucacccccacauggcggguguacucaacuggcucaaacguauuccagaccagagcugggugcuu
gaucggugcugaacacguaaacaauagcuaugaaugcgauauucccaucggugccgggaucugcgcuagcuaucag
acacagaccaauuccccccggcgagcacgaucuguagcaucccagucuauuauugccuacacuacugucauugggg
ccgagaauagcgucgcauauucaaauaauucuauugcaauacccaccaacuucacaaucuccguaacuacagaaau
acuuccaguuuccaugacaaagacaucaguggauuguacaauguauauaugcggagauuccacagaauguucaaau
uugcucuugcaguacggcuccuucugcacccagcucaacagggcccuuacagguauugcugucgaacaggacaaga
acacacaagaagucuucgcccaagucaaacagauauacaaaacuccucccauaaaggaucuuuggggcuucaacuu
uagucagauccucccagacccuucaaaaccaucuaaacgaucauuuauugaagaucugcuguucaacaaggucacu
cuugccgaugcuggauucauuaagcaauacggugacugccuuggugauauugcugcccgagaucugaucugugccc
agaaauucaacgggcucacuguacucccuccacugcucacagacgaaaugauugcacaguacacaagugcccuguu
ggcaggcacaaucacuagcggcuggaccuuuggcgcaggugcagcacuccaaauaccuuuugccaugcagauggcc
uaucgguuuaaugggauaggcgugacucaaaauguccucuacgaaaaccaaaaguugauagcuaaccaauucaauu
cagcaaucgggaagauacaggauucacugucuaguacugcuagugcccuugguaagcugcaggacguugucaacca
gaaugcucaagcucugaauacauugguuaagcagcucucuaguaauuuuggggccaucucuucaguacuuaaugau
auuuugagccgauuggacaaaguggaagcugaaguacagaucgacaggcugauaacaggccggcuccaaucccucc
aaacauacgugacacaacaacucauacgcgcagccgaaauccgagccagcgcuaaccuggcagcuaccaagauguc
agaaugcguucugggccagaguaaacgcguagauuucugcgggaaaggguaccaccugauguccuuuccacaaucu
gcaccucacggggucgucuuuuugcauguaacauauguacccgcacaagagaagaauuuuacuaccgcuccugcca
ucugucaugacgggaaagcucauuuuccucgcgaagguguguuuguaucuaaugguacacauugguuugcuacaca
gcggaauuucuaugaaccccagaucauuacaacugacaacacuuuuguuuccgggaauugugacguggucauagga
aucguaaauaacacuguauaugauccccuccaaccagagcuggacucuuuuaaagaagaacuggauaaauauuuca
agaaccacacaagucccgacguggaccuuggggacauaagugguauuaacgcaucugugguuaacauucaaaagga
aaucgacagacucaacgagguggccaaaaaccugaacgaaagcuugauagaucuccaggaguugggcaaguaugaa
caguacauuaaauggccauugguacauauggcuuggcuuuaucgcuggccuuaucgccaucguaaugguuacauca
ugcugugcugcaugaccuccugcuguucuuguuugaaaggguguuguucuugugguaguuguugcaaguuugacga
agaugauuccgaaccuguucuuaaagggguaaagcuucacuauaca
SEQ ID NO: 4 (B.1.351-PP-D614G Antigen Encoding RNA Sequence)
auguuucugcucacaaccaaacgcacuauguuuguuuuccucgugcugcucccuuugguaaguucucaguguguaa
acuucacaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugccaauccagugcuuccauuuaaugaugggguuuacuuug
ccaguaccgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucggggug
uauuaccauaagaauaauaaauccuggauggagucugaguuccggguuuauaguagugcuaauaauugcacuuucg
aauacgugucccaaccauuccucauggaccuugagggcaaacaggggaauuuuaaaaacuugcgcgaauuugucuu
uaagaauaucgacggauacuuuaagaucuauaguaaacacacuccuaucaaccucguucggggccuuccccaaggc
uuuucugcucucgaaccccucguagacuugccaauugggauaaauaucacucgcuuucaaacuuugcacaucagcu
accugacacccggcgacucuucuucugguuggaccgccggcgccgcugccuauuauguugguuaccuucagccacg
aacauucuugcucaaguauaacgagaauggcaccauuaccgacgccgucgauugugcauuggaucccuugucugaa
acaaaauguaccuugaaguccuuuaccguagagaaaggcauauaccagacuuccaacuuccgaguucagccuacag
aauccauugugagauuucccaacaucacaaaccucugcccuuucccccggugaaguauuuaauguacacgcuuguu
agucuaugccuggaauaggaagcgcauaucaaauugcguggccgauuauucaguccucuauaauagcgcauccuuc
aguacuuucaagugcuacggcguuucccccaccaaacucaaucgaucuuuguuccaccaagucuaugcugacaguu
uugucauacgaggcgacgaaguacgccagauugcccccgggcagacagguaacauugcugauuauaauuauaaacu
cccagaugacuuuacuggaugcgucauagccuggaauuccaacaaucuugauuccaagguuggugggaauuauaau
uaccuuuaucgacuguucagaaagaguaacuugaaaccauuugagagagacauauccaccgagauuuaccaggcag
gcaguacuccuuguaacggcguuaagggauuuaacugcuauuuuccuuugcaauccuauggcuuucaaccaacaua
cgggguuggcuaucaacccuaucgagugguuguccugagcuuugaacuuuugcacgcucccgccacagucugcgga
ccaaaaaagaguacaaaucuugucaagaauaagugcguaaauuucaauuucaauggccuuacaggaacaggcgugc
ugacugagucaaacaagaaguuccugccauuucagcaguuuggggggauauagcagacacaacugaccgcuguacg
cgauccucagacuuuggagaucuuggacaucacucccuguuuuucggagggguaucugucauccacccccggaacu
aauacaucaaaucaggucgcuguguuguaccaaggcgucaacugcacagaaguccccguugcuauacacgcagacc
agcucacccccacauggcggguguacucaacuggcucaaacguauuccagaccagagcugggugcuugaucggugc
ugaacacguaaacaauagcuaugaaugcgauauucccaucggugccgggaucugcgcuagcuaucagacacagacc
aauuccccccggcgagcacgaucuguagcaucccagucuauuauugccuacacuaugucauugggcguggagaaua
gcgucgcauauucaaauaauucuauugcaauacccaccaacuucacaaucuccguaacuacagaaauacuuccagu
uuccaugacaaagacaucaguggauuguacaauguauauaugcggagauuccacagaauguucaaauuugcucuug
caguacggcuccuucugcacccagcucaacagggcccuuacagguauugcugucgaacaggacaagaacacacaag
aagucuucgcccaagucaaacagauauacaaaacuccucccauaaaggauuuuggggcuucaacucuuagucagau
ccucccagacccuucaaaaccaucuaaacgaucauuuauugaagaucugcuguucaacaaggucacucuugccgau
gcuggauucauuaagcaauacggugacugccuuggugauauugcugcccgagaucugaucugugcccagaaauuca
acgggcucacuguacucccuccacugcucacagacgaaaugauugcacaguacacaagugcccuguuggcaggcac
aaucacuagcggcuggaccuuuggcgcaggugcagcacuccaaauaccuuuugccaugcagauggccuaucgguuu
aaugggauaggcgugacucaaaauguccucuacgaaaaccaaaaguugauagcuaaccaauucaauucagcaaucg
ggaagauacaggauucacugucuaguacugcuagugcccuugguaagcugcaggacguugucaaccagaaugcuca
cgauuggacccacccgaagcugaaguacagaucgacaggcugauaacaggccggcuccaaucccuccaaacauacg
ugacacaacaacucauacgcgcagccgaaauccgagccagcgcuaaccuggcagcuaccaagaugucagaaugcgu
ucugggccagaguaaacgcguagauuucugcgggaaaggguaccaccugauguccuuuccacaaucugcaccucac
ggggucgucuuuuugcauguaacauauguacccgcacaagagaagaauuuuacuaccgcuccugccaucugucaug
acgggaaagcucauuuuccucgcgaagguguguuuguaucuaaugguacacauugguuugucacaccagggaauuu
cuaugaaccccagaucauuacaacugacaacacuuuuguuuccgggaauugugacguggucauaggaaucguaaau
aacacuguauaugauccccuccaaccagagcuggacucuuuuaaagaagaacuggauaaauauuucaagaaccaca
caagucccgacguggaccuuggggacauaagugguauuaacgcaucugugguuaacauucaaaaggaaaucgacag
acucaacgagguggccaaaaaccugaacgaaagcuugauagaucuccaggaguugggcaaguaugaacaguacauu
aaauggccaugguacauauggcuuggcuuuaucgcuggccuuaucgccaucguaaugguuacaaucaugcugugcu
gcaugaccuccugcuguucuuguuugaaaggguguuguucuugugguaguuguugcaaguuugacgaagaugauuc
cgaaccuguucuuaaagggguaaagcuucacuauaca
SEQ ID NO: 5 (B.1.1.7-PP-D614G Antigen Encoding RNA Sequence)
auguuucugcucacaaccaaacgcacuauguuuguuuuccucgugcugcucccuuugguaaguucucaguguguaa
accugacaacacgaacccaguugccuccagcuuauaccaacucauuuacucgcggaguauauuaucccgauaaggu
cuuuagaaguagcguguugcacucuacacaggaucuguuuugcccuuuuuccaguccccccaacguuuuuucauca
auaagcggaacaaauggaacaaaaagauuugacaauccagugcuuccauuuaaugaugggguuuacuuugccagua
ccgaaaagucaaacauaauccggggguggaucuuuggaaccacuuuggacucuaagacacagucucuccucauagu
aaacaacgccaccaauguugucauaaaaguaugcgaauuucaguuuugcaacgaucccucuuucgggguguaccau
aagaauaauaaauccuggauggagucugaguuccggguuuauaguagugcuaauaauugcacuuucgaauacgugu
cccaaccauuccucauggaccuugagggcaaacaggggaauuuuaaaaacuugcgcgaauuugucuuuaagaauau
cgacggauacuuuaagaucuauaguaaacacacuccuaucaaccucguucgggcccaucuuccccaagguuuuugu
cucgaaccccucguagacuugccaauugggauaaauaucacucgcuuucacaacuuuguugcccuccacaggagcu
accugacacccggcgaccccucuuccccuucugguuggacccccgggcgccgcugccucccauuuuuuuuucacca
aacauucuugcucaaguauaacgagaauggcaccauuaccgacgccgucgauugugcauuggaucccuugucugaa
acaaaauguaccuugaaguccuuuaccguagagaaaggcauauaccagacuuccaacuuccgaguucagccuacag
aauccauugugagauuucccaacaucacaaacccucugccccuuucggugaaguauuuccaauguacacgcuuguu
agucuaugccuggaauaggaagcgcauaucaaauugcguggccgauuauucaguccucuauaauagcgcauccuuc
aguacuuucaagugcuacggcguuucccccaccaaacucaaugaucuuucguucaccaagucuaugccugacaguu
uugucauacgaggcgacgaaguacgccagauugcccccgggcagacagguaaaauugcugauuauaauuauaaacu
cccagaugacuuuacuggaugcgucauagccuggaauuccaacaaucuugauuccaagguuggugggaauuauaau
uaccuuuaucgacuguucagaaagaguaacuugaaaccauuugagagagacauauccaccgagauuuaccaggcag
gcaguacuccuuguaacggcguugagggauuuaacugcuauuuucccuuugccaaccuacugguuucaaccacaua
cgggguuggcuaucaacccuaucgagugguuguccugagcuuugaacuuucugcacgcucccccacagucugcgga
ccaaaaaagaguacaaaucuugucaagaauaagugcguaaauuucaauuucaauggccuuacaggaacaggcgugc
ugacugagucaaacaagaaguuccugccauuucagcaguuuggggggauauagacgacacacacugacgcuguacg
cgauccucagacuuuggagaucuuggacaucacucccuguuuuucggagggguaucuguccaucacccccggaacu
aauacaucaaaucaggucgcuguguuguaccaaggcgucaacugcacagaaguccccguugcuauacacgcagauc
agcucacccccacauggcggguguacucaacuggcucaaacguauuccagaccagagcugggugcuugaucggugc
ugaacacguaaacaauagcuaugaaugcgauauucccaucggugccgggaucugcgcuagcuaucagacacagacc
aauucccaucggcgagcacgaucuguagcaucccagucuauuauugccuacacuaugucauugggcgccgagaaua
gcgucgcauauucaaauaauucuauugcaauacccaucaacuucacaaucuccguaacuacagaaauacuuccagu
uuccaugacaaagacaucaguggauuguacaauguauauaugcggagauuccacagaauguucaaauuugcucuug
caguacggcuccuucugcacccagcucaacagggcccuuacagguauugcugucgaacaggacaagaacacacaag
aagucuucgcccaagucaaacagauauacaaaacuccucccauaaaggauuuuggggcuucaacuuuagucacgau
ccucccagacccuucaaaaccaucuaaacgaucauuuauugaagaucugcuguucaacaaggucacucuugccgau
gcuggauucauuaagcaauacggugacugccuuggugauauugcugcccgagaucugaucugugcccagaaauuca
acgggcucacuguacucccuccacugcucacagacgaaaugauugcacaguacacaagugcccuguuggcaggcac
aaucacuagcggcuggaccuuuggcgcaggugcagcacuccaaauaccuuuugccaugcagauggccuaucgguuu
aaugggauaggcgugacucaaaauguccucuacgaaaaccaaaaguugauagcuaaccaauucaauucagcaaucg
ggaagauacaggauucacugucuaguacugcuagugcccuugguaagcugcaggacguugucaaccagaaugcuca
cgauuggacccacccgaagcugaaguacagaucgacaggcugauaacaggccggcuccaaucccuccaaacauacg
ugacacaacaacucauacgcgcagccgaaauccgagccagcgcuaaccuggcagcuaccaagaugucagaaugcgu
ucugggccagaguaaacgcguagauuucugcgggaaaggguaccaccugauguccuuuccacaaucugcaccucac
ggggucgucuuuuugcauguaacauauguacccgcacaagagaagaauuuuacuaccgcuccugccaucugucaug
acgggaaagcucauuuuccucgcgaagguguguuuguaucuaaugguacacauugguuugucacacaggcgaauuu
cuaugaaccccagaucauuacaacucacaacacuuuuguuuccgggaauugugacguggucauaggaaucguaaau
aacacuguauaugauccccuccaaccagagcuggacucuuuuaaagaagaacuggauaaauauuucaagaaccaca
caagucccgacguggaccuuggggacauaagugguauuaacgcaucugugguuaacauucaaaaggaaaucgacag
acucaacgagguggccaaaaaccugaacgaaagcuugauagaucuccaggaguugggcaaguaugaacaguacauu
aaauggccaugguacauauggcuuggcuuuaucgcuggcccuuaucgccauguaacugguaccaauccaugugugu
gcaugaccuccugcuguucuuguuugaaaggguguuguucuugugguaguuguugcaaguuugacgaagaugauuc
cgaaccuguucuuaaagggguaaagcuucacuauaca
SEQ ID NO: 6 (Delta.AY1-S2P-wtFur Antigen Encoding RNA Sequence)
auguuucugcucacaaccaaacgcacuauguuuguuuuccucgugcugcucccuuugguaaguucucaguguguaa
accugagaacacgaacccaguugccuccagcuuauaccaacucauuuacucgcggaguauauuaucccgauaaggu
cuuuagaaguagcguguugcacucuacacaggaucuguucuugcccuucuuuaguaacguuaccugguuucaugca
auacaugugagcggaacaaauggaacaaaaagauuugacaauccaccgugcuuccauuuaaugaugggguuuuuug
ccaguaucgaaaagucaaacauaauccggggguggaucuuuggaaccacuuuggacucuaagacacagucucuccu
cauaguaaacaacgccaccaauguugucauaaaaguaugcgaauuucaguuuugcaaccgaucccuuuucgacgug
uauuaccauaagaauaauaaauccuggauggagucugggguuuauaguagugcuaauaauugcacuuucgaauacg
ugucccaaccauuccucauggaccuugagggcaaacaggggaauuuuaaaaacuugcgcgaauuugucuuuaagaa
uaucgacggauacuuuaagaucuauaguaaacacacuccuaucaaccucguucgggaucuucccccaaggcuuuuu
gcucucgaaccccucguagacuugccaauugggauaaauaucacucgcuuucaaacucuuguugcccuccacagga
gcuaccugacacccggcgacucuucuucugguuugaccgccggcgccgcugccuauuauguugguuaccuucagcc
acgaacauucuugcucaaguauaacgagaauggcaccauuaccgacgccgucgauugugcauuggaucccuugucu
gaaacaaaauguaccuugaaguccuuuaccguagagaaaggcauauaccagacuuccaacuuccgaguucagccua
cagaauccaucguacgauuucccaacaucacaaaccucugcccuuucggugaaguauuuaaugcuacacgcuucgc
uucagucuaugccuggaauaggaagcgcauaucaaauugcguggccgauuauucaguccucuauaauagcgcaucc
uucaguacuuucaagugcuacggcguuucccccaccaaacucaaugaucuuuguucaccaaccgucuaugcugaca
guuuugucauacgaggcgacgaaguacgccagauugcccccgggcagacagguaauauugcugauuauaauuauaa
acucccagaugacuuuacuggaugcgucauagccuggaauuccaacaaucuagauuccaagguuggugggaauuau
aauuaccguuaucgacuguucagaaagaguaacuugaaaccauuugagagagacauauccaccgagauuuaccagg
caggcaguaagccuuguaacggcguugagggauuuaacugcuauuuuccuuugcaauccuauggcuuucaaccaac
aaacgggguuggcuaucaacccuaucgagugguuguccucagcuuugaacuuuugcacgcucccgccacagucugc
ggaccaaaaaagaguacaaaucuugucaagaauaagugcguaaauuucaauuucaauggccuuacaggaacaggcg
ugcugacugagucaaacaagaauuuccugccauuucagcaguuuggggggauauagcagacacaacucgacgcugu
acgcgauccucagacuuuggagaucuuggacaucacucccuguuuuucggagggguaucugcucaucacccccgga
acuaauacaucaaaucaggucgcuguguuguaccaaggugucaacugcacagaaguccccguugcuauacacgcag
accagcucacccccacauggcggguguacucaacuggcucaaacguauuccagaccagagcugggugcuugaucgg
ugcugaacacgugaacaauagcuaugaaugcgauauucccaucggugccgggaucugcgcuagcuaucagacacag
accaauucccgcagggggcucgcucuguagcaucccagucuauuauugccuacacuaugucaucugggcgccgaga
auagcgucgcauauucaaauaauucuauugcaauacccaccaacuucacaaucuccguaacuacagaaauacuucc
aguuuccaugacaaagacaucaguggauuguacaauguauauaugcggagauuccacagaauguucaaauuugcuc
uugcaguacggcuccuucugcacccagcucaacagggcacuuacagguauugcugucgaacaggacaagaacacac
aagaagucuucgcccaagucaaacagauauacaaaacuccucccauaaaggauuuuggggccuucaacuuuaguca
gauccucccagacccuucaaaaccaucuaaacgaucauuuauugaagaucugcuguucaacaaggucacucuugcc
gaugcuggauucauuaagcaauacggugacugccuuggugauauugcugcccgagaucugaucugugcccagaaau
ucaacgggcucacuguacucccuccacugcucacagacgaaaugauugcacaguacacaagugcccuguuggcagg
cacaaucacuagcggcuggaccuuuggcgcaggugcagcacuccaaauaccuuuugccaugcagauggccuaucgg
uuuaaugggauaggcgugacucaaaauguccucuacgaaaaccaaaaguugauagcuaaccaauucaauucagcaa
ucgggaagauacaggauucacugucuaguacugcuagugcccuugguaagcugcagaacguugucaaccagaaugc
ucaagcucugaauacauugguuaagcagcucucuaguaauuuuggggccaucucuucaguacuuaaugauauuuug
agccgauuggacccaccugaagcugaaguacagaucgacaggcugauaacaggccggcuccaaucccuccaaacau
acgugacacaacaacucauacgcgcagccgaaauccgagccagcgcuaaccuggcagcuaccaagaugucagaaug
cguucugggccagaguaaacgcguagauuucugcgggaaaggguaccaccugauguccuuuccacaaucugcaccu
cacggggucgucuuuuugcauguaacauacguacccgcacaagagaagaauuuuacuaccgcuccugccaucuguc
augacgggaaagcucauuuuccucgcgaagguguguuuguaucuaaugguacacauugguuugucacacagcggaa
uuucuaugaaccccagaucauuacaacugacaacacuuuuguuuccgggaauugugacguggucauaggaaucgua
aauaacacuguauaugauccccuccaaccagagcuggacucuuuuaaagaagaacuggauaaauauuucaagaacc
acacaagucccgacguggaccuuggggacauaagugguauuaacgcaucugugguuaacauucaaaaggaaaucga
cagacucaacgagguggccaaaaaccugaacgaaagcuugauagaucuccaggaguugggcaaguaugaacaguac
auuaaauggccaugguacauauggcuuggcuuuaucgcuggccuuaucgccaucguaaugguuacaaucaugcugu
gcugcaugaccuccugcuguucuuguuugaaaggguguuguucuugugguaguuguugcaaguuugacgaagauga
gcugcaugaccuccugcuguucuuguuugaaaggguuuuuuuuuugaaaaaa
uuccgaaccuguucuuaaggggguaaagcuucacuauacauga
SEQ ID NO: 7 (Delta.AY1-S2P-wtFur-newKozak Antigen Encoding RNA Sequence)
auguuucugcucacaaccaaacgcacuauguuuuuuuccucgugcugcucccuuugguaaguucucagugugcuaa
accugagaacacgaacccaguugccuccagcuuauaccaacucauuuacucgcggaguauauuaucccgauaaggu
cuuuagaaguagcguguugcacucuacacaggaucuguucuugcccuuuuuaguaacgcuuacccugguuucauca
auacaugugagcggaacaaauggaacaaaaagauuugacaauccagugcuuccccauuuaaugaugggguuuuuug
ccaguaucgaaaagucaaacauaauccggggguggaucuuuggaaccacuuuggacucuaagacacagucucuccu
cauaguaaacaacgccaccaauguugucauaaaaguaugcgaauuucaguuuugcaacgaucccuuucucgacgug
uauuaccauaagaauaauaaauccuggauggagucugggguuuauaguagugcuaauaauugcacuuucgaauacg
ugucccaaccauuccucauggaccuugagggcaaacaggggaauuuuaaaaacuugcgcgaauuugucuuuaagaa
uaucgacggauacuuuaagaucuauaguaaacacacuccuaucaaccucguucgggaucuuccccaagcguuuucu
gcucucgaaccccucguagacuugccaauugggauaaauaucacucgcuuucaaacuuuguugcccuccaccagga
gcuaccugacacccggcgacucuucuucugguuugaccgccggcgccgcugccuauuauguugguuaccuucagcc
acgaacauucuugcucaaguauaacgagaauggcaccauuaccgacgccgucgauugugcauuggauccccuuguu
gaaacaaaauguaccuugaaguccuuuaccguagagaaaggcauauaccagacuuccaacuuccgaguucagccua
cagaauccaucguacgauuucccaacaucacaaaccucugcccuuucggugaaguauuuaaugcuacacgcuucgc
uucagucuaugccuggaauaggaagcgcauaucaaauugcguggccgauuauucaguccucuauaauagcgcaucc
uucaguacuuucaagugcuacggcguuucccccaccaaacucaaugaucuuuguucaccaacgucuaugccugaca
guuuugucauacgaggcgacgaaguacgccagauugcccccgggcagacagguaauauugcugauuauaauuauaa
acucccagaugacuuuacuggaugcgucauagccuggaauuccaacaaucuagauuccaagguuggugggaauuau
aauuaccguuaucgacuguucagaaagaguaacuugaaaccauuugagagagacauauccaccgagauuuaccagg
caggcaguaagccuuguaacggcguugagggauuuaacugcuauucccccuuccuuugcaaucuauggcuuuaaaa
aaacgggguuggcuaucaacccuaucgagugguuguccucagcuuugaacuuuugcacgcucccgccacagucugc
ggaccaaaaaagaguacaaaucuugucaagaauaagugcguaaauuucaauuucaauggccuuacaggaacaggcg
ugcugacugagucaaacaagaauuuccugccauuucagcaguuuggggggauauagcagacacaacugaccgcugu
acgcgauccucagacuuuggagaucuuggacaucacucccuguuuuucggagggguaucugucauccacccccgga
acuaauacaucaaaucaggucgcuguguuguaccaaggugucaacugcacagaaguccccguugcuauacacgcag
accagcucacccccacauggcggguguacucaacuggcucaaacguauuccagaccagagcugggugcuugaucgg
ugcugaacacgugaacaauagcuaugaaugcgauauucccaucggugccgggaucugcgcuagcuaucagacacag
accaauucccgcagggggcucgcucuguagcaucccagucuauuauugccuacacuaugucauugggcgcccgaga
auagcgucgcauauucaaauaauucuauugcaauacccaccaacuucacaaucuccguaacuacagaaauacuucc
aguuuccaugacaaagacaucaguggauuguacaauguauauaugcggagauuccacagaauguucaaauuugcuc
uugcaguacggcuccuucugcacccagcucaacagggcacuuacagguauugcugucgaacaggacaagaacacac
aagaagucuucgcccaagucaaacagauauacaaaacuccucccauaaaggauuuuggggccuucaacuuuaguca
gauccucccagacccuucaaaaccaucuaaacgaucauuuauugaagaucugcuguucaacaaggucacucuugcc
gaugcuggauucauuaagcaauacggugacugccuuggugauauugcugcccgagaucugaucugugcccagaaau
ucaacgggcucacuguacucccuccacugcucacagacgaaaugauugcacaguacacaagugcccuguuggcagg
cacaaucacuagcggcuggaccuuuggcgcaggugcagcacuccaaauaccuuuugccaugcagauggccuaucgg
uuuaaugggauaggcgugacucaaaauguccucuacgaaaaccaaaaguugauagcuaaccaauucaauucagcaa
ucgggaagauacaggauucacugucuaguacugcuagugcccuugguaagcugcagaacguugucaaccagaaugc
ucaagcucugaauacauugguuaagcagcucucuaguaauuuuggggccaucucuucaguacuuaaugauauuuug
agccgauuggacccaccugaagcugaaguacagaucgacaggcugauaacaggccggcuccaaucccuccaaacau
acgugacacaacaacucauacgcgcagccgaaauccgagccagcgcuaaccuggcagcuaccaagaugucagaaug
cguucugggccagaguaaacgcguagauuucuggggaaaggguaccaccugauguccuuucccacaaucugcaccu
cacggggucgucuuuuugcauguaacauacguacccgcacaagagaagaauuuuacuaccgcuccugccaucuguc
augacgggaaagcucauuuuccucgcgaagguguguuuguaucuaaugguacacauugguuugucacacagcggaa
uuucuaugaaccccagaucauuacaacugacaacacuuuuguuuccgggaauugugacguggucauaggaaucgua
aauaacacuguauaugauccccuccaaccagagcuggacucuuuuaaagaagaacuggauaaauauuucaagaacc
acacaagucccgacguggaccuuggggacauaagugguauuaacgcaucugugguuaacauucaaaaggaaaucga
cagacucaacgagguggccaaaaaccugaacgaaagcuugauagaucuccaggaguugggcaaguaugaacaguac
auuaacccauggccaugguacauauggcuuggcuuuaucgcuggccuuaugccaucguaaugguacaaucaugugu
gcugcaugaccuccugcuguucuuguuugaaaggguguuguucuugugguaguuguugcaaguuugacgaagauga
uuccgaaccuguucuuaaggggguaaagcuucacuauacauga
SEQ ID NO: 8 RSV-F Antigen Encoding RNA Sequence
AUGGAGCUGCUCAUCCUUAAAGCGAAUGCUAUAACUACUAUACUUACAGCUGUAACCUUUUGCUUCGCCUCUGGCC
AAAACAUUACUGAGGAGUUUUAUCAAUCCACUUGCAGCGCAGUCUCAAAAGGAUAUCUGUCAGCAUUGCGCACCGG
GUGGUACACCAGCGUUAUCACUAUCGAGCUUUCCAACAUUAAAGAGAAUAAGUGUAACGGCACAGACGCGAAAGUC
AAACUCAUUAAGCAAGAACUUGAUAAAUACAAAAAUGCAGUUACUGAACUUCAGUUGCUCAUGCAAUCAACCCCAG
CAACGAACAAUAGGGCCAGGAGAGAAUUGCCGAGGUUUAUGAACUACACACUUAAUAACGCUAAAAAGACUAACGU
UACGCUCUCUAAGAAGCGGAAGCGCAGGUUUCUCGGGUUCCUUCUGGGGGUUGGCAGUGCAAUAGCAUCCGGCGUC
GCGGUAUCAAAGGUGCUUCAUCUUGAAGGAGAAGUUAACAAAAUCAAAAGCGCCUUGCUUUCAACUAAUAAGGCAG
UAGUAUCAUUGUCUAACGGUGUCAGCGUCUUGACUUCCAAGGUUUUGGAUUUGAAAAACUAUAUCGACAAACAACU
UCUCCCGAUCGUUAACAAGCAGUCAUGCAGUAUCUCCAACAUCGAAACCGUCAUCGAAUUCCAACAGAAAAACAAU
AGACUGCUUGAAAUUACUAGAGAGUUUUCAGUGAACGCUGGCGUUACCACUCCGGUAUCCACUUAUAUGCUCACUA
AUAGCGAACUGCUGUCUCUGAUAAAUGACAUGCCAAUAACUAACGACCAGAAGAAACUUAUGAGUAACAACGUCCA
GAUCGUGAGACAGCAAUCAUAUAGCAUUAUGAGCAUAAUUAAGGAGGAGGUCCUUGCAUACGUAGUCCAGCUCCCA
CUGUACGGGGUUAUCGACACGCCAUGUUGGAAGCUUCAUACUUCCCCCUUGUGCACCACGAACACGAAGGAAGGGU
CUAACAUUUGUCUCACGCGCACUGAUCGGGGGUGGUACUGUGACAACGCCGGGUCAGUGUCAUUUUUCCCUCAGGC
CGAGACCUGCAAGGUCCAAUCAAACCGGGUAUUCUGUGAUACUAUGAACUCCCUGACUCUGCCUUCUGAAGUUAAC
CUGUGUAAUGUAGAUAUAUUCAAUCCUAAAUACGACUGCAAGAUAAUGACCAGCAAAACCGACGUGUCCUCAUCUG
UCAUCACUUCCCUUGGUGCUAUAGUAAGCUGCUAUGGCAAAACGAAAUGCACCGCGAGUAAUAAAAAUCGCGGUAU
CAUCAAGACAUUUAGUAACGGCUGCGAUUAUGUUUCCAACAAAGGUGUUGACACCGUAUCUGUGGGGAAUACCCUG
UAUUACGUAAAUAAGCAGGAAGGGAAAUCUCUCUACGUGAAGGGGGAACCGAUAAUCAACUUUUAUGACCCGCUGG
UUUUUCCGUCCGACGAAUUUGACGCGAGUAUCUCCCAAGUCAACGAGAAAAUUAACCAAAGCCUCGCGUUCAUAAG
AAAAUCCGAUGAGCUGCUGCAUAAUGUAAACGCGGGCAAAUCAACUACCAACAUCAUGAUUACAACAAUAAUCAUC
GUGAUAAUCGUGAUCCUGCUUUCACUUAUCGCUGUCGGGCUUCUUCUCUAUUGUAAGGCCCGGAGUACUCCCGUGA
CCUUGUCUAAGGAUCAGCUCUCAGGUAUCAACAAUAUUGCAUUCAGCAAUUGA
SEQ ID NO: 9 RSV-G Antigen Encoding RNA Sequence
AUGUCCAAAAAUAAAGACCAGCGAACGGCCAAGACUCUGGAGAGGACUUGGGAUACCCUGAAUCAUCUGUUGUUUA
UUUCAAGUUGCCUUUAUAAAUUGAAUCUCAAAUCCGUCGCGCAAAUCACGCUGAGCAUACUCGCAAUGAUUAUCUC
AACUUCCUUGAUAAUUGCCGCCAUAAUCUUCAUUGCAAGUGCAAAUCAUAAAGUGACUCCUACAACCGCAAUAAUA
CAGGACGCGACCAGCCAAAUUAAGAACACGACUCCCACGUAUCUCACCCAAAAUCCCCAACUCGGAAUUAGCCCAA
GUAAUCCGUCAGAGAUUACUUCACAAAUCACCACCAUACUGGCGAGCACAACUCCAGGCGUAAAAUCCACCCUCCA
AUCUACUACCGUCAAGACUAAAAAUACCACUACUACUCAGACACAACCUAGUAAACCUACGACUAAGCAGCGCCAG
AAUAAACCCCCAAGCAAACCAAACAACGACUUCCAUUUUGAAGUUUUCAACUUCGUGCCUUGUUCUAUCUGCUCUA
AUAAUCCAACAUGCUGGGCCAUCUGCAAGCGCAUUCCGAAUAAGAAACCCGGUAAGAAAACUACAACGAAACCUAC
CAAGAAGCCUACCCUGAAAACGACAAAGAAAGACCCGAAACCGCAAACAACCAAAAGUAAAGAAGUGCCCACAACU
AAACCCACAGAAGAACCAACGAUAAACACGACCAAAACCAACAUAAUAACUACGCUGCUUACCAGCAACACGACAG
GCAACCCGGAAUUGACCAGUCAGAUGGAAACUUUUCAUUCAACCAGCAGCGAAGGUAAUCCAAGUCCUAGUCAGGU
GUCUACGACAUCUGAAUAUCCAUCUCAGCCUAGUUCCCCUCCGAACACGCCGCGCCAGUGAUAA
SEQ ID NO: 10 RSV-F Full Length Amino Acid Sequence-F [Human respiratory syncytial
virus BI NCBI GenBank: QED08852.1

| | |
|---|---|
| 1 | mellihrssa ifltlainal yltssqnite efyqstcsav srgylsalrt gwytsvitie |
| 61 | lsniketkcn gtdtkvklik qeldkyknav telqllmqnt pavnnrarre apqymnytin |
| 121 | ttknlnvsis kkrkrrflgf llgvgsaias giavskvlhl egevnkikna lqstnkavvs |
| 181 | lsngvsvlts kvldlknyin nqllpivnkq scrisnietv iefqqknsrl leitrefsvn |
| 241 | agvttplsty mltnsellsl indmpitndq kklmssnvqi vrqqsysims iikeevlayv |
| 301 | vqlpiygvid tpcwklhtsp lcttnikegs nicltrtdrg wycdnagsvs ffpqadtckv |
| 361 | qsnrvfcdtm nsltlpsevs lentdifnsk ydckimtskt disssvitsl gaivscygkt |
| 421 | kctasnknrg iiktfsngcd yvsnkgvdtv svgntlyyvn klegknlyvk gepiinyydp |
| 481 | lvfpsdefda sisqvnekin qslafirrsd ellhnvntgk sttnimitai iiviivvlls |
| 541 | liaiglllyc kakntpvtls kdqlsginni afsk |

SEQ ID NO: 11 RSV-F Partial Amino Acid Sequence-F protein, partial |Human
respiratory syncytial virus B] NCBI GenBank: CBW45391.1
MELLIHRSSAIFLTLAINALYLTSSQNITEEFYQSTCSAVSRGYLSALRTGWYTSVITIELSNIKETKCNGTDTKV
KLIKQELDKYKNAVTELQLLMQNTPAANNRARREAPQYMNYTINTTKNLNVSISKKRKRRFLGFLLGVGSAIASGI
AVSKVLHLEGEVNK
SEQ ID NO: 12 RSV-G Amino Acid Sequence- Human respiratory syncytial virus A (strain
A2) attachment glycoprotein [Human orthopneumovirus] NCBI Reference Sequence:
YP_009518856.1
MSKNKDQRTAKTLERTWDTLNHLLFISSCLYKLNLKSVAQITLSILAMIISTSLIIAAIIFIASANHKVTPTTAII
QDATSQIKNTTPTYLTQNPQLGISPSNPSEITSQITTILASTTPGVKSTLQSTTVKTKNTTTTQTQPSKPTTKQRQ
NKPPSKPNNDFHFEVFNFVPCSICSNNPTCWAICKRIPNKKPGKKTTTKPTKKPTLKTTKKDPKPQTTKSKEVPTT
KPTEEPTINTTKTNIITTLLTSNTTGNPELTSQMETFHSTSSEGNPSPSQVSTTSEYPSQPSSPPNTPRQ
SEQ ID NO: 13 Influenza H3 Antigen Encoding RNA Sequence
AUGAAAACAAUUAUCGCUCUCAGUUAUAUUUUCUGCCUUCCUUUGGGGCAAGACUUGCCGGGCAAUGAUAAUAGUA
CAGCGACUCUCUGUCUCGGACACCACGCAGUACCGAACGGCACACUUGUGAAGACAAUCACAGACGAUCAAAUCGA
AGUGACGAAUGCAACAGAGCUUGUUCAAUCAUCAUCUACCGGCAAAAUCUGCAAUAAUCCGCAUAGAAUCCUUGAU
GGAAUCGACUGCACCCUGAUUGACGCACUGUUGGGAGAUCCUCAUUGCGAUGUGUUUCAGAAUGAGACAUGGGACC
UUUUUGUCGAGAGGAGCAAAGCGUUUUCCAACUGCUACCCGUAUGACGUGCCUGAUUACGCGUCACUCCGCUCACU
UGUAGCAUCAAGCGGUACUCUGGAGUUCAUCACCGAAGGAUUCACCUGGACGGGCGUAACUCAGAACGGCGGUUCU
AACGCAUGUAAGAGGGGGCCAGGGUCCGGCUUCUUCUCACGGCUCAACUGGUUGACUAAGUCUGGUUCAACAUACC
CGGUCCUUAACGUUACAAUGCCGAAUAAUGACAACUUUGAUAAACUCUACAUCUGGGGCAUUCACCAUCCAUCCAC
AAAUCAAGAACAGACAAGUUUGUACGUUCAGGCGUCAGGGCGCGUUACCGUGAGUACAAGAAGAUCACAGCAAACA
AUUAUACCCAAUAUUGGGUCCCGACCCUGGGUAAGAGGACUGUCCUCUCGCAUCUCCAUAUACUGGACCAUUGUCA
AACCGGGCGACGUCCUGGUUAUCAAUAGUAAUGGAAACCUUAUUGCUCCGCGCGGCUAUUUCAAAAUGCGAACUGG
AAAGUCAAGUAUAAUGCGCUCAGACGCACCGAUCGAUACUUGUAUCAGUGAAUGCAUCACCCCUAACGGGUCCAUA
CCGAACGAUAAGCCCUUCCAGAAUGUGAAUAAAAUCACGUAUGGAGCAUGCCCCAAAUACGUGAAGCAAAACACCC
UCAAGUUGGCUACGGGUAUGCGCAACGUCCCAGAAAAACAAACGCGAGGCUUGUUUGGGGCGAUAGCAGGUUUUAU
CGAGAACGGCUGGGAAGGAAUGAUCGAUGGGUGGUACGGCUUUCGCCAUCAAAACUCAGAAGGAACUGGGCAGGCC
GCAGAUCUUAAGUCUACGCAAGCGGCGAUAGAUCAAAUUAAUGGCAAGUUGAAUAGGGUGAUAGAGAAGACGAACG
AGAAGUUCCAUCAAAUAGAGAAAGAAUUCAGUGAAGUAGAGGGGCGAAUUCAGGAUUUGGAAAAAUAUGUCGAGGA
UACUAAGAUCGACCUGUGGAGCUAUAACGCAGAGCUUCUGGUAGCACUUGAGAACCAGCAUACUAUUGAUCUCACC
GAUUCCGAGAUGAACAAGCUUUUUGAGAAGACCAGGAGACAGUUGCGCGAGAAUGCCGAAGAAAUGGGUAACGGUU
GUUUUAAGAUUUAUCACAAAUGUGACAACGCGUGUAUUGAGUCCAUUAGGAAUGGUACAUAUGAUCACGAUGUGUA
UCGCGAUGAAGCACUUAACAAUCGGUUCCAAAUAAAGGGCGUGGAGCUCAAGAGUGGGUAUAAAGAUUGGAUCCUC
UGGAUCUCAUUCGCGAUUUCCUGCUUCCUCCUGUGCGUUGUCUUGCUGGGCUUUAUUAUGUGGGCAUGUCAGAGAG
GUAACAUCCGGUGCAACAUAUGUAUCUGAUAA
SEQ ID NO: 14 Influenza A Haemagglutinin Amino Acid Sequence-hemagglutinin
[Influenza A virus (A/New York/392/2004(H3N2))] NCBI Reference Sequence:
YP_308839.1
MKTIIALSYILCLVFAQKLPGNDNSTATLCLGHHAVPNGTIVKTITNDQIEVTNATELVQSSSTGGICDSPHQILD
GENCTLIDALLGDPQCDGFQNKKWDLFVERSKAYSNCYPYDVPDYASLRSLVASSGTLEFNNESFNWTGVTQNGTS
SACKRRSNNSFFSRLNWLTHLKFKYPALNVTMPNNEKFDKLYIWGVHHPGTDNDQISLYAQASGRITVSTKRSQQT
VIPSIGSRPRIRDVPSRISIYWTIVKPGDILLINSTGNLIAPRGYFKIRSGKSSIMRSDAPIGKCNSECITPNGSI
PNDKPFQNVNRITYGACPRYVKQNTLKLATGMRNVPEKQTRGIFGAIAGFIENGWEGMVDGWYGFRHQNSEGTGQA
ADLKSTQAAINQINGKLNRLIGKTNEKFHQIEKEFSEVEGRIQDLEKYVEDTKIDLWSYNAELLVALENQHTIDLT
DSEMNKLFERTKKQLRENAEDMGNGCFKIYHKCDNACIGSIRNGTYDHDVYRDEALNNRFQIKGVELKSGYKDWIL
WISFAISCFLLCVALLGFIMWACQKGNIRCNICI
SEQ ID NO: 15 Influenza B Haemagglutinin Amino Acid Sequence-hemagglutinin
[Influenza B virus (B/Lee/1940)] NCBI Reference Sequence: NP_056660.1
MKAIIVLLMVVTSNADRICTGITSSNSPHVVKTATQGEVNVTGVIPLTTTPTKSHFANLKGTQTRGKLCPNCFNCT
DLDVALGRPKCMGNTPSAKVSILHEVKPATSGCFPIMHDRTKIRQLPNLLRGYENIRLSTSNVINTETAPGGPYKV
GTSGSCPNVANGNGFFNTMAWVIPKDNNKTAINPVTVEVPYICSEGEDQITVWGFHSDDKTQMERLYGDSNPQKFT
SSANGVTTHYVSQIGGFPNQTEDEGLKQSGRIVVDYMVQKPGKTGTIVYQRGILLPQKVWCASGRSKVIKGSLPLI
GEADCLHEKYGGLNKSKPYYTGEHAKAIGNCPIWVKTPLKLANGTKYRPPAKLLKERGFFGAIAGFLEGGWEGMIA
GWHGYTSHGAHGVAVAADLKSTQEAINKITKNLNYLSELEVKNLQRLSGAMNELHDEILELDEKVDDLRADTISSQ
IELAVLLSNEGIINSEDEHLLALERKLKKMLGPSAVEIGNGCFETKHKCNQTCLDRIAAGTFNAGDFSLPTFDSLN
ITAASLNDDGLDNHTILLYYSTAASSLAVTLMIAIFIVYMVSRDNVSCSICL
SEQ ID NO: 16 Zika Envelope (E) Amino Acid Sequence-envelope protein, partial [Zika
virus] NCBI GenBank: AHL43463.1
QPENLEYRIMLSVHGSQHSGMIVNDTGHETDENRAKVEVTPNSPRATATLGGFGSLGLDCEPRTGLDFSDLYYLTM
NNKHWLVHKEWFHDIPLPWHAGADTGTPHWNNKEALVEFKDAHAKRQTVVVLGSQEGAVHTALAGALEAEMDGAKG
RLFSGHLKCRLKMDKLRLKGVSYSLCTAAFTFTKVPAETLHGTVTVEVQYAGTDGPCKVPAQMAVDMQTLTPVGRL
ITANPIITESTENSRMMLELDPT
SEQ ID NOS: 17-61: See Table 2
SEQ ID NO: 62 VEEV RNA Sequence
AUAGGCGGCGCAUGAGAGAAGCCCAGACCAAUUACCUACCCAAAAUGGAGAAAGUUCACGUUGACAUCGAGGAAGA
CAGCCCAUUCCUCAGAGCUUUGCAGCGGAGCUUCCCGCAGUUUGAGGUAGAAGCCAAGCAGGUCACUGAUAAUGAC
CAUGCUAAUGCCAGAGCGUUUUCGCAUCUGGCUUCAAAACUGAUCGAAACGGAGGUGGACCCAUCCGACACGAUCC
UUGACAUUGGAAGUGCGCCCGCCCGCAGAAUGUAUUCUAAGCACAAGUAUCAUUGUAUCUGUCCGAUGAGAUGUGC
GGAAGAUCCGGACAGAUUGUAUAAGUAUGCAACUAAGCUGAAGAAAAACUGUAAGGAAAUAACUGAUAAGGAAUUG
GACAAGAAAAUGAAGGAGCUGGCCGCCGUCAUGAGCGACCCUGACCUGGAAACUGAGACUAUGUGCCUCCACGACG
ACGAGUCGUGUCGCUACGAAGGGCAAGUCGCUGUUUACCAGGAUGUAUACGCGGUUGACGGACCGACAAGUCUCUA
UCACCAAGCCAAUAAGGGAGUUAGAGUCGCCUACUGGAUAGGCUUUGACACCACCCCUUUUAUGUUUAAGAACUUG
GCUGGAGCAUAUCCAUCAUACUCUACCAACUGGGCCGACGAAACCGUGUUAACGGCUCGUAACAUAGGCCUAUGCA
GCUCUGACGUUAUGGAGCGGUCACGUAGAGGGAUGUCCAUUCUUAGAAAGAAGUAUUUGAAACCAUCCAACAAUGU
UCUAUUCUCUGUUGGCUCGACCAUCUACCACGAGAAGAGGGACUUACUGAGGAGCUGGCACCUGCCGUCUGUAUUU
CACUUACGUGGCAAGCAAAAUUACACAUGUCGGUGUGAGACUAUAGUUAGUUGCGACGGGUACGUCGUUAAAAGAA
UAGCUAUCAGUCCAGGCCUGUAUGGGAAGCCUUCAGGCUAUGCUGCUACGAUGCACCGCGAGGGAUUCUUGUGCUG
CAAAGUGACAGACACAUUGAACGGGGAGAGGGUCUCUUUUCCCGUGUGCACGUAUGUGCCAGCUACAUUGUGUGAC
CAAAUGACUGGCAUACUGGCAACAGAUGUCAGUGCGGACGACGCGCAAAAACUGCUGGUUGGGCUCAACCAGCGUA
UAGUCGUCAACGGUCGCACCCAGAGAAACACCAAUACCAUGAAAAAUUACCUUUUGCCCGUAGUGGCCCAGGCAUU
UGCUAGGUGGGCAAAGGAAUAUAAGGAAGAUCAAGAAGAUGAAAGGCCACUAGGACUACGAGAUAGACAGUUAGUC
AUGGGGUGUUGUUGGGCUUUUAGAAGGCACAAGAUAACAUCUAUUUAUAAGCGCCCGGAUACCCAAACCAUCAUCA
AAGUGAACAGCGAUUUCCACUCAUUCGUGCUGCCCAGGAUAGGCAGUAACACAUUGGAGAUCGGGCUGAGAACAAG
AAUCAGGAAAAUGUUAGAGGAGCACAAGGAGCCGUCACCUCUCAUUACCGCCGAGGACGUACAAGAAGCUAAGUGC
GCAGCCGAUGAGGCUAAGGAGGUGCGUGAAGCCGAGGAGUUGCGCGCAGCUCUACCACCUUUGGCAGCUGAUGUUG
AGGAGCCCACUCUGGAGGCAGACGUCGACUUGAUGUUACAAGAGGCUGGGGCCGGCUCAGUGGAGACACCUCGUGG
CUUGAUAAAGGUUACCAGCUACGAUGGCGAGGACAAGAUCGGCUCUUACGCUGUGCUUUCUCCGCAGGCUGUACUC
AAGAGUGAAAAAUUAUCUUGCAUCCACCCUCUCGCUGAACAAGUCAUAGUGAUAACACACUCUGGCCGAAAAGGGC
GUUAUGCCGUGGAACCAUACCAUGGUAAAGUAGUGGUGCCAGAGGGACAUGCAAUACCCGUCCAGGACUUUCAAGC
UCUGAGUGAAAGUGCCACCAUUGUGUACAACGAACGUGAGUUCGUAAACAGGUACCUGCACCAUAUUGCCACACAU
GGAGGAGCGCUGAACACUGAUGAAGAAUAUUACAAAACUGUCAAGCCCAGCGAGCACGACGGCGAAUACCUGUACG
ACAUCGACAGGAAACAGUGCGUCAAGAAAGAACUAGUCACUGGGCUAGGGCUCACAGGCGAGCUGGUGGAUCCUCC
CUUCCAUGAAUUCGCCUACGAGAGUCUGAGAACACGACCAGCCGCUCCUUACCAAGUACCAACCAUAGGGGUGUAU
GGCGUGCCAGGAUCAGGCAAGUCUGGCAUCAUUAAAAGCGCAGUCACCAAAAAAGAUCUAGUGGUGAGCGCCAAGA
AAGAAAACUGUGCAGAAAUUAUAAGGGACGUCAAGAAAAUGAAAGGGCUGGACGUCAAUGCCAGAACUGUGGACUC
AGUGCUCUUGAAUGGAUGCAAACACCCCGUAGAGACCCUGUAUAUUGACGAAGCUUUUGCUUGUCAUGCAGGUACU
CUCAGAGCGCUCAUAGCCAUUAUAAGACCUAAAAAGGCAGUGCUCUGCGGGGAUCCCAAACAGUGCGGUUUUUUUA
ACAUGAUGUGCCUGAAAGUGCAUUUUAACCACGAGAUUUGCACACAAGUCUUCCACAAAAGCAUCUCUCGCCGUUG
CACUAAAUCUGUGACUUCGGUCGUCUCAACCUUGUUUUACGACAAAAAAAUGAGAACGACGAAUCCGAAAGAGACU
AAGAUUGUGAUUGACACUACCGGCAGUACCAAACCUAAGCAGGACGAUCUCAUUCUCACUUGUUUCAGAGGGUGGG
UGAAGCAGUUGCAAAUAGAUUACAAAGGCAACGAAAUAAUGACGGCAGCUGCCUCUCAAGGGCUGACCCGUAAAGG
UGUGUAUGCCGUUCGGUACAAGGUGAAUGAAAAUCCUCUGUACGCACCCACCUCAGAACAUGUGAACGUCCUACUG
ACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACC
GGACCCUACCGACGUCUUCCAGAAUAAGGCAAACGUGUGUUGGGCCAAGGCUUUAGUGCCGGUGCUGAAGACCGCU
GGCAUAGACAUGACCACUGAACAAUGGAACACUGUGGAUUAUUUUGAAACGGACAAAGCUCACUCAGCAGAGAUAG
UAUUGAACCAACUAUGCGUGAGGUUCUUUGGACUCGAUCUGGACUCCGGUCUAUUUUCUGCACCCACUGUUCCGUU
AUCCAUUAGGAAUAAUCACUGGGAUAACUCCCCGUCGCCUAACAUGUACGGGCUGAAUAAAGAAGUGGUCCGUCAG
CUCUCUCGCAGGUACCCACAACUGCCUCGGGCAGUUGCCACUGGAAGAGUCUAUGACAUGAACACUGGUACACUGC
GCAAUUAUGAUCCGCGCAUAAACCUAGUACCUGUAAACAGAAGACUGCCUCAUGCUUUAGUCCUCCACCAUAAUGA
ACACCCACAGAGUGACUUUUCUUCAUUCGUCAGCAAAUUGAAGGGCAGAACUGUCCUGGUGGUCGGGGAAAAGUUG
UCCGUCCCAGGCAAAAUGGUUGACUGGUUGUCAGACCGGCCUGAGGCUACCUUCAGAGCUCGGCUGGAUUUAGGCA
UCCCAGGUGAUGUGCCCAAAUAUGACAUAAUAUUUGUUAAUGUGAGGACCCCAUAUAAAUACCAUCACUAUCAGCA
GUGUGAAGACCAUGCCAUUAAGCUUAGCAUGUUGACCAAGAAAGCUUGUCUGCAUCUGAAUCCCGGCGGAACCUGU
GUCAGCAUAGGUUAUGGUUACGCUGACAGGGCCAGCGAAAGCAUCAUUGGUGCUAUAGCGCGGCAGUUCAAGUUUU
CCCGGGUAUGCAAACCGAAAUCCUCACUUGAAGAGACGGAAGUUCUGUUUGUAUUCAUUGGGUACGAUCGCAAGGC
CCGUACGCACAAUCCUUACAAGCUUUCAUCAACCUUGACCAACAUUUAUACAGGUUCCAGACUCCACGAAGCCGGA
UGUGCACCCUCAUAUCAUGUGGUGCGAGGGGAUAUUGCCACGGCCACCGAAGGAGUGAUUAUAAAUGCUGCUAACA
GCAAAGGACAACCUGGCGGAGGGGUGUGCGGAGCGCUGUAUAAGAAAUUCCCGGAAAGCUUCGAUUUACAGCCGAU
CGAAGUAGGAAAAGCGCGACUGGUCAAAGGUGCAGCUAAACAUAUCAUUCAUGCCGUAGGACCAAACUUCAACAAA
GUUUCGGAGGUUGAAGGUGACAAACAGUUGGCAGAGGCUUAUGAGUCCAUCGCUAAGAUUGUCAACGAUAACAAUU
ACAAGUCAGUAGCGAUUCCACUGUUGUCCACCGGCAUCUUUUCCGGGAACAAAGAUCGACUAACCCAAUCAUUGAA
CCAUUUGCUGACAGCUUUAGACACCACUGAUGCAGAUGUAGCCAUAUACUGCAGGGACAAGAAAUGGGAAAUGACU
CUCAAGGAAGCAGUGGCUAGGAGAGAAGCAGUGGAGGAGAUAUGCAUAUCCGACGACUCUUCAGUGACAGAACCUG
AUGCAGAGCUGGUGAGGGUGCAUCCGAAGAGUUCUUUGGCUGGAAGGAAGGGCUACAGCACAAGCGAUGGCAAAAC
UUUCUCAUAUUUGGAAGGGACCAAGUUUCACCAGGCGGCCAAGGAUAUAGCAGAAAUUAAUGCCAUGUGGCCCGUU
GCAACGGAGGCCAAUGAGCAGGUAUGCAUGUAUAUCCUCGGAGAAAGCAUGAGCAGUAUUAGGUCGAAAUGCCCCG
UCGAAGAGUCGGAAGCCUCCACACCACCUAGCACGCUGCCUUGCUUGUGCAUCCAUGCCAUGACUCCAGAAAGAGU
ACAGCGCCUAAAAGCCUCACGUCCAGAACAAAUUACUGUGUGCUCAUCCUUUCCAUUGCCGAAGUAUAGAAUCACU
GGUGUGCAGAAGAUCCAAUGCUCCCAGCCUAUAUUGUUCUCACCGAAAGUGCCUGCGUAUAUUCAUCCAAGGAAGU
AUCUCGUGGAAACACCACCGGUAGACGAGACUCCGGAGCCAUCGGCAGAGAACCAAUCCACAGAGGGGACACCUGA
ACAACCACCACUUAUAACCGAGGAUGAGACCAGGACUAGAACGCCUGAGCCGAUCAUCAUCGAAGAGGAAGAAGAG
GAUAGCAUAAGUUUGCUGUCAGAUGGCCCGACCCACCAGGUGCUGCAAGUCGAGGCAGACAUUCACGGGCCGCCCU
CUGUAUCUAGCUCAUCCUGGUCCAUUCCUCAUGCAUCCGACUUUGAUGUGGACAGUUUAUCCAUACUUGACACCCU
GGAGGGAGCUAGCGUGACCAGCGGGGCAACGUCAGCCGAGACUAACUCUUACUUCGCAAAGAGUAUGGAGUUUCUG
GCGCGACCGGUGCCUGCGCCUCGAACAGUAUUCAGGAACCCUCCACAUCCCGCUCCGCGCACAAGAACACCGUCAC
UUGCACCCAGCAGGGCCUGCUCGAGAACCAGCCUAGUUUCCACCCCGCCAGGCGUGAAUAGGGUGAUCACUAGAGA
GGAGCUCGAGGCGCUUACCCCGUCACGCACUCCUAGCAGGUCGGUCUCGAGAACCAGCCUGGUCUCCAACCCGCCA
GGCGUAAAUAGGGUGAUUACAAGAGAGGAGUUUGAGGCGUUCGUAGCACAACAACAAUGACGGUUUGAUGCGGGUG
CAUACAUCUUUUCCUCCGACACCGGUCAAGGGCAUUUACAACAAAAAUCAGUAAGGCAAACGGUGCUAUCCGAAGU
GGUGUUGGAGAGGACCGAAUUGGAGAUUUCGUAUGCCCCGCGCCUCGACCAAGAAAAAGAAGAAUUACUACGCAAG
AAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCA
UCCUGUUCCUUUGUAUUCAUCUAGUGUGAACCGUGCCUUUUCAAGCCCCAAGGUCGCAGUGGAAGCCUGUAACGCC
AUGUUGAAAGAGAACUUUCCGACUGUGGCUUCUUACUGUAUUAUUCCAGAGUACGAUGCCUAUUUGGACAUGGUUG
ACGGAGCUUCAUGCUGCUUAGACACUGCCAGUUUUUGCCCUGCAAAGCUGCGCAGCUUUCCAAAGAAACACUCCUA
UUUGGAACCCACAAUACGAUCGGCAGUGCCUUCAGCGAUCCAGAACACGCUCCAGAACGUCCUGGCAGCUGCCACA
AAAAGAAAUUGCAAUGUCACGCAAAUGAGAGAAUUGCCCGUAUUGGAUUCGGCGGCCUUUAAUGUGGAAUGCUUCA
AGAAAUAUGCGUGUAAUAAUGAAUAUUGGGAAACGUUUAAAGAAAACCCCAUCAGGCUUACUGAAGAAAACGUGGU
AAAUUACAUUACCAAAUUAAAAGGACCAAAAGCUGCUGCUCUUUUUGCGAAGACACAUAAUUUGAAUAUGUUGCAG
GACAUACCAAUGGACAGGUUUGUAAUGGACUUAAAGAGAGACGUGAAAGUGACUCCAGGAACAAAACAUACUGAAG
AACGGCCCAAGGUACAGGUGAUCCAGGCUGCCGAUCCGCUAGCAACAGCGUAUCUGUGCGGAAUCCACCGAGAGCU
GGUUAGGAGAUUAAAUGCGGUCCUGCUUCCGAACAUUCAUACACUGUUUGAUAUGUCGGCUGAAGACUUUGACGCU
AUUAUAGCCGAGCACUUCCAGCCUGGGGAUUGUGUUCUGGAAACUGACAUCGCGUCGUUUGAUAAAAGUGAGGACG
ACGCCAUGGCUCUGACCGCGUUAAUGAUUCUGGAAGACUUAGGUGUGGACGCAGAGCUGUUGACGCUGAUUGAGGC
GGCUUUCGGCGAAAUUUCAUCAAUACAUUUGCCCACUAAAACUAAAUUUAAAUUCGGAGCCAUGAUGAAAUCUGGA
AUGUUCCUCACACUGUUUGUGAACACAGUCAUUAACAUUGUAAUCGCAAGCAGAGUGUUGAGAGAACGGCUAACCG
GAUCACCAUGUGCAGCAUUCAUUGGAGAUGACAAUAUCGUGAAAGGAGUCAAAUCGGACAAAUUAAUGGCAGACAG
GUGCGCCACCUGGUUGAAUAUGGAAGUCAAGAUUAUAGAUGCUGUGGUGGGCGAGAAAGCGCCUUAUUUCUGUGGA
GGGUUUAUUUUGUGUGACUCCGUGACCGGCACAGCGUGCCGUGUGGCAGACCCCCUAAAAAGGCUGUUUAAGCUUG
GCAAACCUCUGGCAGCAGACGAUGAACAUGAUGAUGACAGGAGAAGGGCAUUGCAUGAAGAGUCAACACGCUGGAA
CCGAGUGGGUAUUCUUUCAGAGCUGUGCAAGGCAGUAGAAUCAAGGUAUGAAACCGUAGGAACUUCCAUCAUAGUU
AUGGCCAUGACUACUCUAGCUAGCAGUGUUAAAUCAUUCAGCUACCUGAGAGGGGCCCCUAUAACUCUCUACGGCU
AACCUGAAUGGACUACGACAUAGUCUAGUCCGCCAAG
SEQ ID NO: 63 VEEV RNA polymerase Amino Acid Sequence (NCBI Accession:
AXP98866.1)
RELPVLDSAAFNVECFKKYACNNEYWETFKENPIRLTEENVVNYITKLKGP
SEQ ID NO: 64 VEEV RNA polymerase Amino Acid Sequence (NCBI Accession:
AXP98867.1)
TQMRELPVLDSAAFNVECFKKYACNNEYWETFKENPIRLTE
SEQ ID NO: 65 WT SARS-COV-2 spike-surface glycoprotein [Severe acute respiratory
syndrome coronavirus 2] NCBI GenBank: BCN86353.1

| | |
|---|---|
| 1 | MFLLTTKRTM FVFLVLLPLV SSQCVNLTTR TQLPPAYTNS FTRGVYYPDK VFRSSVLHST |
| 61 | QDLFLPFFSN VTWFHAIHVS GTNGTKRFDN PVLPFNDGVY FASTEKSNII RGWIFGTTLD |
| 121 | SKTQSLLIVN NATNVVIKVC EFQFCNDPFL GVYYHKNNKS WMESEFRVYS SANNCTFEYV |
| 181 | SQPFLMDLEG KQGNFKNLRE FVFKNIDGYF KIYSKHTPIN LVRDLPQGFS ALEPLVDLPI |
| 241 | GINITRFQTL LALHRSYLTP GDSSSGWTAG AAAYYVGYLQ PRTFLLKYNE NGTITDAVDC |
| 301 | ALDPLSETKC TLKSFTVEKG IYQTSNFRVQ PTESIVRFPN ITNLCPFGEV FNATRFASVY |
| 361 | AWNRKRISNC VADYSVLYNS ASFSTFKCYG VSPTKLNDLC FTNVYADSFV IRGDEVRQIA |
| 421 | PGQTGKIADY NYKLPDDFTG CVIAWNSNNL DSKVGGNYNY LYRLFRKSNL KPFERDISTE |
| 481 | IYQAGSTPCN GVEGFNCYFP LOSYGFQPTN GVGYQPYRVV VLSFELLHAP ATVCGPKKST |
| 541 | NLVKNKCVNF NFNGLTGTGV LTESNKKFLP FQQFGRDIAD TTDAVRDPQT LEILDITPCS |
| 601 | FGGVSVITPG TNTSNQVAVL YQDVNCTEVP VAIHADQLTP TWRVYSTGSN VFQTRAGCLI |
| 661 | GAEHVNNSYE CDIPIGAGIC ASYQTQTNSP RRARSVASQS IIAYTMSLGA ENSVAYSNNS |
| 721 | IAIPTNFTIS VTTEILPVSM TKTSVDCTMY ICGDSTECSN LLLQYGSFCT QLNRALTGIA |
| 781 | VEQDKNTQEV FAQVKQIYKT PPIKDFGGFN FSQILPDPSK PSKRSFIEDL LFNKVTLADA |
| 841 | GFIKQYGDCL GDIAARDLIC AQKFNGLTVL PPLLTDEMIA QYTSALLAGT ITSGWTFGAG |
| 901 | AALQIPFAMQ MAYRFNGIGV TQNVLYENQK LIANQFNSAI GKIQDSLSST ASALGKLQDV |
| 906 | VNQNAQALNT LVKQLSSNFG AISSVLNDIL SRLDKVEAEV QIDRLITGRL QSLQTYVTQQ |
| 1021 | LIRAAEIRAS ANLAATKMSE CVLGQSKRVD FCGKGYHLMS FPQSAPHGVV FLHVTYVPAQ |
| 1081 | EKNFTTAPAI CHDGKAHFPR EGVFVSNGTH WFVTORNFYE PQIITTDNTF VSGNCDVVIG |
| 1141 | IVNNTVYDPL QPELDSFKEE LDKYFKNHTS PDVDLGDISG INASVVNIQK EIDRLNEVAK |
| 1201 | NLNESLIDLQ ELGKYEQYIK WPWYIWLGFI AGLIAIVMVT IMLCCMTSCC SCLKGCCSCG |
| 1261 | SCCKFDEDDS EPVLKGVKLH YT |

SEQ ID NO: 66 Polyprotein Amino Acid Sequence [Venezuelan equine encephalitis virus]
(GenBank: ALE15116.1)
MKAITARRILQGLGHYLKAEGKVECYRTLHPVPLYSSSVNRAFSSPKVAVEACNAMLKENFPTVASYCIIPEYDAY
LDMIDGASCCLDTASFCPAKLRSFPKKHSYLEPTIRSAVPSAIQNTLQNVLAAATKRNCNVTQMRELPVLDSAAFN
VECFKKYACNNEYWKTFKENPIRLTEENVINYITKLKGPKAAALYAKTHNLNMLQDIPMDRFVMDLKRDVKVTPGT
KHTEERPKVQVIQAADPLATAYLCGIHRELVRRLNAVLLPNIHTLFDMSAEDFDAIIAEHFQPGDCVLETDIASFD
KSEDDAMALTAMMILEDLGVDAELLTLIEAAFGEISSIHLPTKTKFKFGAMMKSGMFLTLFVNTVINIVIASRVLR
ERLTGSPCAAFIGDDNIVKGVKSDKLMADRCATWLNMEVKIIDAVVGEKAPYFCGGFILCDSVTGTACRVADPLKR
LFKLGKPLAADDEHDDDRRRALHEESTRWNRVGILPELCKAVESRYETVGTSVIVMAMATLASSVKSFSYLRGAPI
TLYG

The following sequences (SEQ ID NOS: 67-73 and 77-83) are formatted to signify vector backbone and antigen open reading frames as follows: lower case letter signify the VEEV replicon backbone sequence; UPPER CASE letters signify spike open reading frame; bold signifies start codons; and underlined signifies mutated codons relative to the parental Wuhan spike sequence.

SEQ ID NO: 67 Full-length VEEV + Delta V5 Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguacgagggauguccauuuua
gaaagaaguauuugaaaccauccaacaauguucuauucucuguuggcucgaccaucccuaccagagaagagggacuuauga
ggagcuggcaccugccgucuguauuucacuuacguggcaagcaaaauuacacccaugucggugugagacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugccacguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagacgugaaaccaauuaucuugcaucacccucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucacugcaggua
cucucagagcgcucauagccauuauaagaccuaaaaaggcagugcucugggggaucccaaacaguggccguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacucguggauuuu
uugaaacggacaaagcucacucagcagagauaguauugaaccaacuaugcgugagguuuuuggacuccgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcuggugccccgauuu
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccccggguaugca
aaccgaaauccucacuugaagagacggaaguucuguuuguauucauuggguacgaugcaaggcccgcuacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccccggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaagzgaagggcuaca
gcacaagcgauggcaaaacuuucucauauuuggaagggaccaaguuucaccaggggccaaggauauagcagcaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccaccggggg
caacgucagccgagacuaacucuuacuucgcaaagaguauggaguuucuggggaccggugccugcgccucccgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucaccacucccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccagugcgaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucaagaaauaucgcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaaucacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugacuccccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaacccguaggaacuuccaucauaguaugg
ccaugacuacucuagcuagcaguguuaaaucauucagcuaccugagaggggccccccuauaacucucuagguaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGACAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUACCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGGGGUGUA
UUACCAUAAGAAUAAUAAAUCCUGGAUGGAGUCUGAGUUCCGGGUUUAUAGUAGUGCUAAUAAUUGCACUUUCGAAUACGU
GUCCCAACCAUUCCUCAUGGACCUUGAGGGCAAACAGGGGAAUUUUAAAAACUUGCGCGAAUUUGUCUUUAAGAAUAUCGA
CGGAUACUUUAAGAUCUAUAGUAAACACACUCCUAUCAACCUCGUUCGGGAUCUUCCCCAAGGCUUUUCUGCUCUCGAACC
CCUCGUAGACUUGCCAAUUGGGAUAAAUAUCACUCGCUUUCAAACUUUGCUUGCCCUCCACAGGAGCUACCUGACACCCGG
CGACUCUUCUUCUGGUUGGACCGCCGGCGCCGCUGCCUAUUAUGUUGGUUACCUUCAGCCACGAACAUUCUUGCUCAAGUA
UAACGAGAAUGGCACCAUUACCGACGCCGUCGAUUGUGCAUUGGAUCCCUUGUCUGAAACAAAAUGUACCUUGAAGUCCUU
UACCGUAGAGAAAGGCAUAUACCAGACUUCCAACUUCCGAGUUCAGCCUACAGAAUCCAUUGUGAGAUUUCCCAACAUCAC
AAACCUCUGCCCUUUCGGUGAAGUAUUUAAUGCUACACGCUUCGCUUCAGUCUAUGCCUGGAAUAGGAAGCGCAUAUCAAA
UUGCGUGGCCGAUUAUUCAGUCCUCUAUAAUAGCGCAUCCUUCAGUACUUUCAAGUGCUACGGCGUUUCCCCCACCAAACU
CAAUGAUCUUUGCUUCACCAACGUCUAUGCUGACAGUUUUGUCAUACGAGGCGACGAAGUACGCCAGAUUGCCCCCGGGCA
GACAGGUAAAAUUGCUGAUUAUAAUUAUAAACUCCCAGAUGACUUUACUGGAUGCGUCAUAGCCUGGAAUUCCAACAAUCU
UGAUUCCAAGGUUGGUGGGAAUUAUAAUUACCUUUAUCGACUGUUCAGAAAGAGUAACUUGAAACCAUUUGAGAGAGACAU
AUCCACCGAGAUUUACCAGGCAGGCAGUACUCCUUGUAACGGCGUUGAGGGAUUUAACUGCUAUUUUCCUUUGCAAUCCUA
UGGCUUUCAACCAACAAACGGGGUUGGCUAUCAACCCUAUCGAGUGGUUGUCCUGAGCUUUGAACUUUUGCACGCUCCCGC
CACAGUCUGCGGACCAAAAAAGAGUACAAAUCUUGUCAAGAAUAAGUGCGUAAAUUUCAAUUUCAAUGGCCUUACAGGAAC
AGGCGUGCUGACUGAGUCAAACAAGAAGUUCCUGCCAUUUCAGCAGUUUGGGCGGGAUAUAGCAGACACAACUGACGCUGU
ACGCGAUCCUCAGACUUUGGAGAUCUUGGACAUCACUCCCUGUUCUUUCGGAGGGGUAUCUGUCAUCACCCCCGGAACUAA
UACAUCAAAUCAGGUCGCUGUGUUGUACCAAGAUGUCAACUGCACAGAAGUCCCCGUUGCUAUACACGCAGACCAGCUCAC
CCCCACAUGGCGGGUGUACUCAACUGGCUCAAACGUAUUCCAGACCAGAGCUGGGUGCUUGAUCGGUGCUGAACACGUAAA
CAAUAGCUAUGAAUGCGAUAUUCCCAUCGGUGCCGGGAUCUGCGCUAGCUAUCAGACACAGACCAAUUCCCCCCGGCGAGC
ACGAUCUGUAGCAUCCCAGUCUAUUAUUGCCUACACUAUGUCAUUGGGCGCCGAGAAUAGCGUCGCAUAUUCAAAUAAUUC
UAUUGCAAUACCCACCAACUUCACAAUCUCCGUAACUACAGAAAUACUUCCAGUUUCCAUGACAAAGACAUCAGUGGAUUG
UACAAUGUAUAUAUGCGGAGAUUCCACAGAAUGUUCAAAUUUGCUCUUGCAGUACGGCUCCUUCUGCACCCAGCUCAACAG
GGCCCUUACAGGUAUUGCUGUCGAACAGGACAAGAACACACAAGAAGUCUUCGCCCAAGUCAAACAGAUAUACAAAACUCC
UCCCAUAAAGGAUUUUGGCGGCUUCAACUUUAGUCAGAUCCUCCCAGACCCUUCAAAACCAUCUAAACGAUCAUUUAUUGA
AGAUCUGCUGUUCAACAAGGUCACUCUUGCCGAUGCUGGAUUCAUUAAGCAAUACGGUGACUGCCUUGGUGAUAUUGCUGC
CCGAGAUCUGAUCUGUGCCCAGAAAUUCAACGGGCUCACUGUACUCCCUCCACUGCUCACAGACGAAAUGAUUGCACAGUA
CACAAGUGCCCUGUUGGCAGGCACAAUCACUAGCGGCUGGACCUUUGGCGCAGGUGCAGCACUCCAAAUACCUUUUGCCAU
GCAGAUGGCCUAUCGGUUUAAUGGGAUAGGCGUGACUCAAAAUGUCCUCUACGAAAACCAAAAGUUGAUAGCUAACCAAUU
CAAUUCAGCAAUCGGGAAGAUACAGGAUUCACUGUCUAGUACUGCUAGUGCCCUUGGUAAGCUGCAGGACGUUGUCAACCA
GAAUGCUCAAGCUCUGAAUACAUUGGUUAAGCAGCUCUCUAGUAAUUUUGGGGCCAUCUCUUCAGUACUUAAUGAUAUUUU
GAGCCGAUUGGACAAAGUGGAAGCUGAAGUACAGAUCGACAGGCUGAUAACAGGCCGGCUCCAAUCCCUCCAAACAUACGU
GACACAACAACUCAUACGCGCAGCCGAAAUCCGAGCCAGCGCUAACCUGGCAGCUACCAAGAUGUCAGAAUGCGUUCUGGG
CCAGAGUAAACGCGUAGAUUUCUGCGGGAAAGGGUACCACCUGAUGUCCUUUCCACAAUCUGCACCUCACGGGGUCGUCUU
UUUGCAUGUAACAUAUGUACCCGCACAAGAGAAGAAUUUUACUACCGCUCCUGCCAUCUGUCAUGACGGGAAAGCUCAUUU
UCCUCGCGAAGGUGUGUUUGUAUCUAAUGGUACACAUUGGUUUGUCACACAGCGGAAUUUCUAUGAACCCCAGAUCAUUAC
AACUGACAACACUUUUGUUUCCGGGAAUUGUGACGUGGUCAUAGGAAUCGUAAAUAACACUGUAUAUGAUCCCCUCCAACC
AGAGCUGGACUCUUUUAAAGAAGAACUGGAUAAAUAUUUCAAGAACCACACAAGUCCCGACGUGGACCUUGGGGACAUAAG
UGGUAUUAACGCAUCUGUGGUUAACAUUCAAAAGGAAAUCGACAGACUCAACGAGGUGGCCAAAAACCUGAACGAAAGCUU
GAUAGAUCUCCAGGAGUUGGGCAAGUAUGAACAGUACAUUAAAUGGCCAUGGUACAUAUGGCUUGGCUUUAUCGCUGGCCU
UAUCGCCAUCGUAAUGGUUACAAUCAUGCUGUGCUGCAUGACCUCCUGCUGUUCUUGUUUGAAAGGGUGUUGUUCUUGUGG
UAGUUGUUGCAAGUUUGACGAAGAUGAUUCCGAACCUGUUCUUAAAGGGGUAAAGCUUCACUAUACAugauaaccgcggug
ucaaaaaccgcguggacgugguuaacaucccugcugggaggaucagccguaauuauuauaauuggcuuggugcuggcuacu
auuguggccauguacgugcugaccaaccagaaacauaauugaauacagcagcaauuggcaagcugcuuacauagaacucgc
ggcgauuggcaugccgccuuaaaauuuuuauuuuauuuuuucuuuucuuuuccgaaucggauuuuguuuuuaauauuucaa
aaaaaaaaaaaaaaaaaaaaaaaaaaaaaaaaaaaaaaaaaaa
SEQ ID NO: 68 Full-length VEEV + K995P-V996P RNA Sequence Antigen Encoding RNA
Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucugucccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagaggcgauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagccacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguacugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggccccuguacucacagagugacccaaaauuuuugcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugagccaagccuuuugcuugucaucagua
cucucagagcgcucauagccauuauaagaccuaaaaaggcagugcucugggggaucccccaaacaguggguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguuuuuggaccucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccgggccuaugca
aaccgaaauccucacuugaagagacggaaguucuguuuguauucauuggguaccgaugcccaaggcccguaccacaaucuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagcccucaguagcgauuccacuguuguccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcugcgaaggaagggcuaca
gcacaagcgauggcaaaacuuucucauauuuggaagggaccaaguuucaccaggggcccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccugggggcccaguagcgugaccaggggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuacccccgucaccacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucccaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcuggcagcuuuccaaaaaacacuccccuauuugg
aacccacaauacgaucggcagugccuucagcgauccagaacacgcuccagaacguccuggcagcugccacaaaaagaaauu
gcaaugucaccaaaugagagaauugcccguauuggauucggggccucuuaauguggaaccugcuucagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuuggcgaaacuuucaucaauacauuugcccacuaaaacuaauuuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcacaggcaguagaaucaagguaugaaaccguaggaacuuccaucauaguaugg
ccaugacuacucuagcuagcaguguuaaaucauucagcuaccugagaggggccccuauaacucucuuagguauaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGACAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUACCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGGGGUGUA
UUACCAUAAGAAUAAUAAAUCCUGGAUGGAGUCUGAGUUCCGGGUUUAUAGUAGUGCUAAUAAUUGCACUUUCGAAUACGU
GUCCCAACCAUUCCUCAUGGACCUUGAGGGCAAACAGGGGAAUUUUAAAAACUUGCGCGAAUUUGUCUUUAAGAAUAUCGA
CGGAUACUUUAAGAUCUAUAGUAAACACACUCCUAUCAACCUCGUUCGGGAUCUUCCCCAAGGCUUUUCUGCUCUCGAACC
CCUCGUAGACUUGCCAAUUGGGAUAAAUAUCACUCGCUUUCAAACUUUGCUUGCCCUCCACAGGAGCUACCUGACACCCGG
CGACUCUUCUUCUGGUUGGACCGCCGGCGCCGCUGCCUAUUAUGUUGGUUACCUUCAGCCACGAACAUUCUUGCUCAAGUA
UAACGAGAAUGGCACCAUUACCGACGCCGUCGAUUGUGCAUUGGAUCCCUUGUCUGAAACAAAAUGUACCUUGAAGUCCUU
UACCGUAGAGAAAGGCAUAUACCAGACUUCCAACUUCCGAGUUCAGCCUACAGAAUCCAUUGUGAGAUUUCCCAACAUCAC
AAACCUCUGCCCUUUCGGUGAAGUAUUUAAUGCUACACGCUUCGCUUCAGUCUAUGCCUGGAAUAGGAAGCGCAUAUCAAA
UUGCGUGGCCGAUUAUUCAGUCCUCUAUAAUAGCGCAUCCUUCAGUACUUUCAAGUGCUACGGCGUUUCCCCCACCAAACU
CAAUGAUCUUUGCUUCACCAACGUCUAUGCUGACAGUUUUGUCAUACGAGGCGACGAAGUACGCCAGAUUGCCCCCGGGCA
GACAGGUAAAAUUGCUGAUUAUAAUUAUAAACUCCCAGAUGACUUUACUGGAUGCGUCAUAGCCUGGAAUUCCAACAAUCU
UGAUUCCAAGGUUGGUGGGAAUUAUAAUUACCUUUAUCGACUGUUCAGAAAGAGUAACUUGAAACCAUUUGAGAGAGACAU
AUCCACCGAGAUUUACCAGGCAGGCAGUACUCCUUGUAACGGCGUUGAGGGAUUUAACUGCUAUUUUCCUUUGCAAUCCUA
UGGCUUUCAACCAACAAACGGGGUUGGCUAUCAACCCUAUCGAGUGGUUGUCCUGAGCUUUGAACUUUUGCACGCUCCCGC
CACAGUCUGCGGACCAAAAAAGAGUACAAAUCUUGUCAAGAAUAAGUGCGUAAAUUUCAAUUUCAAUGGCCUUACAGGAAC
AGGCGUGCUGACUGAGUCAAACAAGAAGUUCCUGCCAUUUCAGCAGUUUGGGCGGGAUAUAGCAGACACAACUGACGCUGU
ACGCGAUCCUCAGACUUUGGAGAUCUUGGACAUCACUCCCUGUUCUUUCGGAGGGGUAUCUGUCAUCACCCCCGGAACUAA
UACAUCAAAUCAGGUCGCUGUGUUGUACCAAGAUGUCAACUGCACAGAAGUCCCCGUUGCUAUACACGCAGACCAGCUCAC
CCCCACAUGGCGGGUGUACUCAACUGGCUCAAACGUAUUCCAGACCAGAGCUGGGUGCUUGAUCGGUGCUGAACACGUAAA
CAAUAGCUAUGAAUGCGAUAUUCCCAUCGGUGCCGGGAUCUGCGCUAGCUAUCAGACACAGACCAAUUCCCCCCGGCGAGC
ACGAUCUGUAGCAUCCCAGUCUAUUAUUGCCUACACUAUGUCAUUGGGCGCCGAGAAUAGCGUCGCAUAUUCAAAUAAUUC
UAUUGCAAUACCCACCAACUUCACAAUCUCCGUAACUACAGAAAUACUUCCAGUUUCCAUGACAAAGACAUCAGUGGAUUG
UACAAUGUAUAUAUGCGGAGAUUCCACAGAAUGUUCAAAUUUGCUCUUGCAGUACGGCUCCUUCUGCACCCAGCUCAACAG
GGCCCUUACAGGUAUUGCUGUCGAACAGGACAAGAACACACAAGAAGUCUUCGCCCAAGUCAAACAGAUAUACAAAACUCC
UCCCAUAAAGGAUUUUGGCGGCUUCAACUUUAGUCAGAUCCUCCCAGACCCUUCAAAACCAUCUAAACGAUCAUUUAUUGA
AGAUCUGCUGUUCAACAAGGUCACUCUUGCCGAUGCUGGAUUCAUUAAGCAAUACGGUGACUGCCUUGGUGAUAUUGCUGC
CCGAGAUCUGAUCUGUGCCCAGAAAUUCAACGGGCUCACUGUACUCCCUCCACUGCUCACAGACGAAAUGAUUGCACAGUA
CACAAGUGCCCUGUUGGCAGGCACAAUCACUAGCGGCUGGACCUUUGGCGCAGGUGCAGCACUCCAAAUACCUUUUGCCAU
GCAGAUGGCCUAUCGGUUUAAUGGGAUAGGCGUGACUCAAAAUGUCCUCUACGAAAACCAAAAGUUGAUAGCUAACCAAUU
CAAUUCAGCAAUCGGGAAGAUACAGGAUUCACUGUCUAGUACUGCUAGUGCCCUUGGUAAGCUGCAGGACGUUGUCAACCA
GAAUGCUCAAGCUCUGAAUACAUUGGUUAAGCAGCUCUCUAGUAAUUUUGGGGCCAUCUCUUCAGUACUUAAUGAUAUUUU
GAGCCGAUUGGACCCACCCGAAGCUGAAGUACAGAUCGACAGGCUGAUAACAGGCCGGCUCCAAUCCCUCCAAACAUACGU
GACACAACAACUCAUACGCGCAGCCGAAAUCCGAGCCAGCGCUAACCUGGCAGCUACCAAGAUGUCAGAAUGCGUUCUGGG
CCAGAGUAAACGCGUAGAUUUCUGCGGGAAAGGGUACCACCUGAUGUCCUUUCCACAAUCUGCACCUCACGGGGUCGUCUU
UUUGCAUGUAACAUAUGUACCCGCACAAGAGAAGAAUUUUACUACCGCUCCUGCCAUCUGUCAUGACGGGAAAGCUCAUUU
UCCUCGCGAAGGUGUGUUUGUAUCUAAUGGUACACAUUGGUUUGUCACACAGCGGAAUUUCUAUGAACCCCAGAUCAUUAC
AACUGACAACACUUUUGUUUCCGGGAAUUGUGACGUGGUCAUAGGAAUCGUAAAUAACACUGUAUAUGAUCCCCUCCAACC
AGAGCUGGACUCUUUUAAAGAAGAACUGGAUAAAUAUUUCAAGAACCACACAAGUCCCGACGUGGACCUUGGGGACAUAAG
UGGUAUUAACGCAUCUGUGGUUAACAUUCAAAAGGAAAUCGACAGACUCAACGAGGUGGCCAAAAACCUGAACGAAAGCUU
GAUAGAUCUCCAGGAGUUGGGCAAGUAUGAACAGUACAUUAAAUGGCCAUGGUACAUAUGGCUUGGCUUUAUCGCUGGCCU
UAUCGCCAUCGUAAUGGUUACAAUCAUGCUGUGCUGCAUGACCUCCUGCUGUUCUUGUUUGAAAGGGUGUUGUUCUUGUGG
UAGUUGUUGCAAGUUUGACGAAGAUGAUUCCGAACCUGUUCUUAAAGGGGUAAAGCUUCACUAUACAugauaaccgcggug
ucaaaaaccgcguggacgugguuaacaucccugcugggaggaucagccguaauuauuauaauuggcuuggugcuggcuacu
auuguggccauguacgugcugaccaaccagaaacauaauugaauacagcagcaauuggcaagcugcuuacauagaacucgc
ggcgauuggcaugccgccuuaaaauuuuuauuuuauuuuuucuuuucuuuuccgaaucggauuuuguuuuuaauauuucaa
aaaaaaaaaaaaaaaaaaaaaaaaaaaaaaaaaaaaaaaaaaa
SEQ ID NO: 69 Full-length VEEV + D614G Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugggaagaucccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugccagacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugucgcacguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccaguaccgauggcg
aggacaagaucggcuccuuaccgcugugcuuucucccccgcaggcuguacucaagagugaaaaauuaucuugcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagccuuuuguugucaugcaggua
cucucagagcgcucauagccauuauaagaccuaaaaaggcagugcucugggggaucccaccaacaguggguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacugucggauuuu
uugaaacggacaaagcucacucagcagagauaguauugaaccaacuaugcgugagguuuuuggacuccgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaagcuuuuccccggguaugca
aaccgaaauccucacuugaagagacggaaguucuguuuguauucauuggguacgauccaaggcccguacgccacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguucguccccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaaggcaagggcuaca
gcacaagcgauggcaaaacuuucucauauuuggaagggaccaaguuucaccaggggccaaggauauagccagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuuguugugcauccaugccaugacuccacgaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcccgugaccaggggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggccgcuuaccccgucaccacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuuccaagccccaagguccaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuuccggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccuggucugaauauggaagucaagauuauagaugcugugguggggagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguagcgaacuuccaucauaguaugg
ccaugacuacucuagcuagcaguguuaaaucauucagcuaccugagaggggccccuauaacucucuacggcuaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGACAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUACCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGGGGUGUA
UUACCAUAAGAAUAAUAAAUCCUGGAUGGAGUCUGAGUUCCGGGUUUAUAGUAGUGCUAAUAAUUGCACUUUCGAAUACGU
GUCCCAACCAUUCCUCAUGGACCUUGAGGGCAAACAGGGGAAUUUUAAAAACUUGCGCGAAUUUGUCUUUAAGAAUAUCGA
CGGAUACUUUAAGAUCUAUAGUAAACACACUCCUAUCAACCUCGUUCGGGAUCUUCCCCAAGGCUUUUCUGCUCUCGAACC
CCUCGUAGACUUGCCAAUUGGGAUAAAUAUCACUCGCUUUCAAACUUUGCUUGCCCUCCACAGGAGCUACCUGACACCCGG
CGACUCUUCUUCUGGUUGGACCGCCGGCGCCGCUGCCUAUUAUGUUGGUUACCUUCAGCCACGAACAUUCUUGCUCAAGUA
UAACGAGAAUGGCACCAUUACCGACGCCGUCGAUUGUGCAUUGGAUCCCUUGUCUGAAACAAAAUGUACCUUGAAGUCCUU
UACCGUAGAGAAAGGCAUAUACCAGACUUCCAACUUCCGAGUUCAGCCUACAGAAUCCAUUGUGAGAUUUCCCAACAUCAC
AAACCUCUGCCCUUUCGGUGAAGUAUUUAAUGCUACACGCUUCGCUUCAGUCUAUGCCUGGAAUAGGAAGCGCAUAUCAAA
UUGCGUGGCCGAUUAUUCAGUCCUCUAUAAUAGCGCAUCCUUCAGUACUUUCAAGUGCUACGGCGUUUCCCCCACCAAACU
CAAUGAUCUUUGCUUCACCAACGUCUAUGCUGACAGUUUUGUCAUACGAGGCGACGAAGUACGCCAGAUUGCCCCCGGGCA
GACAGGUAAAAUUGCUGAUUAUAAUUAUAAACUCCCAGAUGACUUUACUGGAUGCGUCAUAGCCUGGAAUUCCAACAAUCU
UGAUUCCAAGGUUGGUGGGAAUUAUAAUUACCUUUAUCGACUGUUCAGAAAGAGUAACUUGAAACCAUUUGAGAGAGACAU
AUCCACCGAGAUUUACCAGGCAGGCAGUACUCCUUGUAACGGCGUUGAGGGAUUUAACUGCUAUUUUCCUUUGCAAUCCUA
UGGCUUUCAACCAACAAACGGGGUUGGCUAUCAACCCUAUCGAGUGGUUGUCCUGAGCUUUGAACUUUUGCACGCUCCCGC
CACAGUCUGCGGACCAAAAAAGAGUACAAAUCUUGUCAAGAAUAAGUGCGUAAAUUUCAAUUUCAAUGGCCUUACAGGAAC
AGGCGUGCUGACUGAGUCAAACAAGAAGUUCCUGCCAUUUCAGCAGUUUGGGCGGGAUAUAGCAGACACAACUGACGCUGU
ACGCGAUCCUCAGACUUUGGAGAUCUUGGACAUCACUCCCUGUUCUUUCGGAGGGGUAUCUGUCAUCACCCCCGGAACUAA
UACAUCAAAUCAGGUCGCUGUGUUGUACCAAGAUGUCAACUGCACAGAAGUCCCCGUUGCUAUACACGCAGGCCAGCUCAC
CCCCACAUGGCGGGUGUACUCAACUGGCUCAAACGUAUUCCAGACCAGAGCUGGGUGCUUGAUCGGUGCUGAACACGUAAA
CAAUAGCUAUGAAUGCGAUAUUCCCAUCGGUGCCGGGAUCUGCGCUAGCUAUCAGACACAGACCAAUUCCCCCCGGCGAGC
ACGAUCUGUAGCAUCCCAGUCUAUUAUUGCCUACACUAUGUCAUUGGGCGCCGAGAAUAGCGUCGCAUAUUCAAAUAAUUC
UAUUGCAAUACCCACCAACUUCACAAUCUCCGUAACUACAGAAAUACUUCCAGUUUCCAUGACAAAGACAUCAGUGGAUUG
UACAAUGUAUAUAUGCGGAGAUUCCACAGAAUGUUCAAAUUUGCUCUUGCAGUACGGCUCCUUCUGCACCCAGCUCAACAG
GGCCCUUACAGGUAUUGCUGUCGAACAGGACAAGAACACACAAGAAGUCUUCGCCCAAGUCAAACAGAUAUACAAAACUCC
UCCCAUAAAGGAUUUUGGCGGCUUCAACUUUAGUCAGAUCCUCCCAGACCCUUCAAAACCAUCUAAACGAUCAUUUAUUGA
AGAUCUGCUGUUCAACAAGGUCACUCUUGCCGAUGCUGGAUUCAUUAAGCAAUACGGUGACUGCCUUGGUGAUAUUGCUGC
CCGAGAUCUGAUCUGUGCCCAGAAAUUCAACGGGCUCACUGUACUCCCUCCACUGCUCACAGACGAAAUGAUUGCACAGUA
CACAAGUGCCCUGUUGGCAGGCACAAUCACUAGCGGCUGGACCUUUGGCGCAGGUGCAGCACUCCAAAUACCUUUUGCCAU
GCAGAUGGCCUAUCGGUUUAAUGGGAUAGGCGUGACUCAAAAUGUCCUCUACGAAAACCAAAAGUUGAUAGCUAACCAAUU
CAAUUCAGCAAUCGGGAAGAUACAGGAUUCACUGUCUAGUACUGCUAGUGCCCUUGGUAAGCUGCAGGACGUUGUCAACCA
GAAUGCUCAAGCUCUGAAUACAUUGGUUAAGCAGCUCUCUAGUAAUUUUGGGGCCAUCUCUUCAGUACUUAAUGAUAUUUU
GAGCCGAUUGGACAAAGUGGAAGCUGAAGUACAGAUCGACAGGCUGAUAACAGGCCGGCUCCAAUCCCUCCAAACAUACGU
GACACAACAACUCAUACGCGCAGCCGAAAUCCGAGCCAGCGCUAACCUGGCAGCUACCAAGAUGUCAGAAUGCGUUCUGGG
CCAGAGUAAACGCGUAGAUUUCUGCGGGAAAGGGUACCACCUGAUGUCCUUUCCACAAUCUGCACCUCACGGGGUCGUCUU
UUUGCAUGUAACAUAUGUACCCGCACAAGAGAAGAAUUUUACUACCGCUCCUGCCAUCUGUCAUGACGGGAAAGCUCAUUU
UCCUCGCGAAGGUGUGUUUGUAUCUAAUGGUACACAUUGGUUUGUCACACAGCGGAAUUUCUAUGAACCCCAGAUCAUUAC
AACUGACAACACUUUUGUUUCCGGGAAUUGUGACGUGGUCAUAGGAAUCGUAAAUAACACUGUAUAUGAUCCCCUCCAACC
AGAGCUGGACUCUUUUAAAGAAGAACUGGAUAAAUAUUUCAAGAACCACACAAGUCCCGACGUGGACCUUGGGGACAUAAG
UGGUAUUAACGCAUCUGUGGUUAACAUUCAAAAGGAAAUCGACAGACUCAACGAGGUGGCCAAAAACCUGAACGAAAGCUU
GAUAGAUCUCCAGGAGUUGGGCAAGUAUGAACAGUACAUUAAAUGGCCAUGGUACAUAUGGCUUGGCUUUAUCGCUGGCCU
UAUCGCCAUCGUAAUGGUUACAAUCAUGCUGUGCUGCAUGACCUCCUGCUGUUCUUGUUUGAAAGGGUGUUGUUCUUGUGG
UAGUUGUUGCAAGUUUGACGAAGAUGAUUCCGAACCUGUUCUUAAAGGGGUAAAGCUUCACUAUACAuaguaaccgcggug
ucaaaaaccgcguggacgugguuaacaucccugcugggaggaucagccguaauuauuauaauuggcuuggugcuggcuacu
auuguggccauguacgugcugaccaaccagaaacauaauugaauacagcagcaauuggcaagcugcuuacauagaacucgc
ggcgauuggcaugccgccuuaaaauuuuuauuuuauuuuuucuuuucuuuuccgaaucggauuuuguuuuuaauauuucaa
aaaaaaaaaaaaaaaaaaaaaaaaaaaaaaaaaaaaaaaaaaa
SEQ ID NO: 70 Full-length VEEV + B.1.351-PP-D614G Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugggaagaucccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacuccccuaccaacuggggag
aaaccguguuaacggcucguaacauaggccuaugcagcucugacguuauggagcggucacguagagggaucguccauuuua
gaaagaaguauuugaaaccauccaacaauguucuauucucuguuggcucgaccaucuaccagagaagcagggacuuacuga
ggagcuggcaccugccgucuguauuucacuuacguggcaagcaaaauuacacaugucggugugagacuaccuaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccguccgugcaguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguucaccaguacgauggcg
aggacaagaucggcucuuacgcugugcuuucuccgcaggcuguacucaagagugaaaaauuauccccuugcaucacccucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucaugccaggua
cucucagagcgcucauagccauuauaagaccuaaaaaggcagugcucugggggaucccaaacaguggccguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacugucggauuuu
uugaaacggacaaagcucacucagcagagauaguauugaaccaacuaugcgugagguuuuuggacucgauccuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuacccuucagagcucccggcuggaag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggggcaaccugugucagca
uagguuaugguuacgcugacagggccagcgaaagcaucauuggugcuauaggggcaguucaccaguuuucccggguaugca
aaccgaaauccucacuugaagagacggaaguucuguuuguauucauuggguacgaugcaaggcccgcuacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggcacaacccuggggggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacugccuuguccaciggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcugcgaaggaagggcuaca
gcacaagcgauggcaaaacuuucucauauuuggaagggaccaaguuucaccaggggccaaggaucauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagccgugacccaggggg
caacgucagccgagacuaacucuuacuucgcaaagaguauggaguuucuggggaccggugccugcccgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucaccacucccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccagugcgaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcuggcagcuuccuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaaugugcgaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauaccauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguacggaacuuccaucauaguaugg
ccaugacuacucuagcuagcaguguuaaaucauucagcuaccugagaggggccccccuauaacucucuagguaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACUUCACAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGCCAAUCCAGUGCUUCCAUUUAAUGAUGGGGU
UUACUUUGCCAGUACCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGGGGUGUA
UUACCAUAAGAAUAAUAAAUCCUGGAUGGAGUCUGAGUUCCGGGUUUAUAGUAGUGCUAAUAAUUGCACUUUCGAAUACGU
GUCCCAACCAUUCCUCAUGGACCUUGAGGGCAAACAGGGGAAUUUUAAAAACUUGCGCGAAUUUGUCUUUAAGAAUAUCGA
CGGAUACUUUAAGAUCUAUAGUAAACACACUCCUAUCAACCUCGUUCGGGGCCUUCCCCAAGGCUUUUCUGCUCUCGAACC
CCUCGUAGACUUGCCAAUUGGGAUAAAUAUCACUCGCUUUCAAACUUUGCACAUCAGCUACCUGACACCCGGCGACUCUUC
UUCUGGUUGGACCGCCGGCGCCGCUGCCUAUUAUGUUGGUUACCUUCAGCCACGAACAUUCUUGCUCAAGUAUAACGAGAA
UGGCACCAUUACCGACGCCGUCGAUUGUGCAUUGGAUCCCUUGUCUGAAACAAAAUGUACCUUGAAGUCCUUUACCGUAGA
GAAAGGCAUAUACCAGACUUCCAACUUCCGAGUUCAGCCUACAGAAUCCAUUGUGAGAUUUCCCAACAUCACAAACCUCUG
CCCUUUCGGUGAAGUAUUUAAUGCUACACGCUUCGCUUCAGUCUAUGCCUGGAAUAGGAAGCGCAUAUCAAAUUGCGUGGC
CGAUUAUUCAGUCCUCUAUAAUAGCGCAUCCUUCAGUACUUUCAAGUGCUACGGCGUUUCCCCCACCAAACUCAAUGAUCU
UUGCUUCACCAACGUCUAUGCUGACAGUUUUGUCAUACGAGGCGACGAAGUACGCCAGAUUGCCCCCGGGCAGACAGGUAA
CAUUGCUGAUUAUAAUUAUAAACUCCCAGAUGACUUUACUGGAUGCGUCAUAGCCUGGAAUUCCAACAAUCUUGAUUCCAA
GGUUGGUGGGAAUUAUAAUUACCUUUAUCGACUGUUCAGAAAGAGUAACUUGAAACCAUUUGAGAGAGACAUAUCCACCGA
GAUUUACCAGGCAGGCAGUACUCCUUGUAACGGCGUUAAGGGAUUUAACUGCUAUUUUCCUUUGCAAUCCUAUGGCUUUCA
ACCAACAUACGGGGUUGGCUAUCAACCCUAUCGAGUGGUUGUCCUGAGCUUUGAACUUUUGCACGCUCCCGCCACAGUCUG
CGGACCAAAAAAGAGUACAAAUCUUGUCAAGAAUAAGUGCGUAAAUUUCAAUUUCAAUGGCCUUACAGGAACAGGCGUGCU
GACUGAGUCAAACAAGAAGUUCCUGCCAUUUCAGCAGUUUGGGCGGGAUAUAGCAGACACAACUGACGCUGUACGCGAUCC
UCAGACUUUGGAGAUCUUGGACAUCACUCCCUGUUCUUUCGGAGGGGUAUCUGUCAUCACCCCCGGAACUAAUACAUCAAA
UCAGGUCGCUGUGUUGUACCAAGGCGUCAACUGCACAGAAGUCCCCGUUGCUAUACACGCAGACCAGCUCACCCCCACAUG
GCGGGUGUACUCAACUGGCUCAAACGUAUUCCAGACCAGAGCUGGGUGCUUGAUCGGUGCUGAACACGUAAACAAUAGCUA
UGAAUGCGAUAUUCCCAUCGGUGCCGGGAUCUGCGCUAGCUAUCAGACACAGACCAAUUCCCCCCGGCGAGCACGAUCUGU
AGCAUCCCAGUCUAUUAUUGCCUACACUAUGUCAUUGGGCGUGGAGAAUAGCGUCGCAUAUUCAAAUAAUUCUAUUGCAAU
ACCCACCAACUUCACAAUCUCCGUAACUACAGAAAUACUUCCAGUUUCCAUGACAAAGACAUCAGUGGAUUGUACAAUGUA
UAUAUGCGGAGAUUCCACAGAAUGUUCAAAUUUGCUCUUGCAGUACGGCUCCUUCUGCACCCAGCUCAACAGGGCCCUUAC
AGGUAUUGCUGUCGAACAGGACAAGAACACACAAGAAGUCUUCGCCCAAGUCAAACAGAUAUACAAAACUCCUCCCAUAAA
GGAUUUUGGCGGCUUCAACUUUAGUCAGAUCCUCCCAGACCCUUCAAAACCAUCUAAACGAUCAUUUAUUGAAGAUCUGCU
GUUCAACAAGGUCACUCUUGCCGAUGCUGGAUUCAUUAAGCAAUACGGUGACUGCCUUGGUGAUAUUGCUGCCCGAGAUCU
GAUCUGUGCCCAGAAAUUCAACGGGCUCACUGUACUCCCUCCACUGCUCACAGACGAAAUGAUUGCACAGUACACAAGUGC
CCUGUUGGCAGGCACAAUCACUAGCGGCUGGACCUUUGGCGCAGGUGCAGCACUCCAAAUACCUUUUGCCAUGCAGAUGGC
CUAUCGGUUUAAUGGGAUAGGCGUGACUCAAAAUGUCCUCUACGAAAACCAAAAGUUGAUAGCUAACCAAUUCAAUUCAGC
AAUCGGGAAGAUACAGGAUUCACUGUCUAGUACUGCUAGUGCCCUUGGUAAGCUGCAGGACGUUGUCAACCAGAAUGCUCA
AGCUCUGAAUACAUUGGUUAAGCAGCUCUCUAGUAAUUUUGGGGCCAUCUCUUCAGUACUUAAUGAUAUUUUGAGCCGAUU
GGACCCACCCGAAGCUGAAGUACAGAUCGACAGGCUGAUAACAGGCCGGCUCCAAUCCCUCCAAACAUACGUGACACAACA
ACUCAUACGCGCAGCCGAAAUCCGAGCCAGCGCUAACCUGGCAGCUACCAAGAUGUCAGAAUGCGUUCUGGGCCAGAGUAA
ACGCGUAGAUUUCUGCGGGAAAGGGUACCACCUGAUGUCCUUUCCACAAUCUGCACCUCACGGGGUCGUCUUUUUGCAUGU
AACAUAUGUACCCGCACAAGAGAAGAAUUUUACUACCGCUCCUGCCAUCUGUCAUGACGGGAAAGCUCAUUUUCCUCGCGA
AGGUGUGUUUGUAUCUAAUGGUACACAUUGGUUUGUCACACAGCGGAAUUUCUAUGAACCCCAGAUCAUUACAACUGACAA
CACUUUUGUUUCCGGGAAUUGUGACGUGGUCAUAGGAAUCGUAAAUAACACUGUAUAUGAUCCCCUCCAACCAGAGCUGGA
CUCUUUUAAAGAAGAACUGGAUAAAUAUUUCAAGAACCACACAAGUCCCGACGUGGACCUUGGGGACAUAAGUGGUAUUAA
CGCAUCUGUGGUUAACAUUCAAAAGGAAAUCGACAGACUCAACGAGGUGGCCAAAAACCUGAACGAAAGCUUGAUAGAUCU
CCAGGAGUUGGGCAAGUAUGAACAGUACAUUAAAUGGCCAUGGUACAUAUGGCUUGGCUUUAUCGCUGGCCUUAUCGCCAU
CGUAAUGGUUACAAUCAUGCUGUGCUGCAUGACCUCCUGCUGUUCUUGUUUGAAAGGGUGUUGUUCUUGUGGUAGUUGUUG
CAAGUUUGACGAAGAUGAUUCCGAACCUGUUCUUAAAGGGGUAAAGCUUCACUAUACAugauaaccgcggugucaaaaacc
gcguggacgugguuaacaucccugccugggaggaucagccguaauuauuauaauuggcuuggucuggcuacuauuguggcc
auguacgugcugaccaaccagaaacauaauugaauacagcagcaauuggcaagcugcuuacauagaacucgcggcgauugg
caugccgccuuaaaauuuuuauuuuauuuuuucuuuucuuuuccgaaucggauuuuguuuuuaauauuucaaaaaaaaaaa
aaaaaaaaaaaaaaaaaaaaaaaaaaaaaaaaaaaa
SEQ ID NO: 71 Full-length VEEV + B.1.1.7-PP-D614G Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugggaagauccggcacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagcggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugcagacuauagucaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuuccgugucgcacgucaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuucguugucaugcaggua
cucucagagcgcucauagccauuauaagaccuaaaaaggcagugcucugcggggaucccaaacaguggguucuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacaccuguggauuuu
uugaaacggacaaagcucacucagcagagauaguauugaaccaacuaugcgugagguuuuuggacucccgaucuggacucc
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaugcaaggcccgucacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauucccccacuguuguccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggcaaggaagggcuaca
gcacaagcgauggcaaaacuuucucauauuuggaagggaccaaguuucaccaggggccaaggacuauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggacguagcgugacccaggggg
caacgucagccgagacuaacucuuacuucgcaaagaguauggaguuucuggggaccggugccugcccgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucaccacuccucagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuuccaagccccaagguccaguggaagccugua
acgccauguugaaagagaacuuuccgacuguggcuucuuacuguauuauuccagaguacgaugccuauuuggacaugguug
acggagcuucaugcugcuuagacacugccaguuuuugccccugcaaagcugcgcagcuuuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucgggcgccuuuaauguggaaucccgcuucagaaauaugugua
auaaugaauauugggaaacguuuaaagaaaaccccaucaggcuuacugaagcaaaacgugguaaauacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcugugguggggaagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaaucauaguaugg
ccaugacuacucuagcuagcaguguuaaaucauucagcuaccugagaggggccccuauaacucucuacggcuaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGACAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUAAGCGGAACAAAUGGAACAAAAAGAUUUGACAAUCCAGUGCUUCCAUUUAAUGAUGGGGUUUACUU
UGCCAGUACCGAAAAGUCAAACAUAAUCCGGGGGUGGAUCUUUGGAACCACUUUGGACUCUAAGACACAGUCUCUCCUCAU
AGUAAACAACGCCACCAAUGUUGUCAUAAAAGUAUGCGAAUUUCAGUUUUGCAACGAUCCCUUUCUCGGGGUGUACCAUAA
GAAUAAUAAAUCCUGGAUGGAGUCUGAGUUCCGGGUUUAUAGUAGUGCUAAUAAUUGCACUUUCGAAUACGUGUCCCAACC
AUUCCUCAUGGACCUUGAGGGCAAACAGGGGAAUUUUAAAAACUUGCGCGAAUUUGUCUUUAAGAAUAUCGACGGAUACUU
UAAGAUCUAUAGUAAACACACUCCUAUCAACCUCGUUCGGGAUCUUCCCCAAGGCUUUUCUGCUCUCGAACCCCUCGUAGA
CUUGCCAAUUGGGAUAAAUAUCACUCGCUUUCAAACUUUGCUUGCCCUCCACAGGAGCUACCUGACACCCGGCGACUCUUC
UUCUGGUUGGACCGCCGGCGCCGCUGCCUAUUAUGUUGGUUACCUUCAGCCACGAACAUUCUUGCUCAAGUAUAACGAGAA
UGGCACCAUUACCGACGCCGUCGAUUGUGCAUUGGAUCCCUUGUCUGAAACAAAAUGUACCUUGAAGUCCUUUACCGUAGA
GAAAGGCAUAUACCAGACUUCCAACUUCCGAGUUCAGCCUACAGAAUCCAUUGUGAGAUUUCCCAACAUCACAAACCUCUG
CCCUUUCGGUGAAGUAUUUAAUGCUACACGCUUCGCUUCAGUCUAUGCCUGGAAUAGGAAGCGCAUAUCAAAUUGCGUGGC
CGAUUAUUCAGUCCUCUAUAAUAGCGCAUCCUUCAGUACUUUCAAGUGCUACGGCGUUUCCCCCACCAAACUCAAUGAUCU
UUGCUUCACCAACGUCUAUGCUGACAGUUUUGUCAUACGAGGCGACGAAGUACGCCAGAUUGCCCCCGGGCAGACAGGUAA
AAUUGCUGAUUAUAAUUAUAAACUCCCAGAUGACUUUACUGGAUGCGUCAUAGCCUGGAAUUCCAACAAUCUUGAUUCCAA
GGUUGGUGGGAAUUAUAAUUACCUUUAUCGACUGUUCAGAAAGAGUAACUUGAAACCAUUUGAGAGAGACAUAUCCACCGA
GAUUUACCAGGCAGGCAGUACUCCUUGUAACGGCGUUGAGGGAUUUAACUGCUAUUUUCCUUUGCAAUCCUAUGGCUUUCA
ACCAACAUACGGGGUUGGCUAUCAACCCUAUCGAGUGGUUGUCCUGAGCUUUGAACUUUUGCACGCUCCCGCCACAGUCUG
CGGACCAAAAAAGAGUACAAAUCUUGUCAAGAAUAAGUGCGUAAAUUUCAAUUUCAAUGGCCUUACAGGAACAGGCGUGCU
GACUGAGUCAAACAAGAAGUUCCUGCCAUUUCAGCAGUUUGGGCGGGAUAUAGACGACACAACUGACGCUGUACGCGAUCC
UCAGACUUUGGAGAUCUUGGACAUCACUCCCUGUUCUUUCGGAGGGGUAUCUGUCAUCACCCCCGGAACUAAUACAUCAAA
UCAGGUCGCUGUGUUGUACCAAGGCGUCAACUGCACAGAAGUCCCCGUUGCUAUACACGCAGAUCAGCUCACCCCCACAUG
GCGGGUGUACUCAACUGGCUCAAACGUAUUCCAGACCAGAGCUGGGUGCUUGAUCGGUGCUGAACACGUAAACAAUAGCUA
UGAAUGCGAUAUUCCCAUCGGUGCCGGGAUCUGCGCUAGCUAUCAGACACAGACCAAUUCCCAUCGGCGAGCACGAUCUGU
AGCAUCCCAGUCUAUUAUUGCCUACACUAUGUCAUUGGGCGCCGAGAAUAGCGUCGCAUAUUCAAAUAAUUCUAUUGCAAU
ACCCAUCAACUUCACAAUCUCCGUAACUACAGAAAUACUUCCAGUUUCCAUGACAAAGACAUCAGUGGAUUGUACAAUGUA
UAUAUGCGGAGAUUCCACAGAAUGUUCAAAUUUGCUCUUGCAGUACGGCUCCUUCUGCACCCAGCUCAACAGGGCCCUUAC
AGGUAUUGCUGUCGAACAGGACAAGAACACACAAGAAGUCUUCGCCCAAGUCAAACAGAUAUACAAAACUCCUCCCAUAAA
GGAUUUUGGCGGCUUCAACUUUAGUCAGAUCCUCCCAGACCCUUCAAAACCAUCUAAACGAUCAUUUAUUGAAGAUCUGCU
GUUCAACAAGGUCACUCUUGCCGAUGCUGGAUUCAUUAAGCAAUACGGUGACUGCCUUGGUGAUAUUGCUGCCCGAGAUCU
GAUCUGUGCCCAGAAAUUCAACGGGCUCACUGUACUCCCUCCACUGCUCACAGACGAAAUGAUUGCACAGUACACAAGUGC
CCUGUUGGCAGGCACAAUCACUAGCGGCUGGACCUUUGGCGCAGGUGCAGCACUCCAAAUACCUUUUGCCAUGCAGAUGGC
CUAUCGGUUUAAUGGGAUAGGCGUGACUCAAAAUGUCCUCUACGAAAACCAAAAGUUGAUAGCUAACCAAUUCAAUUCAGC
AAUCGGGAAGAUACAGGAUUCACUGUCUAGUACUGCUAGUGCCCUUGGUAAGCUGCAGGACGUUGUCAACCAGAAUGCUCA
AGCUCUGAAUACAUUGGUUAAGCAGCUCUCUAGUAAUUUUGGGGCCAUCUCUUCAGUACUUAAUGAUAUUUUGGCCCGAUU
GGACCCACCCGAAGCUGAAGUACAGAUCGACAGGCUGAUAACAGGCCGGCUCCAAUCCCUCCAAACAUACGUGACACAACA
ACUCAUACGCGCAGCCGAAAUCCGAGCCAGCGCUAACCUGGCAGCUACCAAGAUGUCAGAAUGCGUUCUGGGCCAGAGUAA
ACGCGUAGAUUUCUGCGGGAAAGGGUACCACCUGAUGUCCUUUCCACAAUCUGCACCUCACGGGGUCGUCUUUUUGCAUGU
AACAUAUGUACCCGCACAAGAGAAGAAUUUUACUACCGCUCCUGCCAUCUGUCAUGACGGGAAAGCUCAUUUUCCUCGCGA
AGGUGUGUUUGUAUCUAAUGGUACACAUUGGUUUGUCACACAGCGGAAUUUCUAUGAACCCCAGAUCAUUACAACUCACAA
CACUUUUGUUUCCGGGAAUUGUGACGUGGUCAUAGGAAUCGUAAAUAACACUGUAUAUGAUCCCCUCCAACCAGAGCUGGA
CUCUUUUAAAGAAGAACUGGAUAAAUAUUUCAAGAACCACACAAGUCCCGACGUGGACCUUGGGGACAUAAGUGGUAUUAA
CGCAUCUGUGGUUAACAUUCAAAAGGAAAUCGACAGACUCAACGAGGUGGCCAAAAACCUGAACGAAAGCUUGAUAGAUCU
CCAGGAGUUGGGCAAGUAUGAACAGUACAUUAAAUGGCCAUGGUACAUAUGGCUUGGCUUUAUCGCUGGCCUUAUCGCCAU
CGUAAUGGUUACAAUCAUGCUGUGCUGCAUGACCUCCUGCUGUUCUUGUUUGAAAGGGUGUUGUUCUUGUGGUAGUUGUUG
CAAGUUUGACGAAGAUGAUUCCGAACCUGUUCUUAAAGGGGUAAAGCUUCACUAUACAugauaaccgcggugucaaaaacc
gcguggacgugguuaacaucccugcugggaggaucagccguaauuauuauaauuggcuuggugcuggcuacuauuguggcc
auguacgugcugaccaaccagaaacauaauugaauacagcagcaauuggcaagcugcuuacauagaacucgcggcgauugg
caugccgccuuaaaauuuuuauuuuauuuuuucuuuucuuuuccgaaucggauuuuguuuuuaauauuucaaaaaaaaaaa
aaaaaaaaaaaaaaaaaaaaaaaaaaaaaaaaaaaa
SEQ ID NO: 72 Full-length VEEV + Delta.AY1-S2P-wtFur Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagcagggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacccuauaguaguugg
acggguacgucguuaaaagaauagcuaucaguccaggccuguaugggaagccuucaggcuaugcugcuacgaugcaccgcg
agggauucuugugcugcaaagugacagacacauugaacggggacgagggucuuuuucccccgugugcaguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucgugcgcuugauaaagguuaccaguacgauggcg
aggacaagaucggcucuuacgcugugcuuucuccgcaggcuguacucaagagucccgaaaaauccuaucuugcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaaccgcuuuugcuugucaucagua
cucucagagcgcucauagccauuauaagaccuaaaaaggcagugcucugggggauccccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaacuggaacacuguggauuuu
uugaaacggacaaagcucacucagcagagauaguauugaaccaacuaugcgugagguucuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagccccagcugguggauuu
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaagccuuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcaccccucauauaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaaguccaguaggauuccaccuguuguccccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcucggaaggaagggcuaca
gcacaagcgauggcaaaacuuucucauauuuggaagggaccaaguuucaccaggggcccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggaccgggaguagcgugaccaggggg
caacgucagccgagacuaacucuuacuucgcaaagaguauggaguuucuggggaccccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggcuccaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaaaaacacuccucauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucagccaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacauuugccccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagcaugcugcugguggggagaaaggccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagagccgggccccuauaacucucuagguaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGAGAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUAUCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGACGUGUA
UUACCAUAAGAAUAAUAAAUCCUGGAUGGAGUCUGGGGUUUAUAGUAGUGCUAAUAAUUGCACUUUCGAAUACGUGUCCCA
ACCAUUCCUCAUGGACCUUGAGGGCAAACAGGGGAAUUUUAAAAACUUGCGCGAAUUUGUCUUUAAGAAUAUCGACGGAUA
CUUUAAGAUCUAUAGUAAACACACUCCUAUCAACCUCGUUCGGGAUCUUCCCCAAGGCUUUUCUGCUCUCGAACCCCUCGU
AGACUUGCCAAUUGGGAUAAAUAUCACUCGCUUUCAAACUUUGCUUGCCCUCCACAGGAGCUACCUGACACCCGGCGACUC
UUCUUCUGGUUUGACCGCCGGCGCCGCUGCCUAUUAUGUUGGUUACCUUCAGCCACGAACAUUCUUGCUCAAGUAUAACGA
GAAUGGCACCAUUACCGACGCCGUCGAUUGUGCAUUGGAUCCCUUGUCUGAAACAAAAUGUACCUUGAAGUCCUUUACCGU
AGAGAAAGGCAUAUACCAGACUUCCAACUUCCGAGUUCAGCCUACAGAAUCCAUCGUACGAUUUCCCAACAUCACAAACCU
CUGCCCUUUCGGUGAAGUAUUUAAUGCUACACGCUUCGCUUCAGUCUAUGCCUGGAAUAGGAAGCGCAUAUCAAAUUGCGU
GGCCGAUUAUUCAGUCCUCUAUAAUAGCGCAUCCUUCAGUACUUUCAAGUGCUACGGCGUUUCCCCCACCAAACUCAAUGA
UCUUUGCUUCACCAACGUCUAUGCUGACAGUUUUGUCAUACGAGGCGACGAAGUACGCCAGAUUGCCCCCGGGCAGACAGG
UAAUAUUGCUGAUUAUAAUUAUAAACUCCCAGAUGACUUUACUGGAUGCGUCAUAGCCUGGAAUUCCAACAAUCUAGAUUC
CAAGGUUGGUGGGAAUUAUAAUUACCGUUAUCGACUGUUCAGAAAGAGUAACUUGAAACCAUUUGAGAGAGACAUAUCCAC
CGAGAUUUACCAGGCAGGCAGUAAGCCUUGUAACGGCGUUGAGGGAUUUAACUGCUAUUUUCCUUUGCAAUCCUAUGGCUU
UCAACCAACAAACGGGGUUGGCUAUCAACCCUAUCGAGUGGUUGUCCUCAGCUUUGAACUUUUGCACGCUCCCGCCACAGU
CUGCGGACCAAAAAAGAGUACAAAUCUUGUCAAGAAUAAGUGCGUAAAUUUCAAUUUCAAUGGCCUUACAGGAACAGGCGU
GCUGACUGAGUCAAACAAGAAUUUCCUGCCAUUUCAGCAGUUUGGGCGGGAUAUAGCAGACACAACUGACGCUGUACGCGA
UCCUCAGACUUUGGAGAUCUUGGACAUCACUCCCUGUUCUUUCGGAGGGGUAUCUGUCAUCACCCCCGGAACUAAUACAUC
AAAUCAGGUCGCUGUGUUGUACCAAGGUGUCAACUGCACAGAAGUCCCCGUUGCUAUACACGCAGACCAGCUCACCCCCAC
AUGGCGGGUGUACUCAACUGGCUCAAACGUAUUCCAGACCAGAGCUGGGUGCUUGAUCGGUGCUGAACACGUGAACAAUAG
CUAUGAAUGCGAUAUUCCCAUCGGUGCCGGGAUCUGCGCUAGCUAUCAGACACAGACCAAUUCCCGCAGGCGGGCUCGCUC
UGUAGCAUCCCAGUCUAUUAUUGCCUACACUAUGUCAUUGGGCGCCGAGAAUAGCGUCGCAUAUUCAAAUAAUUCUAUUGC
AAUACCCACCAACUUCACAAUCUCCGUAACUACAGAAAUACUUCCAGUUUCCAUGACAAAGACAUCAGUGGAUUGUACAAU
GUAUAUAUGCGGAGAUUCCACAGAAUGUUCAAAUUUGCUCUUGCAGUACGGCUCCUUCUGCACCCAGCUCAACAGGGCACU
UACAGGUAUUGCUGUCGAACAGGACAAGAACACACAAGAAGUCUUCGCCCAAGUCAAACAGAUAUACAAAACUCCUCCCAU
AAAGGAUUUUGGCGGCUUCAACUUUAGUCAGAUCCUCCCAGACCCUUCAAAACCAUCUAAACGAUCAUUUAUUGAAGAUCU
GCUGUUCAACAAGGUCACUCUUGCCGAUGCUGGAUUCAUUAAGCAAUACGGUGACUGCCUUGGUGAUAUUGCUGCCCGAGA
UCUGAUCUGUGCCCAGAAAUUCAACGGGCUCACUGUACUCCCUCCACUGCUCACAGACGAAAUGAUUGCACAGUACACAAG
UGCCCUGUUGGCAGGCACAAUCACUAGCGGCUGGACCUUUGGCGCAGGUGCAGCACUCCAAAUACCUUUUGCCAUGCAGAU
GGCCUAUCGGUUUAAUGGGAUAGGCGUGACUCAAAAUGUCCUCUACGAAAACCAAAAGUUGAUAGCUAACCAAUUCAAUUC
AGCAAUCGGGAAGAUACAGGAUUCACUGUCUAGUACUGCUAGUGCCCUUGGUAAGCUGCAGAACGUUGUCAACCAGAAUGC
UCAAGCUCUGAAUACAUUGGUUAAGCAGCUCUCUAGUAAUUUUGGGGCCAUCUCUUCAGUACUUAAUGAUAUUUUGAGCCG
AUUGGACCCACCUGAAGCUGAAGUACAGAUCGACAGGCUGAUAACAGGCCGGCUCCAAUCCCUCCAAACAUACGUGACACA
ACAACUCAUACGCGCAGCCGAAAUCCGAGCCAGCGCUAACCUGGCAGCUACCAAGAUGUCAGAAUGCGUUCUGGGCCAGAG
UAAACGCGUAGAUUUCUGCGGGAAAGGGUACCACCUGAUGUCCUUUCCACAAUCUGCACCUCACGGGGUCGUCUUUUUGCA
UGUAACAUACGUACCCGCACAAGAGAAGAAUUUUACUACCGCUCCUGCCAUCUGUCAUGACGGGAAAGCUCAUUUUCCUCG
CGAAGGUGUGUUUGUAUCUAAUGGUACACAUUGGUUUGUCACACAGCGGAAUUUCUAUGAACCCCAGAUCAUUACAACUGA
CAACACUUUUGUUUCCGGGAAUUGUGACGUGGUCAUAGGAAUCGUAAAUAACACUGUAUAUGAUCCCCUCCAACCAGAGCU
GGACUCUUUUAAAGAAGAACUGGAUAAAUAUUUCAAGAACCACACAAGUCCCGACGUGGACCUUGGGGACAUAAGUGGUAU
UAACGCAUCUGUGGUUAACAUUCAAAAGGAAAUCGACAGACUCAACGAGGUGGCCAAAAACCUGAACGAAAGCUUGAUAGA
UCUCCAGGAGUUGGGCAAGUAUGAACAGUACAUUAAAUGGCCAUGGUACAUAUGGCUUGGCUUUAUCGCUGGCCUUAUCGC
CAUCGUAAUGGUUACAAUCAUGCUGUGCUGCAUGACCUCCUGCUGUUCUUGUUUGAAAGGGUGUUGUUCUUGUGGUAGUUG
UUGCAAGUUUGACGAAGAUGAUUCCGAACCUGUUCUUAAGGGGGUAAAGCUUCACUAUACAUGAuaaccgcggugucaaaa
accgcguggacgugguuaacaucccugcugggaggaucagccguaauuauuauaauuggcuuggugcuggcuacuauugug
gccauguacgugcugaccaaccagaaacauaauugaauacagcagcaauuggcaagcugcuuacauagaacucgcggcgau
uggcaugccgccuuaaaauuuuuauuuuauuuucuucuuuucuuuuccgaaucggauuuuguuuuuaauauuucaaaaaaa
aaaaaaaaaaaaaaaaaaaaaaaaaaaaaaaaaaaaaaa
SEQ ID NO: 73 Full-length VEEV + AY1-S2P-wtFur-newKozak Antigen Encoding RNA Sequence
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucaccguagagggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagccacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguacugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggccuguaccucaagagugacaaaauuaucuugcaucacccucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguaucauugacgaagcuuuuguugucaugcaggua
cucucagagcgcucauagccauuauaagaccuaaaaaggcagugcucccugggggaucccaaacaguggguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacacauggaacacuguggauuuu
uugaaacggacaaagcucacucagcagagauaguauugaaccaacuaugcgugagguuuuuggacucgaucugcgacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaauccccggggaaccugugucagca
uagguuaugguuacgcugacagggccagcgaaagcaucauuggugcuauaggggcagccuucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacacgcaaaggacacaccuggggggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacugucuguccacggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcugcgaaggaagggcuaca
gcacaagcgauggcaaaacuuucucauauuuggaagggaccaaguuucaccaggggccaaggauauagccagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacagucuuauccauaccuugacacccuggagggaguagcgugaccaggggg
caacgucagccgagacuaacucuuacuucgcaaagaguauggaccguuucuggggaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccacagguccaguggaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcccuggcagcuuuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaaugucggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaauccaagguaugaaaccguaggaacuuccaucauaguaugg
ccaugacuacucuagcuagcaguguuaaaucauucagcuaccugagaggggccccuauaacucucuacggcuaaccugaau
ggacuacgacauagucuaguccgccgccaccAUGUUUCUGCUCACAACCAAACGCACUAUGUUUGUUUUCCUCGUGCUGCU
CCCUUUGGUAAGUUCUCAGUGUGUAAACCUGAGAACACGAACCCAGUUGCCUCCAGCUUAUACCAACUCAUUUACUCGCGG
AGUAUAUUAUCCCGAUAAGGUCUUUAGAAGUAGCGUGUUGCACUCUACACAGGAUCUGUUCUUGCCCUUCUUUAGUAACGU
UACCUGGUUUCAUGCAAUACAUGUGAGCGGAACAAAUGGAACAAAAAGAUUUGACAAUCCAGUGCUUCCAUUUAAUGAUGG
GGUUUACUUUGCCAGUAUCGAAAAGUCAAACAUAAUCCGGGGGUGGAUCUUUGGAACCACUUUGGACUCUAAGACACAGUC
UCUCCUCAUAGUAAACAACGCCACCAAUGUUGUCAUAAAAGUAUGCGAAUUUCAGUUUUGCAACGAUCCCUUUCUCGACGU
GUAUUACCAUAAGAAUAAUAAAUCCUGGAUGGAGUCUGGGGUUUAUAGUAGUGCUAAUAAUUGCACUUUCGAAUACGUGUC
CCAACCAUUCCUCAUGGACCUUGAGGGCAAACAGGGGAAUUUUAAAAACUUGCGCGAAUUUGUCUUUAAGAAUAUCGACGG
AUACUUUAAGAUCUAUAGUAAACACACUCCUAUCAACCUCGUUCGGGAUCUUCCCCAAGGCUUUUCUGCUCUCGAACCCCU
CGUAGACUUGCCAAUUGGGAUAAAUAUCACUCGCUUUCAAACUUUGCUUGCCCUCCACAGGAGCUACCUGACACCCGGCGA
CUCUUCUUCUGGUUUGACCGCCGGCGCCGCUGCCUAUUAUGUUGGUUACCUUCAGCCACGAACAUUCUUGCUCAAGUAUAA
CGAGAAUGGCACCAUUACCGACGCCGUCGAUUGUGCAUUGGAUCCCUUGUCUGAAACAAAAUGUACCUUGAAGUCCUUUAC
CGUAGAGAAAGGCAUAUACCAGACUUCCAACUUCCGAGUUCAGCCUACAGAAUCCAUCGUACGAUUUCCCAACAUCACAAA
CCUCUGCCCUUUCGGUGAAGUAUUUAAUGCUACACGCUUCGCUUCAGUCUAUGCCUGGAAUAGGAAGCGCAUAUCAAAUUG
CGUGGCCGAUUAUUCAGUCCUCUAUAAUAGCGCAUCCUUCAGUACUUUCAAGUGCUACGGCGUUUCCCCCACCAAACUCAA
UGAUCUUUGCUUCACCAACGUCUAUGCUGACAGUUUUGUCAUACGAGGCGACGAAGUACGCCAGAUUGCCCCCGGGCAGAC
AGGUAAUAUUGCUGAUUAUAAUUAUAAACUCCCAGAUGACUUUACUGGAUGCGUCAUAGCCUGGAAUUCCAACAAUCUAGA
UUCCAAGGUUGGUGGGAAUUAUAAUUACCGUUAUCGACUGUUCAGAAAGAGUAACUUGAAACCAUUUGAGAGAGACAUAUC
CACCGAGAUUUACCAGGCAGGCAGUAAGCCUUGUAACGGCGUUGAGGGAUUUAACUGCUAUUUUCCUUUGCAAUCCUAUGG
CUUUCAACCAACAAACGGGGUUGGCUAUCAACCCUAUCGAGUGGUUGUCCUCAGCUUUGAACUUUUGCACGCUCCCGCCAC
AGUCUGCGGACCAAAAAAGAGUACAAAUCUUGUCAAGAAUAAGUGCGUAAAUUUCAAUUUCAAUGGCCUUACAGGAACAGG
CGUGCUGACUGAGUCAAACAAGAAUUUCCUGCCAUUUCAGCAGUUUGGGCGGGAUAUAGCAGACACAACUGACGCUGUACG
CGAUCCUCAGACUUUGGAGAUCUUGGACAUCACUCCCUGUUCUUUCGGAGGGGUAUCUGUCAUCACCCCCGGAACUAAUAC
AUCAAAUCAGGUCGCUGUGUUGUACCAAGGUGUCAACUGCACAGAAGUCCCCGUUGCUAUACACGCAGACCAGCUCACCCC
CACAUGGCGGGUGUACUCAACUGGCUCAAACGUAUUCCAGACCAGAGCUGGGUGCUUGAUCGGUGCUGAACACGUGAACAA
UAGCUAUGAAUGCGAUAUUCCCAUCGGUGCCGGGAUCUGCGCUAGCUAUCAGACACAGACCAAUUCCCGCAGGCGGGCUCG
CUCUGUAGCAUCCCAGUCUAUUAUUGCCUACACUAUGUCAUUGGGCGCCGAGAAUAGCGUCGCAUAUUCAAAUAAUUCUAU
UGCAAUACCCACCAACUUCACAAUCUCCGUAACUACAGAAAUACUUCCAGUUUCCAUGACAAAGACAUCAGUGGAUUGUAC
AAUGUAUAUAUGCGGAGAUUCCACAGAAUGUUCAAAUUUGCUCUUGCAGUACGGCUCCUUCUGCACCCAGCUCAACAGGGC
ACUUACAGGUAUUGCUGUCGAACAGGACAAGAACACACAAGAAGUCUUCGCCCAAGUCAAACAGAUAUACAAAACUCCUCC
CAUAAAGGAUUUUGGCGGCUUCAACUUUAGUCAGAUCCUCCCAGACCCUUCAAAACCAUCUAAACGAUCAUUUAUUGAAGA
UCUGCUGUUCAACAAGGUCACUCUUGCCGAUGCUGGAUUCAUUAAGCAAUACGGUGACUGCCUUGGUGAUAUUGCUGCCCG
AGAUCUGAUCUGUGCCCAGAAAUUCAACGGGCUCACUGUACUCCCUCCACUGCUCACAGACGAAAUGAUUGCACAGUACAC
AAGUGCCCUGUUGGCAGGCACAAUCACUAGCGGCUGGACCUUUGGCGCAGGUGCAGCACUCCAAAUACCUUUUGCCAUGCA
GAUGGCCUAUCGGUUUAAUGGGAUAGGCGUGACUCAAAAUGUCCUCUACGAAAACCAAAAGUUGAUAGCUAACCAAUUCAA
UUCAGCAAUCGGGAAGAUACAGGAUUCACUGUCUAGUACUGCUAGUGCCCUUGGUAAGCUGCAGAACGUUGUCAACCAGAA
UGCUCAAGCUCUGAAUACAUUGGUUAAGCAGCUCUCUAGUAAUUUUGGGGCCAUCUCUUCAGUACUUAAUGAUAUUUUGAG
CCGAUUGGACCCACCUGAAGCUGAAGUACAGAUCGACAGGCUGAUAACAGGCCGGCUCCAAUCCCUCCAAACAUACGUGAC
ACAACAACUCAUACGCGCAGCCGAAAUCCGAGCCAGCGCUAACCUGGCAGCUACCAAGAUGUCAGAAUGCGUUCUGGGCCA
GAGUAAACGCGUAGAUUUCUGCGGGAAAGGGUACCACCUGAUGUCCUUUCCACAAUCUGCACCUCACGGGGUCGUCUUUUU
GCAUGUAACAUACGUACCCGCACAAGAGAAGAAUUUUACUACCGCUCCUGCCAUCUGUCAUGACGGGAAAGCUCAUUUUCC
UCGCGAAGGUGUGUUUGUAUCUAAUGGUACACAUUGGUUUGUCACACAGCGGAAUUUCUAUGAACCCCAGAUCAUUACAAC
UGACAACACUUUUGUUUCCGGGAAUUGUGACGUGGUCAUAGGAAUCGUAAAUAACACUGUAUAUGAUCCCCUCCAACCAGA
GCUGGACUCUUUUAAAGAAGAACUGGAUAAAUAUUUCAAGAACCACACAAGUCCCGACGUGGACCUUGGGGACAUAAGUGG
UAUUAACGCAUCUGUGGUUAACAUUCAAAAGGAAAUCGACAGACUCAACGAGGUGGCCAAAAACCUGAACGAAAGCUUGAU
AGAUCUCCAGGAGUUGGGCAAGUAUGAACAGUACAUUAAAUGGCCAUGGUACAUAUGGCUUGGCUUUAUCGCUGGCCUUAU
CGCCAUCGUAAUGGUUACAAUCAUGCUGUGCUGCAUGACCUCCUGCUGUUCUUGUUUGAAAGGGUGUUGUUCUUGUGGUAG
UUGUUGCAAGUUUGACGAAGAUGAUUCCGAACCUGUUCUUAAGGGGGUAAAGCUUCACUAUACAUGAuaaccgcgguguca
aaaaccgcguggacgugguuaacaucccugcugggaggaucagccguaauuauuauaauuggcuuggugcuggcuacuauu
guggccauguacgugcugaccaaccagaaacauaauugaauacagcagcaauuggcaagcugcuuacauagaacucgcggc
gauuggcaugccgccuuaaaauuuuuauuuuauuuuuucuuuucuuuuccgaaucggauuuuguuuuuaauauuucaaaaa
aaaaaaaaaaaaaaaaaaaaaaaaaaaaaaaaaaaaaaaa

The following sequences (SEQ ID NOS: 74-76) are formatted to signify vector backbone and antigen open reading frames as follows: lower case letter signify the VEEV replicon backbone sequence; and UPPER CASE letters signify antigen open reading frame.

SEQ ID NO: 74 Full-length VEEV + RSV-F Antigen Encoding RNA Sequence
auaggcggcgcaugagagaagcccagaccaauuaccuacccaaaauggagaaaguucacguugacaucgaggaaga
cagcccauuccucagagcuuugcagcggagcuucccgcaguuugagguagaagccaagcaggucacugauaaugac
caugcuaaugccagagcguuuucgcaucuggcuucaaaacugaucgaaacggagguggacccauccgacacgaucc
uugacauuggaagugcgcccgcccgcagaauguauucuaagcacaaguaucauuguaucuguccgaugagaugugc
ggaagauccggacagauuguauaaguaugcaacuaagcugaagaaaaacuguaaggaaauaacugauaaggaauug
gacaagaaaaugaaggagcuggccgccgucaugagcgacccugaccuggaaacugagacuaugugccuccacgacg
acgagucgugucgcuacgaagggcaagucgcuguuuaccaggauguauacgcgguugacggaccgacaagucucua
ucaccaagccaauaagggaguuagagucgccuacuggauaggcuuugacaccaccccuuuuauguuuaagaacuug
gcuggagcauauccaucauacucuaccaacugggccgacgaaaccguguuaacggcucguaacauaggccuaugca
gcucugacguuauggagcggucacguagagggauguccauucuuagaaagaaguauuugaaaccauccaacaaugu
ucuauucucuguuggcucgaccaucuaccacgagaagagggacuuacugaggagcuggcaccugccgucuguauuu
cacuuacguggcaagcaaaauuacacaugucggugugagacuauaguuaguugcgacggguacgucguuaaaagaa
uagcuaucaguccaggccuguaugggaagccuucaggcuaugcugcuacgaugcaccgcgagggauucuugugcug
caaagugacagacacauugaacggggagagggucucuuuucccgugugcacguaugugccagcuacauugugugac
caaaugacuggcauacuggcaacagaugucagugcggacgacgcgcaaaaacugcugguugggcucaaccagcgua
uagucgucaacggucgcacccagagaaacaccaauaccaugaaaaauuaccuuuugcccguaguggcccaggcauu
ugcuaggugggcaaaggaauauaaggaagaucaagaagaugaaaggccacuaggacuacgagauagacaguuaguc
augggguguuguugggcuuuuagaaggcacaagauaacaucuauuuauaagcgcccggauacccaaaccaucauca
aagugaacagcgauuuccacucauucgugcugcccaggauaggcaguaacacauuggagaucgggcugagaacaag
aaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguug
aggagcccacucuggaggcagacgucgacuugauguuacaagaggcuggggccggcucaguggagacaccucgugg
cuugauaaagguuaccagcuacgauggcgaggacaagaucggcucuuacgcugugcuuucuccgcaggcuguacuc
aagagugaaaaauuaucuugcauccacccucucgcugaacaagucauagugauaacacacucuggccgaaaagggc
guuaugccguggaaccauaccaugguaaaguaguggugccagagggacaugcaauacccguccaggacuuucaagc
ucugagugaaagugccaccauuguguacaacgaacgugaguucguaaacagguaccugcaccauauugccacacau
ggaggagcgcugaacacugaugaagaauauuacaaaacugucaagcccagcgagcacgacggcgaauaccuguacg
acaucgacaggaaacagugcgucaagaaagaacuagucacugggcuagggcucacaggcgagcugguggauccucc
cuuccaugaauucgccuacgagagucugagaacacgaccagccgcuccuuaccaaguaccaaccauagggguguau
ggcgugccaggaucaggcaagucuggcaucauuaaaagcgcagucaccaaaaaagaucuaguggugagcgccaaga
aagaaaacugugcagaaauuauaagggacgucaagaaaaugaaagggcuggacgucaaugccagaacuguggacuc
agugcucuugaauggaugcaaacaccccguagagacccuguauauugacgaagcuucuuguugucaugcagguacu
cucagagcgcucauagccauuauaagaccuaaaaaggcagugcucugcggggaucccaaacagugcgguuuuuuua
acaugaugugccugaaagugcauuuuaaccacgagauuugcacacaagucuuccacaaaagcaucucucgccguug
cacuaaaucugugacuucggucgucucaaccuuguuuuacgacaaaaaaaugagaacgacgaauccgaaagagacu
aagauugugauugacacuaccggcaguaccaaaccuaagcagcgacgaucucauucucacuuguuucagagggggg
ugaagcaguugcaaauagauuacaaaggcaacgaaauaaugacggcagcugccucucaagggcugacccguaaagg
uguguaugccguucgguacaaggugaaugaaaauccucuguacgcacccaccucagaacaugugaacguccuacug
acccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcu
ggcauagacaugaccacugaacaauggaacacuguggauuauuuugaaacggacaaagcucacucagcagagauag
uauugaaccaacuaugcgugagguucuuuggacucgaucuggacuccggucuauuuucugcacccacuguuccguu
auccauuaggaauaaucacugggauaacuccccgucgccuaacauguacgggcugaauaaagaagugguccgucag
cucucucgcagguacccacaacugccucgggcaguugccacuggaagagucuaugacaugaacacugguacacugc
gcaauuaugauccgcgcauaaaccuaguaccuguaaacagaagacugccucaugcuuuaguccuccaccauaauga
acacccacagagugacuuuucuucauucgucagcaaauugaagggcagaacuguccugguggucggggaaaaguug
uccgucccaggcaaaaugguugacugguugucagaccggccugaggcuaccuucagagcucggcuggauuuaggca
ucccaggugaugugcccaaauaugacauaauauuuguuaaugugaggaccccauauaaauaccaucacuaucagca
gugugaagaccaugccauuaagcuuagcauguugaccaagaaagcuugucugcaucugaaucccggcggaaccugu
gucagcauagguuaugguuacgcugacagggccagcgaaagcaucauuccggugcuauaggggcaguucaaguuuu
cccggguaugcaaaccgaaauccucacuugaagagacggaaguucugucuuguauucauuggguacgauccaaggc
ccguacgcacaauccuuacaagcuuucaucaaccuugaccaacauuuauacagguuccagacuccacgaagccgga
ugugcacccucauaucauguggugcgaggggauauugccacggccaccgaaggagugauuauaaaugcugcuaaca
gcaaaggacaaccuggcggaggggugugcggagcgcuguauaagaaauucccggaaagcuucgauuuacagccgau
cgaaguaggaaaagcgcgacuggucaaaggugcagcuaaacauaucauucaugccguaggaccaaacuucaacaaa
guuucggagguugaaggugacaaacaguuggcagaggcuuaugaguccaucgcuaagauugucaacgauaacaauu
acaagucaguagcgauuccacuguuguccaccggcaucuuuuccgggaacaaagaucgacuaacccaaucauugaa
ccauuugcugacagcuuuagacaccacugaugcagauguagccauauacugcagggacaagaaaugggaaaugacu
cucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaac
uuucucauauuuggaagggaccaaguuucaccaggcggccaaggauauagcagaaauuaaugccauguggcccguu
gcaacggaggccaaugagcagguaugcauguauauccucggagaaagcaugagcaguauuaggucgaaaugccccg
ucgaagagucggaagccuccacaccaccuagcacgcugccuugcuugugcauccaugccaugacuccagaaagagu
acagcgccuaaaagccucacguccagaacaaauuacugugugcucauccuuuccauugccgaaguauagaaucacu
ggugugcagaagauccaaugcucccagccuauauuguucucaccgaaagugccugcguauauucauccaaggaagu
aucucguggaaacaccaccgguagacgagacuccggagccaucggcagagaaccaauccacagaggggacaccuga
acaaccaccacuuauaaccgaggaugagaccaggacuagaacgccugagccgaucaucaucgaagaggaagaagag
gauagcauaaguuugcugucagauggcccgacccaccaggugcugcaagucgaggcagacauucacgggccgcccu
cuguaucuagcucauccugguccauuccucaugcauccgacuuugauguggacaguuuauccauacuugacacccu
ggagggagcuagcgugaccagcggggcaacgucagccgagacuaacucuuacuucgcaaagaguauggaguuucug
gcgcgaccggugccugcgccucgaacaguauucaggaacccuccacaucccgcuccgcgcacaagaacaccgucac
uugcacccagcagggccugcucgagaaccagccuaguuuccaccccgccaggcgugaauagggugaucacuagaga
ggagcucgaggcgcuuaccccgucacgcacuccuagcaggucggucucgagaaccagccuggucuccaacccgcca
ggcguaaauagggugauuacaagagaggaguuugaggcguucguagcacaacaacaaugacgguuugaugcgggug
cauacaucuuuuccuccgacaccggucaagggcauuuacaacaaaaaucaguaaggcaaacggugcuauccgaagu
gguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugca
uccuguuccuuuguauucaucuagugugaaccgugccuuuucaagcccccaagguccaguggaagccuguaacgcc
auguugaaagagaacuuuccgacuguggcuucuuacuguauuuucccccagaguacgaugccuauuuggaauguug
acggagcuucaugcugcuuagacacugccaguuuuugcccugcaaagcugcgcagcuuuccaaagaaacacuccua
uuuggaacccacaauacgaucggcagugccuucagcgauccagaacacgcuccagaacguccuggcagcugccaca
aaaagaaauugcaaugucacgcaaaugagagaauugcccguauuccggauucggggccuuuaauguggaauguuca
agaaauaugcguguaauaaugaauauugggaaacguuuaaagaaaaccccaucaggcuuacugaagaaaacguggu
aaauuacauuaccaaauuaaaaggaccaaaagcugcugcucuuuuugcgaagacacauaauuugaauauguugcag
gacauaccaauggacagguuuguaauggacuuaaagagagacgugaaagugacuccaggaacaaaacauacugaag
aacggcccaagguacaggugauccaggcugccgauccgcuagcaacagcguaucugugcggaauccaccgagagcu
gguuaggagauuaaaugcgguccugcuuccgaacauucauacacuguuugauaugucggcugaagacuuugacgcu
auuauagccgagcacuuccagccuggggauuguguucuggaaacugacaucgcgucguuugauaaaagugaggacg
acgccauggcucugaccgcguuaaugauucuggaagacuuagguguggacgcagagcuguugacgcugauugaggc
ggcuuucggcgaaauuucaucaauacauuugcccacuaaaacuaaauuuaaauucggagccaugaugaaaucugga
auguuccucacacuguuugugaacacagucauuaacauuguaaucgcaagcagaguguugagagaacggcuaaccg
gaucaccaugugcagcauucauuggagaugacaauaucgugaaaggagucaaaucggacaaauuaauggcagacag
gugcgccaccugguugaauauggaagucaagauuauagaugcuguggugggcgagaaagcgccuuauuucugugga
ggguuuauuuugugugacuccgugaccggcacagcgugccguguggcagacccccuaaaaaggcuguuuaagcuug
gcaaaccucuggcagcagacgaugaacaugaugaugacaggagaagggcauugcaugaagagucaacacgcuggaa
ccgaguggguauucuuucagagcugugcaaggcaguagaaucaagguaugaaaccguaggaacuuccaucauaguu
auggccaugacuacucuagcuagcaguguuaaaucauucagcuaccugagaggggccccuauaacucucuacggcu
aaccugaauggacuacgacauagucuaguccgccaagAUGGAGCUGCUCAUCCUUAAAGCGAAUGCUAUAACUACU
AUACUUACAGCUGUAACCUUUUGCUUCGCCUCUGGCCAAAACAUUACUGAGGAGUUUUAUCAAUCCACUUGCAGCG
CAGUCUCAAAAGGAUAUCUGUCAGCAUUGCGCACCGGGUGGUACACCAGCGUUAUCACUAUCGAGCUUUCCAACAU
UAAAGAGAAUAAGUGUAACGGCACAGACGCGAAAGUCAAACUCAUUAAGCAAGAACUUGAUAAAUACAAAAAUGCA
GUUACUGAACUUCAGUUGCUCAUGCAAUCAACCCCAGCAACGAACAAUAGGGCCAGGAGAGAAUUGCCGAGGUUUA
UGAACUACACACUUAAUAACGCUAAAAAGACUAACGUUACGCUCUCUAAGAAGCGGAAGCGCAGGUUUCUCGGGUU
CCUUCUGGGGGUUGGCAGUGCAAUAGCAUCCGGCGUCGCGGUAUCAAAGGUGCUUCAUCUUGAAGGAGAAGUUAAC
AAAAUCAAAAGCGCCUUGCUUUCAACUAAUAAGGCAGUAGUAUCAUUGUCUAACGGUGUCAGCGUCUUGACUUCCA
AGGUUUUGGAUUUGAAAAACUAUAUCGACAAACAACUUCUCCCGAUCGUUAACAAGCAGUCAUGCAGUAUCUCCAA
CAUCGAAACCGUCAUCGAAUUCCAACAGAAAAACAAUAGACUGCUUGAAAUUACUAGAGAGUUUUCAGUGAACGCU
GGCGUUACCACUCCGGUAUCCACUUAUAUGCUCACUAAUAGCGAACUGCUGUCUCUGAUAAAUGACAUGCCAAUAA
CUAACGACCAGAAGAAACUUAUGAGUAACAACGUCCAGAUCGUGAGACAGCAAUCAUAUAGCAUUAUGAGCAUAAU
UAAGGAGGAGGUCCUUGCAUACGUAGUCCAGCUCCCACUGUACGGGGUUAUCGACACGCCAUGUUGGAAGCUUCAU
ACUUCCCCCUUGUGCACCACGAACACGAAGGAAGGGUCUAACAUUUGUCUCACGCGCACUGAUCGGGGGUGGUACU
GUGACAACGCCGGGUCAGUGUCAUUUUUCCCUCAGGCCGAGACCUGCAAGGUCCAAUCAAACCGGGUAUUCUGUGA
UACUAUGAACUCCCUGACUCUGCCUUCUGAAGUUAACCUGUGUAAUGUAGAUAUAUUCAAUCCUAAAUACGACUGC
AAGAUAAUGACCAGCAAAACCGACGUGUCCUCAUCUGUCAUCACUUCCCUUGGUGCUAUAGUAAGCUGCUAUGGCA
AAACGAAAUGCACCGCGAGUAAUAAAAAUCGCGGUAUCAUCAAGACAUUUAGUAACGGCUGCGAUUAUGUUUCCAA
CAAAGGUGUUGACACCGUAUCUGUGGGGAAUACCCUGUAUUACGUAAAUAAGCAGGAAGGGAAAUCUCUCUACGUG
AAGGGGGAACCGAUAAUCAACUUUUAUGACCCGCUGGUUUUUCCGUCCGACGAAUUUGACGCGAGUAUCUCCCAAG
UCAACGAGAAAAUUAACCAAAGCCUCGCGUUCAUAAGAAAAUCCGAUGAGCUGCUGCAUAAUGUAAACGCGGGCAA
AUCAACUACCAACAUCAUGAUUACAACAAUAAUCAUCGUGAUAAUCGUGAUCCUGCUUUCACUUAUCGCUGUCGGG
CUUCUUCUCUAUUGUAAGGCCCGGAGUACUCCCGUGACCUUGUCUAAGGAUCAGCUCUCAGGUAUCAACAAUAUUG
CAUUCAGCAAUUGAggcgcgccauacagcagcaauuggcaagcugcuuacauagaacucgcggcgauuggcaugcc
aaaaaaaaaaaaaaaaaaaaaaaaaaaaaaaaa
SEQ ID NO: 75 Full-length VEEV + RSV-G Antigen Encoding RNA Sequence
auaggcggcgcaugagagaagcccagaccaauuaccuacccaaaauggagaaaguucacguugacaucgaggaaga
cagcccauuccucagagcuuugcagcggagcuucccgcaguuugagguagaagccaagcaggucacugauaaugac
caugcuaaugccagagcguuuucgcaucuggcuucaaaacugaucgaaacggagguggacccauccgacacgaucc
uugacauuggaagugcgcccgcccgcagaauguauucuaagcacaaguaucauuguaucuguccgaugagaugugc
ggaagauccggacagauuguauaaguaugcaacuaagcugaagaaaaacuguaaggaaauaacugauaaggaauug
gacaagaaaaugaaggagcuggccgccgucaugagcgacccugaccuggaaacugagacuaugugccuccacgacg
acgagucgugucgcuacgaagggcaagucgcuguuuaccaggauguauacgcgguugacggaccgacaagucucua
ucaccaagccaauaagggaguuagagucgccuacuggauaggcuuugacaccaccccuuuuauguuuaagaacuug
gcuggagcauauccaucauacucuaccaacugggccgacgaaaccguguuaacggcucguaacauaggccuaugca
gcucugacguuauggagcggucacguagagggauguccauucuuagaaagaaguauuugaaaccauccaacaaugu
ucuauucucuguuggcucgaccaucuaccacgagaagagggacuuacugaggagcuggcaccugccgucuguauuu
cacuuacguggcaagcaaaauuacacaugucggugugagacuauaguuaguugcgacggguacgucguuaaaagaa
uagcuaucaguccaggccuguaugggaagccuucaggcuaugcugccuacgaugcaccgcgagggauuuugugcug
caaagugacagacacauugaacggggagagggucucuuuucccgugucgcacguaugugccaguacauugugugac
caaaugacuggcauacuggcaacagaugucagugcggacgacgcgcaaaaacugcugguugggcucaaccagcgua
uagucgucaacggucgcacccagagaaacaccaauaccaugaaaaauuaccuuuugcccguaguggcccaggcauu
ugcuaggugggcaaaggaauauaaggaagaucaagaagaugaaaggccacuaggacuacgagauagacaguuaguc
augggguguuguugggcuuuuagaaggcacaagauaacaucuauuuauaagcgcccggauacccaaaccaucauca
aagugaacagcgauuuccacucauucgugcugcccaggauaggcaguaacacauuggagaucgggcugagaacaag
aaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguug
aggagcccacucuggaggcagacgucgacuugauguuacaagaggcuggggccggcucaguggagacaccucgugg
cuugauaaagguuaccagcuacgauggcgaggacaagaucggcucuuacgcugugcuuucuccgcaggcuguacuc
aagagugaaaaauuaucuugcauccacccucucgcugaacaagucauagugauaacacacucuggccgaaaagggc
guuaugccguggaaccauaccaugguaaaguaguggugccagagggacaugcaauacccguccaggacuuucaagc
ucugagugaaagugccaccauuguguacaacgaacgugaguucguaaacagguaccugcaccauauugccacacau
ggaggagcgcugaacacugaugaagaauauuacaaaacugucaagcccagcgagcacgacggcgaauaccuguacg
acaucgacaggaaacagugcgucaagaaagaacuagucacugggcuagggcucacaggcgagcugguggauccucc
cuuccaugaauucgccuacgagagucugagaacacgaccagccgcuccuuaccaaguaccaaccauagggguguau
ggcgugccaggaucaggcaagucuggcaucauuaaaagcgcagucaccaaaaaagaucuaguggugagcgccaaga
aagaaaacugugcagaaauuauaagggacgucaagaaaaugaaagggcuggacgucaaugccagaacuguggacuc
agugcucuugaauggaugcaaacaccccguagagacccuguauauucgacgaagcuuuuguugucaugcagguacu
cucagagcgcucauagccauuauaagaccuaaaaaggcagugcucuggcgggaucccaaacagugcgguuuuuuua
acaugaugugccugaaagugcauuuuaaccacgagauuugcacacaagucuuccacaaaagcaucucucgccguug
cacuaaaucugugacuucggucgucucaaccuuguuuuacgacaaaaaaaugagaacgacgaauccgaaagagacu
aagauugugauugacacuaccggcaguaccaaaccuaagccaggacgaucucauucucacuuguuucagagggggg
ugaagcaguugcaaauagauuacaaaggcaacgaaauaaugacggcagcugccucucaagggcugacccguaaagg
uguguaugccguucgguacaaggugaaugaaaauccucuguacgcacccaccucagaacaugugaacguccuacug
acccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcu
ggcauagacaugaccacugaacaauggaacacuguggauuauuuugaaacggacaaagcucacucagcagagauag
uauugaaccaacuaugcgugagguucuuuggacucgaucuggacuccggucuauuuucugcacccacuguuccguu
auccauuaggaauaaucacugggauaacuccccgucgccuaacauguacgggcugaauaaagaagugguccgucag
cucucucgcagguacccacaacugccucgggcaguugccacuggaagagucuaugacaugaacacugguacacugc
gcaauuaugauccgcgcauaaaccuaguaccuguaaacagaagacugccucaugcuuuaguccuccaccauaauga
acacccacagagugacuuuucuucauucgucagcaaauugaagggcagaacuguccugguggucggggaaaaguug
uccgucccaggcaaaaugguugacugguugucagaccggccugaggcuaccuucagagcucggcuggauuuaggca
ucccaggugaugugcccaaauaugacauaauauuuguuaaugugaggaccccauauaaauaccaucacuaucagca
gugugaagaccaugccauuaagcuuagcauguugaccaagaaagcuugucugcaucugaaucccggcggaaccugu
gucagcauagguuaugguuacgcugacagggccagcgccaaagcaucauuggugcuauaggggcaguucaaguuuu
cccggguaugcaaaccgaaauccucacuugaagagacggaaguucuguuuguauucauuggguacgaucgcaaggc
ccguacgcacaauccuuacaagcuuucaucaaccuugaccaacauuuauacagguuccagacuccacgaagccgga
ugugcacccucauaucauguggugcgaggggauauugccacggccaccgaaggagugauuauaaaugcugcuaaca
gcaaaggacaaccuggcggaggggugugcggagcgcuguauaagaaauucccggaaagcuucgauuuacagccgau
cgaaguaggaaaagcgcgacuggucaaaggugcagcuaaacauaucauucaugccguaggaccaaacuucaacaaa
guuucggagguugaaggugacaaacaguuggcagaggcuuaugaguccaucgcuaagauugucaacgauaacaauu
acaagucaguagcgauuccacuguuguccaccggcaucuuuuccgggaacaaagaucgacuaacccaaucauugaa
ccauuugcugacagcuuuagacaccacugaugcagauguagccauauacugcagggacaagaaaugggaaaugacu
cucaaggaagcaguggcuaggagagaagcaguggaggagauaugcauauccgacgacucuucagugacagaaccug
augcagagcuggugagggugcauccgaagaguuuucuggcuggaaggaagggcuacagcacaagcgauggcaaaac
uuucucauauuuggaagggaccaaguuucaccaggggccaaggaucauagcagaaauuaaugccauguggcccguu
gcaacggaggccaaugagcagguaugcauguauauccucggagaaagcaugagcaguauuaggucgaaaugccccg
ucgaagagucggaagccuccacaccaccuagcacgcugccuugcuugugcauccaugccaugacuccagaaagagu
acagcgccuaaaagccucacguccagaacaaauuacugugugcucauccuuuccauugccgaaguauagaaucacu
ggugugcagaagauccaaugcucccagccuauauuguucucaccgaaagugccugcguauauucauccaaggaagu
aucucguggaaacaccaccgguagacgagacuccggagccaucggcagagaaccaauccacagaggggacaccuga
acaaccaccacuuauaaccgaggaugagaccaggacuagaacgccugagccgaucaucaucgaagaggaagaagag
gauagcauaaguuugcugucagauggcccgacccaccaggugcugcaagucgaggcagacauucacgggccgcccu
cuguaucuagcucauccugguccauuccucaugcauccgacuuugauguggacaguuuauccauacuugacacccu
ggagggagcuagcgugaccagcggggcaacgucagccgagacuaacucuuacuucgcaaagaguauggaguuucug
gcgcgaccggugccugcgccucgaacaguauucaggaacccuccacaucccgcuccgcgcacaagaacaccgucac
uugcacccagcagggccugcucgagaaccagccuaguuuccaccccgccaggcgugaauagggugaucacuagaga
ggagcucgaggcgcuuaccccgucacgcacuccuagcaggucggucucgagaaccagccuggucuccaacccgcca
ggcguaaauagggugauuacaagagaggaguuugaggcguucguagcacaacaacaaugacgguuugaugcgggug
cauacaucuuuuccuccgacaccggucaagggcauuuacaacaaaaaucaguaaggcaaacggugcuauccgaagu
gguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugca
uccuguuccuuuguauucaucuagugugaaccgugccuuuucaagccccaaggucgcaguggaagccuguaacgcc
auguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccua
uuuggaacccacaauacgaucggcagugccuucagcgauccagaacacgcuccagaacguccuggcagcugccaca
aaaagaaauugcaaugucacgcaaaugagagaauugcccguauuggccauucggggccuuuaauguggaauguuca
agaaauaugcguguaauaaugaauauugggaaacguuuaaagaaaaccccaucaggcuuacugaagaaaacguggu
aaauuacauuaccaaauuaaaaggaccaaaagcugcugcucuuuuugcgaagacacauaauuugaauauguugcag
gacauaccaauggacagguuuguaauggacuuaaagagagacgugaaagugacuccaggaacaaaacauacugaag
aacggcccaagguacaggugauccaggcugccgauccgcuagcaacagcguaucugugcggaauccaccgagagcu
gguuaggagauuaaaugcgguccugcuuccgaacauucauacacuguuugauaugucggcugaagacuuugacgcu
auuauagccgagcacuuccagccuggggauuguguucuggaaacugacaucgcgucguuugauaaaagugaggacg
acgccauggcucugaccgcguuaaugauucuggaagacuuagguguggacgcagagcuguugacgcugauugaggc
ggcuuucggcgaaauuucaucaauacauuugcccacuaaaacuaaauuuaaauucggagccaugaugaaaucugga
auguuccucacacuguuugugaacacagucauuaacauuguaaucgcaagcagaguguugagagaacggcuaaccg
gaucaccaugugcagcauucauuggagaugacaauaucgugaaaggagucaaaucggacaaauuaauggcagacag
gugcgccaccugguugaauauggaagucaagauuauagaugcuguggugggcgagaaagcgccuuauuucugugga
ggguuuauuuugugugacuccgugaccggcacagcgugccguguggcagacccccuaaaaaggcuguuuaagcuug
gcaaaccucuggcagcagacgaugaacaugaugaugacaggagaagggcauugcaugaagagucaacacgcuggaa
ccgaguggguauucuuucagagcugugcaaggcaguagaaucaagguaugaaaccguaggaacuuccaucauaguu
auggccaugacuacucuagcuagcaguguuaaaucauucagcuaccugagaggggccccuauaacucucuacggcu
aaccugaauggacuacgacauagucuaguccgccaagAUGUCCAAAAAUAAAGACCAGCGAACGGCCAAGACUCUG
GAGAGGACUUGGGAUACCCUGAAUCAUCUGUUGUUUAUUUCAAGUUGCCUUUAUAAAUUGAAUCUCAAAUCCGUCG
CGCAAAUCACGCUGAGCAUACUCGCAAUGAUUAUCUCAACUUCCUUGAUAAUUGCCGCCAUAAUCUUCAUUGCAAG
UGCAAAUCAUAAAGUGACUCCUACAACCGCAAUAAUACAGGACGCGACCAGCCAAAUUAAGAACACGACUCCCACG
UAUCUCACCCAAAAUCCCCAACUCGGAAUUAGCCCAAGUAAUCCGUCAGAGAUUACUUCACAAAUCACCACCAUAC
UGGCGAGCACAACUCCAGGCGUAAAAUCCACCCUCCAAUCUACUACCGUCAAGACUAAAAAUACCACUACUACUCA
GACACAACCUAGUAAACCUACGACUAAGCAGCGCCAGAAUAAACCCCCAAGCAAACCAAACAACGACUUCCAUUUU
GAAGUUUUCAACUUCGUGCCUUGUUCUAUCUGCUCUAAUAAUCCAACAUGCUGGGCCAUCUGCAAGCGCAUUCCGA
AUAAGAAACCCGGUAAGAAAACUACAACGAAACCUACCAAGAAGCCUACCCUGAAAACGACAAAGAAAGACCCGAA
ACCGCAAACAACCAAAAGUAAAGAAGUGCCCACAACUAAACCCACAGAAGAACCAACGAUAAACACGACCAAAACC
AACAUAAUAACUACGCUGCUUACCAGCAACACGACAGGCAACCCGGAAUUGACCAGUCAGAUGGAAACUUUUCAUU
CAACCAGCAGCGAAGGUAAUCCAAGUCCUAGUCAGGUGUCUACGACAUCUGAAUAUCCAUCUCAGCCUAGUUCCCC
UCCGAACACGCCGCGCCAGUGAUAAccgcggugucaaaaaccgcguggacgugguuaacaucccugcugggaggau
cagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuu
uauuuuuucuuuucuuuuccgaaucggauuuuguuuuuaauauuuaaaaaaaaaaaaaaaaaaaaaaaaaaaaaaa
aaaaaaaaaaaaaaa
SEQ ID NO: 76 Full Length VEEV + Influenza H3 Antigen Encoding RNA Sequence
auaggcggcgcaugagagaagcccagaccaauuaccuacccaaaauggagaaaguucacguugacaucgaggaaga
cagcccauuccucagagcuuugcagcggagcuucccgcaguuugagguagaagccaagcaggucacugauaaugac
caugcuaaugccagagcguuuucgcaucuggcuucaaaacugaucgaaacggagguggacccauccgacacgaucc
uugacauuggaagugcgcccgcccgcagaauguauucuaagcacaaguaucauuguaucuguccgaugagaugugc
ggaagauccggacagauuguauaaguaugcaacuaagcugaagaaaaacuguaaggaaauaacugauaaggaauug
gacaagaaaaugaaggagcuggccgccgucaugagcgacccugaccuggaaacugagacuaugugccuccacgacg
acgagucgugucgcuacgaagggcaagucgcuguuuaccaggauguauacgcgguugacggaccgacaagucucua
ucaccaagccaauaagggaguuagagucgccuacuggauaggcuuugacaccaccccuuuuauguuuaagaacuug
gcuggagcauauccaucauacucuaccaacugggccgacgaaaccguguuaacggcucguaacauaggccuaugca
gcucugacguuauggagcggucacguagagggauguccauucuuagaaagaaguauuugaaaccauccaacaaugu
ucuauucucuguuggcucgaccaucuaccacgagaagagggacuuacugaggagcuggcaccugccgucuguauuu
cacuuacguggcaagcaaaauuacacaugucggugugagacuauaguuaguugcgacggguacgucguuaaaagaa
uagcuaucaguccaggccuguaugggaagccuucaggccuaugcugcuacgaugcaccgcgagggauuuugugcug
caaagugacagacacauugaacggggagagggucucuuuucccgcugugcacguaugugccaguacauugugugac
caaaugacuggcauacuggcaacagaugucagugcggacgacgcgcaaaaacugcugguugggcucaaccagcgua
uagucgucaacggucgcacccagagaaacaccaauaccaugaaaaauuaccuuuugcccguaguggcccaggcauu
ugcuaggugggcaaaggaauauaaggaagaucaagaagaugaaaggccacuaggacuacgagauagacaguuaguc
augggguguuguugggcuuuuagaaggcacaagauaacaucuauuuauaagcgcccggauacccaaaccaucauca
aagugaacagcgauuuccacucauucgugcugcccaggauaggcaguaacacauuggagaucgggcugagaacaag
aaucaggaaaauguuagaggagcacaaggagccgucaccucucauuaccgccgaggacguacaagaagcuaagugc
gcagccgaugaggcuaaggaggugcgugaagccgaggaguugcgcgcagcucuaccaccuuuggcagcugauguug
aggagcccacucuggaggcagacgucgacuugauguuacaagaggcuggggccggcucaguggagacaccucgugg
cuugauaaagguuaccagcuacgauggcgaggacaagaucggcucuuacgcugugcuuucuccgcaggcuguacuc
aagagugaaaaauuaucuugcauccacccucucgcugaacaagucauagugauaacacacucuggccgaaaagggc
guuaugccguggaaccauaccaugguaaaguaguggugccagagggacaugcaauacccguccaggacuuucaagc
ucugagugaaagugccaccauuguguacaacgaacgugaguucguaaacagguaccugcaccauauugccacacau
ggaggagcgcugaacacugaugaagaauauuacaaaacugucaagcccagcgagcacgacggcgaauaccuguacg
acaucgacaggaaacagugcgucaagaaagaacuagucacugggcuagggcucacaggcgagcugguggauccucc
cuuccaugaauucgccuacgagagucugagaacacgaccagccgcuccuuaccaaguaccaaccauagggguguau
ggcgugccaggaucaggcaagucuggcaucauuaaaagcgcagucaccaaaaaagaucuaguggugagcgccaaga
aagaaaacugugcagaaauuauaagggacgucaagaaaaugaaagggcuggacgucaaugccagaacuguggacuc
agugcucuugaauggaugcaaacaccccguagagacccuguauauugaccgaagcuuuuguugucaugcagguacu
cucagagcgcucauagccauuauaagaccuaaaaaggcagugcucugcggggaucccaaacagugcgguuuuuuua
acaugaugugccugaaagugcauuuuaaccacgagauuugcacacaagucuuccacaaaagcaucucucgccguug
cacuaaaucugugacuucggucgucucaaccuuguuuuacgacaaaaaaaugagaacgacgaauccgaaagagacu
aagauugugauugacacuaccggcaguaccaaaccuaagcagcgacgaucucauucucacuuguuucagagggggg
ugaagcaguugcaaauagauuacaaaggcaacgaaauaaugacggcagcugccucucaagggcugacccguaaagg
uguguaugccguucgguacaaggugaaugaaaauccucuguacgcacccaccucagaacaugugaacguccuacug
acccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagacc
ggacccuaccgacgucuuccagaauaaggcaaacguguguugggccaaggcuuuagugccggugcugaagaccgcu
ggcauagacaugaccacugaacaauggaacacugugcgauuuuuugaaacggacaaagcucacucagcagagauag
uauugaaccaacuaugcgugagguucuuuggacucgaucuggacuccggucuauuuucugcacccacuguuccguu
auccauuaggaauaaucacugggauaacuccccgucgccuaacauguacgggcugaauaaagaagugguccgucag
cucucucgcagguacccacaacugccucgggcaguugccacuggaagagucuaugacaugaacacugguacacugc
gcaauuaugauccgcgcauaaaccuaguaccuguaaacagaagacugccucaugcuuuaguccuccaccauaauga
acacccacagagugacuuuucuucauucgucagcaaauugaagggcagaacuguccugguggucggggaaaaguug
uccgucccaggcaaaaugguugacugguugucagaccggccugaggcuaccuucagagcucggcuggauuuaggca
ucccaggugaugugcccaaauaugacauaauauuuguuaaugugaggaccccauauaaauaccaucacuaucagca
gugugaagaccaugccauuaagcuuagcauguugaccaagaaagcuugucugcaucugaaucccggcggaaccugu
gucagcauagguuaugguuacgcugacagggccagcgaaaccgcaucauuggugcuauaggggcaguucaaguuuu
cccggguaugcaaaccgaaauccucacuugaagagacggaaguucuguuuguauucauuggguacgaucgcaaggc
ccguacgcacaauccuuacaagcuuucaucaaccuugaccaacauuuauacagguuccagacuccacgaagccgga
ugugcacccucauaucauguggugcgaggggauauugccacggccaccgaaggagugauuauaaaugcugcuaaca
gcaaaggacaaccuggcggaggggugugcggagcgcuguauaagaaauucccggaaagcuucgauuuacagccgau
cgaaguaggaaaagcgcgacuggucaaaggugcagcuaaacauaucauucaugccguaggaccaaacuucaacaaa
guuucggagguugaaggugacaaacaguuggcagaggcuuaugaguccaucgcuaagauugucaacgauaacaauu
acaagucaguagcgauuccacuguuguccaccggcaucuuuuccgggaacaaagaucgacuaacccaaucauugaa
ccauuugcugacagcuuuagacaccacugaugcagauguagccauauacugcagggacaagaaaugggaaaugacu
cucaaggaagcaguggcuaggagagaagcaguggaggagauaugcauauccgacgacucuucagugacagaaccug
augcagagcuggugagggugcauccgaagaguucuuuggcuggaaggaagggcuacagcacaagcgauggcaaaac
uuucucauauuuggaagggaccaaguuucaccaggcggccaaggauauagcagaaauuaaugccauguggcccguu
gcaacggaggccaaugagcagguaugcauguauauccucggagaaagcaugagcaguauuaggucgaaaugccccg
ucgaagagucggaagccuccacaccaccuagcacgcugccuugcuugugcauccaugccaugacuccagaaagagu
acagcgccuaaaagccucacguccagaacaaauuacugugugcucauccuuuccauugccgaaguauagaaucacu
ggugugcagaagauccaaugcucccagccuauauuguucucaccgaaagugccugcguauauucauccaaggaagu
aucucguggaaacaccaccgguagacgagacuccggagccaucggcagagaaccaauccacagaggggacaccuga
acaaccaccacuuauaaccgaggaugagaccaggacuagaacgccugagccgaucaucaucgaagaggaagaagag
gauagcauaaguuugcugucagauggcccgacccaccaggugcugcaagucgaggcagacauucacgggccgcccu
cuguaucuagcucauccugguccauuccucaugcauccgacuuugauguggacaguuuauccauacuugacacccu
ggagggagcuagcgugaccagcggggcaacgucagccgagacuaacucuuacuucgcaaagaguauggaguuucug
gcgcgaccggugccugcgccucgaacaguauucaggaacccuccacaucccgcuccgcgcacaagaacaccgucac
uugcacccagcagggccugcucgagaaccagccuaguuuccaccccgccaggcgugaauagggugaucacuagaga
ggagcucgaggcgcuuaccccgucacgcacuccuagcaggucggucucgagaaccagccuggucuccaacccgcca
ggcguaaauagggugauuacaagagaggaguuugaggcguucguagcacaacaacaaugacgguuugaugcgggug
cauacaucuuuuccuccgacaccggucaagggcauuuacaacaaaaaucaguaaggcaaacggugcuauccgaagu
gguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugca
uccuguuccuuuguauucaucuagugugaaccgugccuuuucaagccccaaggucccaguggaagccuguaacgcc
auguugaaagagaacuuuccgacuguggcuucuacuaguauuauuccagaguacgaugccuauuuggacaugguug
acggagcuucaugcugcuuagacacugccaguuuuugcccugcaaagcugcgcagcuuuccaaagaaacacuccua
uuuggaacccacaauacgaucggcagugccuucagcgauccagaacacgcuccagaacguccuggcagcugccaca
aaaagaaauugcaaugucacgcaaaugagagaacuugcccguauuggauucggggccuuuaauguggaaugcuuca
agaaauaugcguguaauaaugaauauugggaaacguuuaaagaaaaccccaucaggcuuacugaagaaaacguggu
aaauuacauuaccaaauuaaaaggaccaaaagcugcugcucuuuuugcgaagacacauaauuugaauauguugcag
gacauaccaauggacagguuuguaauggacuuaaagagagacgugaaagugacuccaggaacaaaacauacugaag
aacggcccaagguacaggugauccaggcugccgauccgcuagcaacagcguaucugugcggaauccaccgagagcu
gguuaggagauuaaaugcgguccugcuuccgaacauucauacacuguuugauaugucggcugaagacuuugacgcu
auuauagccgagcacuuccagccuggggauuguguucuggaaacugacaucgcgucguuugauaaaagugaggacg
acgccauggcucugaccgcguuaaugauucuggaagacuuagguguggaccgcagaguguugacgcugauugaggc
ggcuuucggcgaaauuucaucaauacauuugcccacuaaaacuaaauuuaaauucggagccaugaugaaaucugga
auguuccucacacuguuugugaacacagucauuaacauuguaaucgcaagcagaguguugagagaacggcuaaccg
gaucaccaugugcagcauucauuggagaugacaauaucgugaaaggagucaaaucggacaaauuaauggcagacag
gugcgccaccugguugaauauggaagucaagauuauagaugcuguggugggcgacgaaagcgccuuuuucugugga
ggguuuauuuugugugacuccgugaccggcacagcgugccguguggcagacccccuaaaaaggcuguuuaagcuug
gcaaaccucuggcagcagacgaugaacaugaugaugacaggagaagggcauugcaugaagagucaacacgcuggaa
ccgaguggguauucuuucagagcugugcaaggcaguagaaucaagguaugaaaccguaggaacuuccaucauaguu
auggccaugacuacucuagcuagcaguguuaaaucauucagcuaccugagaggggccccuauaacucucuacggcu
aaccugaauggacuacgacauagucuaguccgccaagAUGAAAACAAUUAUCGCUCUCAGUUAUAUUUUCUGCCUU
CCUUUGGGGCAAGACUUGCCGGGCAAUGAUAAUAGUACAGCGACUCUCUGUCUCGGACACCACGCAGUACCGAACG
GCACACUUGUGAAGACAAUCACAGACGAUCAAAUCGAAGUGACGAAUGCAACAGAGCUUGUUCAAUCAUCAUCUAC
CGGCAAAAUCUGCAAUAAUCCGCAUAGAAUCCUUGAUGGAAUCGACUGCACCCUGAUUGACGCACUGUUGGGAGAU
CCUCAUUGCGAUGUGUUUCAGAAUGAGACAUGGGACCUUUUUGUCGAGAGGAGCAAAGCGUUUUCCAACUGCUACC
CGUAUGACGUGCCUGAUUACGCGUCACUCCGCUCACUUGUAGCAUCAAGCGGUACUCUGGAGUUCAUCACCGAAGG
AUUCACCUGGACGGGCGUAACUCAGAACGGCGGUUCUAACGCAUGUAAGAGGGGGCCAGGGUCCGGCUUCUUCUCA
CGGCUCAACUGGUUGACUAAGUCUGGUUCAACAUACCCGGUCCUUAACGUUACAAUGCCGAAUAAUGACAACUUUG
AUAAACUCUACAUCUGGGGCAUUCACCAUCCAUCCACAAAUCAAGAACAGACAAGUUUGUACGUUCAGGCGUCAGG
GCGCGUUACCGUGAGUACAAGAAGAUCACAGCAAACAAUUAUACCCAAUAUUGGGUCCCGACCCUGGGUAAGAGGA
CUGUCCUCUCGCAUCUCCAUAUACUGGACCAUUGUCAAACCGGGCGACGUCCUGGUUAUCAAUAGUAAUGGAAACC
UUAUUGCUCCGCGCGGCUAUUUCAAAAUGCGAACUGGAAAGUCAAGUAUAAUGCGCUCAGACGCACCGAUCGAUAC
UUGUAUCAGUGAAUGCAUCACCCCUAACGGGUCCAUACCGAACGAUAAGCCCUUCCAGAAUGUGAAUAAAAUCACG
UAUGGAGCAUGCCCCAAAUACGUGAAGCAAAACACCCUCAAGUUGGCUACGGGUAUGCGCAACGUCCCAGAAAAAC
AAACGCGAGGCUUGUUUGGGGCGAUAGCAGGUUUUAUCGAGAACGGCUGGGAAGGAAUGAUCGAUGGGUGGUACGG
CUUUCGCCAUCAAAACUCAGAAGGAACUGGGCAGGCCGCAGAUCUUAAGUCUACGCAAGCGGCGAUAGAUCAAAUU
AAUGGCAAGUUGAAUAGGGUGAUAGAGAAGACGAACGAGAAGUUCCAUCAAAUAGAGAAAGAAUUCAGUGAAGUAG
AGGGGCGAAUUCAGGAUUUGGAAAAAUAUGUCGAGGAUACUAAGAUCGACCUGUGGAGCUAUAACGCAGAGCUUCU
GGUAGCACUUGAGAACCAGCAUACUAUUGAUCUCACCGAUUCCGAGAUGAACAAGCUUUUUGAGAAGACCAGGAGA
CAGUUGCGCGAGAAUGCCGAAGAAAUGGGUAACGGUUGUUUUAAGAUUUAUCACAAAUGUGACAACGCGUGUAUUG
AGUCCAUUAGGAAUGGUACAUAUGAUCACGAUGUGUAUCGCGAUGAAGCACUUAACAAUCGGUUCCAAAUAAAGGG
CGUGGAGCUCAAGAGUGGGUAUAAAGAUUGGAUCCUCUGGAUCUCAUUCGCGAUUUCCUGCUUCCUCCUGUGCGUU
GUCUUGCUGGGCUUUAUUAUGUGGGCAUGUCAGAGAGGUAACAUCCGGUGCAACAUAUGUAUCUGAUAAccgcggu
gucaaaaaccgcguggacgugguuaacaucccuccgcugggaggaucagccguaauuuuauaauuggcuuggugug
gcuacuauuguggccauguacgugcugaccaaccagaaacauaauugaauacagcagcaauuggcaagcugcuuac
guuuuuaauauuucaaaaaaaaaaaaaaaaaaaaaaaaaaaaaaaaaaaaaaaaaaaaa
SEQ ID NO: 77 Vector Backbone RNA Sequence 1
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguucauggagcggucacguagagggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcccaaaauuacacaugucggugugagacuauaguaguugg
acggguacgucguuaaaagaauagcuaucaguccaggccuguaugggaagccuucaggcuaugcugcuacgaugcaccgcg
agggauucuugugcugcaaagugacagacacauugaacccggggagagggucucuuuucccgugugcaguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaaguaagucgcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacacccucguggcuugauaaagguuaccaguacgauggcg
aggacaagaucggcucuuacgcugugcuuucuccgcaggcugucacucaagagugaaaaauuaucuugcaucacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccugccuauauugaccgaagcuuuuguugucaucagua
cucucagagcgcucauagccauuauaagaccuaaaaaggcagugcucugggggccaucccaaacaguggguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagaccaugaccacugaacaauggaacacuguggauuuu
uugaaacggacaaagcucacucagcagagauaguauugaaccaacuacugcgugagguuuuuggacucgaucuggacuccg
gucuauuuucugcacccacuguuccguuauccauuaggaauaauccacugggauaacuccccgucgccuaacauguacggg
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucccaaguuuucccggguaugca
aaccgaaauccucacuugaagagacggaaguucuguuuguauucauuggguacgaucccaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcugcgaaggaagggcuaca
gcacaagcgauggcaaaacuuucucauauuuggaagggaccaaguuucaccaggggcccaaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuuccgacacccuggagggaguagcgugaccaggggg
caacgucagccgagacuaacucuuacuucgcaaagaguauggaguuccccucuggggaccggugccuggcucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgagcgcgcuuaccccgucaccacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaacccgugccuuuucaagccccaagguccaguggaagccugua
acgccauguugaaagagaacuuuccgacuguggcuucuuacuguauuauuccagaguacgaugccuauuuggacaugguug
acggagcuucaugcugcuuagacacugccaguuuuugccccugcaaagcugcgcagcuuuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucccggggccuuuaauguggaaugcuucagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgdddaaauuucaucaauacauuugcccacuaaaaaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugdaaaccguaggaacuuccaucauaguaugg
ccaugacuacucuagcuagcaguguuaaaucauucagcuaccugagaggggccccuauaacucucuacggcuaaccugaau
ggacuacgacauagucuaguccgccaagXugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaa
aaaaaa
SEQ ID NO: 78 Vector Backbone RNA Sequence 2
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauugucaucuguccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggacuguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauucacacaugucggugugcagacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggggggucucuuuuccccccgugugcacguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagucccccccgaaaaauuuuugcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguacuauugacgaagcuuuuguugucaugcaggua
cucucagagcgcucauagccauuauaagaccuaaaaaggcagugcucugggggccaucccaaacaguggguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugacgguuuuuggacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcacguucaaguuuucccgggcuaugca
aaccgaaauccucacuugaagagacggaaguucuguuuguauucauuggguaccgaugcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcucggaaggaagggcuaca
gcacaagcgauggcaaaacuuucucauauuuggaagggaccaaguuucaccaggggccaaggcauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacccacccuggagggaguagcgugaccaggggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaccaccgugccuuuucaagccccaagguccaguggaaccugua
acgccauguugaaagagaacuuuccgacugugccccgcuuccccccuuacuguauuuuccagaguagauguaugaauguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaacugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgcagcacuuccccaccuggggauucuuuugg
aaacugacaucgcgucguuugauaaaagugaggacgacgccauggcucugaccgcguuaaugauucuggaagacuuaggug
uggacgcagagcuguugacgcugauugaggggcuuuccccggcgaaauuucaucaaucccacauuugcccacuaaaaaaaa
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucccuagguaaccugaau
ggacuacgacauagucuaguccgccaagXugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaaa
aaaa
SEQ ID NO: 79 Vector Backbone RNA Sequence 3
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucugucccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucacccaagccaauaagggaguagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuacuggagcggucacguagagggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggccugugagacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucccuuuucccgugugcaguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguaccaagaaguaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcugccuacucaagagugaaaaauuuuucccccgcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccugcuauauugacgaagcuuuuguugucaugcaggua
cucucagagcgcucauagccauuauaagaccuaaaaaggcagugcuccugggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugacacaauggaacacuguggauuuu
uugaaacggacaaagcucacucagcagagauaguauugaaccaacuaugcgugaggcuuuuuggacucgaucuggacuccg
gucuauuuucugcacccacuguuccguuauccauuaggaauaaucacugggauacacuccccgucgccuaacauguagggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguccuuucccggguaugca
aaccgaaauccucacuugaagagacggaaguucuguuuguauucauuggguacgaugcaaggccccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccacggaucccuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaaggaacgggcuaca
gcacaagcgauggcaaaacuuucucauauuuggaagggaccaaguuucaccaggggccaaggauauagcacgaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuuguugugcauccaugccaugacucccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgcugacccaggggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagcccccaagguccaguggaagccugua
acgccauguugaaagagacccccacuuuccgacugcccccuggcuucuuacuguauuuucccccgaguaguuuucccccuu
acggagcuucaugcugcuuagacacugccaguuuuugcccugcaaagcuggcagcuuuccaaaaaacacuccuaccuuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucaccgaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauauagccgacccccgacuccagcccggggauuuucccuu
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacauuugcccacuaaaacucaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcugugguggggagaaagccgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguuaugg
ccaugacuacucuagcuagcaguguuaaaucauucagcuaccugagaggggccccuauaacucucuacggcuaaccugaau
ggacuacgacauagucuaguccgccaagXuaguaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaaaa
aaaaaa
SEQ ID NO: 80 Vector Backbone RNA Sequence 4
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauugucaucuguccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacccuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugccacguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaaguggcagcccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaacuuaucuugcaucacccucucg
cugaacaagucauagugauaacacacucuggccgaaaagggguuaugccguggaaccauacccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuucuguugucaugcaggua
cucucagagcgcucauagccauuauaagaccuaaaaaggcagugcucugggggaucccaacacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggcaacacuguggauuuu
uugaaacggacaaagcucacucagcagagauaguauugaaccaacuaugcgugagguuuuugcgacucgaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccccuucagccagcugguggauuu
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcccaguucaaguuuucccggguaugca
aaccgaaauccucacuugaagagacggaaguucuguuuguauucauuggguacgaucgcaaggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccugggccgggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauucccccacuguuguccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcucggaaggaagggcuaca
gcacaagcgauggcaaaacuuucucauauuuggaagggaccaaguuucaccaggggccacaggauauagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauuccaucaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccugggggaguagcgugcccaccaggggg
caacgucagccgagacuaacucuuacuucgcaaagaguauggaguuucuggggaccggugccugcgccccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacccacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccacguggaagccugua
acgccauguugaaagagaacuuuccgacuguggcuucuuacuguauuauuccagaguacgaugccuauuuggacaugguug
acggagcuucaugcugcuuagacacugccaguuuuugcccugcccaaagcuggcagcuuuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccucuuaauguggaaugcuucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacguauauagccgccccagacuccagugcccccccgggauuuuuu
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacacuuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguacgaaucaagguaugaaaccguaggaacuuccaucauaguaugg
ccaugacuacucuagcuagcaguguuaaaucauucagcuaccugagaggggccccuauaacucucuacggcuaaccugaau
ggacuacgacauagucuaguccgccaagXugauaaccgcggugucaaaaaccgcguggacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuuguuuuuaauauuucaaaaaaaaaaaaaaaaaaaaaaaaaaaaaaaaaaaaaaa
aaaaaa
SEQ ID NO: 81 Vector Backbone RNA Sequence 5
auaggcggcgcaugagagaagcccagaccaauuaccuacccaaaauggagaaaguucacguugacaucgaggaagac
agcccauuccucagagcuuugcagcggagcuucccgcaguuugagguagaagccaagcaggucacugauaaugacca
ugcuaaugccagagcguuuucgcaucuggcuucaaaacugaucgaaacggagguggacccauccgacacgauccuug
acauuggaagugcgcccgcccgcagaauguauucuaagcacaaguaucaucuguaucuguccgaugagaugugggaa
gauccggacagauuguauaaguaugcaacuaagcugaagaaaaacuguaaggaaauaacugauaaggaauuggacaa
gaaaaugaaggagcuggccgccgucaugagcgacccugaccuggaaacugagacuaugugccuccacgacgacgagu
cgugucgcuacgaagggcaagucgcuguuuaccaggauguauacgcgguugacggaccgacaagucucuaucaccaa
gccaauaagggaguuagagucgccuacuggauaggcuuugacaccaccccuuuuauguuuaagaacuuggcuggagc
auauccaucauacucuaccaacugggccgacgaaaccguguuaacggcucguaacauaggccuaugcagcucugacg
uuauggagcggucacguagagggauguccauucuuagaaagaaguauuugaaaccccauccaacaauguucuauuuu
guuggcucgaccaucuaccacgagaagagggacuuacugaggagcuggcaccugccgucuguauuucacuuacgugg
caagcaaaauuacacaugucggugugagacuauaguuaguugcgacggguacgucgcuuaaaagaauaguaucaguc
caggccuguaugggaagccuucaggcuaugcugcuacgaugcaccgcgagggauucuugugcugcaaagugacagac
acauugaacggggagagggucucuuuucccgugugcacguaugugccaguacauugugucgaccaaaugacuggcau
acuggcaacagaugucagugcggacgacgcgcaaaaacugcugguugggcucaaccagcguauagucgucaacgguc
gcacccagagaaacaccaauaccaugaaaaauuaccuuuugcccguaguggcccaggcauuugcuaggugggcaaag
gaauauaaggaagaucaagaagaugaaaggccacuaggacuacgagauagacaguuagucaugggguguuguugggc
uuuuagaaggcacaagauaacaucuauuuauaagcgcccggauacccaaaccaucaucaaagugaacagcgauuucc
acucauucgugcugcccaggauaggcaguaacacauuggagaucgggcugagaacaagaaucaggaaaauguuagag
gagcacaaggagccgucaccucucauuaccgccgaggacguacaagaagcuaagugcgcagccgaugaggcuaagga
ggugcgugaagccgaggaguugcgcgcagcucuaccaccuuuggcagcugauguugaggagcccacucuggaggcag
acgucgacuugauguuacaagaggcuggggccggcucaguggagacaccucguggcuugauaaaggucuaccaguac
gauggcgaggacaagaucggcucuuacgcugugcuuucuccgcaggcuguacucaagagugaaaaccauuuuugcau
ccacccucucgcugaacaagucauagugauaacacacucuggccgaaaagggguuaugccguggacaccauaccaug
guaaaguaguggugccagagggacaugcaauacccguccaggacuuucaagcucugagugaaagugccaccauugug
uacaacgaacgugaguucguaaacagguaccugcaccauauugccacacauggaggagcgcugaacacugaugaaga
auauuacaaaacugucaagcccagcgagcacgacggcgaauaccuguacgacaucgacaggaaacagugcgucaaga
aagaacuagucacugggcuagggcucacaggcgagcugguggauccucccuuccaugaauucgccuacgagagucug
agaacacgaccagccgcuccuuaccaaguaccaaccauagggguguauggcgugccaggaucaggcaagucuggcau
cauuaaaagcgcagucaccaaaaaagaucuaguggugagcgccaagaaagaaaacugugcagaaauuauaagggacg
ucaagaaaaugaaagggcuggacgucaaugccagaacuguggacucagugcucuugaauggaugcaaacaccccgua
gagacccuguauauugacgaagcuuuugcuugucaugcagguacucucagaggcucauagccaucuauaagaccuaa
aaaggcagugcucugcggggaucccaaacagugcgguuuuuuuaacaugaugugccugaaagugcauuuuaaccacg
agauuugcacacaagucuuccacaaaagcaucucucgccguugcacuaaaucugugacuuccggugucucaaccuug
uuuuacgacaaaaaaaugagaacgacgaauccgaaagagacuaagauugugauugacacuaccggcaguaccaaacc
uaagcaggacgaucucauucucacuuguuucagagggugggugaagcaguugcaaauagauuacaaaggcaacgaaa
uaaugacggcagcugccucucaagggcugacccguaaagguguguaugccguucgguacaaggugaaugaaaauccu
cuguacgcacccaccucagaacaugugaacguccuacugacccgcacggaggaccgcaucguguggaaaacacuagc
cggcgacccauggauaaaaacacugacugccaaguacccugggaauuucacugccacgauagaggaguggcaagcag
agcaugaugccaucaugaggcacaucuuggagagaccggacccuaccgacgucuuccagaauaaggcaaacgugugu
ugggccaaggcuuuagugccggugcugaagaccgcuggcauagacaugaccacugaacaauggaacacuguggauua
uuuugaaacggacaaagcucacucagcagagauaguauugaaccaacuaugcgugagguucuuuggacucgaucugg
acuccggucuauuuucugcacccacuguuccguuauccauuaggaauaaucacugggauaacuccccgucgccuaac
auguacgggcugaauaaagaagugguccgucagcucucucgcagguacccacaacugccucgggcaguugccacugg
aagagucuaugacaugaacacugguacacugcgcaauuaugauccgcgcauaaaccuaguaccuguaaacagaagac
ugccucaugcuuuaguccuccaccauaaugaacacccacagagugacuuuucuucauucgucagcaaauugaagggc
agaacuguccugguggucggggaaaaguuguccgucccaggcaaaaugguugacugguugucagaccggccugaggc
uaccuucagagcucggcuggauuuaggcaucccaggugaugugcccaaauaugacauaauauuuguuaaugugagga
ccccauauaaauaccaucacuaucagcagugugaagaccaugccauuaagcuuagcauguugaccaagaaagcuugu
cugcaucugaaucccggcggaaccugugucagcauagguuaugguuacgcugacagggccagcgaaagcaucauugg
ugcuauagcgcggcaguucaaguuuucccggguaugcaaaccgaaauccucacuugaagagacggaaguucuguuug
uauucauuggguacgaucgcaaggcccguacgcacaauccuuacaagcuuucaucaaccuugaccaacauuuauaca
gguuccagacuccacgaagccggaugugcacccucauaucauguggugcgaggggauauugccacggccaccgaagg
agugauuauaaaugcugcuaacagcaaaggacaaccuggcggaggggugugcggagcgcuguauaagaaauucccgg
aaagcuucgauuuacagccgaucgaaguaggaaaagcgcgacuggucaaaggugcagcuaaacauaucauucaugcc
guaggaccaaacuucaacaaaguuucggagguugaaggugacaaacaguuggcagaggcuuaugaguccaucgcuaa
gauugucaacgauaacaauuacaagucaguagcgauuccacuguuguccaccggcaucuuuuccgggaacaaagauc
gacuaacccaaucauugaaccauuugcugacagcuuuagacaccacugaugcagauguagccauauacugcagggac
aagaaaugggaaaugacucucaaggaagcaguggcuaggagagaagcaguggaggagauaugcauauccgacgacuc
uucagugacagaaccugaugcagagcuggugagggugcauccgaagaguuuuuggcuggaaggaagggcuaccagca
caagcgauggcaaaacuuucucauauuuggaagggaccaaguuucaccagcgggccaaggauauagcagaaauuaau
gccauguggcccguugcaacggaggccaaugagcagguaugcauguauauccucggagaaagcaugagcaguauuag
gucgaaaugccccgucgaagagucggaagccuccacaccaccuagcacgcugccuugcuugugcauccaugccauga
cuccagaaagaguacagcgccuaaaagccucacguccagaaaaauuacugugugcucauccuuuccaucugccgaag
uauagaaucacuggugugcagaagauccaaugcucccagccuauauuguucucaccgaaagugccugcguauauuca
uccaaggaaguaucucguggaaacaccaccgguagacgagacuccggagccaucggcagagaaccaauccacagagg
ggacaccugaacaaccaccacuuauaaccgaggaugagaccaggacuagaacgccugagccgaucaucaucgaagag
gaagaagaggauagcauaaguuugcugucagauggcccgacccaccaggugcugcaagucgaggcagacauucacgg
gccgcccucuguaucuagcucauccugguccauuccucaugcaucacccccuuugauguggacaguccccccuuuuu
acacccuggagggagcuagcgugaccagcggggcaacgucagccgagacuaacucuuacuucgcaaagaguauggag
uuucuggcgcgaccggugccugcgccucgaacaguauucaggaacccuccacaucccgcuccgcgcacaagaacacc
gucacuugcacccagcagggccugcucgagaaccagccuaguuuccaccccgccaggcgugaauagggugaucacua
gagaggagcucgaggcgcuuaccccgucacgcacuccuagcaggucggucucgagaaccagccuggucuccaacccg
ccaggcguaaauagggugauuacaagagaggaguuugaggcguucguagcacaacaacaaugacggcuuugaugggg
ugcauacaucuuuuccuccgacaccggucaagggcauuuacaacaaaaaucaguaaggcaaacggugcuauccgaag
ugguguuggagaggaccgaauuggagauuucguaugccccgcgccucgaccaagaaaaagaagaauuacuacgcaag
aaauuacaguuaaaucccacaccugcuaacagaagcagauaccaguccaggaagguggagaacaugaaagccauaac
agcuagacguauucugcaaggccuagggcauuauuugaaggcagaaggaaaaguggagugcuaccgaacccugcauc
cuguuccuuuguauucaucuagugugaaccgugccuuuucaagccccaagguccacguggaagccuguaacgccaug
uugaaagagaacuuuccgacuguggcuucuuacuguauuauuccagaguagaugccccccccccuaugacaguugag
agcuucaugcugcuuagacacugccaguuuuugcccugcaaagcuggcagcuuuccaaaaaacacccuccuauuugg
aacccacaauacgaucggcagugccuucagcgauccagaacacgcuccagaacguccuggcagcugccacaaaaaga
aauugcaaugucacgcaaaugagagaauugcccguauuggauucggggccuuuaauguggacaugcuucaagaaaua
ugcguguaauaaugaauauugggaaacguuuaaagaaaaccccaucaggcuuacugaagaaaacgugguaaauuaca
uuaccaaauuaaaaggaccaaaagcugcugcucuuuuugcgaagacacauaauuugaauauguugcaggacauacca
auggacagguuuguaauggacuuaaagagagacgugaaagugacuccaggaacaaaacauacugaagaacggcccaa
gguacaggugauccaggcugccgauccgcuagcaacagcguaucugugcggaauccaccgagagcugguuaggagau
uaaaugcgguccugcuuccgaacauucauacacuguuugauaugucggcugaagacuuugaccccccuauaugcgag
cacuuccagccuggggauuguguucuggaaacugacaucgcgucguuugauaaaagugaggacgacgccauggcucu
gaccgcguuaaugauucuggaagacuuagguguggacgcagagcuguugacgcugaucugaggggcuuucggcgaaa
uuucaucaauacauuugcccacuaaaacuaaauuuaaauucggagccaugaugaaaucuggaauguuccucacacug
uuugugaacacagucauuaacauuguaaucgcaagcagaguguugagagaacggcuaaccggaucaccaugugcagc
auucauuggagaugacaauaucgugaaaggagucaaaucggacaaauuaauggcagacaggugcgccaccugguuga
auauggaagucaagauucccaucccccagccccaugcugugguggggagccaaagguuucccuuuccccgggggggg
uccgugaccggcacagcgugccguguggcagacccccuaaaaaggcuguuuaagcuuggcaaaccucuggcagcaga
cgaugaacaugaugaugacaggagaagggcauugcaugaagagucaacacgcuggaaccgaguggguauuuucucag
agcugugcaaggcaguagaaucaagguaugaaaccguaggaacuuccaucauaguuauggccaugacuacucuagcu
agcaguguuaaaucauucagcuaccugagaggggccccuauaacucucuacggcuaaccugaaugcgacuacacaua
gucuaguccgccaagXugauaaccgcggugucaaaaaccgcguggacgugguuaacaucccugcugggaggaucagc
cguaauuauuauaauuggcuuggugcuggcuacuauuguggccauguacgugcugaccaaccagaaacauaauugaa
uacagcagcaauuggcaagcugcuuacauagaacucgcggcgauuggcaugccgccuuaaaauuuuuauuuuauuuu
uucuuuucuuuuccgaaucggauuuuguuuuuaauauuucaaaaaaaaaaaaaaaaaaaaaaaaaaaaaaaaaaaaa
aaaaaaaa
SEQ ID NO: 82 Vector Backbone RNA Sequence 6
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugggaagaucccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuccauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugcugccagua
cauugugugaccaaaugacuggcauacuggcaacagaugucagugggacgacgcgcaaaaacugcuggcuugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccaguaccgauggcg
aggacaagaucggcucuuacgcugugcuuucuccgcaggcuguacucaagagugaaaaauuaucuugccaucacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugcucaugcaggua
cucucagagcgcucauagccauuauaagaccuaaaaaggcagugcucugggggaucccaaacagucgcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacugcuggauuuu
uugaaacggacaaagcucacucagcagagauaguauugaaccaacuaugcgugagguuuuuggacucgcaucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccucucagagcuggucccggauuu
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggggaaccugucgucagca
uagguuaugguuacgcugacagggccagcgaaagcaucauuggugcuauaggggcaguucaaguuuucccccggguaugca
aaccgaaauccucacuugaagagacggaaguucuguuuguauucauuggguacgauccaaggcccgccuagcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguugucccccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaaggacagggcuaca
gcacaagcgauggcaaaacuuucucauauuuggaagggaccaaguuucaccaggggccaaggauaucagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugacccagcgggg
caacgucagccgagacuaacucuuacuucgcaaagaguauggaguuucuggggaccggugccugcgccucgcaacacguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucaccacuccuacgcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccaguggcaagccugua
acgccauguugaaagagaacuuuccgacuguggcuucuuacuguauuauuccagaguacgaugccuauuuggacaugguug
acggagcuucaugcugcuuagacacugccaguuuuugcccugcaaagcuggcagcuuuccaaagaaacacuccucauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucaagaaauaucgcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauauagccgagacuccaggcccccccccccccccgggggg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacauuucgcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucaucaguaugg
ccaugacuacucuagcuagcaguguuaaaucauucagcuaccugagaggggccccuauaacucucuacggcuaaccugaau
ggacuacgacauagucuaguccgccaagAUGUUUCUGCUCACAACCAAACGCACUAUGUUUGUUUUCCUCGUGCUGCUCCC
UUUGGUAAGUUCUCAGUGUGUAAACCUGAGAACACGAACCCAGUUGCCUCCAGCUUAUACCAACUCAUUUACUCGCGGAGU
AUAUUAUCCCGAUAAGGUCUUUAGAAGUAGCGUGUUGCACUCUACACAGGAUCUGUUCUUGCCCUUCUUUAGUAACGUUAC
CUGGUUUCAUGCAAUACAUGUGAGCGGAACAAAUGGAACAAAAAGAUUUGACAAUCCAGUGCUUCCAUUUAAUGAUGGGGU
UUACUUUGCCAGUAUCGAAAAGUCAAACAUAAUCCGGGGGUGGAUCUUUGGAACCACUUUGGACUCUAAGACACAGUCUCU
CCUCAUAGUAAACAACGCCACCAAUGUUGUCAUAAAAGUAUGCGAAUUUCAGUUUUGCAACGAUCCCUUUCUCGACGUGUA
UUACCAUAAGAAUAAUAAAUCCUGGAUGGAGUCUGGGGUUUAUAGUAGUGCUAAUAAUUGCACUUUCGAAUACGUGUCCCA
ACCAUUCCUCAUGGACCUUGAGGGCAAACAGGGGAAUUUUAAAAACUUGCGCGAAUUUGUCUUUAAGAAUAUCGACGGAUA
CUUUAAGAUCUAUAGUAAACACACUCCUAUCAACCUCGUUCGGGAUCUUCCCCAAGGCUUUUCUGCUCUCGAACCCCUCGU
AGACUUGCCAAUUGGGAUAAAUAUCACUCGCUUUCAAACUUUGCUUGCCCUCCACAGGAGCUACCUGACACCCGGCGACUC
UUCUUCUGGUUUGACCGCCGGCGCCGCUGCCUAUUAUGUUGGUUACCUUCAGCCACGAACAUUCUUGCUCAAGUAUAACGA
GAAUGGCACCAUUACCGACGCCGUCGAUUGUGCAUUGGAUCCCUUGUCUGAAACAAAAUGUACCUUGAAGUCCUUUACCGU
AGAGAAAGGCAUAUACCAGACUUCCAACUUCCGAGUUCAGCCUACAGAAUCCAUCGUACGAUUUCCCAACAUCACAAACCU
CUGCCCUUUCGGUGAAGUAUUUAAUGCUACACGCUUCGCUUCAGUCUAUGCCUGGAAUAGGAAGCGCAUAUCAAAUUGCGU
GGCCGAUUAUUCAGUCCUCUAUAAUAGCGCAUCCUUCAGUACUUUCAAGUGCUACGGCGUUUCCCCCACCAAACUCAAUGA
UCUUUGCUUCACCAACGUCUAUGCUGACAGUUUUGUCAUACGAGGCGACGAAGUACGCCAGAUUGCCCCCGGGCAGACAGG
UAAUAUUGCUGAUUAUAAUUAUAAACUCCCAGAUGACUUUACUGGAUGCGUCAUAGCCUGGAAUUCCAACAAUCUAGAUUC
CAAGGUUGGUGGGAAUUAUAAUUACCGUUAUCGACUGUUCAGAAAGAGUAACUUGAAACCAUUUGAGAGAGACAUAUCCAC
CGAGAUUUACCAGGCAGGCAGUAAGCCUUGUAACGGCGUUGAGGGAUUUAACUGCUAUUUUCCUUUGCAAUCCUAUGGCUU
UCAACCAACAAACGGGGUUGGCUAUCAACCCUAUCGAGUGGUUGUCCUCAGCUUUGAACUUUUGCACGCUCCCGCCACAGU
CUGCGGACCAAAAAAGAGUACAAAUCUUGUCAAGAAUAAGUGCGUAAAUUUCAAUUUCAAUGGCCUUACAGGAACAGGCGU
GCUGACUGAGUCAAACAAGAAUUUCCUGCCAUUUCAGCAGUUUGGGCGGGAUAUAGCAGACACAACUGACGCUGUACGCGA
UCCUCAGACUUUGGAGAUCUUGGACAUCACUCCCUGUUCUUUCGGAGGGGUAUCUGUCAUCACCCCCGGAACUAAUACAUC
AAAUCAGGUCGCUGUGUUGUACCAAGGUGUCAACUGCACAGAAGUCCCCGUUGCUAUACACGCAGACCAGCUCACCCCCAC
AUGGCGGGUGUACUCAACUGGCUCAAACGUAUUCCAGACCAGAGCUGGGUGCUUGAUCGGUGCUGAACACGUGAACAAUAG
CUAUGAAUGCGAUAUUCCCAUCGGUGCCGGGAUCUGCGCUAGCUAUCAGACACAGACCAAUUCCCGCAGGCGGGCUCGCUC
UGUAGCAUCCCAGUCUAUUAUUGCCUACACUAUGUCAUUGGGCGCCGAGAAUAGCGUCGCAUAUUCAAAUAAUUCUAUUGC
AAUACCCACCAACUUCACAAUCUCCGUAACUACAGAAAUACUUCCAGUUUCCAUGACAAAGACAUCAGUGGAUUGUACAAU
GUAUAUAUGCGGAGAUUCCACAGAAUGUUCAAAUUUGCUCUUGCAGUACGGCUCCUUCUGCACCCAGCUCAACAGGGCACU
UACAGGUAUUGCUGUCGAACAGGACAAGAACACACAAGAAGUCUUCGCCCAAGUCAAACAGAUAUACAAAACUCCUCCCAU
AAAGGAUUUUGGCGGCUUCAACUUUAGUCAGAUCCUCCCAGACCCUUCAAAACCAUCUAAACGAUCAUUUAUUGAAGAUCU
GCUGUUCAACAAGGUCACUCUUGCCGAUGCUGGAUUCAUUAAGCAAUACGGUGACUGCCUUGGUGAUAUUGCUGCCCGAGA
UCUGAUCUGUGCCCAGAAAUUCAACGGGCUCACUGUACUCCCUCCACUGCUCACAGACGAAAUGAUUGCACAGUACACAAG
UGCCCUGUUGGCAGGCACAAUCACUAGCGGCUGGACCUUUGGCGCAGGUGCAGCACUCCAAAUACCUUUUGCCAUGCAGAU
GGCCUAUCGGUUUAAUGGGAUAGGCGUGACUCAAAAUGUCCUCUACGAAAACCAAAAGUUGAUAGCUAACCAAUUCAAUUC
AGCAAUCGGGAAGAUACAGGAUUCACUGUCUAGUACUGCUAGUGCCCUUGGUAAGCUGCAGAACGUUGUCAACCAGAAUGC
UCAAGCUCUGAAUACAUUGGUUAAGCAGCUCUCUAGUAAUUUUGGGGCCAUCUCUUCAGUACUUAAUGAUAUUUUGAGCCG
AUUGGACCCACCUGAAGCUGAAGUACAGAUCGACAGGCUGAUAACAGGCCGGCUCCAAUCCCUCCAAACAUACGUGACACA
ACAACUCAUACGCGCAGCCGAAAUCCGAGCCAGCGCUAACCUGGCAGCUACCAAGAUGUCAGAAUGCGUUCUGGGCCAGAG
UAAACGCGUAGAUUUCUGCGGGAAAGGGUACCACCUGAUGUCCUUUCCACAAUCUGCACCUCACGGGGUCGUCUUUUUGCA
UGUAACAUACGUACCCGCACAAGAGAAGAAUUUUACUACCGCUCCUGCCAUCUGUCAUGACGGGAAAGCUCAUUUUCCUCG
CGAAGGUGUGUUUGUAUCUAAUGGUACACAUUGGUUUGUCACACAGCGGAAUUUCUAUGAACCCCAGAUCAUUACAACUGA
CAACACUUUUGUUUCCGGGAAUUGUGACGUGGUCAUAGGAAUCGUAAAUAACACUGUAUAUGAUCCCCUCCAACCAGAGCU
GGACUCUUUUAAAGAAGAACUGGAUAAAUAUUUCAAGAACCACACAAGUCCCGACGUGGACCUUGGGGACAUAAGUGGUAU
UAACGCAUCUGUGGUUAACAUUCAAAAGGAAAUCGACAGACUCAACGAGGUGGCCAAAAACCUGAACGAAAGCUUGAUAGA
UCUCCAGGAGUUGGGCAAGUAUGAACAGUACAUUAAAUGGCCAUGGUACAUAUGGCUUGGCUUUAUCGCUGGCCUUAUCGC
CAUCGUAAUGGUUACAAUCAUGCUGUGCUGCAUGACCUCCUGCUGUUCUUGUUUGAAAGGGUGUUGUUCUUGUGGUAGUUG
UUGCAAGUUUGACGAAGAUGAUUCCGAACCUGUUCUUAAGGGGGUAAAGCUUCACUAUACAUGAuaaccgcggugucaaaa
accgcguggacgugguuaacaucccugcugggaggaucagccguaauuauuauaauuggcuuggugcuggcuacuauugug
gccauguacgugcugaccaaccagaaacauaauugaauacagcagcaauuggcaagcugcuuacauagaacucgcggcgau
uggcaugccgccuuaaaauuuuuauuuuauuuuuucuuuucuuuuccgaaucggauuuuguuuuuaauauuucaaaaaaaa
aaaaaaaaaaaaaaaaaaaaaaaaaaaaaaaaaaaaaaa
SEQ ID NO: 83 Vector Backbone RNA Sequence 7
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugacgaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaucguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacucaucaguaguugg
acggguacgucguuaaaagaauagcuaucaguccaggccuguaugggaagccuucaggcuaugcugcucacgaugcaccgg
agggauucuugugcugcaaagugacagacacauugaacggggagaggguuuuuucccgugugcacgcuaugcugcccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcucugauaaagguuaccaguacgauggcg
aggacaagaucggcucuuacgcugugcuuucuccgcaggcuguacucaagcccagugaaaaauuccccuuugcaucacucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuugcuugccucaucagua
cucucagagcgcucauagccauuauaagaccuaaaaaggcagugcucugggggaucccaaacagugcggcuuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguuuuuggacucgauccuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuuccccccggguauga
aaccgaaauccucacuugaagagacggaaguucuguuuguauucauuggguacgaugcaacggcccguacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaaggaacgggcuaca
gcacaagcgauggcaaaacuuucucauauuuggaagggaccaaguuucaccaggggccaaggauauagccagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccccaggggg
caacgucagccgagacuaacucuuacuucgcaaagaguauggaguuucuggggaccggugccugcgccuccgacacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauucuacaaaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccccccaagguccaguggaagcua
acgccauguugaaagagaacuuuccgacuguggcuuccccccuuacugccuauuuucgaguacccccccccgauguaugau
acggagcuucaugcugcuuagacacugccaguuuuugcccugcaaagcuggcagcuuuccaaagcaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucccagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauauagccccgagcacuuccaccuggggacccuuuuuugg
aaacugacaucgcgucguuugauaaaagugaggacgacgccauggcucugaccgcguuaaugauucuggaagacuuaggug
uggacgcagagcuguugacgcugauugaggcggcuuucggcgaaauuucaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaaccuuccaucauaguaugg
ccaugacuacucuagcuagcaguguuaaaucauucagcuaccugagaggggccccuauaacucucuacggcuaaccugaau
ggacuacgacauagucuaguccgccgccaccXuaaccgggugucaaaaaccgcguggcacgugguuaacaucccugcuggg
aggaucagccguaauuauuauaauuggcuuggugcuggcuacuauuguggccauguacgugcugaccaaccagaaacauaa
uugaauacagcagcaauuggcaagcugcuuacauagaacucgcggcgauuggcaugccgccuuaaaauuuuuauuuuauuu
uuucuuuucuuuuccgaaucggauuuuguuuuaauauuucaaaaaaaaaaaaaaaaaaaaaaaaaaaaaaaaaaaaaaaa
aaaaaa
SEQ ID NO: 84 VEE-EVD68-IRES-VLP
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugccagacuauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugucgccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaaucccuaucuugcaucacccucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguuguccaugcaggua
cucucagagcgcucauagccauuauaagaccuaaaaaggcagugcucugggggaucccaaacagugcggcuuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggcauuuu
uugaaacggacaaagcucacucagcagagauaguauugaaccaacuaugcgugagguuuuuggacucgaucuggaccuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccgccgguaugca
aaccgaaauccucacuugaagagacggaaguucuguuuguauucauuggguacgaugcaaggcccguacgcaccaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguugucccccacggauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaacggaagggcuaca
gcacaagcgauggcaaaacuuucucauauuuggaagggaccaaguuucaccaggggccaaggauaucagcagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccagccgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagcccccaagguccaguggaagccugua
acgccauguugaaagagaacuuuccgacuguggcuucuuacuguauuuuccagaguagacccuguaucccccccccgauuu
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaauguucaagaaauaugccccugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccaccuggccccggauuuuuugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacauuugcccacucaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguaugg
ccaugacuacucuagcuagcaguguuaaaucauucagcuaccugagaggggccccuauaacucucuacggcuaaccugaau
ggacuacgacauagucuaguccgccaagAUGGGUGCUCAGGUAACCAGGCAGCAAACCGGUACUCAUGAAAAUGCCAACAU
AGCUACUAAUGGCUCCCAUAUUACGUACAAUCAAAUCAAUUUCUACAAGGAUAGUUACGCUGCGUCCGCUUCUAAGCAGGA
CUUCAGUCAGGAUCCUAGCAAGUUUACGGAACCCGUAGUUGAAGGCCUUAAGGCAGGGGCACCUGUCCUUAAGUCACCGAG
UGCGGAGGCUUGCGGUUACUCUGACCGAGUACUGCAGCUUAAGCUCGGGAACUCUGCCAUAGUUACGCAGGAAGCGGCAAA
CUAUUGCUGCGCGUACGGGGAGUGGCCGAACUACCUGCCAGACCAUGAGGCGGUCGCUAUAGACAAGCCAACACAACCUGA
GACAGCCACGGAUCGGUUCUAUACUCUUAAAAGCGUAAAAUGGGAGACUGGCUCCACAGGAUGGUGGUGGAAGCUCCCAGA
UGCCCUUAACAAUAUCGGGAUGUUUGGCCAGAAUGUUCAACACCAUUACCUGUAUCGCAGUGGCUUCCUCAUUCACGUCCA
GUGUAAUGCCACAAAGUUUCAUCAGGGGGCUCUCCUUGUGGUGGCGAUCCCAGAGCAUCAGAGGGGUGCACAUAAUACUAA
UACUAGUCCUGGUUUCGAUGAUAUAAUGAAAGGGGAGGAAGGAGGGACGUUUAAUCAUCCUUAUGUCCUGGAUGACGGGAC
CUCAUUGGCGUGUGCGACGAUCUUCCCUCACCAGUGGAUUAAUCUCCGGACCAAUAACAGUGCGACUAUCGUACUUCCAUG
GAUGAACGCGGCUCCGAUGGAUUUUCCCCUGAGGCAUAAUCAGUGGACAUUGGCUAUUAUUCCGGUCGUACCCCUGGGUAC
UAGAACCACUAGCUCAAUGGUUCCCAUAACUGUAUCUAUUGCGCCAAUGUGCUGUGAAUUUAAUGGGCUCCGGCACGCUAU
CACACAAGGCGUUCCUACGUAUCUCUUGCCAGGCUCAGGUCAGUUCCUCACUACUGAUGACCAUAGCUCCGCACCUGCCCU
CCCCUGUUUUAACCCAACACCCGAGAUGCAUAUCCCAGGGCAAGUCCGAAACAUGCUUGAGGUUGUUCAGGUAGAAUCUAU
GAUGGAGAUCAAUAACACAGAGAGUGCGGUAGGGAUGGAGCGCCUUAAGGUUGACAUCUCCGCAUUGACCGAUGUUGACCA
ACUUUUGUUUAACAUUCCCCUGGAUAUACAGCUCGAUGGCCCCUUGCGGAACACGUUGGUCGGAAAUAUCUCCAGGUACUA
CACUCAUUGGUCCGGCAGUCUCGAAAUGACAUUUAUGUUUUGCGGCAGUUUCAUGGCAGCGGGCAAACUGAUCCUGUGUUA
UACACCCCCAGGCGGUAGUUGUCCAACGACGCGAGAGACGGCGAUGCUCGGCACACAUAUAGUGUGGGAUUUUGGCUUGCA
AUCCUCAGUUACCCUCAUCAUACCGUGGAUAAGCGGCAGCCAUUAUAGAAUGUUCAACAAUGACGCUAAAAGUACGAACGC
CAAUGUGGGAUAUGUGACCUGCUUUAUGCAGACGAAUCUCAUCGUACCUUCUGAGUCCUCAGACACAUGCAGUUUGAUAGG
UUUCAUAGCCGCAAAGGACGAUUUCAGUCUUAGACUUAUGCGGGACAGUCCGGACAUUGGUCAACUGGAUCACCUUCAUGC
UGCGGAGGCAGCAUAUCAGAUCGAAUCAAUAAUUAAAACUGCUACCGACACAGUCAAGUCCGAGAUAAACGCUGAACUGGG
CGUCGUCCCGAGUCUUAAUGCAGUGGAAACCGGAGCCACUUCUAAUACUGAGCCAGAAGAAGCAAUUCAAACUCGAACUGU
GAUCAACCAACACGGUGUAAGCGAGACUUUGGUAGAAAAUUUCCUCUCCAGAGCCGCCUUGGUAUCAAAAAGAAGUUUUGA
GUAUAAAGACCACACGAGCUCUACAGCACGCGCAGACAAGAACUUCUUUAAAUGGACGAUAAAUACCAGAAGUUUUGUACA
GCUCCGCAGGAAAUUGGAGCUCUUCACAUACCUCCGAUUUGACGCGGAAAUAACAAUUUUGACCACAGUUGCGGUUAAUGG
UAGUGGAAAUAACACGUACGUAGGCUUGCCUGAUCUGACACUGCAGGCCAUGUUUGUCCCUACUGGUGCACUCACUCCGGA
GAAACAGGACUCCUUCCAUUGGCAGAGCGGGUCAAAUGCGUCAGUGUUCUUCAAAAUCUCCGAUCCCCCCGCGAGGAUCAC
UAUUCCCUUUAUGUGUAUAAAUAGCGCCUAUAGCGUUUUUUACGAUGGCUUUGCCGGCUUUGAAAAGAAUGGGUUGUACGG
GAUUAAUCCGGCCGAUACGAUAGGUAACCUGUGUGUACGCAUAGUUAACGAACACCAGCCAGUGGGUUUCACUGUAACCGU
UCGAGUGUACAUGAAACCUAAGCACAUCAAGGCUUGGGCACCAAGGCCACCGAGAACCCUCCCAUACAUGAGCAUUGCUAA
UGCAAAUUAUAAGGGAAAAGAGAGAGCACCGAACGCGUUGUCUGCAAUUAUCGGCAAUCGGGACUCAGUCAAGACUAUGCC
ACAUAAUAUAGUCAAUACCugaccccucucccuccccccccccuaacguuacuggccgaagccgcuuggaauaaggccggu
gugcguuugucuauauguuauuuuccaccauauugccgucuuuuggcaaugugagggcccggaaaccuggcccugucuucu
ugacgagcauuccuaggggucuuuccccucucgccaaaggaaugcaaggucuguugaaugucgugaaggaagcaguuccuc
uggaagcuucuugaagacaaacaacgucuguagcgacccuuugcaggcagcggaaccccccaccuggcgacaggugccucu
gcggccaaaagccacguguauaagauacaccugcaaaggcggcacaaccccagugccacguugugaguuggauaguugugg
aaagagucaaauggcucuccucaagcguauucaacaaggggcugaaggaugcccagaagguaccccauuguaugggaucug
aucuggggccucggugcacaugcuuuacauguguuuagucgagguuaaaaaacgucuaggccccccgaaccacggggacgu
gguuuuccuuugaaaaacacgaugauaauauggccacaaccAUGGGUCCAGGCUUUGACUUUGCUCAGGCGAUAAUGAAGA
AAAACACGGUUAUCGCACGAACUGAAAAGGGCGAAUUUACCAUGCUCGGCGUGUACGAUCGAGUCGCGGUUAUCCCGACAC
ACGCUUCCGUGGGGGAAACCAUAUAUAUCAACGAUGUAGAAACCAAAGUCCUCGACGCGUGUGCACUGAGGGAUCUUACAG
ACACUAACCUGGAGAUCACAAUAGUGAAGCUGGAUCGAAAUCAGAAGUUCCGAGACAUCCGCCAUUUUUUGCCUAGAUAUG
AAGACGACUACAAUGAUGCUGUACUGUCCGUGCAUACUUCAAAGUUUCCUAACAUGUACAUCCCUGUUGGCCAGGUCACUA
AUUACGGUUUUCUUAACCUGGGUGGGACGCCUACUCAUAGGAUACUGAUGUACAAUUUUCCUACUAGAGCUGGACAAUGUG
GUGGCGUAGUGACUACCACCGGGAAAGUCAUUGGCAUACACGUAGGAGGUAACGGGGCGCAGGGAUUCGCGGCCAUGCUUC
UGCACAGCUAUUUCUCCGACACACAAGGUGAAAUAGUUUCAUCAGAGAAAUCCGGUGUGUGCAUUAACGCUCCCGCGAAAA
CUAAACUUCAGCCCAGCGUGUUUCAUCAAGUAUUCGAAGGAAGCAAGGAACCGGCUGUACUGAACCCCAAGGACCCCCGGC
UUAAAACGGAUUUCGAAGAAGCGAUAUUUUCAAAAUAUACUGGUAACAAAAUCAUGCUGAUGGAUGAGUAUAUGGAAGAAG
CUGUGGACCACUAUGUAGGGUGCCUGGAACCGCUCGACAUCUCUGUGGACCCGAUCCCACUCGAGUCCGCUAUGUACGGCA
UGGACGGCCUCGAGGCUUUGGACCUUACAACUAGCGCGGGCUUUCCGUAUCUUUUGCAAGGUAAGAAAAAGCGCGACAUCU
UUAACCGCCACACCAGGGAUACGAGCGAGAUGACAAAAAUGCUUGAAAAAUAUGGGGUCGAUCUUCCUUUUGUCACUUUCG
UGAAGGACGAAUUGAGAUCCCGAGAAAAGGUCGAGAAAGGUAAGUCUCGCCUCAUUGAAGCCAGUUCACUUAAUGAUAGUG
UUGCGAUGCGAGUUGCUUUUGGUAACCUUUACGCAACAUUUCAUAACAAUCCAGGCACGGCUACAGGAUCAGCAGUAGGUU
GCGACCCAGACAUCUUUUGGUCAAAAAUCCCCAUUCUGCUGGACGGUGAAAUUUUUGCCUUUGACUAUACCGGAUACGACG
CAUCCUUGUCCCCUGUAUGGUUCGCAUGUCUCAAAAAAGUCCUGAUAAAACUCGGUUACACUCACCAGACUAGUUUUAUAG
ACUAUCUGUGUCAUAGUGUUCACCUCUACAAAGAUAAAAAAUAUAUUGUGAACGGUGGUAUGCCGUCUGGUAGUUCCGGAA
CUUCCAUAUUUAACACAAUGAUUAAUAAUAUCAUUAUAAGGACGCUUCUCAUCAGGGUCUACAAGGGUAUCGAUCUGGAUC
AAUUCAAGAUGAUAGCAUACGGCGACGACGUCAUUGCUUCUUACCCCCAUAAGAUUGAUCCAGGUCUGCUGGCGGAAGCCG
GCAAGCAAUAUGGACUGGUUAUGACACCCGCUGACAAAGGAACCAGUUUCAUCGACACGAAUUGGGAAAACGUGACGUUCC
UGAAGCGAUACUUCAGAGCAGACGAUCAAUAUCCCUUUCUUAUCCAUCCCGUUAUGCCAAUGAAGGAGAUACACGAGUCAA
UCCGAUGGACAAAAGACCCACGGAACACACAAGAUCACGUCCGAUCACUCUGUUAUCUUGCCUGGCACAAUGGGGAGGAGG
CGUAUAAUGAGUUCUGCCGGAAGAUUCGAAGCGUACCAGUAGGCCGAGCACUGACUCUCCCUGCUUAUUCAAGUCUGCGGC
GGAAGUGGUUGGAUUCCUUCuaguaaccgcggugucaaaaaccgcguggacgugguuaacaucccugcugggaggaucagc
cguaauuauuauaauuggcuuggugcuggcuacuauuguggccauguacgugcugaccaaccagaaacauaauugaauaca
gcagcaauuggcaagcugcuuacauagaacucgcggcgauuggcaugccgccuuaaauuuuuuauuuuauuuuuucuuuuc
uuuuccgaaucggauuuuuuuuuaauauuucaaaaaaaaaaaaaaaaaaaaaaaaaaaaaaaaaaaaaaaaaaaaa
SEQ ID NO: 85 VEE-IL-18952-p1-IRES-3cd
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguauccuguccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcgcgucacguagagggauguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaccguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuuccgugugcacguauguccgccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagucacgauggcg
aggacaagaucggcucuuacgcugugcuuucuccgcaggcuguacucaagagugaaaaauuaucuugcauccacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucaucgcaggua
cucucagagcgcucauagccauuauaagaccuaaaaaggcagugcucugggggaucccaaacaguggguuuuccuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguuuuuggacucgaucuggaccuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggggaaccugucgucagca
uagguuaugguuacgcugacagggccagcgaaagcaucauuggugcuauaggggcaguucaaguuuucccccggguaugca
aaccgaaauccucacuugaagagacggaaguucuguuuguauucauuggguacgaugcaaggcccguacgccacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccccuggggggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggccuggaaggaagggcuaca
gcacaagcgauggcaaaacuuucucauauuuggaagggaccaaguuucaccaggggccaaggauauagccagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccagccgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccccaguggaaccugua
acgccauguugaaagagaacuuuccgacuguggcuucuuacuguauuauuccagaguacgaugccuauuuggacaugguug
acggagcuucaugcugcuuagacacugccaguuuuugcccugcccaaagcuggcagcuuuccaaaaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaauguccucaagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuuccaucaauacauuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccauccauaguaugg
ccaugacuacucuagcuagcaguguuaaaucauucagcuaccugagaggggccccuauaacucucuacggcuaaccugaau
ggacuacgacauagucuaguccgccaagAUGGGUGCUCAGGUAACCAGGCAGCAAACCGGUACUCAUGAAAAUGCCAACAU
AGCUACUAAUGGCUCCCAUAUUACGUACAAUCAAAUCAAUUUCUACAAGGAUAGUUACGCUGCGUCCGCUUCUAAGCAGGA
CUUCAGUCAGGAUCCUAGCAAGUUUACGGAACCCGUAGUUGAAGGCCUUAAGGCAGGGGCACCUGUCCUUAAGUCACCGAG
UGCGGAGGCUUGCGGUUACUCUGACCGAGUACUGCAGCUUAAGCUCGGGAACUCUGCCAUAGUUACGCAGGAAGCGGCAAA
CUAUUGCUGCGCGUACGGGGAGUGGCCGAACUACCUGCCAGACCAUGAGGCGGUCGCUAUAGACAAGCCAACACAACCUGA
GACAGCCACGGAUCGGUUCUAUACUCUUAAAAGCGUAAAAUGGGAGACUGGCUCCACAGGAUGGUGGUGGAAGCUCCCAGA
UGCCCUUAACAAUAUCGGGAUGUUUGGCCAGAAUGUUCAACACCAUUACCUGUAUCGCAGUGGCUUCCUCAUUCACGUCCA
GUGUAAUGCCACAAAGUUUCAUCAGGGGGCUCUCCUUGUGGUGGCGAUCCCAGAGCAUCAGAGGGGUGCACAUAAUACUAA
UACUAGUCCUGGUUUCGAUGAUAUAAUGAAAGGGGAGGAAGGAGGGACGUUUAAUCAUCCUUAUGUCCUGGAUGACGGGAC
CUCAUUGGCGUGUGCGACGAUCUUCCCUCACCAGUGGAUUAAUCUCCGGACCAAUAACAGUGCGACUAUCGUACUUCCAUG
GAUGAACGCGGCUCCGAUGGAUUUUCCCCUGAGGCAUAAUCAGUGGACAUUGGCUAUUAUUCCGGUCGUACCCCUGGGUAC
UAGAACCACUAGCUCAAUGGUUCCCAUAACUGUAUCUAUUGCGCCAAUGUGCUGUGAAUUUAAUGGGCUCCGGCACGCUAU
CACACAAGGCGUUCCUACGUAUCUCUUGCCAGGCUCAGGUCAGUUCCUCACUACUGAUGACCAUAGCUCCGCACCUGCCCU
CCCCUGUUUUAACCCAACACCCGAGAUGCAUAUCCCAGGGCAAGUCCGAAACAUGCUUGAGGUUGUUCAGGUAGAAUCUAU
GAUGGAGAUCAAUAACACAGAGAGUGCGGUAGGGAUGGAGCGCCUUAAGGUUGACAUCUCCGCAUUGACCGAUGUUGACCA
ACUUUUGUUUAACAUUCCCCUGGAUAUACAGCUCGAUGGCCCCUUGCGGAACACGUUGGUCGGAAAUAUCUCCAGGUACUA
CACUCAUUGGUCCGGCAGUCUCGAAAUGACAUUUAUGUUUUGCGGCAGUUUCAUGGCAGCGGGCAAACUGAUCCUGUGUUA
UACACCCCCAGGCGGUAGUUGUCCAACGACGCGAGAGACGGCGAUGCUCGGCACACAUAUAGUGUGGGAUUUUGGCUUGCA
AUCCUCAGUUACCCUCAUCAUACCGUGGAUAAGCGGCAGCCAUUAUAGAAUGUUCAACAAUGACGCUAAAAGUACGAACGC
CAAUGUGGGAUAUGUGACCUGCUUUAUGCAGACGAAUCUCAUCGUACCUUCUGAGUCCUCAGACACAUGCAGUUUGAUAGG
UUUCAUAGCCGCAAAGGACGAUUUCAGUCUUAGACUUAUGCGGGACAGUCCGGACAUUGGUCAACUGGAUCACCUUCAUGC
UGCGGAGGCAGCAUAUCAGAUCGAAUCAAUAAUUAAAACUGCUACCGACACAGUCAAGUCCGAGAUAAACGCUGAACUGGG
CGUCGUCCCGAGUCUUAAUGCAGUGGAAACCGGAGCCACUUCUAAUACUGAGCCAGAAGAAGCAAUUCAAACUCGAACUGU
GAUCAACCAACACGGUGUAAGCGAGACUUUGGUAGAAAAUUUCCUCUCCAGAGCCGCCUUGGUAUCAAAAAGAAGUUUUGA
GUAUAAAGACCACACGAGCUCUACAGCACGCGCAGACAAGAACUUCUUUAAAUGGACGAUAAAUACCAGAAGUUUUGUACA
GCUCCGCAGGAAAUUGGAGCUCUUCACAUACCUCCGAUUUGACGCGGAAAUAACAAUUUUGACCACAGUUGCGGUUAAUGG
UAGUGGAAAUAACACGUACGUAGGCUUGCCUGAUCUGACACUGCAGGCCAUGUUUGUCCCUACUGGUGCACUCACUCCGGA
GAAACAGGACUCCUUCCAUUGGCAGAGCGGGUCAAAUGCGUCAGUGUUCUUCAAAAUCUCCGAUCCCCCCGCGAGGAUCAC
UAUUCCCUUUAUGUGUAUAAAUAGCGCCUAUAGCGUUUUUUACGAUGGCUUUGCCGGCUUUGAAAAGAAUGGGUUGUACGG
GAUUAAUCCGGCCGAUACGAUAGGUAACCUGUGUGUACGCAUAGUUAACGAACACCAGCCAGUGGGUUUCACUGUAACCGU
UCGAGUGUACAUGAAACCUAAGCACAUCAAGGCUUGGGCACCAAGGCCACCGAGAACCCUCCCAUACAUGAGCAUUGCUAA
UGCAAAUUAUAAGGGAAAAGAGAGAGCACCGAACGCGUUGUCUGCAAUUAUCGGCAAUCGGGACUCAGUCAAGACUAUGCC
ACAUAAUAUAGUCAAUACCugaccccucucccuccccccccccuaacguuacuggccgaagccgcuuggaauaaggccggu
gugcguuugucuauauguuauuuuccaccauauugccgucuuuuggcaaugugagggcccggaaccuggcccugucuuccu
ugacgagcauuccuaggggucuuuccccucucgccaaaggaaugcaaggucuguugaaugucgugaaggaagcaguuccuc
uggaagcuucuugaagacaaacaacgucuguagcgacccuuugcaggcagcggaaccccccaccuggcgacaggugccucu
gcggccaaaagccacguguauaagauacaccugcaaaggcggcacaaccccagugccacguugugaguuggauaguugugg
aaagagucaaauggcucuccucaagcguauucaacaaggggcugaaggaugcccagaagguaccccauuguaugggaucug
aucuggggccucggugcacaugcuuuacauguguuuagucgagguuaaaaaacgucuaggccccccgaaccacggggacgu
gguuuuccuuugaaaaacacgaugauaauauggccacaaccAUGGGUCCAGGCUUUGACUUUGCUCAGGCGAUAAUGAAGA
AAAACACGGUUAUCGCACGAACUGAAAAGGGCGAAUUUACCAUGCUCGGCGUGUACGAUCGAGUCGCGGUUAUCCCGACAC
ACGCUUCCGUGGGGGAAACCAUAUAUAUCAACGAUGUAGAAACCAAAGUCCUCGACGCGUGUGCACUGAGGGAUCUUACAG
ACACUAACCUGGAGAUCACAAUAGUGAAGCUGGAUCGAAAUCAGAAGUUCCGAGACAUCCGCCAUUUUUUGCCUAGAUAUG
AAGACGACUACAAUGAUGCUGUACUGUCCGUGCAUACUUCAAAGUUUCCUAACAUGUACAUCCCUGUUGGCCAGGUCACUA
AUUACGGUUUUCUUAACCUGGGUGGGACGCCUACUCAUAGGAUACUGAUGUACAAUUUUCCUACUAGAGCUGGACAAUGUG
GUGGCGUAGUGACUACCACCGGGAAAGUCAUUGGCAUACACGUAGGAGGUAACGGGGCGCAGGGAUUCGCGGCCAUGCUUC
UGCACAGCUAUUUCUCCGACACACAAGGUGAAAUAGUUUCAUCAGAGAAAUCCGGUGUGUGCAUUAACGCUCCCGCGAAAA
CUAAACUUCAGCCCAGCGUGUUUCAUCAAGUAUUCGAAGGAAGCAAGGAACCGGCUGUACUGAACCCCAAGGACCCCCGGC
UUAAAACGGAUUUCGAAGAAGCGAUAUUUUCAAAAUAUACUGGUAACAAAAUCAUGCUGAUGGAUGAGUAUAUGGAAGAAG
CUGUGGACCACUAUGUAGGGUGCCUGGAACCGCUCGACAUCUCUGUGGACCCGAUCCCACUCGAGUCCGCUAUGUACGGCA
UGGACGGCCUCGAGGCUUUGGACCUUACAACUAGCGCGGGCUUUCCGUAUCUUUUGCAAGGUAAGAAAAAGCGCGACAUCU
UUAACCGCCACACCAGGGAUACGAGCGAGAUGACAAAAAUGCUUGAAAAAUAUGGGGUCGAUCUUCCUUUUGUCACUUUCG
UGAAGGACGAAUUGAGAUCCCGAGAAAAGGUCGAGAAAGGUAAGUCUCGCCUCAUUGAAGCCAGUUCACUUAAUGAUAGUG
UUGCGAUGCGAGUUGCUUUUGGUAACCUUUACGCAACAUUUCAUAACAAUCCAGGCACGGCUACAGGAUCAGCAGUAGGUU
GCGACCCAGACAUCUUUUGGUCAAAAAUCCCCAUUCUGCUGGACGGUGAAAUUUUUGCCUUUGACUAUACCGGAUACGACG
CAUCCUUGUCCCCUGUAUGGUUCGCAUGUCUCAAAAAAGUCCUGAUAAAACUCGGUUACACUCACCAGACUAGUUUUAUAG
ACUAUCUGUGUCAUAGUGUUCACCUCUACAAAGAUAAAAAAUAUAUUGUGAACGGUGGUAUGCCGUCUGGUAGUUCCGGAA
CUUCCAUAUUUAACACAAUGAUUAAUAAUAUCAUUAUAAGGACGCUUCUCAUCAGGGUCUACAAGGGUAUCGAUCUGGAUC
AAUUCAAGAUGAUAGCAUACGGCGACGACGUCAUUGCUUCUUACCCCCAUAAGAUUGAUCCAGGUCUGCUGGCGGAAGCCG
GCAAGCAAUAUGGACUGGUUAUGACACCCGCUGACAAAGGAACCAGUUUCAUCGACACGAAUUGGGAAAACGUGACGUUCC
UGAAGCGAUACUUCAGAGCAGACGAUCAAUAUCCCUUUCUUAUCCAUCCCGUUAUGCCAAUGAAGGAGAUACACGAGUCAA
UCCGAUGGACAAAAGACCCACGGAACACACAAGAUCACGUCCGAUCACUCUGUUAUCUUGCCUGGCACAAUGGGGAGGAGG
CGUAUAAUGAGUUCUGCCGGAAGAUUCGAAGCGUACCAGUAGGCCGAGCACUGACUCUCCCUGCUUAUUCAAGUCUGCGGC
GGAAGUGGUUGGAUUCCUUCuaguaaccgcggugucaaaaaccgcguggacgugguuaacaucccugcugggaggaucagc
cguaauuauuauaauuggcuuggugcuggcuacuauucguggccauguacgugcugaccaaccagaaacauaaugaauaca
gcagcaauuggcaagcugcuuacauagaacucgcggcgauuggcaugccgccuuaaaauuuuuauuuuauuuuuucuuuuc
uuuuccgaaucggauuuuguuuuuaauauuucaaaaaaaaaaaaaaaaaaaaaaaaaaaaaaaaaaaaaaaaaaaaa
SEQ ID NO: 86 VEE-JPOC10-396-p1-IRES-3cd
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauugucaucuguccgaugagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuccauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugcugcacguaugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugacuaaagguuaccaguacgauggcg
aggacaagaucggcucuuacgcugugcuuucuccgcaggcuguacucaagagugaaaaaucuaucuugcaucacccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucacugcaggua
cucucagagcgcucauagccauuauaagaccuaaaaaggcagugcucugggggaucccaaacagugggccuuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacugugcgauuuu
uugaaacggacaaagcucacucagcagagauaguauugaaccaacuaugcgugagguuuuuggacucgacucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucgguggaucuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccgcgcguaugca
aaccgaaauccucacuugaagagacggaaguucuguuuguauucauuggguacgauccaaggcccguacgccacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggggggccggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccaccggcaucuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuucuuggcuggaaggaagggcuaca
gcacaagcgauggcaaaacuuucucauauuuggaagggaccaaguuucaccaggggccaaggauauagccagaaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccugggggagucccagcgugaccaggggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagcccccaagguccaguggaagccugua
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaauccgcuucagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuuccccaucaauacauuugcccacuaaaaaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuacuagaugcuguggugggcgagaaaggccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaacccguaggaacuuccaucauaguaugg
ccaugacuacucuagcuagcaguguuaaaucauucagcuaccugagaggggccccuauaacucucuacggcuaaccugaau
ggacuacgacauagucuaguccgccaagAUGGGUGCACAAGUAACCCGGCAGCAGACUGGAACGCACGAGAAUGCCAACAU
UGCGACGAACGGUUCCCAUAUUACCUAUAACCAAAUAAAUUUCUAUAAAGACUCUUACGCGGCUAGCGCAAGUAAACAGGA
CUUUUCCCAGGAUCCAUCAAAAUUUACAGAACCAGUUGUGGAGGGGUUGAAGGCAGGAGCCCCCGUGCUCAAGUCCCCAUC
CGCCGAAGCUUGUGGCUAUUCUGAUCGCGUCUUGCAACUGAAACUCGGAAACUCAGCGAUCGUUACUCAAGAGGCUGCUAA
CUAUUGUUGUGCCUACGGUGAGUGGCCCAACUACUUGCCAGAUCACGAAGCCGUAGCAAUCGACAAACCAACACAGCCUGA
AACUGCUACCGACAGGUUUUAUACUCUCAAAUCCGUCAAGUGGGAAGCCGGAUCAACCGGAUGGUGGUGGAAACUCCCGGA
CGCGCUGAAUAAUAUAGGUAUGUUUGGCCAGAAUGUGCAACACCACUAUCUGUAUCGGUCUGGAUUCUUGAUCCAUGUGCA
GUGCAACGCGACUAAAUUCCAUCAAGGAGCUCUCUUGGUCGUUGCCAUACCCGAGCAUCAGAGGGGCGCAUAUAAUACGAA
CACUUCUCCUGGGUUUGAUGAUAUCAUGAAGGGAGAGGAAGGCGGAACGUUCAAUCACCCCUAUGUGUUGGAUGAUGGCAC
UUCACUUGCAUGCGCGACCAUCUUUCCCCAUCAAUGGAUAAACCUGCGCACAAAUAACAGUGCUACGAUUGUGCUUCCAUG
GAUGAACGCGGCGCCGAUGGAUUUCCCUCUUCGGCAUAACCAGUGGACACUUGCGAUUAUCCCGGUAGUUCCUUUGGGAAC
CCGGACGAUGUCUAGCAUGGUCCCCAUUACCGUGUCAAUUGCCCCCAUGUGUUGUGAGUUCAAUGGCCUCCGCCACGCAAU
UACGCAGGGUGUUCCCACUUAUCUCCUCCCGGGAUCCGGACAAUUCUUGACCACGGACGAUCACUCAAGUGCACCUGUCCU
GCCUUGCUUCAGUCCCACUCCUGAAAUGCAUAUACCGGGCCAAGUUCGAAAUAUGUUGGAAGUUGUCCAAGUCGAAAGUAU
GAUGGAGAUUAACAACACGGAAUCCGCUGUAGGCAUGGAACGACUGAAAGUGGACAUAUCCGCCCUCACGGACGUCGAUCA
ACUCCUGUUCAACAUCCCCCUUGAUAUUCAGCUCGAUGGCCCUCUCAGGAAUACGCUGGUAGGUAAUAUAUCUCGAUAUUA
UACACACUGGUCUGGUAGCCUGGAGAUGACGUUUAUGUUUUGUGGCUCAUUCAUGGCGACGGGUAAACUGAUACUGUGCUA
CACGCCGCCUGGUGGCAGCUGCCCAACUACACGCGAGACAGCGAUGCUGGGGACCCAUAUCGUGUGGGAUUUCGGUCUGCA
AAGUUCUGUGACACUCAUAAUCCCUUGGAUAAGUGGGAGUCACUACAGGAUGUUUAACAACGAUGCAAAAUCUACUAACGC
CAAUGUGGGUUACGUGACCUGCUUUAUGCAAACGAACUUGAUUGUUCCGAGCGAAUCAAGUGAUACGUGUUCUCUCAUUGG
GUUCAUUGCGGCUAAAGACGAUUUCUCCCUCCGACUUAUGCGCGAUUCCCCAGACAUUGGUCAGCUGGAUCAUCUUGAUGC
UGCUGAGGCAGCUUAUCAAAUUGAGAGCAUCAUCAAGACCGCGACCGACACUGUGAAAUCCGAGAUAAAUGCGGAGCUUGG
AGUUGUACCGUCCUUGAAUGCGGUCGAGACUGGUGCUACCAGUAAUACGGAGCCUGAAGAGGCCAUACAAACGAGGACCGU
UAUCAAUCAACAUGGCGUUUCCGAAACCCUCGUUGAAAAUUUCCUCAGCCGCGCGGCACUUGUCUCUAAGAGGUCUUUCGA
AUACAAAAACCACACGUCCAGUGAAGCGCAAACCGAUAAAAAUUUCUUCAAGUGGACUAUCAAUACUCGAAGCUUCGUUCA
GCUCCGGCGGAAGCUGGAACUGUUUACCUACCUCCGCUUCGAUGCAGAAAUUACUAUACUGACCACAGUGGCAGUAAAUGG
CUCAAGUAGUAACGCGUACAUGGGGCUGCCUGACCUUACGUUGCAAGCGAUGUUCGUUCCUACAGGCGCGCUCACACCCAA
ACAGCAGGAUAGCUUCCACUGGCAGAGUGGCAGCAACGCAUCUGUGUUUUUCAAAAUAAGCGACCCCCCUGCAAGGAUGAC
AAUUCCAUUCAUGUGUAUAAAUUCUGCUUAUUCCAUCUUUUACGACGGUUUUGCCGGUUUCGAAAAGUCUGGGCUCUAUGG
CAUCAACCCAGCGGAUACGAUCGGCAAUCUUUGUGUCCGGAUAGUAAAUGAGCACCAACCCAUAGGCUUCACUGUCACAGU
GCGGGUCUACAUGAAACCGAAACACAUCAAAGCAUGGGCUCCACGACCACCCCGAACGCUCCCUUAUAUGUCCAUUGCGAA
UGCCAACUACAGAGGAAAGGAACGGGCGCCUAACGCGCUCAAUGCAAUUAUAGGAAACAGGGAUAGUGUCAAGACUAUGCC
GCAUAAUAUAGUAACGACCugaccccucucccuccccccccccuaacguuacuggccgaagccgcuuggaauaaggccggu
gugcguuugucuauauguuauuuuccaccauauugccgucuuuuggcaaugugagggcccggaaaccuggcccugucuucu
ugacgagcauuccuaggggucuuuccccucucgccaaaggaaugcaaggucuguugaaugucgugaaggaagcaguuccuc
uggaagcuucuugaagacaaacaacgucuguagcgacccuuugcaggcagcggaaccccccaccuggcgacaggugccucu
gcggccaaaagccacguguauaagauacaccugcaaaggcggcacaaccccagugccacguugugaguuggauaguugugg
aaagagucaaauggcucuccucaagcguauucaacaaggggcugaaggaugcccagaagguaccccauuguaugggaucug
aucuggggccucggugcacaugcuuuacauguguuuagucgagguuaaaaaacgucuaggccccccgaaccacggggacgu
gguuuuccuuugaaaaacacgaugauaauauggccacaaccAUGGGACCGGGAUUCGACUUUGCGCAAGCGAUUAUGAAGA
AGAACACAGUAGUCGCACGAACCGAGAAAGGCGAGUUUACUAUGUUGGGUGUGUAUGAUAGGGUUGCCGUAAUACCAACAC
ACGCAAGCGUCGGUGAGACAGUCUAUAUCAACGACGUCGAGACCAAAGUUUUGGAUGCAUGCGCACUCCGCGAUUUGACAG
ACACAAACUUGGAGAUUACAAUAGUUAAGCUCGACAGAAACCAGAAAUUCCGGGAUAUUCGGCACUUUUUGCCCAAAUAUG
AAGAUGACUACAAUGACGCGGUCCUUUCAGUUCACACUAGCAAGUUCCCAAACAUGUAUAUUCCCGUAGGUCAGGUAACCA
AUUAUGGAUUUCUCAACUUGGGAGGCACUCCAACGCACAGGAUACUCAUGUAUAACUUCCCAACCCGCGCUGGACAGUGCG
GAGGGGUUGUCACAACGACCGGUAAGGUCAUCGGCAUCCACGUUGGAGGUAAUGGAGCGCAGGGUUUUGCCGCCAUGUUGC
UCCAUUCCUACUUCACAGAUACCCAAGGAGAGAUUGUAAGCAACGAGAAAAGUGGGGUAUGCAUAAAUGCGCCUGUGAAGA
CGAAACUUCAACCCUCCGUCUUUCACCAGGUCUUUGAAGGAAGUAAAGAGCCAGCUGUCCUUAAUUCUAAAGACCCUAGAC
UCAAAACGGACUUUGAGGAGGCGAUUUUUUCAAAGUACACUGGAAACAAGAUUAUGCUUAUGGACGAAUAUAUGGAGGAAG
CCGUAGACCAUUACGUUGGAUGUCUGGAACCGUUGGAUAUUUCUGUUGAUCCUAUUCCUUUGGAAUCAGCUAUGUACGGGA
UGGACGGGCUUGAGGCACUCGACCUGACCACCUCCGCUGGGUUUCCCUAUCUGCUGCAAGGGAAAAAAAAGCGGGAUAUUU
UUAAUAGACAUACUAGAGACACGACGGAAAUGACUAAAAUGCUGGAGAAGUAUGGCGUGGAUCUGCCUUUCGUCACCUUCG
UAAAAGAUGAACUCCGGAGCAAGGAAAAAGUCGAAAAGGGAAAAAGUCGGCUUAUUGAGGCUUCUUCUCUCAACGACUCAG
UGGCGAUGCGAGUUGCUUUUGGCAACCUCUACGCCACAUUUCACAAUAAUCCGGGAACGGCAACUGGCAGUGCCGUAGGCU
GCGACCCCGACAUUUUCUGGAGUAAAAUUCCGAUACUCUUGGACGGUGAAAUUUUUGCCUUCGACUAUACGGGAUAUGACG
CGUCCUUGUCCCCGGUUUGGUUUGCAUGUCUGAAAAAGGUCCUUGUUAAGUUGGGUUACACCCAUCAAACUUCUUUCAUAG
AUUAUUUGUGCCAUUCUGUUCAUUUGUAUAAGGACAGGAAGUACAUUGUAAACGGGGGGAUGCCUUCUGGUUCCUCUGGUA
CCUCCAUUUUCAAUACAAUGAUUAAUAACAUAAUUAUCCGGACUCUCCUUAUAAAAGUUUACAAGGGAAUCGACUUGGAUC
AGUUUAAGAUGAUAGCCUAUGGAGAUGACGUAAUUGCGUCUUAUCCUCACAAGAUCGACCCCGGCCUGUUGGCGGAAGCGG
GUAAGCAUUAUGGUCUGGUUAUGACGCCUGCUGAUAAGGGGACCAGUUUCGUGGAUACGAACUGGGAAAACGUAACCUUCU
UGAAACGGUAUUUUAGGGCUGAUGACCAAUAUCCGUUCCUCAUUCAUCCCGUAAUGCCGAUGAAGGAGAUACAUGAGAGUA
UUCGGUGGACGAAAGAUCCGCGAAAUACACAAGACCACGUACGGUCACUGUGUUACCUGGCAUGGCAUAACGGCGAAGAGG
CCUACAAUGAAUUCUGUCGGAAAAUUCGAAGUGUGCCCGUCGGACGAGCUUUGACUCUUCCGGCUUAUUCCUCUCUCAGAA
GGAAAUGGCUUGAUAGUUUCugauaaccgcggugucaaaaaccgcguggacgugguuaacaucccugcugggaggaucagc
cguaauuauuauaauuggcuuggugcuggcuacuauuguggccauguagugccugaccaaccagaaacauaauugaauaca
gcagcaauuggcaagcugcuuacauagaacucgcggcgauuggcaugccgccuuaaaauuuuuauuuuauuuuuucuuuuc
uuuuccgaaucggauuuuguuuuuaauauuucaaaaaaaaaaaaaaaaaaaaaaaaaaaaaaaaaaaaaaaaaaaaaaaa
SEQ ID NO: 87 VEE-KY-14-18953-p1-IRES-3cd
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaucgagaugugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuccauaguaguugg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugcugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacuccccaagagugaaaaauuccuuugcaucacccucu
cugaacaagucauagugauaacacacucuggccgaaaagggcguuaugccgugcaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagccacccuguauauugacgaagcuuuugcuugucaucagua
cucucagagcgcucauagccauuauaagaccuaaaaaggcagugcucugcggggaucccaaacagugcgguuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuauu
uugaaacggacaaagcucacucagcagagauaguauugaaccaacuaugcgugagguuuuuggacucgacucuggacuccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccggccguaugca
aaccgaaauccucacuugaagagacggaaguucuguuuguauucauuggguacgaugcaaggcccgcuacgcacaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccacccggcauuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggcaaggaagggcuaca
gcacaagcgauggcaaaacuuucucauauuuggaagggaccaaguuucaccaggggccaaggauauagcagaaaucuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccccaggggg
caacgucagccgagacuaacucuuacuucgcaaagaguauggaguuucuggggaccggugccugcccgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucaccacucccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaaggucgcaguggaagccugua
acgccauguugaaagagaacuuuccgacuguggcuucuuacuguauuauuccaaguacgaugccauauuuggacaugguug
acggagcuucaugcugcuuagacacugccaguuuuugcccugcaaagcuggcagcuuucccccaaaaaaacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucccagaaauaugcgugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacacuuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
agcuuggcaaaccucuggcagcagacgaugaacaugaugaugacaggagaagggcauugcaugaagagucaacacgcugga
accgaguggguauucuuucagagcugugcaaggcaguagaaucaagguaugaacaccguaggaacuuccaucauaguaugg
ccaugacuacucuagcuagcaguguuaaaucauucagcuaccugagaggggccccccuauaacucucuagguaaccugaau
ggacuacgacauagucuaguccgccaagAUGGGGGCACAAGUGACUCGGCAACAAACGGGUACACAUGAAAAUGCCAAUAU
UGCCACAAAUGGCAGUCACAUUACUUACAACCAAAUCAAUUUUUACAAAGAUUCCUAUGCGGCGUCCGCUAGCAAACAAGA
CUUUAGUCAGGAUCCCUCUAAGUUUACUGAACCCGUUGUCGAGGGGCUGAAGGCAGGAGCCCCCGUGCUCAAGUCACCAAG
UGCGGAAGCGUGUGGGUACAGCGAUCGCGUAUUGCAAUUGAAACUGGGGAAUUCUGCAAUCGUUACCCAGGAGGCUGCCAA
UUACUGUUGUGCUUACGGAGAAUGGCCGAAUUACUUGCCUGACCACGAAGCUGUUGCGAUAGACAAGCCUACUCAACCCGA
GACAGCAACUGAUAGGUUCUAUACGCUCAAAUCAGUCAAGUGGGAGACAGAAUCUACGGGAUGGUGGUGGAAAUUGCCAGA
CGCUCUGAACAACAUUGGCAUGUUCGGGCAGAACGUGCAGCAUCACUAUCUGUAUAGAUCCGGGUUUUUGAUUCACGUACA
AUGCAACGCUACCAAGUUUCAUCAGGGUGCUCUUCUGGUCGUCGCGAUACCCGAACACCAGCGGGGGGCACACAACACAAC
CACUUCUCCUGGGUUCGAUGACAUUAUGAAGGGCGAAGAGGGAGGGACAUUCAACCACCCAUACGUUCUGGAUGAUGGGAC
UUCCCUUGCUUGCGCGACUAUUUUCCCACAUCAAUGGAUUAACCUUCGAACAAACAAUAGCGCGACUAUCGUGCUGCCAUG
GAUGAACGCCGCGCCGAUGGACUUUCCAUUGAGGCACAACCAAUGGACGCUUGCAAUUAUACCUGUGGUACCGCUCGGUAC
UCGGACUGUAUCCAGUAUGGUGCCUAUCACUGUAAGUAUUGCACCUAUGUGCUGCGAAUUUAACGGGCUUAGGCAUGCAAU
AACCCAAGGCGUCCCUACGUAUUUGCUGCCGGGAUCCGGUCAGUUUCUGACAACGGAUGAUCACAGCAGCGCACCGGUCCU
GCCUUGUUUUAAUCCAACCCCAGAAAUGCACAUCCCUGGCCAAGUAAGAAACAUGUUGGAGGUUAUACAAGUCGAGAGUAU
GAUGGAGAUAAAUAACACGGAAAAUGCGGUCGGCAUGCAGAGGCUCAAGGUGGACAUUUCUGUCCUCACGGAUGUUGACCA
GUUGUUGUUCAAUAUCCCACUCGACAUUCAGCUCGAUGGGCCUCUUAGAAAUACCCUCGUUGGGAACAUUUCUAGAUAUUA
CACACAUUGGUCCGGUUCCUUGGAGAUGACUUUUAUGUUUUGUGGCUCAUUUAUGGCAACCGGCAAGCUCAUCUUGUGCUA
UACUCCUCCCGGGGGGUCUUGCCCCACAACAAGGGAAACAGCGAUGCUGGGGACCCACAUUGUAUGGGAUUUUGGUCUCCA
AAGCAGUGUUACCUUGGUUAUACCGUGGAUAAGUGGAUCCCAUUAUCGGAUGUUUAACAACGACGCCAAGUCAACCAACGC
AAAUGUAGGGUAUGUAACCUGUUUCAUGCAAACCAAUCUUAUAGUGCCUUCUGAAUCAAGCAAUACAUGCUCCCUGAUUGG
AUUUGUGGCAGCGAAGGACGAUUUUAGUCUGAGACUUAUGCGGGACUCUCCAGACAUCGGACAGCUUGAACAUCUUCACGA
GGCGGAGGCAGCUUACCAGAUAGAGAGCAUUAUCAAAACCGCGACGGACACCGUCAAAUCUGAAAUUAAUGCAGAGCUGGG
AGUGGUACCCUCACUGAAUGCUGUGGAAACCGGUGCAUCUUCUAACACAGAACCAGAGGAAGCUAUUCAAACGCGCACGGU
AAUUAACCAACACGGCGUCAGCGAAACACUUGUAGAAAACUUUCUUAGCAGAGCCGCACUCGUGUCCAAACGAAGCUUUGA
GUAUAAAAACCACACGUCCAGCGAAGCAAGAACCGAUAAGAAUUUUUAUAAAUGGACUAUCAACACUAAGUCAUUUGUGCA
GCUUAGACGGAAACUGGAGCUCUUCACCUACCUCCGAUUUGACGCGGAGAUCACGAUUCUCACGACCGUCGCCGUAGGAUC
AAAUAACUCCACAUAUAUGGGUCUGCCAGACCUCACCCUGCAAGCUAUGUUUGUCCCCACCGGCGCUCUCACCCCGGAGAA
ACAGGAUUCAUUCCAUUGGCAAUCUGGGAGUAAUGCAAGUGUUUUUUUCAAAGUUAGUGACCCUCCAGCUCGAAUGACGAU
UCCUUUCAUGUGCAUAAAUUCCGCUUAUAGCGUGUUUUAUGACGGAUUUGCGGGUUUUGAGAAGAACGGCUUGUAUGGUAU
AAACCCGGCCGAUACAAUAGGCAACCUGUGUGUAAGAAUCGUUAACGAGCACCAACCGAUCGGUUUCACGGUUACGGUGCG
CGUGUACAUGAAGCCUAAACAUAUCAAAGCUUGGGCACCGAGGCCCCCCCGAACGCUUCCUUACAUGAGCAUCGCAAAUGC
AAAUUACAAGGGGAAAGAGAGGGCCCCCAAUGCUCUCAACGCAAUUAUAGGGAAUAGAGAAAGCGUAAAAACGAUGCCCCA
UGACAUAAGACUGGUUAAUACCugaccccucucccuccccccccccuaacguuacuggccgaagccgcuuggaauaaggcc
ggugugcguuugucuauauguuauuuuccaccauauugccgucuuuuggcaaugugagggcccggaaaccuggcccugucu
ucuugacgagcauuccuaggggucuuuccccucucgccaaaggaaugcaaggucuguugaaugucgugaaggaagcaguuc
cucuggaagcuucuugaagacaaacaacgucuguagcgacccuuugcaggcagcggaaccccccaccuggcgacaggugcc
ucugcggccaaaagccacguguauaagauacaccugcaaaggggcacaaccccagugcccacguugugaguuggauaguug
uggaaagagucaaauggcucuccucaagcguauucaacaaggggcugaaggaugcccagaagguaccccauuguaugggau
cugaucuggggccucggugcacaugcuuuacauguguuuagucgagguuaaaaaacgucuaggccccccgaaccacgggga
cgugguuuuccuuugaaaaacacgaugauaauauggccacaaccAUGGGACCUGGAUUUGACUUCGCCCAGGCGAUUAUGA
AGAAGAAUACUGUGAUCGCACGCACCGAGAAGGGGGAGUUUACCAUGCUCGGUAUACACGAUCGGGUGGCGGUUAUUCCAA
CGCACGCUAGUGUAGGGGAGACAAUCUAUAUCAAUGACGUUGAGACAAAAGUGCUUGACGCCUGUGCCUUGAGGGACCUCA
CAGAUACCAAUCUUGAGAUUACGAUUGUUAAACUCGAUAGAAACCAAAAAUUCAGAGAUAUUCGGCACUUCUUGCCGCGGU
AUGAAGACGAUUAUAAUGACGCAGUUCUGAGUGUGCACACUUCAAAGUUUCCCAAUAUGUAUAUACCCGUGGGACAGGUGA
CUAAUUAUGGGUUUCUGAAUCUUGGUGGCACUCCGACACACAGGAUAUUGAUGUACAAUUUCCCGACAAGGGCUGGGCAAU
GUGGUGGUGUGGUUACAACUACGGGUAAAGUUAUCGGCAUUCAUGUUGGUGGCAACGGGGCUCAAGGCUUCGCUGCCAUGC
UUCUUCACUCUUACUUCAUCGACACCCAGGGCGAGAUUGUUUCAAAUGAAAAAAGUGGUGUGUGCAUCAAUGCCCCUGCCA
AAACCAAACUCCAACCUUCAGUAUUCCACCAGGUCUUUGAGGGAAGUAAAGAACCCGCUGUACUUAAUUCCAAGGACCCUC
GGUUGAAAACGGACUUUGAGGAGGCGAUCUUUUCCAAAUAUACGGGGAACAAGAUUAUGUUGAUGGAUGAAUAUAUGGAAG
AGGCGGUCGACCACUACGUGGGCUGCCUCGAGCCGUUGGAUAUAUCUAUUGAUCCUAUACCACUGGAGUCAGCGAUGUACG
GGAUGGACGGCCUUGAAGCGCUUGAUUUGACGACUAGCGCUGGGUUUCCUUAUCUCCUUCAGGGCAAGAAGAAACGGGACA
UUUUUAAUCGCCAGACCCGAGAUACAACCGAGAUGACACGCAUGCUGGAAAAAUACGGCGUCGAUUUGCCGUUUGUUACCU
UCGUUAAAGAUGAGCUCCGGUCUCGAGAGAAAGUUGAAAAGGGGAAGUCCCGACUCAUCGAGGCAAGUUCCUUGAAUGACU
CAGUUGCUAUGCGAGUAGCCUUUGGCAACCUCUAUGCGACGUUCCAUAAUAAUCCCGGGACGGCGACUGGCAGUGCUGUCG
GUUGCGAUCCAGACAUAUUCUGGAGCAAAAUCCCCAUUCUCUUGGACGGUGAGAUCUUCGCCUUCGAUUACACAGGGUACG
ACGCGAGUCUGAGCCCAGUCUGGUUCGCAUGUCUUAAGAAAGUGCUGAUCAAACUUGGAUACACACACCAAACCAGUUUUA
UUGAUUAUUUGUGCCAUUCAGUACAUCUGUACAAGGACAGAAAGUACAUAGUGAACGGUGGUAUGCCCUCUGGCUCUAGCG
GUACGUCUAUCUUCAACACGAUGAUUAAUAACAUUAUAAUGCGCACCCUUCUGAUUCGGGUCUACAAGGGGAUAGAUCUGG
AUCAGUUUAAGAUGAUAGCGUAUGGUGACGAUGUGAUAGCAAGCUACCCACAUAAGAUUGACCCCGGUCUGCUCGCAGAGG
CGGGUAAACACUAUGGACUGCUUAUGACCCCUGCGGAUAAGGGGACGAGUUUCGUAGAUACGAACUGGGAGAAUGUGACAU
UUCUUAAAAGGUAUUUUCGCGCGGACCACCAGUACCCAUUUUUGAUCCACCCUGUGAUGCCUAUGAAGGAAAUCCAUGAAA
GCAUUAGGUGGACAAAGGACCCUCGAAACACUCAGGAUCAUGUGCGCUCUUUGUGUUACCUUGCUUGGCACAACGGGGAAG
AAGCUUAUAAUGAGUUCUGUCGCAAGAUACGAAGCGUACCAGUUGGGAGAGCGCUUACCUUGCCUGCGUAUUCUUCCCUCA
GACGCAAAUGGCUGGAUAGUUUCugauaaccgcggugucaaaaaccgcguggacgugguuaacaucccugcugggaggauc
agccguaauuauuauaauuggcuuggugcuggcuacuauuguggccauguacgugcugaccaaccagaaacauaauugaau
acagcagcaauuggcaagcugcuuacauagaacucgcggcgauuggcaugccgccuuaaaauuuuuauuuuauuuuuucuu
uucuuuuccgaaucggauuuuuuuuuaauauuucaaaaaaaaaaaaaaaaaaaaaaaaaaaaaaaaaaaaaaaaaaaaa
SEQ ID NO: 88 VEE-NZ-2010-541-p1-IRES-3cd
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugcugggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggacuguccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguagcuugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguacugugccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaaguaagugcgcagccgaugaggccuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcaucaccccccucu
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucaugcagcgua
cucucagagcgcucauagccauuauaagaccuaaaaaggcagugcucugggggaucccaaacaguggguccuuuuuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggacuuuu
uugaaacggacaaagcucacucagcagagauaguauugaaccaacuaugcgugagguuuuuggacucgaucuggacucccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccugaggcuaccuucagagcucggcuggauuuag
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauaggggcaguucaaguuuucccggguacucgca
aaccgaaauccucacuugaagagacggaaguucuguuuguauucauuggguacgauccaaggcccguacgcacaacuccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggggaggggcugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccacggauuucccuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggaaggaagggcucaca
gcacaagcgauggcaaaacuuucucauauuuggaagggaccaaguuucaccaggggccaaggauauagcagaaauuaacug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggagcuagcgugaccaggcggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcauuauuugaaggcagaaggaaaaguggagugcuacc
gaacccugcauccuguuccuuuguauucaucuagugugaaccgugccuuuucaagccccaagguccaguggaacgccugua
acgccauguugaaagagaacuuuccgacuguggcuucuuacuguauuauuccagaguacgaugccuauuuggacaugguug
acggagcuucaugcugcuuagacacugccaguuuuugcccugcaaagcugcgcagcuuuccaaagaaacacuccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucaagaaauaugcgcugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauuauagccgagcacuuccagccuggggauuguguucugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacaucuugcccacuaaaacuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguccggggggagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaaucauaguaugg
ccaugacuacucuagcuagcaguguuaaaucauucagcuaccugagaggggccccuauaacucucuacggcuaaccugaau
ggacuacgacauagucuaguccgccaagAUGGGUGCUCAAGUCACUCGCCAGCAAACCGGGACACACGAAAAUGCUAACAU
CGCAACUAAUGGCAGCCACAUAACGUACAACCAGAUCAAUUUUUAUAAGGAUUCAUACGCAGCAUCAGCCAGUAAGCAAGA
CUUCUCACAAGACCCAUCAAAGUUCACCGAACCAGUAGUAGAGGGACUGAAAGCGGGUGCUCCGGUACUGAAGUCCCCCUC
AGCUGAAGCUUGUGGCUACAGCGAUAGGGUUCUUCAACUCAAACUUGGGAAUAGCGCGAUUGUCACACAGGAAGCGGCCAA
CUACUGCUGCGCAUAUGGGGAAUGGCCCAAUUAUUUGCCUGAUCAUGAGGCAGUAGCUAUUGACAAACCUACUCAGCCAGA
AACCGCCACUGAUCGAUUUUAUACUCUGAAAAGCGUAAAGUGGGAGACUGAAAGCACAGGAUGGUGGUGGAAAUUGCCUGA
UGCGCUCAAUAAUAUUGGAAUGUUUGGGCAAAAUGUUCAAUACCACUACCUCUAUAGAUCCGGCUUCCUCAUUCACGUUCA
GUGUAACGCGACUAAGUUCCACCAAGGCGCCCUUUUGGUCGUGGCAAUUCCCGAGCACCAACGAGGGGCUUAUAACACUAC
AACUUCACCCGGGUUCACGGACAUAAUGAAAGGUGAGGAGGGGGGAACGUUCUCCCAUCCGUAUGUCUUGGACGACGGCAC
GUCCCUCGCCUGCGCUACGAUCUUUCCUCACCAAUGGAUAAAUCUCCGGACGAACAAUAGCGCCACAAUAGUGUUGCCUUG
GAUGAACGCGGCCCCUAUGGAUUUCCCUCUGCGCCACAAUCAGUGGACCCUUGCAAUAAUCCCGGUUGUACCACUUGGAAC
CCGCACGGUUAGCUCUAUGGUGCCGAUUACAGUAUCCAUUGCGCCGAUGUGCUGCGAAUUCAAUGGACUGCGACACGCGAU
CACUCAAGGAGUCCCUACCUAUCUGCUGCCGGGCUCCGGACAGUUCCUCACGACGGACGACCAUAGCUCAGCACCUGUACU
GCCUUGCUUUAAUCCAACUCCCGAGAUGCAUAUCCCAGGCCAGGUUCGAAACAUGCUGGAAGUUGUACAAGUCGAGAGUAU
GAUGGAAAUAAAUAAUACUGAGAAUGCGGUCGGCAUGCAAAGACUGAAAGUCGAUAUCUCCGUACUUACCGAUGUCGACCA
GCUCCUCUUUAACAUUCCACUUGAUAUACAGCUGGACGGCCCGCUUCGAAAUACCCUUGUCGGGAAUAUAUCCCGGUAUUA
UACGCAUUGGUCUGGGUCUCUCGAAAUGACCUUUAUGUUUUGUGGGAGCUUUAUGGCAACCGGAAAGCUGAUUUUGUGCUA
UACUCCACCAGGCGGUAGUUGCCCCACGACACGCGAAACUGCAAUGUUGGGGACGCAUAUCGUUUGGGAUUUCGGCCUUCA
AUCCUCAGUCACCCUUGUGAUUCCGUGGAUUAGUGGUUCUCAUUAUCGCAUGUUUAAUAAUGAUGCCAAGUCUACUAAUGC
UAACGUUGGCUAUGUGACGUGCUUUAUGCAGACCAAUCUCAUCGUACCCAGUGAAAGUAGCAACACGUGCUCUCUGAUCGG
UUUCGUGGCUGCGAAGGACGAUUUCAGCCUUCGACUUAUGAGGGAUUCCCCCGAUAUCCGGCAAUUGGACCAUCUUGAUGC
AGCCGAAGCUGCAUACCAGAUAGAGUCAAUCAUUAAAACGGCAACCGAUACCGUGAAAUCAGAAAUUAAUGCCGAACUGGG
GGUGGUCCCCUCACUGAAUGCUGUUGAAACGGGAGCAUCAAGCAACACCGAGCCAGAGGAGGCGAUACAGACGCGGACCGU
GAUAAAUCAGCACGGGGUCUCCGAAACUUUGGUCGAGAACUUCCUUUCUCGAGCAGCUCUGGUUUCAAAGAGGUCCUUUGA
AUAUAAGAACCACGCCAGUUCAGAAGCGCAGACAGAUAAGAACUUCUUCAAAUGGACAAUAAAUACGAAGAGCUUCGUGCA
GCUCAGACGGAAACUGGAACUCUUUACUUACUUGAGAUUCGAUGCGGAGGUGACUAUACUGACUACAGUGGCAGUCAGCAG
UAGCAAUAGUACCUAUACCGGUCUCCCUGACUUGACCCUUCAAGCGAUGUUCGUACCGACGGGAGCGCUUACACCAGAGAA
ACAGGACAGUUUCCACUGGCAAUCUGCAUCAAACGCGAGUGUUUUCUUUAAAAUUUCCGAUCCUCCGGCAAGAAUGACUAU
CCCAUUCAUGUGUAUUAACUCCGCAUACUCUGUCUUCUACGAUGGAUUCGCCGGUUUCGAAAAGAGCGGACUCUAUGGAAU
UAACCCAGCUGAUACAAUAGGUAACCUCUGUGUGCGCAUUGUUAACGAACACCAGCCCAUCGGUUUCACUGUCACAGUCCG
GGUAUACAUGAAACCAAAGCAUAUAAAAGCGUGGGCGCCCAGACCACCCCGGACCCUGCCGUACAUGAGCAUCGCCAAUGC
CAAUUACCGAGGCAAAGAGAGGGCCCCCAACGCGCUCAACGCAAUAAUCGGCAACAGGGACAGCGUCAAAACAAUGCCCCA
UAACAUUGUGACUACGugaccccucucccuccccccccccuaacguuacuggccgaagccgcuuggaauaaggccggugug
cguuugucuauauguuauuuuccaccauauugccgucuuuuggcaaugugagggcccggaaaccuggcccugucuucuuga
cgagcauuccuaggggucuuuccccucucgccaaaggaaugcaaggucuguugaaugucgugaaggaagcaguuccucugg
aagcuucuugaagacaaacaacgucuguagcgacccuuugcaggcagcggaaccccccaccuggcgacaggugccucugcg
gccaaaagccacguguauaagcauacaccugcaaaggggcacaaccccagugccacguugugaguuggauaguuguggaaa
gagucaaauggcucuccucaagcguauucaacaaggggcugaaggaugcccagaagguaccccauuguaugggaucugauc
uggggccucggugcacaugcuuuacauguguuuagucgagguuaaaaaacgucuaggccccccgaaccacggggacguggu
uuuccuuugaaaaacacgaugauaauauggccacaaccAUGGGGCCUGGCUUUGAUUUCGCACAGGCCAUAAUGAAGAAAA
ACACCGUUAUUGCACGAACCGAAAAAGGUGAGUUCACAAUGCUCGGUAUUUACGACAGAGUUGCCGUCAUACCUACCCAUG
CAUCCGUAGGGGAAACAAUUUAUAUAAAUGACGUGGAGACUAAAGUUCUCGAUGCCUGCGCCCUCCGCGAUUUGACUGACA
CCAACUUGGAAAUAACAAUUGUUAAGAUGGACCGCAAUCAAAAAUUCCGAGACAUAAGGCACUUUCUCCCCCGAUACGAGG
ACGACUAUAACGAUGCGGUGCUCAGUGUCCACACCUCAAAAUUCCCUAAUAUGUACAUCCCCGUCGGUCAAGUGACUAACU
AUGGAUUUUUGAAUUUGGGAGGCACACCGACUCACCGCAUACUCAUGUACAAUUUCCCCACACGGGCAGGGCAGUGUGGUG
GUGUAGUCACGACAACAGGAAAGGUGAUCGGGAUCCAUGUCGGUGGUAACGGCGCACAAGGGUUUGCUGCAAUGCUUCUCC
ACUCUUAUUUUACUGACACUCAGGGCGAAAUCGUCAGCUCCGAAAAGAGUGGAGUUUGUAUAAAUGCACCCGCUAAAACGA
AGCUGCAACCGAGUGUCUUUCACCAAGUGUUCGAGGGUUCAAAAGAACCCGCUGUACUCAACAGUAAGGACUCCAGGCUCA
AAACAGAUUUUGAGGAAGCUAUCUUCUCUAAAUACACGGGAAAUAAAAUCAUGCUGAUGGAUGAAUAUAUGGAAGAAGCCG
UUGACCAUUACGUUGGGUGUCUGGAGCCGUUGGAUAUCUCAAUUGAUCCCAUCCCCCUUGAAUCUGCUAUGUAUGGCAUGG
ACGGUCUCGAGGCGCUUGAUUUGACGACAUCCGCUGGAUUCCCUUAUUUGUUGCAAGGUAAAAAGAAGCGCGACAUAUUUA
ACAGACAGACUAGGGAUACCACUGAGAUGACUCGCAUGUUGGAGAAAUACGGUGUUGAUCUUCCAUUUGUUACUUUUGUGA
AAGACGAAUUGCGAAGCAGGGAAAAGGUUGAAAAGGGUAAGUCUAGACUUAUCGAAGCCUCUUCAUUGAAUGAUUCAGUGG
CAAUGAGGGUUGCCUUUGGAAACUUGUACGCCACGUUUCAUAAUAAUCCCGGAACGGCGACAGGGUCUGCGGUUGGGUGUG
AUCCCGACGUAUUUUGGAGCAAAAUACCCAUCUUGCUCAACGGUGAAAUAUUUGCCUUCGAUUACACCGGGUAUGACGCGU
CUCUGAGCCCAGUGUGGUUUGCGUGCCUGAAGAAGGUUUUGAUAAAAUUGGGCUAUACACACCAGACUUCUUUUAUUGACU
ACCUGUGCCAUUCUGUUCACCUCUAUAAAGACCGAAAGUAUAUUGUUAAUGGAGGAAUGCCGUCAGGAUCCUCUGGUACUU
CCAUCUUCAAUACUAUGAUCAAUAAUAUCAUUAUACGCACACUGCUCAUCAGAGUGUACAAAGGUAUAGACCUGGAUCAAU
UUAAGAUGAUUGCCUAUGGUGACGACGUGAUUGCGAGCUACCCGCAUAAGAUCGACCCAGGCUUGUUGGCUGAGGCUGGAA
AGCAUUACGGACUCAUCAUGACUCCAGCGGAUAAGGGAACGUCCUUUGUGGAUACCAAUUGGGAAAAUGUUACGUUUCUUA
AACGAUAUUUCAGGGCAGACGAUCAAUACCCGUUCCUUAUCCACCCCGUGAUGCCGAUGAAAGAAAUACACGAAAGCAUUA
GAUGGACUAAAGACCCAAGAAACACGCAAGAUCACGUUAGGAGUCUUUGUUACUUGGCUUGGCAUAAUGGUGAAGAGGCAU
ACAAUGAAUUCUGUAGGAAGAUACGAAGUGUCCCAGUCGGUCGAGCACUCACACUCCCUGCAUACAGCUCUCUGAGACGCA
AAUGGCUCGAUAGUUUCugauaaccgcggugucaaaaaccgcguggacgugguuaacaucccugcugggaggaucagccgu
aauuauuauaauuggcuuggugcuggcuacuauuguggccauguacgugcugaccaaccagaaacauaauugaauacagca
gcaauuggcaagcugcuuacauagaacucgcggcgauuggcaugccgccuuaaaauuuuuauuuuauuuuuucuuuucuuu
uccgaaucggauuuuguuuuuaauauuucaaaaaaaaaaaaaaaaaaaaaaaaaaaaaaaaaaaaaaaaaaaaa
SEQ ID NO: 89 VEE-USA-2018-23087-p1-IRES-3cd
auaggcggcgcaugagagaagcccagaccaauuaccuacccaaaauggagaaaguucacguugacaucgaggaagacagcc
cauuccucagagcuuugcagcggagcuucccgcaguuugagguagaagccaagcaggucacugauaaugaccaugcuaaug
ccagagcguuuucgcaucuggcuucaaaacugaucgaaacggagguggacccauccgacacgauccuugacauuggaagug
cgcccgcccgcagaauguauucuaagcacaaguaucauuguaucuguccgaugagaugugcggaagauccggacagauugu
auaaguaugcaacuaagcugaagaaaaacuguaaggaaauaacugauaaggaauuggacaagaaaaugaaggagcuggccg
ccgucaugagcgacccugaccuggaaacugagacuaugugccuccacgacgacgagucgugucgcuacgaagggcaagucg
cuguuuaccaggauguauacgcgguugacggaccgacaagucucuaucaccaagccaauaagggaguuagagucgccuacu
ggauaggcuuugacaccaccccuuuuauguuuaagaacuuggcuggagcauauccaucauacucuaccaacugggccgacg
aaaccguguuaacggcucguaacauaggccuaugcagcucugacguuauggagcggucacguagagggaugucccauuuua
gaaagaaguauuugaaaccauccaacaauguucuauucucuguuggcucgaccaucuaccacgagaagagggacuuacuga
ggagcuggcaccugccgucuguauuucacuuacguggcaagcaaaauuacacaugucggugugagacuauaguagcuugcg
acggguacgucguuaaaagaauagcuaucaguccaggccuguaugggaagccuucaggcuaugcugcuacgaugcaccgcg
agggauucuugugcugcaaagugacagacacauugaacggggagagggucucuuuucccgugugcacguaugugcccagua
cauugugugaccaaaugacuggcauacuggcaacagaugucagugcggacgacgcgcaaaaacugcugguugggcucaacc
agcguauagucgucaacggucgcacccagagaaacaccaauaccaugaaaaauuaccuuuugcccguaguggcccaggcau
uugcuaggugggcaaaggaauauaaggaagaucaagaagaugaaaggccacuaggacuacgagauagacaguuagucaugg
gguguuguugggcuuuuagaaggcacaagauaacaucuauuuauaagcgcccggauacccaaaccaucaucaaagugaaca
gcgauuuccacucauucgugcugcccaggauaggcaguaacacauuggagaucgggcugagaacaagaaucaggaaaaugu
uagaggagcacaaggagccgucaccucucauuaccgccgaggacguacaagaagcuaagugcgcagccgaugaggcuaagg
aggugcgugaagccgaggaguugcgcgcagcucuaccaccuuuggcagcugauguugaggagcccacucuggaggcagacg
ucgacuugauguuacaagaggcuggggccggcucaguggagacaccucguggcuugauaaagguuaccagcuacgauggcg
aggacaagaucggcucuuacgcugugcuuucuccgcaggcuguacucaagagugaaaaauuaucuugcaucaccccucucg
cugaacaagucauagugauaacacacucuggccgaaaagggcguuaugccguggaaccauaccaugguaaaguaguggugc
cagagggacaugcaauacccguccaggacuuucaagcucugagugaaagugccaccauuguguacaacgaacgugaguucg
uaaacagguaccugcaccauauugccacacauggaggagcgcugaacacugaugaagaauauuacaaaacugucaagccca
gcgagcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaaguac
caaccauagggguguauggcgugccaggaucaggcaagucuggcaucauuaaaagcgcagucaccaaaaaagaucuagugg
ugagcgccaagaaagaaaacugugcagaaauuauaagggacgucaagaaaaugaaagggcuggacgucaaugccagaacug
uggacucagugcucuugaauggaugcaaacaccccguagagacccuguauauugacgaagcuuuuguugucaugccaggua
cucucagagcgcucauagccauuauaagaccuaaaaaggcagugcucugggggaucccaaacagugcgguuuuucuuaaca
ugaugugccugaaagugcauuuuaaccacgagauuugcacacaagucuuccacaaaagcaucucucgccguugcacuaaau
cugugacuucggucgucucaaccuuguuuuacgacaaaaaaaugagaacgacgaauccgaaagagacuaagauugugauug
acacuaccggcaguaccaaaccuaagcaggacgaucucauucucacuuguuucagagggugggugaagcaguugcaaauag
auuacaaaggcaacgaaauaaugacggcagcugccucucaagggcugacccguaaagguguguaugccguucgguacaagg
ugaaugaaaauccucuguacgcacccaccucagaacaugugaacguccuacugacccgcacggaggaccgcaucgugugga
aaacacuagccggcgacccauggauaaaaacacugacugccaaguacccugggaauuucacugccacgauagaggaguggc
aagcagagcaugaugccaucaugaggcacaucuuggagagaccggacccuaccgacgucuuccagaauaaggcaaacgugu
guugggccaaggcuuuagugccggugcugaagaccgcuggcauagacaugaccacugaacaauggaacacuguggauuucu
uugaaacggacaaagcucacucagcagagauaguauugaaccaacuaugcgugagguuuuuggacucgaucuggacucccg
gucuauuuucugcacccacuguuccguuauccauuaggaauaaucacugggauaacuccccgucgccuaacauguacgggc
ugaauaaagaagugguccgucagcucucucgcagguacccacaacugccucgggcaguugccacuggaagagucuaugaca
ugaacacugguacacugcgcaauuaugauccgcgcauaaaccuaguaccuguaaacagaagacugccucaugcuuuagucc
uccaccauaaugaacacccacagagugacuuuucuucauucgucagcaaauugaagggcagaacuguccugguggucgggg
aaaaguuguccgucccaggcaaaaugguugacugguugucagaccggccccugaggcuaccuucagagcuggccuggauuu
gcaucccaggugaugugcccaaauaugacauaauauuuguuaaugugaggaccccauauaaauaccaucacuaucagcagu
gugaagaccaugccauuaagcuuagcauguugaccaagaaagcuugucugcaucugaaucccggcggaaccugugucagca
uagguuaugguuacgcugacagggccagcgaaagcaucauuggugcuauagcgcggcaguucaaguuuucccggguaugca
aaccgaaauccucacuugaagagacggaaguucuguuuguauucauuggguacgaugcaaggcccguacgcaccaauccuu
acaagcuuucaucaaccuugaccaacauuuauacagguuccagacuccacgaagccggaugugcacccucauaucaugugg
ugcgaggggauauugccacggccaccgaaggagugauuauaaaugcugcuaacagcaaaggacaaccuggcggaggggugu
gcggagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcagagg
cuuaugaguccaucgcuaagauugucaacgauaacaauuacaagucaguagcgauuccacuguuguccacggacccuuuuu
ccgggaacaaagaucgacuaacccaaucauugaaccauuugcugacagcuuuagacaccacugaugcagauguagccauau
acugcagggacaagaaaugggaaaugacucucaaggaagcaguggcuaggagagaagcaguggaggagauaugcauauccg
acgacucuucagugacagaaccugaugcagagcuggugagggugcauccgaagaguuuuuggcuggcaaggaagggcuaca
gcacaagcgauggcaaaacuuucucauauuuggaagggaccaaguuucaccaggggccaaggauauagcagacaauuaaug
ccauguggcccguugcaacggaggccaaugagcagguaugcauguauauccucggagaaagcaugagcaguauuaggucga
aaugccccgucgaagagucggaagccuccacaccaccuagcacgcugccuugcuugugcauccaugccaugacuccagaaa
gaguacagcgccuaaaagccucacguccagaacaaauuacugugugcucauccuuuccauugccgaaguauagaaucacug
gugugcagaagauccaaugcucccagccuauauuguucucaccgaaagugccugcguauauucauccaaggaaguaucucg
uggaaacaccaccgguagacgagacuccggagccaucggcagagaaccaauccacagaggggacaccugaacaaccaccac
uuauaaccgaggaugagaccaggacuagaacgccugagccgaucaucaucgaagaggaagaagaggauagcauaaguuugc
ugucagauggcccgacccaccaggugcugcaagucgaggcagacauucacgggccgcccucuguaucuagcucauccuggu
ccauuccucaugcauccgacuuugauguggacaguuuauccauacuugacacccuggagggaguagcgugaccagccgggg
caacgucagccgagacuaacucuuacuucgcaaagaguauggaguuucuggcgcgaccggugccugcgccucgaacaguau
ucaggaacccuccacaucccgcuccgcgcacaagaacaccgucacuugcacccagcagggccugcucgagaaccagccuag
uuuccaccccgccaggcgugaauagggugaucacuagagaggagcucgaggcgcuuaccccgucacgcacuccuagcaggu
cggucucgagaaccagccuggucuccaacccgccaggcguaaauagggugauuacaagagaggaguuugaggcguucguag
cacaacaacaaugacgguuugaugcgggugcauacaucuuuuccuccgacaccggucaagggcauuuacaacaaaaaucag
uaaggcaaacggugcuauccgaagugguguuggagaggaccgaauuggagauuucguaugccccgcgccucgaccaagaaa
aagaagaauuacuacgcaagaaauuacaguuaaaucccacaccugcuaacagaagcagauaccaguccaggaagguggaga
acaugaaagccauaacagcuagacguauucugcaaggccuagggcaucuauuugaaggcagaaggaaaaguggaguguacc
gaacccugcauccuguuccuuuguauucaucuagugugaaccgucgcccuuuucaagccccaagguccaguggaaccugua
acgccauguugaaagagaacuuuccgacuguggcuucuuacuguauuauuccagaguacgaugccuauuuggacauggugg
acggagcuucaugcugcuuagacacugccaguuuuugcccugcaaagcugcgcagcuuuccaaaaaacacucccuauuugg
aacccacaauacgaucggcagugccuucagcgauccagaacacgcuccagaacguccuggcagcugccacaaaaagaaauu
gcaaugucacgcaaaugagagaauugcccguauuggauucggggccuuuaauguggaaugcuucaaaacccccaaugugua
auaaugaauauugggaaacguuuaaagaaaaccccaucaggcuuacugaagaaaacgugguaaauuacauuaccaaauuaa
aaggaccaaaagcugcugcucuuuuugcgaagacacauaauuugaauauguugcaggacauaccaauggacagguuuguaa
uggacuuaaagagagacgugaaagugacuccaggaacaaaacauacugaagaacggcccaagguacaggugauccaggcug
ccgauccgcuagcaacagcguaucugugcggaauccaccgagagcugguuaggagauuaaaugcgguccugcuuccgaaca
uucauacacuguuugauaugucggcugaagacuuugacgcuauauagccgagcacuuccccccccaguggggauuuuuugg
aaacugacaucgcgucguuugauaaaagugaggacgacgccauggcucugaccgcguuaaugauucuggaagacuuaggug
uggacgcagagcuguugacgcugauugaggggcuuucggcgaaauuucaucaauacauuugcccacuaaaaccuaaauuua
aauucggagccaugaugaaaucuggaauguuccucacacuguuugugaacacagucauuaacauuguaaucgcaagcagag
uguugagagaacggcuaaccggaucaccaugugcagcauucauuggagaugacaauaucgugaaaggagucaaaucggaca
aauuaauggcagacaggugcgccaccugguugaauauggaagucaagauuauagaugcuguggugggcgagaaagcgccuu
auuucuguggaggguuuauuuugugugacuccgugaccggcacagcgugccguguggcagacccccuaaaaaggcuguuua
agcuuggcaaaccucuggcagcagacgaugaacaugaugaugacaggagaagggcauugcaugaagagucaacacgcugga
accgaguggguauucuuucagagcugugcaaggcaguagaaucaagguaugaaaccguaggaacuuccaucauaguauagg
ccaugacuacucuagcuagcaguguuaaaucauucagcuaccugagaggggccccuauaacucucuacggcuaaccugaau
ggacuacgacauagucuaguccgccaagAUGGGUGCACAAGUGACGCGCCAGCAGACUGGCACGCACGAGAAUGCUAACGU
AGCCACUAACGGGUCCCAUAUAACAUAUAAUCAAAUAAAUUUUUACAAGGAUUCAUACGCGGCCUCUGCGUCAAAACAGGA
UUUUUCCCAAGAUCCGUCUAAGUUUACGGAGCCGGUCGUGGAAGGUCUGAAAGCUGGAGCACCUGUUCUGAAGAGUCCCUC
CGCGGAAGCGUGUGGCUACUCAGACCGAGUUCUGCAGUUGAAACUUGGGAACAGUGCUAUCGUGACCCAGGAAGCUGCCAA
UUACUGCUGCGCUUAUGGGGAGUGGCCCAAUUACUUGCCUGACCACGAAGCUGUAGCUAUAGAUAAACCGACGCAGCCAGA
AACCGCGACAGACCGGUUCUAUACCCUCCGCUCCGUCAAGUGGGAAGCAACUUCAACAGGCUGGUGGUGGAAACUGCCCGA
CGCGCUUAACAACAUCGGCAUGUUCGGGCAAAAUGUACAACAUCACUACCUCUACAGGAGUGGGUUCCUGAUUCACGUGCA
AUGUAACGCAACUAAAUUCCACCAAGGAGCCCUCCUCGUCGUUGCUAUCCCGGAACAUCAAAGGGGUGCACAUAAUACUAC
AACUUCUCCAGGGUUCGAUGACAUAAUGAAAGGCGAGGCAGGAGGCACUUUCAAUCAUCCCUACGUCCUGGACGAUGGCAC
AUCCCUGGCAUGUGCCACCAUCUUUCCCCAUCAGUGGAUUAAUUUGCGGACCAAUAAUAGCGCUACCAUCGUUCUUCCAUG
GAUGAACGCUGCACCGAUGGAUUUCCCGCUUAGACACAAUCAAUGGACGCUUGCAAUCAUUCCCGUUGUUCCGCUCGGCAC
CCGAACCAUGAGCAGUAUGGUCCCCAUUACUGUGUCUAUAGCGCCGAUGUGUUGCGAAUUUAAUGGACUGAGGCACGCAAU
CACUCAGGGAGUCCCCACGUACUUGCUUCCGGGUAGCGGGCAAUUCUUGACAACUGACGACCAUAGCAGUGCCCCGGUAUU
GCCUUGUUUUAAUCCCACCCCCGAAAUGCAUAUCCCCGGGCAAGUCAGAAAUAUGCUGGAAGUAGUGCAAGUAGAAAGUAU
GAUGGAAAUCAACAAUACAGAGUCAGCCGUAGGCAUGGAACGGCUUAAAGUUGACAUUUCUGCGCUCACGGACGUCGAUCA
AUUGCUGUUUAAUAUCCCCCUUGAUAUCCAGUUGGAUGGUCCACUUAGGAACACGCUUGUCGGAAACAUUAGCCGAUACUA
CACACAUUGGUCCGGUAGUCUCGAGAUGACAUUUAUGUUUUGUGGGUCAUUCAUGGCUACUGGCAAACUGAUACUGUGCUA
CACACCACCAGGGGGCAGCUGUCCAACGACUAGGGAAACUGCUAUGUUGGGUACACACAUUGUCUGGGAUUUCGGUUUGCA
GUCCUCUAUAACGCUGAUUAUACCCUGGAUAUCAGGUUCACAUUACCGCAUGUUUAAUAAUGACGCGAAGUCCACAAACGC
CAAUGUCGGUUACGUUACAUGUUUUAUGCAGACGAACCUGAUAGUGCCCUCAGAGUCUUCUGAUACCUGCUCUCUCAUUGG
UUUUAUUGCCGCUAAGGACGAUUUUUCACUUCGGCUGAUGCGCGAUUCCCCUGACAUACGGCAAAUCGAUCAUCUGCACGG
GGCCGAAGCUGCUUAUCAGAUUGAAUCCAUAAUAAAGACCGCAACCGAUACAGUCAAGAGCGAAAUAAAUGCAGAGUUGGG
UGUGGUACCAUCUCUCAACGCUGUCGAGACAGGGGCAACGUCAAAUACUGAGCCAGAAGAAGCGAUUCAAACAAGGACGGU
AAUAAACCAACAUGGUGUGUCCGAGACCCUCGUUGAAAACUUUCUGAGCAGAGCAGCACUCGUCUCAAAGCGGAGCUUUGA
GUAUAAAGACCACACCUCCUCCACUGCGCAGACGGAUAAGAAUUUCUUUAAGUGGACCAUUAAUACGCGGUCCUUUGUUCA
AUUGAGAAGGAAGUUGGAGCUGUUCACGUAUCUGCGGUUCGAUGCCGAGAUAACAAUCCUGACUACGGUAGCGGUAAAUGG
CUCCAACAACAAUACUUACGUUGGUCUCCCAGAUCUGACACUCCAGGCAAUGUUCGUCCCGACGGGGGCUUUGACGCCAGA
AAAACAAGACUCUUUCCAUUGGCAAAGCGGGAGUAAUGCCUCAGUAUUUUUUAAAAUCUCAGAUCCUCCUGCGCGCAUGAC
CAUUCCAUUUAUGUGUAUAAAUUCAGCCUACAGCGUCUUUUACGAUGGAUUUGCUGGGUUCGAGAAAACGGGGCUUUAUGG
UAUAAAUCCUGCUGAUACGAUUGGUAACCUGUGCGUACGGAUCGUAAAUGAACAUCAGCCUGUCGGCUUCACAGUUACCGU
CCGAGUUUAUAUGAAGCCCAAGCACAUCAAAGCGUGGGCACCUCGGCCACCCCGCACACUGCCGUACAUGUCUAUCGCAAA
CGCAAAUUAUAAAGGUAAAGAACGGGCUCCAAACGCGCUUAACGCUAUAAUCGGGAACCGCGACAGUGUUAAAACUAUGCC
CCAUAAUAUUGUGugaccccucucccuccccccccccuaacguuacuggccgaagccgcuuggaauaaggccggugugcgu
uugucuauauguuauuuuccaccauauugccgucuuuuggcaaugugagggcccggaaaccuggcccugucuucuugacga
gcauuccuaggggucuuuccccucucgccaaaggaaugcaaggucuguugaaugucgugaaggaagcaguuccucuggaag
cuucuugaagacaaacaacgucuguagcgacccuuugcaggcagcggaaccccccaccuggcgacaggugccucugcggcc
aaaagccacguguauaagauacaccugcaaaggcggcacaaccccagugccacguugugaguuggauaguuguggaaagag
ucaaauggcucuccucaagcguauucaacaaggggcugaaggaugcccagaagguaccccauuguaugggaucugaucugg
ggccucggugcacaugcuuuacauguguuuagucgagguuaaaaaacgucuaggccccccgaaccacggggacgugguuuu
ccuuugaaaaacacgaugauaauauggccacaaccAUGGGCCCUGGAUUUGAUUUCGCUCAGGCCAUCAUGAAGAAGAAUA
CGGUAAUAGCAAGAACAGAGAAAGGCGAGUUCACCAUGCUUGGUGUUUAUGAUAGGGUGGCGGUCAUCCCUACGCAUGCAU
CAGUUGGUGAAACAAUCUAUAUAGAUGACGUCGAGACACGGGUUCUCGACGCAUGCGCUUUGCGGGAUCUUACUGAUACGA
AUUUGGAAAUUACUAUUGUUAAACUUGACCGGAAUCAGAAGUUCAGAGACAUCCGCCAUUUUCUCCCGAGAUAUGAAGACG
ACUACAACGACGCCGUUCUGAGUGUCCACACGUCAAAAUUUCCGAAUAUGUAUAUUCCUGUGGGGCAGGUCACUAACUAUG
GAUUUUUGAAUUUGGGCGGCACUCCAACUCAUAGGAUCCUUAUGUAUAACUUCCCUACCAGAGCGGGGCAAUGUGGCGGCG
UGGUUACGACGACAGGCAAAGUAAUCGGUAUUCACGUUGGAGGCAACGGCGCCCAGGGAUUUGCGGCGAUGCUUCUCCAUU
CAUACUUUACGGACACACAAGGCGAGAUAGUAUCUAGCGAGAAAAGUGGUGUUUGCAUAAAUGCGCCAGCAAAAACAAAAU
UGCAGCCAUCCGUGUUCCACCAAGUUUUCGAAGGAAGUAAAGAGCCCGCUGUCUUGAACCCGAAAGAUCCGCGCUUGAAAA
CCGAUUUUGAGGAGGCGAUUUUCAGCAAGUACACUGGAAAUAAGAUUAUGCUUAUGGACGAGUAUAUGGAGGAGGCGGUGG
AUCAUUACGUAGGGUGCUUGGAGCCCCUUGACAUAUCUGUCGACCCCAUACCCUUGGAGUCCGCUAUGUACGGUAUGGACG
GAUUGGAGGCUCUGGACUUGACUACAAGUGCAGGUUUCCCAUAUCUCCUCCAGGGAAAAAAAAAACGGGACAUCUUUAACC
GCCAUACUCGCGAUACCAGCGAGAUGACCAAAAUGCUCGAAAAAUAUGGAGUGGACCUCCCGUUUGUGACGUUUGUUAAAG
ACGAACUGCGAAGCAGGGAAAAAGUGGAAAAGGGGAAAAGCAGGCUCAUCGAAGCUAGUAGUCUGAAUGACAGUGUAGCUA
UGAGAGUCGCGUUUGGAAACCUUUAUGCCACCUUUCAUAACAAUCCAGGCACCGCUACUGGCUCUGCCGUAGGCUGUGAUC
CGGACAUAUUCUGGUCCAAAAUCCCGAUUCUUCUCGACGGGGAGAUAUUCGCCUUCGAUUAUACGGGAUAUGAUGCUUCCC
UCAGCCCGGUCUGGUUCGCAUGCUUGAAAAAGGUGCUCAUAAAGCUGGGCUACACGCACCAGACGUCAUUCAUAGAUUAUC
UGUGCCAUAGCGUGCAUCUUUAUAAGGAUAGAAAGUACAUAGUCAAUGGCGGCAUGCCGUCAGGAAGUUCCGGAACCUCUA
UCUUCAAUACGAUGAUAAACAACAUCAUAAUUCGAACUCUCCUCAUAAGGGUAUACAAAGGAAUAGACUUGGACCAAUUCA
AGAUGAUCGCUUAUGGCGACGAUGUGAUUGCCUCUUACCCACACAAGAUUGACCCCGGACUCCUCGCAGAAGCUGGAAAGC
ACUAUGGCCUUGUUAUGACUCCCGCGGACAAAGGCACGUCCUUUGUGGACACCAACUGGGAGAAUGUUACUUUCCUGAAAC
GCUAUUUCCGGGCGGACGAUCAGUACCCCUUUCUCAUACAUCCAGUUAUGCCGAUGAAGGAGAUUCACGAGUCUAUCCGCU
GGACAAAAGAUCCGCGCAACACGCAGGAUCACGUACGCAGCCUCUGUUACCUCGCGUGGCAUAAUGGAGAAGAAGCAUAUA
ACGAGUUCUGUAGAAAAAUACGCUCAGUGCCAGUAGGGCGCGCCCUCACGUUGCCCGCAUACUCAUCACUGCGACGCAAAU
GGUUGGAUUCCUUUuaaccgcggugucaaaaaccgcguggacgugguuaacaucccugcugggaggaucagccguaauuau
uauaauuggcuuggugcuggcuacuauuguggccauguacgugcugaccaaccagaaacauaauugaauacagcagcaauu
ggcaagcugcuuacauagaacucgcggcgauuggcaugccgccuuaaaauuuuuauuuuauuuuuucuuuucuuuuccgaa
ucggauuuuguuuuuaauauuucaaaaaaaaaaaaaaaaaaaaaaaaaaaaaaaaaaaaaaaaaaaaa

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, wherein the composition comprises:
nanoparticles, wherein the nanoparticles comprise a surface; and
nucleic acids, wherein the nucleic acids comprise a sequence encoding for an RNA polymerase and a tumor protein antigen, wherein at least two of the nucleic acids encode for non-identical tumor protein antigens, and wherein the nucleic acids complex to the surface to form nucleic acid-nanoparticle complexes.

2. The composition of claim 1, wherein the nucleic acid comprises an RNA or a DNA.

3. The composition of claim 1, wherein the nucleic acids encode for a tumor protein antigen comprising epidermal growth factor receptor (EGFR); vascular endothelial growth factor (VEGF); VEGFA; acute myelogenous leukemia Wilms tumor 1 (WT1), preferentially expressed antigen of melanoma (PRAME), PR1, proteinase 3, elastase, cathepsin G, Chronic myelogenous WT1, Myelodysplastic syndrome WT1, Acute lymphoblastic leukemia PRAME, Chronic lymphocytic leukemia survivin, Non-Hodgkin's lymphoma survivin, Multiple myeloma New York esophagus 1 (NY-Esol), Malignant melanoma (MAGE), MAGE-A, MAGE-B, MAGE-C, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MART-1/Melan-A, Tyrosinase, tyrosinase related protein 1 (TRYP-1), glycoprotein 100 (GP100), breast cancer WT1, Herceptin, lung cancer WT1, Prostate-specific antigen (PSA), prostatic acid phosphatase, (PAP) Carcinoembryonic antigen (CEA), mucins (e.g., MUC-1), Renal cell carcinoma (RCC) Fibroblast growth factor (FGF), or programmed cell death protein (PD-1).

4. The composition of claim 1, wherein the RNA polymerase is a self-replicating RNA polymerase.

5. The composition of claim 4, wherein the self-replicating RNA polymerase is under that control of an internal ribosome entry side (IRES).

6. The composition of claim 1, wherein the RNA polymerase is an alphavirus RNA polymerase, an Eastern equine encephalitis virus (EEEV) RNA polymerase, a Western equine encephalitis virus (WEEV) RNA polymerase, a Venezuelan equine encephalitis virus (VEEV) RNA polymerase, a Chikungunya virus (CHIKV) RNA polymerase, a Semliki Forest virus (SFV) RNA polymerase, or a Sindbis virus (SINV) RNA polymerase.

7. The composition of claim 1, wherein the RNA polymerase is Venezuelan equine encephalitis virus (VEEV) RNA polymerase.

8. The composition of claim 1, wherein the composition comprises a first nucleic acid and a second nucleic acid, wherein the first nucleic acid is present at a concentration that is different from the second nucleic acid concentration.

9. The composition of claim 1, wherein the nanoparticles comprise a cationic lipid.

10. The composition of claim 9, wherein the cationic lipid comprises 1,2-dioleoyloxy-3 (trimethylammonium)propane (DOTAP), 3β-[N-(N′,N′-dimethylaminoethane) carbamoyl]cholesterol (DC Cholesterol), dimethyldioctadecylammonium (DDA); 1,2-dimyristoyl 3-trimethylammoniumpropane(DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 3060i10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,1OR,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-016B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,1OR,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12,3,6-bis(4-(bis(2-hydroxydodecyl)amino)butyl)piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9″′″-((((benzene-1,3,5-tricarbonyl)tris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4,1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)₂₀₀₀) carbamate; TT3, N1,N3,N5-tris(3-(didodecylamino)propyl)benzene-1,3,5-tricarboxamide, or a combination thereof.

11. The composition of claim 1, wherein the nanoparticles comprise a hydrophobic core.

12. The composition of claim 11, wherein the hydrophobic core comprises a lipid.

13. The composition of claim 12, wherein the lipid comprises an oil.

14. The composition of claim 11, wherein the hydrophobic core comprises a lipid in liquid phase at 25 degrees Celsius.

15. The composition of claim 12, wherein the lipid comprises α-tocopherol, coconut oil, grapeseed oil, lauroyl polyoxylglyceride, mineral oil, monoacylglycerol, palmkemal oil, olive oil, paraffin oil, peanut oil, propolis, squalene, squalane, soy lecithin, soybean oil, sunflower oil, a triglyceride, vitamin E, or a combination thereof.

16. The composition of claim 15, wherein the triglyceride comprises capric triglyceride, caprylic triglyceride, a caprylic and capric triglyceride, a triglyceride ester, myristic acid triglycerin, or a combination thereof.

17. The composition of claim 11, wherein the hydrophobic core comprises a lipid in solid phase at 25 degrees Celsius.

18. The composition of claim 11, wherein the hydrophobic core comprises glyceryl trimyristate-dynasan.

19. The composition of claim 1, wherein the nanoparticles comprise a hydrophobic core, and wherein the hydrophobic core comprises an inorganic particle.

20. The composition of claim 19, wherein the inorganic particle comprises a metal.

21. The composition of claim 20, wherein the metal comprises a metal salt, a metal oxide, a metal hydroxide, a metal phosphate, or a combination thereof.

22. The composition of claim 21, wherein the metal oxide comprises aluminum oxide, aluminum oxyhydroxide, iron oxide, titanium dioxide, silicon dioxide, or a combination thereof.

23. The composition of any of claim 1, wherein the nanoparticles further comprise a surfactant.

24. The composition of claim 23, wherein the surfactant is a hydrophobic surfactant.

25. The composition of claim 24, wherein the hydrophobic surfactant comprises sorbitan monolaurate, sorbitan monopalmitate, sorbitan monostearate, sorbitan monooleate, sorbitan trioleate, or a combination thereof.

26. The composition of claim 23, wherein the surfactant is a hydrophilic surfactant.

27. The composition of claim 26, wherein the hydrophilic surfactant comprises a polysorbate.

28. The composition of claim 1, wherein the nanoparticles comprise a cationic lipid, an oil, and a surfactant.

29. The composition of claim 1, wherein the composition is lyophilized or is a suspension.

30. A pharmaceutical composition comprising: the composition of claim 1; and a pharmaceutically acceptable excipient.


SEQUENCE LISTING (92 sequences)

SEQ ID NO: 1 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 2 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 3 - RNA, 3846 nt, synthetic construct
  source 1..3846: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 4 - RNA, 3837 nt, synthetic construct
  source 1..3837: mol_type = other RNA
atgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 5 - RNA, 3837 nt, synthetic construct
  source 1..3837: mol_type = other RNA
atgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactataca

SEQ ID NO: 6 - RNA, 3843 nt, synthetic construct
  source 1..3843: mol_type = other RNA
atgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatga

SEQ ID NO: 7 - RNA, 3843 nt, synthetic construct; identical to SEQ ID NO: 6
  source 1..3843: mol_type = other RNA

SEQ ID NO: 8 - RNA, 1725 nt, synthetic construct
  source 1..1725: mol_type = other RNA
atggagctgctcatccttaaagcgaatgctataactactatacttacagctgtaaccttttgcttcgcctctggccaaaacattactgaggagttttatcaatccacttgcagcgcagtctcaaaaggatatctgtcagcattgcgcaccgggtggtacaccagcgttatcactatcgagctttccaacattaaagagaataagtgtaacggcacagacgcgaaagtcaaactcattaagcaagaacttgataaatacaaaaatgcagttactgaacttcagttgctcatgcaatcaaccccagcaacgaacaatagggccaggagagaattgccgaggtttatgaactacacacttaataacgctaaaaagactaacgttacgctctctaagaagcggaagcgcaggtttctcgggttccttctgggggttggcagtgcaatagcatccggcgtcgcggtatcaaaggtgcttcatcttgaaggagaagttaacaaaatcaaaagcgccttgctttcaactaataaggcagtagtatcattgtctaacggtgtcagcgtcttgacttccaaggttttggatttgaaaaactatatcgacaaacaacttctcccgatcgttaacaagcagtcatgcagtatctccaacatcgaaaccgtcatcgaattccaacagaaaaacaatagactgcttgaaattactagagagttttcagtgaacgctggcgttaccactccggtatccacttatatgctcactaatagcgaactgctgtctctgataaatgacatgccaataactaacgaccagaagaaacttatgagtaacaacgtccagatcgtgagacagcaatcatatagcattatgagcataattaaggaggaggtccttgcatacgtagtccagctcccactgtacggggttatcgacacgccatgttggaagcttcatacttcccccttgtgcaccacgaacacgaaggaagggtctaacatttgtctcacgcgcactgatcgggggtggtactgtgacaacgccgggtcagtgtcatttttccctcaggccgagacctgcaaggtccaatcaaaccgggtattctgtgatactatgaactccctgactctgccttctgaagttaacctgtgtaatgtagatatattcaatcctaaatacgactgcaagataatgaccagcaaaaccgacgtgtcctcatctgtcatcacttcccttggtgctatagtaagctgctatggcaaaacgaaatgcaccgcgagtaataaaaatcgcggtatcatcaagacatttagtaacggctgcgattatgtttccaacaaaggtgttgacaccgtatctgtggggaataccctgtattacgtaaataagcaggaagggaaatctctctacgtgaagggggaaccgataatcaacttttatgacccgctggtttttccgtccgacgaatttgacgcgagtatctcccaagtcaacgagaaaattaaccaaagcctcgcgttcataagaaaatccgatgagctgctgcataatgtaaacgcgggcaaatcaactaccaacatcatgattacaacaataatcatcgtgataatcgtgatcctgctttcacttatcgctgtcgggcttcttctctattgtaaggcccggagtactcccgtgaccttgtctaaggatcagctctcaggtatcaacaatattgcattcagcaattga

SEQ ID NO: 9 - RNA, 900 nt, synthetic construct
  source 1..900: mol_type = other RNA
atgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataa

SEQ ID NO: 10 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQSTNKAVVSLSNGVSVLTSKVLDLKNYINNQLLPIVNKQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAIIIVIIVVLLSLIAIGLLLYCKAKNTPVTLSKDQLSGINNIAFSK

SEQ ID NO: 11 - protein, 166 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNK

SEQ ID NO: 12 - protein, 298 aa, synthetic construct
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 13 - RNA, 1704 nt, synthetic construct
  source 1..1704: mol_type = other RNA
atgaaaacaattatcgctctcagttatattttctgccttcctttggggcaagacttgccgggcaatgataatagtacagcgactctctgtctcggacaccacgcagtaccgaacggcacacttgtgaagacaatcacagacgatcaaatcgaagtgacgaatgcaacagagcttgttcaatcatcatctaccggcaaaatctgcaataatccgcatagaatccttgatggaatcgactgcaccctgattgacgcactgttgggagatcctcattgcgatgtgtttcagaatgagacatgggacctttttgtcgagaggagcaaagcgttttccaactgctacccgtatgacgtgcctgattacgcgtcactccgctcacttgtagcatcaagcggtactctggagttcatcaccgaaggattcacctggacgggcgtaactcagaacggcggttctaacgcatgtaagagggggccagggtccggcttcttctcacggctcaactggttgactaagtctggttcaacatacccggtccttaacgttacaatgccgaataatgacaactttgataaactctacatctggggcattcaccatccatccacaaatcaagaacagacaagtttgtacgttcaggcgtcagggcgcgttaccgtgagtacaagaagatcacagcaaacaattatacccaatattgggtcccgaccctgggtaagaggactgtcctctcgcatctccatatactggaccattgtcaaaccgggcgacgtcctggttatcaatagtaatggaaaccttattgctccgcgcggctatttcaaaatgcgaactggaaagtcaagtataatgcgctcagacgcaccgatcgatacttgtatcagtgaatgcatcacccctaacgggtccataccgaacgataagcccttccagaatgtgaataaaatcacgtatggagcatgccccaaatacgtgaagcaaaacaccctcaagttggctacgggtatgcgcaacgtcccagaaaaacaaacgcgaggcttgtttggggcgatagcaggttttatcgagaacggctgggaaggaatgatcgatgggtggtacggctttcgccatcaaaactcagaaggaactgggcaggccgcagatcttaagtctacgcaagcggcgatagatcaaattaatggcaagttgaatagggtgatagagaagacgaacgagaagttccatcaaatagagaaagaattcagtgaagtagaggggcgaattcaggatttggaaaaatatgtcgaggatactaagatcgacctgtggagctataacgcagagcttctggtagcacttgagaaccagcatactattgatctcaccgattccgagatgaacaagctttttgagaagaccaggagacagttgcgcgagaatgccgaagaaatgggtaacggttgttttaagatttatcacaaatgtgacaacgcgtgtattgagtccattaggaatggtacatatgatcacgatgtgtatcgcgatgaagcacttaacaatcggttccaaataaagggcgtggagctcaagagtgggtataaagattggatcctctggatctcattcgcgatttcctgcttcctcctgtgcgttgtcttgctgggctttattatgtgggcatgtcagagaggtaacatccggtgcaacatatgtatctgataa

SEQ ID NO: 14 - protein, 566 aa, synthetic construct
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 15 - protein, 584 aa, synthetic construct
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 16 - protein, 251 aa, synthetic construct
QPENLEYRIMLSVHGSQHSGMIVNDTGHETDENRAKVEVTPNSPRATATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPIITESTENSRMMLELDPT

SEQ ID NO: 17 - protein, 9 aa, synthetic construct
KCDICTDEY

SEQ ID NO: 18 - protein, 9 aa, synthetic construct
YMDGTMSQV

SEQ ID NO: 19 - protein, 9 aa, synthetic construct
MLLAYLYQL

SEQ ID NO: 20 - protein, 9 aa, synthetic construct
AFLPWHRLF

SEQ ID NO: 21 - protein, 9 aa, synthetic construct
SEIWRDIDF

SEQ ID NO: 22 - protein, 9 aa, synthetic construct
YLEPGPVTA

SEQ ID NO: 23 - protein, 9 aa, synthetic construct
KTWGQUWQV

SEQ ID NO: 24 - protein, 9 aa, synthetic construct
ITDQVPFSV

SEQ ID NO: 25 - protein, 10 aa, synthetic construct
VLYRYGSFSV

SEQ ID NO: 26 - protein, 10 aa, synthetic construct
LLDGTATLRL

SEQ ID NO: 27 - protein, 9 aa, synthetic construct
ALLAVGATK

SEQ ID NO: 28 - protein, 9 aa, synthetic construct
MLGTHTMEV

SEQ ID NO: 29 - protein, 9 aa, synthetic construct
LIYRRRLMK

SEQ ID NO: 30 - protein, 9 aa, synthetic construct
ALNFPGSQK

SEQ ID NO: 31 - protein, 9 aa, synthetic construct
AAGIGILTV

SEQ ID NO: 32 - protein, 9 aa, synthetic construct
ILTVILGVL

SEQ ID NO: 33 - protein, 9 aa, synthetic construct
MSLQRQFLR

SEQ ID NO: 34 - protein, 9 aa, synthetic construct
SVYDFFVWL

SEQ ID NO: 35 - protein, 9 aa, synthetic construct
LLGPGRPYR

SEQ ID NO: 36 - protein, 9 aa, synthetic construct
YLSGANLNL

SEQ ID NO: 37 - protein, 9 aa, synthetic construct
KIFGSLAFL

SEQ ID NO: 38 - protein, 10 aa, synthetic construct
VMAGVGSPYV

SEQ ID NO: 39 - protein, 9 aa, synthetic construct
IISAVVGIL

SEQ ID NO: 40 - protein, 9 aa, synthetic construct
LLHETDSAV

SEQ ID NO: 41 - protein, 9 aa, synthetic construct
ALFDIESKV

SEQ ID NO: 42 - protein, 9 aa, synthetic construct
EADPTGHSY

SEQ ID NO: 43 - protein, 9 aa, synthetic construct
SLFRAVITK

SEQ ID NO: 44 - protein, 9 aa, synthetic construct
SAYGEPRKL

SEQ ID NO: 45 - protein, 9 aa, synthetic construct
KMVELVHFL

SEQ ID NO: 46 - protein, 10 aa, synthetic construct
YLOLVFGIEV

SEQ ID NO: 47 - protein, 9 aa, synthetic construct
EVDPIGHLY

SEQ ID NO: 48 - protein, 9 aa, synthetic construct
FLWGPRALV

SEQ ID NO: 49 - protein, 10 aa, synthetic construct
MEVDPIGHLY

SEQ ID NO: 50 - protein, 9 aa, synthetic construct
AARAVFLAL

SEQ ID NO: 51 - protein, 8 aa, synthetic construct
YRPRPRRY

SEQ ID NO: 52 - protein, 9 aa, synthetic construct
VLPDVFIRC

SEQ ID NO: 53 - protein, 9 aa, synthetic construct
QLSLLMWIT

SEQ ID NO: 54 - protein, 9 aa, synthetic construct
SLLMWITQC

SEQ ID NO: 55 - protein, 10 aa, synthetic construct
ASGPGGGAPR

SEQ ID NO: 56 - protein, 10 aa, synthetic construct
QDLTMKYQIF

SEQ ID NO: 57 - protein, 10 aa, synthetic construct
  VARIANT 1: note = E or absent
EAYGLDFYIL

SEQ ID NO: 58 - protein, 9 aa, synthetic construct
SYLDSGIHF

SEQ ID NO: 59 - protein, 9 aa, synthetic construct
ETVSEQSNV

SEQ ID NO: 60 - protein, 9 aa, synthetic construct
FPSDSWCYF

SEQ ID NO: 61 - protein, 9 aa, synthetic construct
EEKLIVVLF

SEQ ID NO: 62 - RNA, 7561 nt, synthetic construct
  source 1..7561: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 63 - protein, 51 aa, synthetic construct
RELPVLDSAAFNVECFKKYACNNEYWETFKENPIRLTEENVVNYITKLKGP

SEQ ID NO: 64 - protein, 41 aa, synthetic construct
TQMRELPVLDSAAFNVECFKKYACNNEYWETFKENPIRLTE

SEQ ID NO: 65 - protein, 1282 aa, synthetic construct
MFLLTTKRTMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 66 - protein, 536 aa, synthetic construct
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 67 - RNA, 11707 nt, synthetic construct
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 68 - RNA, 11707 nt, synthetic construct
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 69 - RNA, 11707 nt, synthetic construct
  source 1..11707: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacaaacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaagatgtcaactgcacagaagtccccgttgctatacacgcaggccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacaaagtggaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 70 - RNA, 11698 nt, synthetic construct
  source 1..11698: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacttcacaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgccaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtattaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcggggccttccccaaggcttttctgctctcgaacccctcgtagacttgccaattgggataaatatcactcgctttcaaactttgcacatcagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaacattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttaagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagaccagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattccccccggcgagcacgatctgtagcatcccagtctattattgcctacactatgtcattgggcgtggagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttgagccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 71 - RNA, 11698 nt, synthetic construct
  source 1..11698: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgacaacacgaacccagttgcctccagcttataccaactcatttactcgcggagtatattatcccgataaggtctttagaagtagcgtgttgcactctacacaggatctgttcttgcccttctttagtaacgttacctggtttcatgcaataagcggaacaaatggaacaaaaagatttgacaatccagtgcttccatttaatgatggggtttactttgccagtaccgaaaagtcaaacataatccgggggtggatctttggaaccactttggactctaagacacagtctctcctcatagtaaacaacgccaccaatgttgtcataaaagtatgcgaatttcagttttgcaacgatccctttctcggggtgtaccataagaataataaatcctggatggagtctgagttccgggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggttggaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccattgtgagatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaaaattgctgattataattataaactcccagatgactttactggatgcgtcatagcctggaattccaacaatcttgattccaaggttggtgggaattataattacctttatcgactgttcagaaagagtaacttgaaaccatttgagagagacatatccaccgagatttaccaggcaggcagtactccttgtaacggcgttgagggatttaactgctattttcctttgcaatcctatggctttcaaccaacatacggggttggctatcaaccctatcgagtggttgtcctgagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaagttcctgccatttcagcagtttgggcgggatatagacgacacaactgacgctgtacgcgatcctcagactttggagatcttggacatcactccctgttctttcggaggggtatctgtcatcacccccggaactaatacatcaaatcaggtcgctgtgttgtaccaaggcgtcaactgcacagaagtccccgttgctatacacgcagatcagctcacccccacatggcgggtgtactcaactggctcaaacgtattccagaccagagctgggtgcttgatcggtgctgaacacgtaaacaatagctatgaatgcgatattcccatcggtgccgggatctgcgctagctatcagacacagaccaattcccatcggcgagcacgatctgtagcatcccagtctattattgcctacactatgtcattgggcgccgagaatagcgtcgcatattcaaataattctattgcaatacccatcaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcccttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcaggacgttgtcaaccagaatgctcaagctctgaatacattggttaagcagctctctagtaattttggggccatctcttcagtacttaatgatattttggcccgattggacccacccgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatatgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactcacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaaaggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 72 - RNA, 11701 nt, synthetic construct
  source 1..11701: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 73 - RNA, 11704 nt, synthetic construct
  source 1..11704: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatgtttctgctcacaaccaaacgcactatgtttgttttcctcgtgctgctccctttggtaagttctcagtgtgtaaacctgagaacacgaacccagttgcctccagcttataccaactcatttactcgcggagtatattatcccgataaggtctttagaagtagcgtgttgcactctacacaggatctgttcttgcccttctttagtaacgttacctggtttcatgcaatacatgtgagcggaacaaatggaacaaaaagatttgacaatccagtgcttccatttaatgatggggtttactttgccagtatcgaaaagtcaaacataatccgggggtggatctttggaaccactttggactctaagacacagtctctcctcatagtaaacaacgccaccaatgttgtcataaaagtatgcgaatttcagttttgcaacgatccctttctcgacgtgtattaccataagaataataaatcctggatggagtctggggtttatagtagtgctaataattgcactttcgaatacgtgtcccaaccattcctcatggaccttgagggcaaacaggggaattttaaaaacttgcgcgaatttgtctttaagaatatcgacggatactttaagatctatagtaaacacactcctatcaacctcgttcgggatcttccccaaggcttttctgctctcgaacccctcgtagacttgccaattgggataaatatcactcgctttcaaactttgcttgccctccacaggagctacctgacacccggcgactcttcttctggtttgaccgccggcgccgctgcctattatgttggttaccttcagccacgaacattcttgctcaagtataacgagaatggcaccattaccgacgccgtcgattgtgcattggatcccttgtctgaaacaaaatgtaccttgaagtcctttaccgtagagaaaggcatataccagacttccaacttccgagttcagcctacagaatccatcgtacgatttcccaacatcacaaacctctgccctttcggtgaagtatttaatgctacacgcttcgcttcagtctatgcctggaataggaagcgcatatcaaattgcgtggccgattattcagtcctctataatagcgcatccttcagtactttcaagtgctacggcgtttcccccaccaaactcaatgatctttgcttcaccaacgtctatgctgacagttttgtcatacgaggcgacgaagtacgccagattgcccccgggcagacaggtaatattgctgattataattataaactcccagatgactttactggatgcgtcatagcctggaattccaacaatctagattccaaggttggtgggaattataattaccgttatcgactgttcagaaagagtaacttgaaaccatttgagagagacatatccaccgagatttaccaggcaggcagtaagccttgtaacggcgttgagggatttaactgctattttcctttgcaatcctatggctttcaaccaacaaacggggttggctatcaaccctatcgagtggttgtcctcagctttgaacttttgcacgctcccgccacagtctgcggaccaaaaaagagtacaaatcttgtcaagaataagtgcgtaaatttcaatttcaatggccttacaggaacaggcgtgctgactgagtcaaacaagaatttcctgccatttcagcagtttgggcgggatatagcagacacaactgacgctgtacgcgatcctcagactttggagatcttggacatcactccctgttctttcggaggggtatctgtcatcacccccggaactaatacatcaaatcaggtcgctgtgttgtaccaaggtgtcaactgcacagaagtccccgttgctatacacgcagaccagctcacccccacatggcgggtgtactcaactggctcaaacgtattccagaccagagctgggtgcttgatcggtgctgaacacgtgaacaatagctatgaatgcgatattcccatcggtgccgggatctgcgctagctatcagacacagaccaattcccgcaggcgggctcgctctgtagcatcccagtctattattgcctacactatgtcattgggcgccgagaatagcgtcgcatattcaaataattctattgcaatacccaccaacttcacaatctccgtaactacagaaatacttccagtttccatgacaaagacatcagtggattgtacaatgtatatatgcggagattccacagaatgttcaaatttgctcttgcagtacggctccttctgcacccagctcaacagggcacttacaggtattgctgtcgaacaggacaagaacacacaagaagtcttcgcccaagtcaaacagatatacaaaactcctcccataaaggattttggcggcttcaactttagtcagatcctcccagacccttcaaaaccatctaaacgatcatttattgaagatctgctgttcaacaaggtcactcttgccgatgctggattcattaagcaatacggtgactgccttggtgatattgctgcccgagatctgatctgtgcccagaaattcaacgggctcactgtactccctccactgctcacagacgaaatgattgcacagtacacaagtgccctgttggcaggcacaatcactagcggctggacctttggcgcaggtgcagcactccaaataccttttgccatgcagatggcctatcggtttaatgggataggcgtgactcaaaatgtcctctacgaaaaccaaaagttgatagctaaccaattcaattcagcaatcgggaagatacaggattcactgtctagtactgctagtgcccttggtaagctgcagaacgttgtcaaccagaatgctcaagctctgaatacattggttaagcagctctctagtaattttggggccatctcttcagtacttaatgatattttgagccgattggacccacctgaagctgaagtacagatcgacaggctgataacaggccggctccaatccctccaaacatacgtgacacaacaactcatacgcgcagccgaaatccgagccagcgctaacctggcagctaccaagatgtcagaatgcgttctgggccagagtaaacgcgtagatttctgcgggaaagggtaccacctgatgtcctttccacaatctgcacctcacggggtcgtctttttgcatgtaacatacgtacccgcacaagagaagaattttactaccgctcctgccatctgtcatgacgggaaagctcattttcctcgcgaaggtgtgtttgtatctaatggtacacattggtttgtcacacagcggaatttctatgaaccccagatcattacaactgacaacacttttgtttccgggaattgtgacgtggtcataggaatcgtaaataacactgtatatgatcccctccaaccagagctggactcttttaaagaagaactggataaatatttcaagaaccacacaagtcccgacgtggaccttggggacataagtggtattaacgcatctgtggttaacattcaaaaggaaatcgacagactcaacgaggtggccaaaaacctgaacgaaagcttgatagatctccaggagttgggcaagtatgaacagtacattaaatggccatggtacatatggcttggctttatcgctggccttatcgccatcgtaatggttacaatcatgctgtgctgcatgacctcctgctgttcttgtttgaaagggtgttgttcttgtggtagttgttgcaagtttgacgaagatgattccgaacctgttcttaagggggtaaagcttcactatacatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 74 - RNA, 9457 nt, synthetic construct
  source 1..9457: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagctgctcatccttaaagcgaatgctataactactatacttacagctgtaaccttttgcttcgcctctggccaaaacattactgaggagttttatcaatccacttgcagcgcagtctcaaaaggatatctgtcagcattgcgcaccgggtggtacaccagcgttatcactatcgagctttccaacattaaagagaataagtgtaacggcacagacgcgaaagtcaaactcattaagcaagaacttgataaatacaaaaatgcagttactgaacttcagttgctcatgcaatcaaccccagcaacgaacaatagggccaggagagaattgccgaggtttatgaactacacacttaataacgctaaaaagactaacgttacgctctctaagaagcggaagcgcaggtttctcgggttccttctgggggttggcagtgcaatagcatccggcgtcgcggtatcaaaggtgcttcatcttgaaggagaagttaacaaaatcaaaagcgccttgctttcaactaataaggcagtagtatcattgtctaacggtgtcagcgtcttgacttccaaggttttggatttgaaaaactatatcgacaaacaacttctcccgatcgttaacaagcagtcatgcagtatctccaacatcgaaaccgtcatcgaattccaacagaaaaacaatagactgcttgaaattactagagagttttcagtgaacgctggcgttaccactccggtatccacttatatgctcactaatagcgaactgctgtctctgataaatgacatgccaataactaacgaccagaagaaacttatgagtaacaacgtccagatcgtgagacagcaatcatatagcattatgagcataattaaggaggaggtccttgcatacgtagtccagctcccactgtacggggttatcgacacgccatgttggaagcttcatacttcccccttgtgcaccacgaacacgaaggaagggtctaacatttgtctcacgcgcactgatcgggggtggtactgtgacaacgccgggtcagtgtcatttttccctcaggccgagacctgcaaggtccaatcaaaccgggtattctgtgatactatgaactccctgactctgccttctgaagttaacctgtgtaatgtagatatattcaatcctaaatacgactgcaagataatgaccagcaaaaccgacgtgtcctcatctgtcatcacttcccttggtgctatagtaagctgctatggcaaaacgaaatgcaccgcgagtaataaaaatcgcggtatcatcaagacatttagtaacggctgcgattatgtttccaacaaaggtgttgacaccgtatctgtggggaataccctgtattacgtaaataagcaggaagggaaatctctctacgtgaagggggaaccgataatcaacttttatgacccgctggtttttccgtccgacgaatttgacgcgagtatctcccaagtcaacgagaaaattaaccaaagcctcgcgttcataagaaaatccgatgagctgctgcataatgtaaacgcgggcaaatcaactaccaacatcatgattacaacaataatcatcgtgataatcgtgatcctgctttcacttatcgctgtcgggcttcttctctattgtaaggcccggagtactcccgtgaccttgtctaaggatcagctctcaggtatcaacaatattgcattcagcaattgaggcgcgccatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 75 - RNA, 8755 nt, synthetic construct
  source 1..8755: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgtccaaaaataaagaccagcgaacggccaagactctggagaggacttgggataccctgaatcatctgttgtttatttcaagttgcctttataaattgaatctcaaatccgtcgcgcaaatcacgctgagcatactcgcaatgattatctcaacttccttgataattgccgccataatcttcattgcaagtgcaaatcataaagtgactcctacaaccgcaataatacaggacgcgaccagccaaattaagaacacgactcccacgtatctcacccaaaatccccaactcggaattagcccaagtaatccgtcagagattacttcacaaatcaccaccatactggcgagcacaactccaggcgtaaaatccaccctccaatctactaccgtcaagactaaaaataccactactactcagacacaacctagtaaacctacgactaagcagcgccagaataaacccccaagcaaaccaaacaacgacttccattttgaagttttcaacttcgtgccttgttctatctgctctaataatccaacatgctgggccatctgcaagcgcattccgaataagaaacccggtaagaaaactacaacgaaacctaccaagaagcctaccctgaaaacgacaaagaaagacccgaaaccgcaaacaaccaaaagtaaagaagtgcccacaactaaacccacagaagaaccaacgataaacacgaccaaaaccaacataataactacgctgcttaccagcaacacgacaggcaacccggaattgaccagtcagatggaaacttttcattcaaccagcagcgaaggtaatccaagtcctagtcaggtgtctacgacatctgaatatccatctcagcctagttcccctccgaacacgccgcgccagtgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 76 - RNA, 9559 nt, synthetic construct
  source 1..9559: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaaaacaattatcgctctcagttatattttctgccttcctttggggcaagacttgccgggcaatgataatagtacagcgactctctgtctcggacaccacgcagtaccgaacggcacacttgtgaagacaatcacagacgatcaaatcgaagtgacgaatgcaacagagcttgttcaatcatcatctaccggcaaaatctgcaataatccgcatagaatccttgatggaatcgactgcaccctgattgacgcactgttgggagatcctcattgcgatgtgtttcagaatgagacatgggacctttttgtcgagaggagcaaagcgttttccaactgctacccgtatgacgtgcctgattacgcgtcactccgctcacttgtagcatcaagcggtactctggagttcatcaccgaaggattcacctggacgggcgtaactcagaacggcggttctaacgcatgtaagagggggccagggtccggcttcttctcacggctcaactggttgactaagtctggttcaacatacccggtccttaacgttacaatgccgaataatgacaactttgataaactctacatctggggcattcaccatccatccacaaatcaagaacagacaagtttgtacgttcaggcgtcagggcgcgttaccgtgagtacaagaagatcacagcaaacaattatacccaatattgggtcccgaccctgggtaagaggactgtcctctcgcatctccatatactggaccattgtcaaaccgggcgacgtcctggttatcaatagtaatggaaaccttattgctccgcgcggctatttcaaaatgcgaactggaaagtcaagtataatgcgctcagacgcaccgatcgatacttgtatcagtgaatgcatcacccctaacgggtccataccgaacgataagcccttccagaatgtgaataaaatcacgtatggagcatgccccaaatacgtgaagcaaaacaccctcaagttggctacgggtatgcgcaacgtcccagaaaaacaaacgcgaggcttgtttggggcgatagcaggttttatcgagaacggctgggaaggaatgatcgatgggtggtacggctttcgccatcaaaactcagaaggaactgggcaggccgcagatcttaagtctacgcaagcggcgatagatcaaattaatggcaagttgaatagggtgatagagaagacgaacgagaagttccatcaaatagagaaagaattcagtgaagtagaggggcgaattcaggatttggaaaaatatgtcgaggatactaagatcgacctgtggagctataacgcagagcttctggtagcacttgagaaccagcatactattgatctcaccgattccgagatgaacaagctttttgagaagaccaggagacagttgcgcgagaatgccgaagaaatgggtaacggttgttttaagatttatcacaaatgtgacaacgcgtgtattgagtccattaggaatggtacatatgatcacgatgtgtatcgcgatgaagcacttaacaatcggttccaaataaagggcgtggagctcaagagtgggtataaagattggatcctctggatctcattcgcgatttcctgcttcctcctgtgcgttgtcttgctgggctttattatgtgggcatgtcagagaggtaacatccggtgcaacatatgtatctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 77 - RNA, 7862 nt, synthetic construct
  source 1..7862: mol_type = other RNA
  misc_difference 7562: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 78 - RNA, 7862 nt, synthetic construct; identical to SEQ ID NO: 77
  source 1..7862: mol_type = other RNA
  misc_difference 7562: note = a, c, g, or u

SEQ ID NO: 79 - RNA, 7862 nt, synthetic construct
  source 1..7862: mol_type = other RNA
  misc_difference 7562: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagntagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 80 - RNA, 7862 nt, synthetic construct; identical to SEQ ID NO: 77
  source 1..7862: mol_type = other RNA
  misc_difference 7562: note = a, c, g, or u

SEQ ID NO: 81 - RNA, 7862 nt, synthetic construct; identical to SEQ ID NO: 77
  source 1..7862: mol_type = other RNA
  misc_difference 7562: note = a, c, g, or u

SEQ ID NO: 82 - RNA, 11701 nt, synthetic construct; identical to SEQ ID NO: 72
  source 1..11701: mol_type = other RNA

SEQ ID NO: 83 - RNA, 7862 nt, synthetic construct
  source 1..7862: mol_type = other RNA
  misc_difference 7565: note = a, c, g, or u
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccntaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 84 - RNA, 12956 nt, synthetic construct
  source 1..12956: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 85 - RNA, 12956 nt, synthetic construct; identical to SEQ ID NO: 84
  source 1..12956: mol_type = other RNA

SEQ ID NO: 86 - RNA, 12956 nt, synthetic construct
  source 1..12956: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgcacaagtaacccggcagcagactggaacgcacgagaatgccaacattgcgacgaacggttcccatattacctataaccaaataaatttctataaagactcttacgcggctagcgcaagtaaacaggacttttcccaggatccatcaaaatttacagaaccagttgtggaggggttgaaggcaggagcccccgtgctcaagtccccatccgccgaagcttgtggctattctgatcgcgtcttgcaactgaaactcggaaactcagcgatcgttactcaagaggctgctaactattgttgtgcctacggtgagtggcccaactacttgccagatcacgaagccgtagcaatcgacaaaccaacacagcctgaaactgctaccgacaggttttatactctcaaatccgtcaagtgggaagccggatcaaccggatggtggtggaaactcccggacgcgctgaataatataggtatgtttggccagaatgtgcaacaccactatctgtatcggtctggattcttgatccatgtgcagtgcaacgcgactaaattccatcaaggagctctcttggtcgttgccatacccgagcatcagaggggcgcatataatacgaacacttctcctgggtttgatgatatcatgaagggagaggaaggcggaacgttcaatcacccctatgtgttggatgatggcacttcacttgcatgcgcgaccatctttccccatcaatggataaacctgcgcacaaataacagtgctacgattgtgcttccatggatgaacgcggcgccgatggatttccctcttcggcataaccagtggacacttgcgattatcccggtagttcctttgggaacccggacgatgtctagcatggtccccattaccgtgtcaattgcccccatgtgttgtgagttcaatggcctccgccacgcaattacgcagggtgttcccacttatctcctcccgggatccggacaattcttgaccacggacgatcactcaagtgcacctgtcctgccttgcttcagtcccactcctgaaatgcatataccgggccaagttcgaaatatgttggaagttgtccaagtcgaaagtatgatggagattaacaacacggaatccgctgtaggcatggaacgactgaaagtggacatatccgccctcacggacgtcgatcaactcctgttcaacatcccccttgatattcagctcgatggccctctcaggaatacgctggtaggtaatatatctcgatattatacacactggtctggtagcctggagatgacgtttatgttttgtggctcattcatggcgacgggtaaactgatactgtgctacacgccgcctggtggcagctgcccaactacacgcgagacagcgatgctggggacccatatcgtgtgggatttcggtctgcaaagttctgtgacactcataatcccttggataagtgggagtcactacaggatgtttaacaacgatgcaaaatctactaacgccaatgtgggttacgtgacctgctttatgcaaacgaacttgattgttccgagcgaatcaagtgatacgtgttctctcattgggttcattgcggctaaagacgatttctccctccgacttatgcgcgattccccagacattggtcagctggatcatcttgatgctgctgaggcagcttatcaaattgagagcatcatcaagaccgcgaccgacactgtgaaatccgagataaatgcggagcttggagttgtaccgtccttgaatgcggtcgagactggtgctaccagtaatacggagcctgaagaggccatacaaacgaggaccgttatcaatcaacatggcgtttccgaaaccctcgttgaaaatttcctcagccgcgcggcacttgtctctaagaggtctttcgaatacaaaaaccacacgtccagtgaagcgcaaaccgataaaaatttcttcaagtggactatcaatactcgaagcttcgttcagctccggcggaagctggaactgtttacctacctccgcttcgatgcagaaattactatactgaccacagtggcagtaaatggctcaagtagtaacgcgtacatggggctgcctgaccttacgttgcaagcgatgttcgttcctacaggcgcgctcacacccaaacagcaggatagcttccactggcagagtggcagcaacgcatctgtgtttttcaaaataagcgacccccctgcaaggatgacaattccattcatgtgtataaattctgcttattccatcttttacgacggttttgccggtttcgaaaagtctgggctctatggcatcaacccagcggatacgatcggcaatctttgtgtccggatagtaaatgagcaccaacccataggcttcactgtcacagtgcgggtctacatgaaaccgaaacacatcaaagcatgggctccacgaccaccccgaacgctcccttatatgtccattgcgaatgccaactacagaggaaaggaacgggcgcctaacgcgctcaatgcaattataggaaacagggatagtgtcaagactatgccgcataatatagtaacgacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggaccgggattcgactttgcgcaagcgattatgaagaagaacacagtagtcgcacgaaccgagaaaggcgagtttactatgttgggtgtgtatgatagggttgccgtaataccaacacacgcaagcgtcggtgagacagtctatatcaacgacgtcgagaccaaagttttggatgcatgcgcactccgcgatttgacagacacaaacttggagattacaatagttaagctcgacagaaaccagaaattccgggatattcggcactttttgcccaaatatgaagatgactacaatgacgcggtcctttcagttcacactagcaagttcccaaacatgtatattcccgtaggtcaggtaaccaattatggatttctcaacttgggaggcactccaacgcacaggatactcatgtataacttcccaacccgcgctggacagtgcggaggggttgtcacaacgaccggtaaggtcatcggcatccacgttggaggtaatggagcgcagggttttgccgccatgttgctccattcctacttcacagatacccaaggagagattgtaagcaacgagaaaagtggggtatgcataaatgcgcctgtgaagacgaaacttcaaccctccgtctttcaccaggtctttgaaggaagtaaagagccagctgtccttaattctaaagaccctagactcaaaacggactttgaggaggcgattttttcaaagtacactggaaacaagattatgcttatggacgaatatatggaggaagccgtagaccattacgttggatgtctggaaccgttggatatttctgttgatcctattcctttggaatcagctatgtacgggatggacgggcttgaggcactcgacctgaccacctccgctgggtttccctatctgctgcaagggaaaaaaaagcgggatatttttaatagacatactagagacacgacggaaatgactaaaatgctggagaagtatggcgtggatctgcctttcgtcaccttcgtaaaagatgaactccggagcaaggaaaaagtcgaaaagggaaaaagtcggcttattgaggcttcttctctcaacgactcagtggcgatgcgagttgcttttggcaacctctacgccacatttcacaataatccgggaacggcaactggcagtgccgtaggctgcgaccccgacattttctggagtaaaattccgatactcttggacggtgaaatttttgccttcgactatacgggatatgacgcgtccttgtccccggtttggtttgcatgtctgaaaaaggtccttgttaagttgggttacacccatcaaacttctttcatagattatttgtgccattctgttcatttgtataaggacaggaagtacattgtaaacggggggatgccttctggttcctctggtacctccattttcaatacaatgattaataacataattatccggactctccttataaaagtttacaagggaatcgacttggatcagtttaagatgatagcctatggagatgacgtaattgcgtcttatcctcacaagatcgaccccggcctgttggcggaagcgggtaagcattatggtctggttatgacgcctgctgataaggggaccagtttcgtggatacgaactgggaaaacgtaaccttcttgaaacggtattttagggctgatgaccaatatccgttcctcattcatcccgtaatgccgatgaaggagatacatgagagtattcggtggacgaaagatccgcgaaatacacaagaccacgtacggtcactgtgttacctggcatggcataacggcgaagaggcctacaatgaattctgtcggaaaattcgaagtgtgcccgtcggacgagctttgactcttccggcttattcctctctcagaaggaaatggcttgatagtttctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 87 - RNA, 12959 nt, synthetic construct
  source 1..12959: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggggcacaagtgactcggcaacaaacgggtacacatgaaaatgccaatattgccacaaatggcagtcacattacttacaaccaaatcaatttttacaaagattcctatgcggcgtccgctagcaaacaagactttagtcaggatccctctaagtttactgaacccgttgtcgaggggctgaaggcaggagcccccgtgctcaagtcaccaagtgcggaagcgtgtgggtacagcgatcgcgtattgcaattgaaactggggaattctgcaatcgttacccaggaggctgccaattactgttgtgcttacggagaatggccgaattacttgcctgaccacgaagctgttgcgatagacaagcctactcaacccgagacagcaactgataggttctatacgctcaaatcagtcaagtgggagacagaatctacgggatggtggtggaaattgccagacgctctgaacaacattggcatgttcgggcagaacgtgcagcatcactatctgtatagatccgggtttttgattcacgtacaatgcaacgctaccaagtttcatcagggtgctcttctggtcgtcgcgatacccgaacaccagcggggggcacacaacacaaccacttctcctgggttcgatgacattatgaagggcgaagagggagggacattcaaccacccatacgttctggatgatgggacttcccttgcttgcgcgactattttcccacatcaatggattaaccttcgaacaaacaatagcgcgactatcgtgctgccatggatgaacgccgcgccgatggactttccattgaggcacaaccaatggacgcttgcaattatacctgtggtaccgctcggtactcggactgtatccagtatggtgcctatcactgtaagtattgcacctatgtgctgcgaatttaacgggcttaggcatgcaataacccaaggcgtccctacgtatttgctgccgggatccggtcagtttctgacaacggatgatcacagcagcgcaccggtcctgccttgttttaatccaaccccagaaatgcacatccctggccaagtaagaaacatgttggaggttatacaagtcgagagtatgatggagataaataacacggaaaatgcggtcggcatgcagaggctcaaggtggacatttctgtcctcacggatgttgaccagttgttgttcaatatcccactcgacattcagctcgatgggcctcttagaaataccctcgttgggaacatttctagatattacacacattggtccggttccttggagatgacttttatgttttgtggctcatttatggcaaccggcaagctcatcttgtgctatactcctcccggggggtcttgccccacaacaagggaaacagcgatgctggggacccacattgtatgggattttggtctccaaagcagtgttaccttggttataccgtggataagtggatcccattatcggatgtttaacaacgacgccaagtcaaccaacgcaaatgtagggtatgtaacctgtttcatgcaaaccaatcttatagtgccttctgaatcaagcaatacatgctccctgattggatttgtggcagcgaaggacgattttagtctgagacttatgcgggactctccagacatcggacagcttgaacatcttcacgaggcggaggcagcttaccagatagagagcattatcaaaaccgcgacggacaccgtcaaatctgaaattaatgcagagctgggagtggtaccctcactgaatgctgtggaaaccggtgcatcttctaacacagaaccagaggaagctattcaaacgcgcacggtaattaaccaacacggcgtcagcgaaacacttgtagaaaactttcttagcagagccgcactcgtgtccaaacgaagctttgagtataaaaaccacacgtccagcgaagcaagaaccgataagaatttttataaatggactatcaacactaagtcatttgtgcagcttagacggaaactggagctcttcacctacctccgatttgacgcggagatcacgattctcacgaccgtcgccgtaggatcaaataactccacatatatgggtctgccagacctcaccctgcaagctatgtttgtccccaccggcgctctcaccccggagaaacaggattcattccattggcaatctgggagtaatgcaagtgtttttttcaaagttagtgaccctccagctcgaatgacgattcctttcatgtgcataaattccgcttatagcgtgttttatgacggatttgcgggttttgagaagaacggcttgtatggtataaacccggccgatacaataggcaacctgtgtgtaagaatcgttaacgagcaccaaccgatcggtttcacggttacggtgcgcgtgtacatgaagcctaaacatatcaaagcttgggcaccgaggcccccccgaacgcttccttacatgagcatcgcaaatgcaaattacaaggggaaagagagggcccccaatgctctcaacgcaattatagggaatagagaaagcgtaaaaacgatgccccatgacataagactggttaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggacctggatttgacttcgcccaggcgattatgaagaagaatactgtgatcgcacgcaccgagaagggggagtttaccatgctcggtatacacgatcgggtggcggttattccaacgcacgctagtgtaggggagacaatctatatcaatgacgttgagacaaaagtgcttgacgcctgtgccttgagggacctcacagataccaatcttgagattacgattgttaaactcgatagaaaccaaaaattcagagatattcggcacttcttgccgcggtatgaagacgattataatgacgcagttctgagtgtgcacacttcaaagtttcccaatatgtatatacccgtgggacaggtgactaattatgggtttctgaatcttggtggcactccgacacacaggatattgatgtacaatttcccgacaagggctgggcaatgtggtggtgtggttacaactacgggtaaagttatcggcattcatgttggtggcaacggggctcaaggcttcgctgccatgcttcttcactcttacttcatcgacacccagggcgagattgtttcaaatgaaaaaagtggtgtgtgcatcaatgcccctgccaaaaccaaactccaaccttcagtattccaccaggtctttgagggaagtaaagaacccgctgtacttaattccaaggaccctcggttgaaaacggactttgaggaggcgatcttttccaaatatacggggaacaagattatgttgatggatgaatatatggaagaggcggtcgaccactacgtgggctgcctcgagccgttggatatatctattgatcctataccactggagtcagcgatgtacgggatggacggccttgaagcgcttgatttgacgactagcgctgggtttccttatctccttcagggcaagaagaaacgggacatttttaatcgccagacccgagatacaaccgagatgacacgcatgctggaaaaatacggcgtcgatttgccgtttgttaccttcgttaaagatgagctccggtctcgagagaaagttgaaaaggggaagtcccgactcatcgaggcaagttccttgaatgactcagttgctatgcgagtagcctttggcaacctctatgcgacgttccataataatcccgggacggcgactggcagtgctgtcggttgcgatccagacatattctggagcaaaatccccattctcttggacggtgagatcttcgccttcgattacacagggtacgacgcgagtctgagcccagtctggttcgcatgtcttaagaaagtgctgatcaaacttggatacacacaccaaaccagttttattgattatttgtgccattcagtacatctgtacaaggacagaaagtacatagtgaacggtggtatgccctctggctctagcggtacgtctatcttcaacacgatgattaataacattataatgcgcacccttctgattcgggtctacaaggggatagatctggatcagtttaagatgatagcgtatggtgacgatgtgatagcaagctacccacataagattgaccccggtctgctcgcagaggcgggtaaacactatggactgcttatgacccctgcggataaggggacgagtttcgtagatacgaactgggagaatgtgacatttcttaaaaggtattttcgcgcggaccaccagtacccatttttgatccaccctgtgatgcctatgaaggaaatccatgaaagcattaggtggacaaaggaccctcgaaacactcaggatcatgtgcgctctttgtgttaccttgcttggcacaacggggaagaagcttataatgagttctgtcgcaagatacgaagcgtaccagttgggagagcgcttaccttgcctgcgtattcttccctcagacgcaaatggctggatagtttctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 88 - RNA, 12953 nt, synthetic construct
  source 1..12953: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaagtcactcgccagcaaaccgggacacacgaaaatgctaacatcgcaactaatggcagccacataacgtacaaccagatcaatttttataaggattcatacgcagcatcagccagtaagcaagacttctcacaagacccatcaaagttcaccgaaccagtagtagagggactgaaagcgggtgctccggtactgaagtccccctcagctgaagcttgtggctacagcgatagggttcttcaactcaaacttgggaatagcgcgattgtcacacaggaagcggccaactactgctgcgcatatggggaatggcccaattatttgcctgatcatgaggcagtagctattgacaaacctactcagccagaaaccgccactgatcgattttatactctgaaaagcgtaaagtgggagactgaaagcacaggatggtggtggaaattgcctgatgcgctcaataatattggaatgtttgggcaaaatgttcaataccactacctctatagatccggcttcctcattcacgttcagtgtaacgcgactaagttccaccaaggcgcccttttggtcgtggcaattcccgagcaccaacgaggggcttataacactacaacttcacccgggttcacggacataatgaaaggtgaggaggggggaacgttctcccatccgtatgtcttggacgacggcacgtccctcgcctgcgctacgatctttcctcaccaatggataaatctccggacgaacaatagcgccacaatagtgttgccttggatgaacgcggcccctatggatttccctctgcgccacaatcagtggacccttgcaataatcccggttgtaccacttggaacccgcacggttagctctatggtgccgattacagtatccattgcgccgatgtgctgcgaattcaatggactgcgacacgcgatcactcaaggagtccctacctatctgctgccgggctccggacagttcctcacgacggacgaccatagctcagcacctgtactgccttgctttaatccaactcccgagatgcatatcccaggccaggttcgaaacatgctggaagttgtacaagtcgagagtatgatggaaataaataatactgagaatgcggtcggcatgcaaagactgaaagtcgatatctccgtacttaccgatgtcgaccagctcctctttaacattccacttgatatacagctggacggcccgcttcgaaatacccttgtcgggaatatatcccggtattatacgcattggtctgggtctctcgaaatgacctttatgttttgtgggagctttatggcaaccggaaagctgattttgtgctatactccaccaggcggtagttgccccacgacacgcgaaactgcaatgttggggacgcatatcgtttgggatttcggccttcaatcctcagtcacccttgtgattccgtggattagtggttctcattatcgcatgtttaataatgatgccaagtctactaatgctaacgttggctatgtgacgtgctttatgcagaccaatctcatcgtacccagtgaaagtagcaacacgtgctctctgatcggtttcgtggctgcgaaggacgatttcagccttcgacttatgagggattcccccgatatccggcaattggaccatcttgatgcagccgaagctgcataccagatagagtcaatcattaaaacggcaaccgataccgtgaaatcagaaattaatgccgaactgggggtggtcccctcactgaatgctgttgaaacgggagcatcaagcaacaccgagccagaggaggcgatacagacgcggaccgtgataaatcagcacggggtctccgaaactttggtcgagaacttcctttctcgagcagctctggtttcaaagaggtcctttgaatataagaaccacgccagttcagaagcgcagacagataagaacttcttcaaatggacaataaatacgaagagcttcgtgcagctcagacggaaactggaactctttacttacttgagattcgatgcggaggtgactatactgactacagtggcagtcagcagtagcaatagtacctataccggtctccctgacttgacccttcaagcgatgttcgtaccgacgggagcgcttacaccagagaaacaggacagtttccactggcaatctgcatcaaacgcgagtgttttctttaaaatttccgatcctccggcaagaatgactatcccattcatgtgtattaactccgcatactctgtcttctacgatggattcgccggtttcgaaaagagcggactctatggaattaacccagctgatacaataggtaacctctgtgtgcgcattgttaacgaacaccagcccatcggtttcactgtcacagtccgggtatacatgaaaccaaagcatataaaagcgtgggcgcccagaccaccccggaccctgccgtacatgagcatcgccaatgccaattaccgaggcaaagagagggcccccaacgcgctcaacgcaataatcggcaacagggacagcgtcaaaacaatgccccataacattgtgactacgtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggggcctggctttgatttcgcacaggccataatgaagaaaaacaccgttattgcacgaaccgaaaaaggtgagttcacaatgctcggtatttacgacagagttgccgtcatacctacccatgcatccgtaggggaaacaatttatataaatgacgtggagactaaagttctcgatgcctgcgccctccgcgatttgactgacaccaacttggaaataacaattgttaagatggaccgcaatcaaaaattccgagacataaggcactttctcccccgatacgaggacgactataacgatgcggtgctcagtgtccacacctcaaaattccctaatatgtacatccccgtcggtcaagtgactaactatggatttttgaatttgggaggcacaccgactcaccgcatactcatgtacaatttccccacacgggcagggcagtgtggtggtgtagtcacgacaacaggaaaggtgatcgggatccatgtcggtggtaacggcgcacaagggtttgctgcaatgcttctccactcttattttactgacactcagggcgaaatcgtcagctccgaaaagagtggagtttgtataaatgcacccgctaaaacgaagctgcaaccgagtgtctttcaccaagtgttcgagggttcaaaagaacccgctgtactcaacagtaaggactccaggctcaaaacagattttgaggaagctatcttctctaaatacacgggaaataaaatcatgctgatggatgaatatatggaagaagccgttgaccattacgttgggtgtctggagccgttggatatctcaattgatcccatcccccttgaatctgctatgtatggcatggacggtctcgaggcgcttgatttgacgacatccgctggattcccttatttgttgcaaggtaaaaagaagcgcgacatatttaacagacagactagggataccactgagatgactcgcatgttggagaaatacggtgttgatcttccatttgttacttttgtgaaagacgaattgcgaagcagggaaaaggttgaaaagggtaagtctagacttatcgaagcctcttcattgaatgattcagtggcaatgagggttgcctttggaaacttgtacgccacgtttcataataatcccggaacggcgacagggtctgcggttgggtgtgatcccgacgtattttggagcaaaatacccatcttgctcaacggtgaaatatttgccttcgattacaccgggtatgacgcgtctctgagcccagtgtggtttgcgtgcctgaagaaggttttgataaaattgggctatacacaccagacttcttttattgactacctgtgccattctgttcacctctataaagaccgaaagtatattgttaatggaggaatgccgtcaggatcctctggtacttccatcttcaatactatgatcaataatatcattatacgcacactgctcatcagagtgtacaaaggtatagacctggatcaatttaagatgattgcctatggtgacgacgtgattgcgagctacccgcataagatcgacccaggcttgttggctgaggctggaaagcattacggactcatcatgactccagcggataagggaacgtcctttgtggataccaattgggaaaatgttacgtttcttaaacgatatttcagggcagacgatcaatacccgttccttatccaccccgtgatgccgatgaaagaaatacacgaaagcattagatggactaaagacccaagaaacacgcaagatcacgttaggagtctttgttacttggcttggcataatggtgaagaggcatacaatgaattctgtaggaagatacgaagtgtcccagtcggtcgagcactcacactccctgcatacagctctctgagacgcaaatggctcgatagtttctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 89 - RNA, 12947 nt, synthetic construct
  source 1..12947: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgcacaagtgacgcgccagcagactggcacgcacgagaatgctaacgtagccactaacgggtcccatataacatataatcaaataaatttttacaaggattcatacgcggcctctgcgtcaaaacaggatttttcccaagatccgtctaagtttacggagccggtcgtggaaggtctgaaagctggagcacctgttctgaagagtccctccgcggaagcgtgtggctactcagaccgagttctgcagttgaaacttgggaacagtgctatcgtgacccaggaagctgccaattactgctgcgcttatggggagtggcccaattacttgcctgaccacgaagctgtagctatagataaaccgacgcagccagaaaccgcgacagaccggttctataccctccgctccgtcaagtgggaagcaacttcaacaggctggtggtggaaactgcccgacgcgcttaacaacatcggcatgttcgggcaaaatgtacaacatcactacctctacaggagtgggttcctgattcacgtgcaatgtaacgcaactaaattccaccaaggagccctcctcgtcgttgctatcccggaacatcaaaggggtgcacataatactacaacttctccagggttcgatgacataatgaaaggcgaggcaggaggcactttcaatcatccctacgtcctggacgatggcacatccctggcatgtgccaccatctttccccatcagtggattaatttgcggaccaataatagcgctaccatcgttcttccatggatgaacgctgcaccgatggatttcccgcttagacacaatcaatggacgcttgcaatcattcccgttgttccgctcggcacccgaaccatgagcagtatggtccccattactgtgtctatagcgccgatgtgttgcgaatttaatggactgaggcacgcaatcactcagggagtccccacgtacttgcttccgggtagcgggcaattcttgacaactgacgaccatagcagtgccccggtattgccttgttttaatcccacccccgaaatgcatatccccgggcaagtcagaaatatgctggaagtagtgcaagtagaaagtatgatggaaatcaacaatacagagtcagccgtaggcatggaacggcttaaagttgacatttctgcgctcacggacgtcgatcaattgctgtttaatatcccccttgatatccagttggatggtccacttaggaacacgcttgtcggaaacattagccgatactacacacattggtccggtagtctcgagatgacatttatgttttgtgggtcattcatggctactggcaaactgatactgtgctacacaccaccagggggcagctgtccaacgactagggaaactgctatgttgggtacacacattgtctgggatttcggtttgcagtcctctataacgctgattataccctggatatcaggttcacattaccgcatgtttaataatgacgcgaagtccacaaacgccaatgtcggttacgttacatgttttatgcagacgaacctgatagtgccctcagagtcttctgatacctgctctctcattggttttattgccgctaaggacgatttttcacttcggctgatgcgcgattcccctgacatacggcaaatcgatcatctgcacggggccgaagctgcttatcagattgaatccataataaagaccgcaaccgatacagtcaagagcgaaataaatgcagagttgggtgtggtaccatctctcaacgctgtcgagacaggggcaacgtcaaatactgagccagaagaagcgattcaaacaaggacggtaataaaccaacatggtgtgtccgagaccctcgttgaaaactttctgagcagagcagcactcgtctcaaagcggagctttgagtataaagaccacacctcctccactgcgcagacggataagaatttctttaagtggaccattaatacgcggtcctttgttcaattgagaaggaagttggagctgttcacgtatctgcggttcgatgccgagataacaatcctgactacggtagcggtaaatggctccaacaacaatacttacgttggtctcccagatctgacactccaggcaatgttcgtcccgacgggggctttgacgccagaaaaacaagactctttccattggcaaagcgggagtaatgcctcagtattttttaaaatctcagatcctcctgcgcgcatgaccattccatttatgtgtataaattcagcctacagcgtcttttacgatggatttgctgggttcgagaaaacggggctttatggtataaatcctgctgatacgattggtaacctgtgcgtacggatcgtaaatgaacatcagcctgtcggcttcacagttaccgtccgagtttatatgaagcccaagcacatcaaagcgtgggcacctcggccaccccgcacactgccgtacatgtctatcgcaaacgcaaattataaaggtaaagaacgggctccaaacgcgcttaacgctataatcgggaaccgcgacagtgttaaaactatgccccataatattgtgtgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggccctggatttgatttcgctcaggccatcatgaagaagaatacggtaatagcaagaacagagaaaggcgagttcaccatgcttggtgtttatgatagggtggcggtcatccctacgcatgcatcagttggtgaaacaatctatatagatgacgtcgagacacgggttctcgacgcatgcgctttgcgggatcttactgatacgaatttggaaattactattgttaaacttgaccggaatcagaagttcagagacatccgccattttctcccgagatatgaagacgactacaacgacgccgttctgagtgtccacacgtcaaaatttccgaatatgtatattcctgtggggcaggtcactaactatggatttttgaatttgggcggcactccaactcataggatccttatgtataacttccctaccagagcggggcaatgtggcggcgtggttacgacgacaggcaaagtaatcggtattcacgttggaggcaacggcgcccagggatttgcggcgatgcttctccattcatactttacggacacacaaggcgagatagtatctagcgagaaaagtggtgtttgcataaatgcgccagcaaaaacaaaattgcagccatccgtgttccaccaagttttcgaaggaagtaaagagcccgctgtcttgaacccgaaagatccgcgcttgaaaaccgattttgaggaggcgattttcagcaagtacactggaaataagattatgcttatggacgagtatatggaggaggcggtggatcattacgtagggtgcttggagccccttgacatatctgtcgaccccatacccttggagtccgctatgtacggtatggacggattggaggctctggacttgactacaagtgcaggtttcccatatctcctccagggaaaaaaaaaacgggacatctttaaccgccatactcgcgataccagcgagatgaccaaaatgctcgaaaaatatggagtggacctcccgtttgtgacgtttgttaaagacgaactgcgaagcagggaaaaagtggaaaaggggaaaagcaggctcatcgaagctagtagtctgaatgacagtgtagctatgagagtcgcgtttggaaacctttatgccacctttcataacaatccaggcaccgctactggctctgccgtaggctgtgatccggacatattctggtccaaaatcccgattcttctcgacggggagatattcgccttcgattatacgggatatgatgcttccctcagcccggtctggttcgcatgcttgaaaaaggtgctcataaagctgggctacacgcaccagacgtcattcatagattatctgtgccatagcgtgcatctttataaggatagaaagtacatagtcaatggcggcatgccgtcaggaagttccggaacctctatcttcaatacgatgataaacaacatcataattcgaactctcctcataagggtatacaaaggaatagacttggaccaattcaagatgatcgcttatggcgacgatgtgattgcctcttacccacacaagattgaccccggactcctcgcagaagctggaaagcactatggccttgttatgactcccgcggacaaaggcacgtcctttgtggacaccaactgggagaatgttactttcctgaaacgctatttccgggcggacgatcagtacccctttctcatacatccagttatgccgatgaaggagattcacgagtctatccgctggacaaaagatccgcgcaacacgcaggatcacgtacgcagcctctgttacctcgcgtggcataatggagaagaagcatataacgagttctgtagaaaaatacgctcagtgccagtagggcgcgccctcacgttgcccgcatactcatcactgcgacgcaaatggttggattccttttaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 90 - protein, 9 aa, synthetic construct
SYLDSGIHS

SEQ ID NO: 91 - protein, 9 aa, synthetic construct
ETVSEESNV

SEQ ID NO: 92 - protein, 9 aa, synthetic construct
EEKLSVVLF